(19) United States
(12) Patent Application Publication
(10) Pub. No.: US 2024/0263238 A1
(43) Pub. Date: Aug. 8, 2024

(54) TREATMENT OF SCHIZOPHRENIA AND BIPOLAR DISORDER

(71) Applicant: Cambridge Enterprise Limited, Cambridge (GB)

(72) Inventor: Sudhakaran PRABAKARAN, Acton, MA (US)

(21) Appl. No.: 18/578,982

(22) PCT Filed: Jul. 14, 2022
(86) PCT No.: PCT/EP2022/069790

§ 371 Date: Jan. 12, 2024

Related U.S. Application Data
(60) Provisional application No. 63/221,821, filed on Jul. 14, 2021.

(51) Int. Cl. C12Q 1/6883; C07K 16/00; C12N 15/113

(52) U.S. Cl. CPC C12Q 1/6883; C07K 16/00; C12N 15/113; C07K 2317/622; C12N 2310/11; C12Q 2600/158

(57) ABSTRACT

The present application features methods of treating schizophrenia and bipolar disorder by targeting dysregulated novel open reading frames (nORF) in which increased or reduced expression of the dysregulated nORF is associated with the schizophrenia or bipolar disorder.

### CROSS-REFERENCE TO RELATED APPLICATIONS

This application claims the benefit of U.S. Provisional Application No. 63/221,821 filed on Jul. 14, 2021, which is incorporated herein by reference in its entirety.

### BACKGROUND OF THE INVENTION

Identifying the cause of schizophrenia and bipolar disorder has been a challenging endeavor, as these disorders are sometimes known to be genetically linked. However, identifying how the genetic basis is linked to schizophrenia and bipolar disorder pathology is unclear. Furthermore, providing an effective therapeutic remains elusive. Accordingly, new methods of diagnosis and treatment are needed to better understand how these genetic dysregulations cause schizophrenia and bipolar disorder.

### SUMMARY OF THE INVENTION

In one aspect, the invention features a method of treating schizophrenia or bipolar disorder in a subject by identifying a sequence of a novel open reading frame (nORF) associated with the schizophrenia or bipolar disorder, wherein the sequence of the nORF is distinct from a canonical open reading frame (cORF) of a gene. The nORF is present in (i) an overlapping region of the cORF in an alternate reading frame, (ii) a 5′ untranslated region (UTR) of the cORF, (iii) a 3′ UTR of the cORF, (iv) an intronic region of the cORF, (v) an intergenic region of the cORF, or (vi) a region not associated with the cORF or the gene, and wherein the nORF has increased expression relative to the nORF in a subject without schizophrenia or bipolar disorder. The method further includes administering to the subject an inhibitor that reduces expression of the nORF to treat the schizophrenia or bipolar disorder.

In another aspect, the invention features method of treating schizophrenia or bipolar disorder in a subject by administering to the subject an inhibitor that reduces expression of a nORF. The subject may have previously been identified with a sequence of the nORF associated with the schizophrenia or bipolar disorder, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the nORF has increased expression relative to the nORF in a subject without schizophrenia or bipolar disorder.

In some embodiments of either of the foregoing aspects, the method reduces expression of the nORF, e.g., by at least 5%, 10%, 15%, 20%, 25%, 30%, 40%, 50%, 60%, 70%, 80%, 90%, 95%, 97%, or 99%. The nORF may exhibit an increase (e.g., by at least 5%, 10%, 15%, 20%, 25%, 30%, 40%, 50%, 60%, 70%, 80%, 90%, 100%, 200%, 300%, 400%, 500%, or more) in expression, e.g., as compared to the nORF in a normal (e.g., without schizophrenia or bipolar disorder) subject.

In some embodiments of either of the above aspects, the inhibitor is a small molecule, a polynucleotide, or a polypeptide. The polynucleotide may include a miRNA, an antisense RNA, an shRNA, or an siRNA. The polypeptide may include an antibody or antigen-binding fragment thereof (e.g., an scFv).

In some embodiments, the inhibitor is encoded by a vector, such as a viral vector. The viral vector may be selected, for example, from the group consisting of a Retroviridae family virus, an adenovirus, a parvovirus, a coronavirus, a rhabdovirus, a paramyxovirus, a picornavirus, an alphavirus, a herpes virus, and a poxvirus. The parvovirus viral vector may be, for example, an adeno-associated virus (AAV) vector.

In some embodiments, the viral vector is a Retroviridae family viral vector (e.g., a lentiviral vector, an alpharetroviral vector, or a gammaretroviral vector). The Retroviridae family viral vector may include one or more of the following: a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

In some embodiments, the viral vector is a pseudotyped viral vector. The pseudotyped viral vector may be selected, for example, from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus. The pseudotyped viral vector may be a lentiviral vector.

In some embodiments, the pseudotyped viral vector includes one or more envelope proteins from a virus selected from vesicular stomatitis virus (VSV), RD114 virus, murine leukemia virus (MLV), feline leukemia virus (FeLV), Venezuelan equine encephalitis virus (VEE), human foamy virus (HFV), walleye dermal sarcoma virus (WDSV), Semliki Forest virus (SFV), Rabies virus, avian leukosis virus (ALV), bovine immunodeficiency virus (BIV), bovine leukemia virus (BLV), Epstein-Barr virus (EBV), Caprine arthritis encephalitis virus (CAEV), Sin Nombre virus (SNV), Cherry Twisted Leaf virus (ChTLV), Simian T-cell leukemia virus (STLV), Mason-Pfizer monkey virus (MPMV), squirrel monkey retrovirus (SMRV), Rous-associated virus (RAV), Fujinami sarcoma virus (FuSV), avian carcinoma virus (MH2), avian encephalomyelitis virus (AEV), Alfa mosaic virus (AMV), avian sarcoma virus CT10, and equine infectious anemia virus (EIAV).

In some embodiments, the pseudotyped viral vector includes a VSV-G envelope protein.

In another aspect, the invention features a method of treating schizophrenia or bipolar disorder in a subject by identifying a sequence of a nORF associated with the schizophrenia or bipolar disorder, wherein the sequence of the nORF is distinct from a cORF of a gene. The nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the nORF has decreased expression relative to the nORF in a subject without schizophrenia or bipolar disorder. The method further includes administering to the subject an activator that increases expression of nORF to treat the schizophrenia or bipolar disorder.

In another aspect, the invention features a method of treating schizophrenia or bipolar disorder in a subject by administering to the subject an activator that increases expression of a nORF. The subject may have previously been identified with a sequence of the nORF associated with the schizophrenia or bipolar disorder, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the nORF has decreased expression relative to the nORF in a subject without schizophrenia or bipolar disorder.

In some embodiments of either of the foregoing aspects, the method increases expression of the nORF, e.g., by at least 5%, 10%, 15%, 20%, 25%, 30%, 40%, 50%, 60%, 70%, 80%, 90%, 100%, 200%, 300%, 400%, 500%, or more. The nORF may exhibit a decrease (e.g., by at least 5%, 10%, 15%, 20%, 25%, 30%, 40%, 50%, 60%, 70%, 80%, 90%, 95%, 97%, or 99%) in expression, e.g., as compared to the nORF in a normal (e.g., without schizophrenia or bipolar disorder) subject.

In some embodiments, the activator is a small molecule, a polynucleotide, or a polypeptide. The polynucleotide may include an antisense RNA. The polypeptide may include an antibody or antigen-binding fragment thereof (e.g., an scFv).

In some embodiments, the activator is encoded by a vector, such as a viral vector. The viral vector may be selected, for example, from the group consisting of a Retroviridae family virus, an adenovirus, a parvovirus, a coronavirus, a rhabdovirus, a paramyxovirus, a picornavirus, an alphavirus, a herpes virus, and a poxvirus. The parvovirus viral vector may be, for example, an AAV vector.

In some embodiments, the viral vector is a Retroviridae family viral vector (e.g., a lentiviral vector, an alpharetroviral vector, or a gammaretroviral vector). The Retroviridae family viral vector may include one or more of the following: a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

In some embodiments, the viral vector is a pseudotyped viral vector. The pseudotyped viral vector may be selected, for example, from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus. The pseudotyped viral vector may be a lentiviral vector.

In some embodiments, the pseudotyped viral vector includes one or more envelope proteins from a virus selected from VSV, RD114 virus, MLV, FeLV, VEE, HFV, WDSV, SFV, Rabies virus, ALV, BIV, BLV, EBV, CAEV, SNV, ChTLV, STLV, MPMV, SMRV, RAV, FuSV, MH2, AEV, AMV, avian sarcoma virus CT10, and EIAV.

In some embodiments, the pseudotyped viral vector includes a VSV-G envelope protein.

In another aspect, the invention features a method of treating schizophrenia or bipolar disorder in a subject by identifying a sequence of a nORF associated with the schizophrenia or bipolar disorder, wherein the sequence of the nORF is distinct from a cORF of a gene. The nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the nORF has decreased expression relative to the nORF in a subject without schizophrenia or bipolar disorder. The method further includes providing a protein encoded by the nORF to the subject treat the schizophrenia or bipolar disorder.

In another aspect, the invention features a method of treating schizophrenia or bipolar disorder in a subject by providing a protein encoded by a nORF to the subject. The subject may have previously been identified with a sequence of the nORF associated with the schizophrenia or bipolar disorder, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the nORF has decreased expression relative to the nORF in a subject without schizophrenia or bipolar disorder.

In some embodiments of either of the foregoing aspects, the method includes restoring the encoded protein product of the nORF. The method may include providing the protein product or a polynucleotide encoding the protein product. The method may include providing a vector (e.g., a viral vector) including the polynucleotide encoding the protein product.

In some embodiments, the viral vector may be selected, for example, from the group consisting of a Retroviridae family virus, an adenovirus, a parvovirus, a coronavirus, a rhabdovirus, a paramyxovirus, a picornavirus, an alphavirus, a herpes virus, and a poxvirus. The parvovirus viral vector may be, for example, an adeno-associated virus (AAV) vector.

In some embodiments, the viral vector is a Retroviridae family viral vector (e.g., a lentiviral vector, an alpharetroviral vector, or a gammaretroviral vector). The Retroviridae family viral vector may include one or more of the following: a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

In some embodiments, the viral vector is a pseudotyped viral vector. The pseudotyped viral vector may be selected, for example, from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus. The pseudotyped viral vector may be a lentiviral vector.

In some embodiments, the pseudotyped viral vector includes one or more envelope proteins from a virus selected from VSV, RD114 virus, MLV, FeLV, VEE, HFV, WDSV, SFV, Rabies virus, ALV, BIV, BLV, EBV, CAEV, SNV, ChTLV, STLV, MPMV, SMRV, RAV, FuSV, MH2, AEV, AMV, avian sarcoma virus CT10, and EIAV.

In some embodiments, the pseudotyped viral vector includes a VSV-G envelope protein.

In some embodiments of any of the above aspects, the encoded protein product of the nORF is less than about 100 amino acids.

In some embodiments, the method further includes performing a statistical analysis between the nORF and the schizophrenia or bipolar disorder. The statistical analysis may measure a positive or negative association between the nORF and the schizophrenia or bipolar disorder.

In some embodiments, the nORF is associated with a transposable element. For example, the nORF may have a positive or negative correlation with a transposable element.

In some embodiments, the nORF is associated with a human accelerated region (HAR). For example, the nORF may have a positive or negative correlation with the HAR.

In some embodiments, the nORF is selected from Table 4. In some embodiments, the nORF has at least 85% (e.g., at least 90%, 95%, 97%, 98%, 99%, or 100%) sequence identity to any one of SEQ ID NOs: 1-21 or a fragment thereof. In some embodiments, the nORF has the sequence of any one of SEQ ID NOs: 1-21.

In some embodiments, the disease is bipolar disorder and the nORF is selected from Table 7. In some embodiments, the nORF has at least 85% (e.g., at least 90%, 95%, 97%, 98%, 99%, or 100%) sequence identity to any one of SEQ ID NOs: 124-163 or a fragment thereof. In some embodiments, the nORF has the sequence of any one of SEQ ID NOs: 124-163.

In some embodiments, the disease is bipolar disorder and the nORF is selected from Table 8. In some embodiments, the nORF has at least 85% (e.g., at least 90%, 95%, 97%, 98%, 99%, or 100%) sequence identity to any one of SEQ ID NOs: 164-207 or a fragment thereof. In some embodiments, the nORF has the sequence of any one of SEQ ID NOs: 164-207.

In some embodiments, the disease is schizophrenia and the nORF is selected from Table 9. In some embodiments, the nORF has at least 85% (e.g., at least 90%, 95%, 97%, 98%, 99%, or 100%) sequence identity to any one of SEQ ID NOs: 208-263 or a fragment thereof. In some embodiments, the nORF has the sequence of any one of SEQ ID NOs: 208-263.

In some embodiments, the disease is schizophrenia and the nORF is selected from Table 10. In some embodiments, the nORF has at least 85% (e.g., at least 90%, 95%, 97%, 98%, 99%, or 100%) sequence identity to any one of SEQ ID NOs: 264-324 or a fragment thereof. In some embodiments, the nORF has the sequence of any one of SEQ ID NOs: 264-324.

### Definitions

As used herein, a “novel open reading frame” or “nORF” refers to an open reading frame that is transcribed in a cell and consists of a sequence that is distinct from a canonical open reading frame (cORF) transcribed from a gene. The nORF may be present in (i) an overlapping region of the cORF in an alternate reading frame, (ii) a 5′ untranslated region (UTR) of the cORF, (iii) a 3′ UTR of the cORF, (iv) an intronic region of the cORF, (v) an intergenic region of the cORF, or (vi) a region not associated with the cORF or the gene. The nORF may be any unannotated genetic sequence that is transcribed in a cell. As used herein, a “canonical open reading frame” or “cORF” refers to an open reading frame that is transcribed in a cell and its associated genetic elements, including the 5′ UTR, the 3′ UTR, the intronic regions, the exonic regions, and the intergenic regions flanking the gene comprising the cORF. A cORF includes either the primary open reading frame that is expressed from a gene, the most abundantly expressed open reading frame expressed from a gene, or an ORF that is annotated in a publicly available database as the primary and/or most abundantly expressed open reading frame from a gene.

### BRIEF DESCRIPTION OF THE DRAWINGS

The patent or application file contains at least one drawing executed in color. Copies of this patent or patent application with color drawing(s) will be provided by the Office upon request and payment of the necessary fee.

FIGS. 1A-1D are a set of schematics and graphs showing the identification of novel open reading frame (nORFs) within neuropsychiatric samples. FIG. 1A shows the workflow used to identify nORF containing transcripts within neuropsychiatric samples. Genomic coordinate matches between nORFs and sample transcripts assessed using GffCompare were filtered to retain only matches of the type “=” or complete intron chain overlap. Next, nORFs with exon boundaries contained within their corresponding transcript matches were selected. Finally, only noncoding transcripts, with biotype not equal to “protein-coding” were retained for further differentially expressed (DE) analysis. FIG. 1B shows the percentage of the total nORF containing transcripts split over their biotypes (e.g., approximately 47% transcripts are retained introns). FIG. 1C shows the 40 and 56 DE transcripts containing 44 and 61 nORFs for bipolar disorder (BD) and schizophrenia (SCZ), respectively. A Venn diagram assessing the 44 BD and 61 SCZ nORFs shows that 14 nORFs are common to DE transcripts in SCZ and BD. FIG. 1D shows the transcript biotype against total transcript percentage or nORFs within DE transcripts. The highest occupancy of nORFs in DE transcripts is within retained introns followed by processed transcripts.

FIGS. 2A-2C are a set of graphs showing nORF enrichment within Human Accelerated Regions (HARs) HARs and SCZ-specific loci. FIG. 2A shows nORFs transcribed within neuropsychiatric samples (blue peaks) and DE in SCZ (red peaks), as evaluated for overlap and enrichment within SCZ-specific loci (black vertical lines—outermost circular panel), using Genomic Loci Annotation and Enrichment Tool (GLANET). If nORF enrichment was identified, the corresponding loci (vertical line) is marked with circular dots—transcribed nORF enrichment as blue circles and DE nORF enrichment as red circles. Two SCZ loci within chromosome 2 are enriched for nORFs DE in SCZ. Similar analyses conducted for BD-specific loci and SCZ-specific copy-number variations (CNVs) showed no nORF enrichment. FIG. 2B shows nORFs transcribed within neuropsychiatric samples and DE in SCZ (left) and BD (right) were defined to be associated with a unique HAR if the unique HAR overlapped the nORF or regions extending 100 Kb upstream or downstream of the nORF. For each HAR-associated nORF, each unique HAR with which the nORF was associated was categorized based on the types of HARs-vHARs, mHARs and pHARs (HARs conserved in vertebrates, mammals, and non-human primates, respectively)—contained within it. Since a unique HAR could contain multiple individual HARs, a single unique HAR could be categorized as containing multiple types of HARs. The number of unique HARs in each category was quantified. FIG. 2C shows nORFs transcribed within neuropsychiatric samples were defined to be associated with a unique HAR if the unique HAR overlapped the nORF or regions extending 100 Kb upstream or downstream of the nORF. Disorder-associated single nucleotide polymorphisms (SNPs) were stratified based on their genome-wide association study P-value (GWAS P-value upper bound). Stratified SNPs were used to determine stratified disorder-associated SNP loci. HAR-associated nORFs (nORF-HARs) were queried for enrichment with stratified disorder-associated SNP loci, using INRICH. The enrichment analysis was also performed for nORFs associated with vHARs (nORF-vHARs), mHARs (nORF-mHARs), or pHARs (nORF-pHARs). The enrichment analysis provided both an empirical P-value (P-value) and a P-value corrected for multiple testing (Corrected P-value). Both values were categorized based on the indicated limits to produce a heatmap. For each set of stratified disorder-associated loci and each set of nORF-HARs, the number of nORFs that overlapped a locus was quantified and is displayed in the relevant cell in the heatmap.

FIGS. 3A-3C are a set of graphs showing translated nORFs in neuropsychiatric samples. FIG. 3A shows that 482 known proteins were identified across CNT, SCZ, and BD samples upon proteomic analysis, of which 408 were common to all three, and 11, 16 and 5 proteins were unique to CNT, SCZ and BD samples, respectively. FIG. 3B shows 21 nORFs were identified as translated of which 17 were common between CNT, SCZ, and BD. Additionally, 2 nORFs were unique to BD and 2 to SCZ and BD, respectively. FIG. 3C shows that the translated nORFs were split according to their annotation type—identified with reference to the transcripts within which the nORFs are contained. 10/21 nORFs are truncations of the main transcript and 6/21 are within pseudogenes.

FIGS. 4A-4C are a set of graphs showing the metadata-specific differences in translated nORFs. FIGS. 4A and 4B show the expression of the 21 translated nORFs were compared for differences (presence/absence evaluated as Yes/No) between (FIG. 4A) gender and (FIG. 4B) incidence of psychosis and suicide. Significance was evaluated using a Chi-squared test for each disorder (right of each bar) or inter-disorder (to the left of the nORF identifiers (ids). FIG. 4C shows 3 unique novel peptides identified via proteogenomic analysis were compared for differences between gender, incidence of psychosis and suicide. Novel peptides with significant differences between the metadata categories evaluated using Chi-squared test were identified at p-value significances. ***<0.001; **<0.01; *<0.05.

FIGS. 5A and 5B are a set of schematics showing the structure prediction for translated nORFs. FIG. 5A shows that the structures were predicted for the 21 translated nORFs, 4 of which are shown along with their nORF IDs (top left). These nORFs were found to be significantly different in BD and SCZ patients for psychosis or suicide. FIG. 5B shows an example of predicted structures for nORFs that are DE in BD (up: top left; down: top right) and SCZ (up: bottom left; down: bottom right) and were found to be associated with HARs. Additionally, the DE nORF tracer_65443 and its parent gene ZEB2 were both within a SCZ-associated locus (SNP locus that involved SNPs with p-value 10⁻⁷<P<10⁻⁶). The parent gene (SCL7A60S) of one DE nORF (tracer_42939) was within a SCZ-associated locus p<10⁻⁷ and was also associated with a BD-associated SNP locus that involved SNPs with 10⁻⁵<P<10⁻⁴.

FIG. 6 is a schematic showing an ideogram of unique HARs. Constructed using www.ncbi.nlm.nih.gov/genome/tools/gdp, the figure illustrates the location of unique HARs within the hg19 genome assembly.

FIGS. 7A and 7B are a set of graphs showing the classification of nORFs. FIG. 7A shows the 248,135 curated nORF entries that were classified with respect to known genes. The translation frame was compared between nORFs and protein-coding genes and the nORF was categorized as in-frame or not in-frame. For not in-frame nORFs, their genomic position in relation to the gene structure was annotated. For nORFs in noncoding region, the overlapping transcripts' biotype was used for categorization. FIG. 7B shows that approximately, 42% of the nORFs in the dataset are in an alternative frame within the CDS of a protein-coding gene.

FIGS. 8A and 8B are a set of graphs showing the psychENCODE sample set. FIG. 8A shows three different studies from the PsychENCODE consortium that were used in analysis. Transcript expression correlation (Spearman's correlation) between the different samples shows greater correlation between samples from the same study than those from different studies. Black cluster-BrainGVEX; Yellow cluster-CMC_HBCC; Pink cluster-CMC. FIG. 8B shows the PCA of gene and transcript expression for the samples highlights the gene-level and transcript-level differences based on the study used. Surrogate variable analysis (SVA) and MARS regression analysis were utilized for additional covariate identification for downstream DE analysis.

FIG. 9 is a schematic showing the Stanley Medical Research Institute (SMRI) samples used in analysis. RNA sequencing (RNA-seq) and mass spectrometry samples for controls (CNT), SCZ and BD were obtained from the Array Collection via SMRI. Available sample numbers are highlighted in the figure.

FIG. 10 is a graph showing the HISAT2 alignment of SMRI samples. HISAT2 alignment summary showing ˜95% alignment rate. The Y-axis shows the proportion of different types of read alignments. Concordant alignment (dark blue) refers to reads that aligned to the reference genome with a specified orientation (forward-reverse) and within a specific distance with respect to each other. Other alignment (light grey) contains reads that mapped concordantly >1-time, discordant alignments as well as single reads of a mate pair that aligned at least 1 time. Unaligned (dark grey) refers to reads that aligned to the genome 0 times.

FIG. 11 is a graph showing the StringTie transcript assembly of SMRI samples, including the number of transcripts identified before and after StringTie merged. Blue bars indicate unannotated transcripts (no corresponding ENSMEBL identifiers (IDs)), and grey bars indicate transcripts with ENSMEBL IDs.

FIG. 12 is a set of graphs showing the identification of confounding factors using sample metadata. Significant differences between disorder and control for each metadata category was evaluated using Mann-Whitney U test in R. Fluphenazine concentration was considered as a covariate for downstream DE analysis. Significance was assigned as * for p-value <0.05, ** for p-value <0.01, *** for p-value <0.001 and “N.S.” for non-significant p-values.

FIG. 13 is a schematic showing the proteogenomic workflow for novel peptide identification. Proteomic samples collected from brain tissue were processed using mass spectrometry, and known proteins were filtered out based on matches of the resultant spectra against the human UniProt database. Any unmapped spectra were processed in two ways: (1) by mapping against the amino acid sequences of nORFs and (2) by processing matched RNA-seq samples isolated from the same brain tissue, assembling the transcripts and six-frame translating their sequences to map against the MS spectra. After additional filtration steps, this proteogenomic workflow allows for the identification of novel peptides or nORF peptides within protein samples.

FIGS. 14A and 14B are a set of graphs showing novel peptide identification. Distribution of MASCOT protein (FIG. 14A) and peptide (FIG. 14B) and protein scores calculated for novel peptides in neuropsychiatric samples using proteogenomic analysis are shown. For novel peptide filtration, a peptide score of greater than 50 and peptide expectation value of less than 0.05 was used.

FIG. 15 is a set of graphs showing nORF peptide identification. Distribution of MASCOT peptide and protein scores calculated for nORF peptides in neuropsychiatric samples using proteogenomic analysis are shown. For nORF peptide filtration, a protein score of greater than 50 and a protein expectation score of less than 0.05 was used. Following this, a peptide score of greater than 50 and peptide expectation value of less than 0.05 was used for further filtration, Finally, nORF peptides expressed in at least 30% of each case/control group were retained.

FIG. 16 is a graph showing nORF amino acid length distribution. For the 21 nORFs identified as translated in neuropsychiatric samples, the distribution plot above shows the length (number of amino acids (aa)) of these nORFs. The majority of the nORFs identified are 200-300 aa in length.

FIGS. 17A and 17B are a set of graphs showing the evaluation of known protein-coding genes containing nORFs. FIG. 17A shows the number of genes with and without nORFs, split according to gene biotype is presented. nORFs are largely localized within protein-coding genes. FIG. 17B shows further evaluation of protein-coding genes was performed using FunRich v3.1.3. The percentage of genes with and without nORFs were evaluated for enrichment of clinical phenotypes. Genes with nORFs are associated significantly with neurological disorders.

FIG. 18 is a schematic showing an ideogram (constructed using www.ncbi.nlm.nih.gov/genome/tools/gdp) illustrating the location of DE nORFs within the hg19 genome assembly.

FIG. 19 is a set of schematics showing nORF enrichment in BD- and SCZ-specific loci. nORFs transcribed within neuropsychiatric samples (blue peaks) and DE in SCZ (red peaks) or DE in BD (green peaks), were evaluated for overlap and enrichment within SCZ-specific CNVs and BD-specific loci (black vertical lines—outermost circular panel), using GLANET. If nORF enrichment was identified, the corresponding loci (vertical line) is marked with circular dots. For BD-specific loci and SCZ-specific CNVs (copy-number variations) showed no nORF enrichment. Enrichment was found within SCZ-specific loci, as described in FIG. 2A.

FIG. 20 is a graph showing types of individual HARs within the sets of associated HARs. Abbreviations: BD, DE nORF-HARs in BD; S1, DE nORF-HARs in SCZ; S2, DE nORF-DE HARs in SCZ.

FIG. 21 is a schematic showing Gene Ontology (GO) enrichment results for transcribed and translated nORFs for all the 248,135 nORFs used. GO terms were obtained using InterProScan. For nORFs with evidence of transcription, structural molecular activity within ribosomes and putative involvement in translation was found. For nORFs that were DE, no enrichment was found. Structural molecular activity as part of the myelin sheath and cytoskeleton, guanosine triphosphate (GTP) binding, GTPase and oxidoreductase activity were found enriched within the translated nORF set.

FIGS. 22A-22D is a set of schematic drawings showing the predicted structure for nORFs. Structures predicted for nORFs using i-TASSER or Raptor-X were visualized with Avogadro or Jena3D viewer. These nORFs are either translated in neuropsychiatric analysis or are associated with HARs in SCD and BD samples.

### DETAILED DESCRIPTION

Described herein are methods of diagnosing and treating schizophrenia or bipolar disorder associated with dysregulated novel open reading frames (nORFs). Schizophrenia or bipolar disorder may be caused by dysregulation (e.g., upregulation or downregulation) in a gene or a genetic variant that is associated with the schizophrenia or bipolar disorder. However, it was previously unclear how schizophrenia or bipolar disorder are caused in which no dysregulation of a canonical gene or a canonical open reading frame (cORF) associated with the gene is present and no variant is known. The present invention is premised, in part, upon the discovery of dysregulation of certain novel open reading frames (nORFs) that are distinct from canonical open reading frames (cORF) of genes. In these instances, the dysregulation (e.g., upregulation or downregulation) imparts a deleterious effect on the nORF, in some instances, with or without substantially impacting a protein encoded by a cORF. In particular, the present invention features methods of treating schizophrenia or bipolar disorder associated with a dysregulated nORF in which differential expression (e.g., increased or decreased expression) of the nORF is observed. With increased or decreased expression, the gene product encoded by the dysregulated nORF is increased or decreased as compared to the nORF, e.g., in a subject without schizophrenia or bipolar disorder. The methods of diagnosis and treatment are described in more detail below.

### Methods of Diagnosis

Genetic testing offers one avenue by which a patient may be diagnosed as having or is at risk of developing schizophrenia or bipolar disorder. For example, a genetic analysis can be used to determine whether a patient has a nORF associated with schizophrenia or bipolar disorder. The nORF may be present in any region of a gene, such as within the cORF, a 5′ untranslated region (UTR) of the cORF, a 3′ UTR of the cORF, an intronic region of the cORF, or an intergenic region of the cORF, The nORF may be present within an overlapping region of the cORF in an alternate reading frame, a 5′ UTR of the cORF, a 3′ UTR of the cORF, an intronic region of the cORF, or an intergenic region of the cORF. The nORF may be present in a region that is not associated with the cORF of the gene.

Exemplary genetic tests that can be used to determine whether a patient has such nORF include polymerase chain reaction (PCR) methods known in the art, such as DNA and RNA sequencing. nORF sequences may be identified de novo, e.g., using computational or statistical methods. Furthermore, nORF sequences may be identified from publicly available databases in genomic sequences in which the nORF was not previously identified and/or annotated as a sequence that was transcribed, and/or translated.

nORF sequences may be identified as being linked to schizophrenia or bipolar disorder by using a statistical analysis between the dysregulated nORF and the schizophrenia or bipolar disorder. The statistical analysis may measure a positive or negative association between the dysregulated nORF and the schizophrenia or bipolar disorder (see, e.g., Example 1). The p-value may be, for example, less than 10⁻³, e.g., less than 10⁻⁴, e.g., less than 10⁻⁵.

nORF sequences may be identified as being linked to schizophrenia or bipolar disorder by using a statistical analysis between the dysregulated nORF and a human accelerated region (HAR), which is a region in the human genome that are conserved throughout vertebrate evolution but are different in humans. nORF sequences may be identified as being linked to schizophrenia or bipolar disorder by using a statistical analysis between the dysregulated nORF and a transposable element (TE), which is a DNA sequence that can change its position within a genome. The statistical analysis may measure a positive or negative association between the dysregulated nORF and the HAR and/or the TE (see, e.g., Example 1). The nORF may have a positive or negative association with the HAR and/or the TE. The p-value may be, for example, less than 10⁻³, e.g., less than 10⁻⁴, e.g., less than 10⁻⁵. To examine the functional importance of a nORF separately from a canonical coding sequence, datasets, such as the Genome Aggregation Database, may be used.

### Methods of Treatment

The invention features methods of treating a subject having a dysregulated nORF that has differential expression (e.g., increased or decreased expression). The dysregulated nORF may exhibit an increase (e.g., by at least 5%, 10%, 15%, 20%, 25%, 30%, 40%, 50%, 60%, 70%, 80%, 90%, 100%, 200%, 300%, 400%, 500%, or more) in expression, e.g., as compared to the nORF in a normal (e.g., without schizophrenia or bipolar disorder) subject. The dysregulated nORF may exhibit a decrease (e.g., by at least 5%, 10%, 15%, 20%, 25%, 30%, 40%, 50%, 60%, 70%, 80%, 90%, 95%, 97%, or 99%) in expression, e.g., as compared to the dysregulated nORF in a normal (e.g., without schizophrenia or bipolar disorder) subject. The subject may be first determined to have the dysregulated nORF and then may subsequently be treated for the schizophrenia or bipolar disorder. The subject may have previously been determined to have the dysregulated nORF and is then treated for the schizophrenia or bipolar disorder. The treatment varies according to the dysregulated nORF associated with the schizophrenia or bipolar disorder. For example, the treatment may include an inhibitor that targets the dysregulated nORF to decrease (e.g., by at least 5%, 10%, 15%, 20%, 25%, 30%, 40%, 50%, 60%, 70%, 80%, 90%, 95%, 97%, or 99%) expression of an upregulated nORF. The treatment may include an activator that targets the dysregulated nORF to increase (e.g., by at least 5%, 10%, 15%, 20%, 25%, 30%, 40%, 50%, 60%, 70%, 80%, 90%, 100%, 200%, 300%, 400%, 500%, or more) expression of a downregulated nORF. Alternatively, or in addition, the treatment may include providing the nORF or a protein encoded by the nORF to restore levels of the nORF.

### Inhibitors

The methods of treatment and diagnosis described herein may include providing an inhibitor that targets the dysregulated nORF. The inhibitor may reduce (e.g., by at least 5%, 10%, 15%, 20%, 25%, 30%, 40%, 50%, 60%, 70%, 80%, 90%, 95%, 97%, or 99%) an amount or activity of the dysregulated nORF, such as to prevent the deleterious effect of the dysregulated nORF. The inhibitor may target the polynucleotide containing the nORF or the protein encoded by the nORF. The inhibitor may be a small molecule, a polynucleotide, or a polypeptide. Suitable small molecules may be determined or identified by using computational analysis based on the structure of the dysregulated nORF as determined by a protein folding algorithm. The small molecule may target any region of the dysregulated nORF. The small molecule may target the nORF or the protein encoded by the nORF. Suitable polypeptides for reducing an activity or amount of the dysregulated nORF include, for example, an antibody or antigen-binding fragment thereof that binds to the dysregulated nORF (e.g., a single chain antibody or antigen-binding fragment thereof). Suitable polynucleotides that can reduce an amount or activity of the dysregulated nORF include RNA. For example, an RNA for reducing an activity or amount of the dysregulated nORF may be, for example, a miRNA, an antisense RNA, an shRNA, or an siRNA. The miRNA, antisense RNA, shRNA, or siRNA may target a region of RNA (e.g., dysregulated nORF gene) to reduce expression of the dysregulated nORF. The polynucleotide may be an aptamer, e.g., an RNA aptamer that binds to and/or reduces an amount and/or activity of the dysregulated nORF or the protein encoded by the dysregulated nORF. The inhibitor may be provided directly or may be provided by a vector (e.g., a viral vector) encoding the inhibitor. The inhibitor may be formulated, e.g., in a pharmaceutical composition containing a pharmaceutically acceptable carrier. The composition can be administered by any suitable method known in the art to the skilled artisan. The composition (e.g., a vector, e.g., a viral vector) may be formulated in a virus or a virus-like particle.

### Nucleic Acid Mediated Knockdown

Using the compositions and methods described herein, a patient with schizophrenia or bipolar disorder may be administered an interfering RNA molecule, a composition containing the same, or a vector encoding the same, so as to reduce or suppress the expression of a dysregulated nORF. Exemplary interfering RNA molecules that may be used in conjunction with the compositions and methods described herein are siRNA molecules, miRNA molecules, shRNA molecules, and antisense RNA molecules, among others. In the case of siRNA molecules, the siRNA may be single stranded or double stranded. miRNA molecules, in contrast, are single-stranded molecules that form a hairpin, thereby adopting a hydrogen-bonded structure reminiscent of a nucleic acid duplex. In either case, the interfering RNA may contain an antisense or “guide” strand that anneals (e.g., by way of complementarity) to the repeat-expanded mutant RNA target. The interfering RNA may also contain a “passenger” strand that is complementary to the guide strand and, thus, may have the same nucleic acid sequence as the RNA target.

siRNA is a class of short (e.g., 20-25 nt) double-stranded non-coding RNA that operates within the RNA interference pathway. siRNA may interfere with expression of the dysregulated nORF gene with complementary nucleotide sequences by degrading mRNA (via the Dicer and RISC pathways) after transcription, thereby preventing translation. miRNA is another short (e.g., about 22 nucleotides) non-coding RNA molecule that functions in RNA silencing and post-transcriptional regulation of gene expression. miRNAs function via base-pairing with complementary sequences within mRNA molecules, thereby leading to cleavage of the mRNA strand into two pieces and destabilization of the mRNA through shortening of its poly(A) tail. shRNA is an artificial RNA molecule with a tight hairpin turn that can be used to silence target gene expression via RNA interference. Antisense RNA are also short single stranded molecules that hybridize to a target RNA and prevent translation by occluding the translation machinery, thereby reducing expression of the target (e.g., the dysregulated nORF).

### Antibody Mediated Knockdown

Using the compositions and methods described herein, a patient with schizophrenia or bipolar disorder may be provided an antibody or antigen-binding fragment thereof, a composition containing the same, a vector encoding the same, or a composition of cells containing a vector encoding the same, so as to suppress or reduce the activity of the dysregulated nORF. In some embodiments of the compositions and methods described herein, an antibody or antigen-biding fragment thereof may be used that binds to and reduces or eliminates the activity of the dysregulated nORF. The antibody may be monoclonal or polyclonal. In some embodiments, the antigen-binding fragment is an antibody that lacks the Fc portion, an F(ab′)2, a Fab, an Fv, or an scFv. The antigen-binding fragment may be an scFv. One of ordinary skill in the art will appreciate that an antibody may include four polypeptides: two identical copies of a heavy chain polypeptide and two copies of a light chain polypeptide. Each of the heavy chains contains one N-terminal variable (V_{H}) region and three C-terminal constant (CH1, CH2 and CH3) regions, and each light chain contains one N-terminal variable (V_{L}) region and one C-terminal constant (C_{L}) region. Thus, one of skill in the art would appreciate that as described herein, a vector that includes a transgene that encodes a polypeptide that is an antibody may be a single transgene that encodes a plurality of polypeptides. Also contemplated is a vector that includes a plurality of transgenes, each transgene encoding a separate polypeptide of the antibody. All variations are contemplated herein. The variable regions of each pair of light and heavy chains form the antigen binding site of an antibody. The transgene which encodes an antibody directed against the dysregulated nORF can include one or more transgene sequences, each of which encodes one or more of the heavy and/or light chain polypeptides of an antibody. In this respect, the transgene sequence which encodes an antibody directed against the dysregulated nORF can include a single transgene sequence that encodes the two heavy chain polypeptides and the two light chain polypeptides of an antibody. Alternatively, the transgene sequence which encodes an antibody directed against the dysregulated nORF can include a first transgene sequence that encodes both heavy chain polypeptides of an antibody, and a second transgene sequence that encodes both light chain polypeptides of an antibody. In yet another embodiment, the transgene sequence which encodes an antibody can include a first transgene sequence encoding a first heavy chain polypeptide of an antibody, a second transgene sequence encoding a second heavy chain polypeptide of an antibody, a third transgene sequence encoding a first light chain polypeptide of an antibody, and a fourth transgene sequence encoding a second light chain polypeptide of an antibody.

In some embodiments, the transgene that encodes the antibody includes a single open reading frame encoding a heavy chain and a light chain, and each chain is separated by a protease cleavage site.

In some embodiments, the transgene encodes a single open reading frame encoding both heavy chains and both light chains, and each chain is separate by protease cleavage site.

In some embodiments, full-length antibody expression can be achieved from a single transgene cassette using 2A peptides, such as foot-and-mouth disease virus (FMDV) equine rhinitis A, porcine teschovirus-1, and Thosea asigna virus 2A peptides, which are used to link two or more genes and allow the translated polypeptide to be self-cleaved into individual polypeptide chains (e.g., heavy chain and light chain, or two heavy chains and two light chains). Thus, in some embodiments, the transgene encodes a 2A peptide in between the heavy and light chains, optionally with a flexible linker flanking the 2A peptide (e.g., GSG linker). The transgene may further include one or more engineered cleavage sequences, e.g., a furin cleavage sequence to remove the 2A peptide residues attached to the heavy chain or light chain. Exemplary 2A peptides are described, e.g., in Chng et al MAbs 7: 403-412, 201f5, and Lin et al. Front. Plant Sci. 9:1379, 2018, the disclosures of which are hereby incorporated by reference in their entirety.

In some embodiments, the antibody is a single-chain antibody or antigen-binding fragment thereof expressed from a single transgene.

### Activators

The methods of treatment and diagnosis described herein may include providing an activator that targets the dysregulated nORF. The activator may increase (e.g., by at least 5%, 10%, 15%, 20%, 25%, 30%, 40%, 50%, 60%, 70%, 80%, 90%, 100%, 200%, 300%, 400%, 500%, or more) an amount or activity of the dysregulated nORF, such as to prevent the deleterious effect of the dysregulated nORF. The activator may target the polynucleotide containing the nORF or the protein encoded by the nORF. The activator may be a small molecule, a polynucleotide, or a polypeptide. Suitable small molecules may be determined or identified by using computational analysis based on the structure of the dysregulated nORF as determined by a protein folding algorithm. The small molecule may target any region of the dysregulated nORF. The small molecule may target the nORF or the protein encoded by the nORF. Suitable polypeptides for increasing an activity or amount of the dysregulated nORF include, for example, an antibody or antigen-binding fragment thereof that binds to the dysregulated nORF (e.g., a single chain antibody or antigen-binding fragment thereof). Suitable polynucleotides that can increase an amount or activity of the dysregulated nORF include RNA. For example, an RNA for increasing an activity or amount of the dysregulated nORF may be, for example, an antisense RNA. The antisense RNA may target a region of RNA (e.g., dysregulated nORF gene) upstream of the primary nORF open reading frame to reduce expression of the upstream nORFs, thereby dedicating the translation machinery to the primary nORF in order to increase expression of the primary nORF. The polynucleotide may be an aptamer, e.g., an RNA aptamer that binds to and/or increases an amount and/or activity of the dysregulated nORF or the protein encoded by the dysregulated nORF. The activator may be provided directly or may be provided by a vector (e.g., a viral vector) encoding the activator. The activator may be formulated, e.g., in a pharmaceutical composition containing a pharmaceutically acceptable carrier. The composition can be administered by any suitable method known in the art to the skilled artisan. The composition (e.g., a vector, e.g., a viral vector) may be formulated in a virus or a virus-like particle.

nORF Replacement

The present invention also features methods of treating schizophrenia or bipolar disorder by administering or providing a nORF or a protein encoded by the nORF. The therapy may restore the encoded protein product of the nORF, such as to replace the nORF that is no longer present due to downregulation. The therapy may include providing the protein product or a polynucleotide encoding the protein product. The method may include providing a vector (e.g., a viral vector) that encodes the protein product. Alternatively, the protein encoded by the nORF may be administered directly, e.g., as an enzyme replacement therapy. The nORF or a polynucleotide encoding the nORF (e.g., a vector, e.g., a viral vector) may be formulated, e.g., in a pharmaceutical composition containing a pharmaceutically acceptable carrier. The composition can be administered by any suitable method known in the art to the skilled artisan. The composition may be formulated in a virus or a virus-like particle.

In some embodiments, the length of the nORF is less than about 100 amino acids (e.g., from about 50 to 100, 50 to 90, 50 to 80, 60 to 90, 60 to 80, 70 to 100, 70 to 90, 70 to 80, 80 to 100, or 90 to 100 amino acids).

### Viral Vectors for Expression

Viral genomes provide a rich source of vectors that can be used for the efficient delivery of exogenous genes into a mammalian cell. The gene to be delivered may include an activator or inhibitor that targets a dysregulated nORF, such as an RNA (e.g., an aptamer, a miRNA, an antisense RNA, an shRNA, or an siRNA). Alternatively, the gene to be delivered may include the nORF for replacement. Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors are a retrovirus (e.g., Retroviridae family viral vector), adenovirus (e.g., Ad5, Ad26, Ad34, Ad35, and Ad48), parvovirus (e.g., an adeno-associated viral (AAV) vector), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses are: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, (1996))). Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030), the teachings of which are incorporated herein by reference.

### Retroviral Vectors

The delivery vector used in the methods described herein may be a retroviral vector. One type of retroviral vector that may be used in the methods and compositions described herein is a lentiviral vector. Lentiviral vectors (LVs), a subset of retroviruses, transduce a wide range of dividing and non-dividing cell types with high efficiency, conferring stable, long-term expression of the transgene encoding the polypeptide or RNA. An overview of optimization strategies for packaging and transducing LVs is provided in Delenda, The Journal of Gene Medicine 6: S125 (2004), the disclosure of which is incorporated herein by reference.

The use of lentivirus-based gene transfer techniques relies on the in vitro production of recombinant lentiviral particles carrying a highly deleted viral genome in which the agent of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, encapsidation, and expression, in which the sequences to be expressed are inserted.

A LV used in the methods and compositions described herein may include one or more of a 5′-Long terminal repeat (LTR), HIV signal sequence, HIV Psi signal 5′-splice site (SD), delta-GAG element, Rev Responsive Element (RRE), 3′-splice site (SA), elongation factor (E F) 1-alpha promoter and 3′-self inactivating LTR (SIN-LTR). The lentiviral vector optionally includes a central polypurine tract (cPPT) and a woodchuck hepatitis virus post-transcriptional regulatory element (WPRE), as described in U.S. Pat. No. 6,136,597, the disclosure of which is incorporated herein by reference as it pertains to WPRE. The lentiviral vector may further include a pHR′ backbone, which may include for example as provided below.

The Lentigen LV described in Lu et al., Journal of Gene Medicine 6:963 (2004) may be used to express the DNA molecules and/or transduce cells. A LV used in the methods and compositions described herein may a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating L TR (SIN-LTR). It will be readily apparent to one skilled in the art that optionally one or more of these regions is substituted with another region performing a similar function.

Enhancer elements can be used to increase expression of modified DNA molecules or increase the lentiviral integration efficiency. The LV used in the methods and compositions described herein may include a nef sequence. The LV used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The LV used in the methods and compositions described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to LV results in a substantial improvement in the level of expression from several different promoters, both in vitro and in vivo. The LV used in the methods and compositions described herein may include both a cPPT sequence and WPRE sequence. The vector may also include an IRES sequence that permits the expression of multiple polypeptides from a single promoter.

In addition to IRES sequences, other elements which permit expression of multiple polypeptides are useful. The vector used in the methods and compositions described herein may include multiple promoters that permit expression more than one polypeptide. The vector used in the methods and compositions described herein may include a protein cleavage site that allows expression of more than one polypeptide. Examples of protein cleavage sites that allow expression of more than one polypeptide are described in Klump et al., Gene Ther.; 8:811 (2001), Osborn et al., Molecular Therapy 12:569 (2005), Szymczak and Vignali, Expert Opin Biol Ther. 5:627 (2005), and Szymczak et al., Nat Biotechnol. 22:589 (2004), the disclosures of which are incorporated herein by reference as they pertain to protein cleavage sites that allow expression of more than one polypeptide. It will be readily apparent to one skilled in the art that other elements that permit expression of multiple polypeptides identified in the future are useful and may be utilized in the vectors suitable for use with the compositions and methods described herein.

The vector used in the methods and compositions described herein may, be a clinical grade vector.

The viral vectors (e.g., retroviral vectors, e.g., lentiviral vectors) may include a promoter operably coupled to the transgene encoding the polypeptide or the polynucleotide encoding the RNA to control expression. The promoter may be a ubiquitous promoter. Alternatively, the promoter may be a tissue specific promoter, such as a myeloid cell-specific or hepatocyte-specific promoter. Suitable promoters that may be used with the compositions described herein include CD11b promoter, sp146/p47 promoter, CD68 promoter, sp146/gp9 promoter, elongation factor 1 α (EF1α) promoter, EF1α short form (EFS) promoter, phosphoglycerate kinase (PGK) promoter, α-globin promoter, and β-globin promoter. Other promoters that may be used include, e.g., DC172 promoter, human serum albumin promoter, alpha1 antitrypsin promoter, thyroxine binding globulin promoter. The DC172 promoter is described in Jacob, et al. Gene Ther. 15:594-603, 2008, hereby incorporated by reference in its entirety.

The viral vectors (e.g., retroviral vectors, e.g., lentiviral vectors) may include an enhancer operably coupled to the transgene encoding the polypeptide or the polynucleotide encoding the RNA to control expression. The enhancer may include a β-globin locus control region (βLCR).

Methods of Measuring nORF Gene Expression

Preferably, the compositions and methods of the disclosure are used to facilitate expression of a nORF at physiologically normal levels in a patient (e.g., a human patient), decrease expression of an upregulated nORF, or increase expression of a downregulated nORF. The therapeutic agents of the disclosure, for example, may reduce the dysregulated nORF expression in a human subject. For example, the therapeutic agents of the disclosure may reduce dysregulated nORF expression e.g., by about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, 98%, or 99%. Alternatively, the therapeutic agents of the disclosure may increase the dysregulated nORF expression in a human subject. For example, the therapeutic agents of the disclosure may increase dysregulated nORF expression, e.g., by at least 5%, 10%, 15%, 20%, 25%, 30%, 40%, 50%, 60%, 70%, 80%, 90%, 100%, 200%, 300%, 400%, 500%, or more.

The expression level of the nORF expressed in a patient can be ascertained, for example, by evaluating the concentration or relative abundance of mRNA transcripts derived from transcription of the nORF. Additionally, or alternatively, expression can be determined by evaluating the concentration or relative abundance of the nORF following transcription and/or translation of an inhibitor that decreases an amount of the dysregulated nORF. Protein concentrations can also be assessed using functional assays, such as MDP detection assays. Expression can be evaluated by a number of methodologies known in the art, including, but not limited to, nucleic acid sequencing, microarray analysis, proteomics, in-situ hybridization (e.g., fluorescence in-situ hybridization (FISH)), amplification-based assays, in situ hybridization, fluorescence activated cell sorting (FACS), northern analysis and/or PCR analysis of mRNAs.

### Nucleic Acid Detection

Nucleic acid-based methods for determining expression (e.g., of an RNA inhibitor or an RNA encoding the nORF) detection that may be used in conjunction with the compositions and methods described herein include imaging-based techniques (e.g., Northern blotting or Southern blotting). Such techniques may be performed using cells obtained from a patient following administration of the polynucleotide encoding the agent. Northern blot analysis is a conventional technique well known in the art and is described, for example, in Molecular Cloning, a Laboratory Manual, second edition, 1989, Sambrook, Fritch, Maniatis, Cold Spring Harbor Press, 10 Skyline Drive, Plainview, NY 11803-2500. Typical protocols for evaluating the status of genes and gene products are found, for example in Ausubel et al., eds., 1995, Current Protocols in Molecular Biology, Units 2 (Northern Blotting), 4 (Southern Blotting), 15 (Immunoblotting) and 18 (PCR Analysis).

Detection techniques that may be used in conjunction with the compositions and methods described herein to evaluate nORF expression further include microarray sequencing experiments (e.g., Sanger sequencing and next-generation sequencing methods, also known as high-throughput sequencing or deep sequencing). Exemplary next generation sequencing technologies include, without limitation, Illumina sequencing, Ion Torrent sequencing, 454 sequencing, SOLiD sequencing, and nanopore sequencing platforms. Additional methods of sequencing known in the art can also be used. For instance, expression at the mRNA level may be determined using RNA-Seq (e.g., as described in Mortazavi et al., Nat. Methods 5:621-628 (2008) the disclosure of which is incorporated herein by reference in their entirety). RNA-Seq is a robust technology for monitoring expression by direct sequencing the RNA molecules in a sample. Briefly, this methodology may involve fragmentation of RNA to an average length of 200 nucleotides, conversion to cDNA by random priming, and synthesis of double-stranded cDNA (e.g., using the Just cDNA DoubleStranded cDNA Synthesis Kit from Agilent Technology). Then, the cDNA is converted into a molecular library for sequencing by addition of sequence adapters for each library (e.g., from Illumina®/Solexa), and the resulting 50-100 nucleotide reads are mapped onto the genome.

Expression levels of the nORF may be determined using microarray-based platforms (e.g., single-nucleotide polymorphism arrays), as microarray technology offers high resolution. Details of various microarray methods can be found in the literature. See, for example, U.S. Pat. No. 6,232,068 and Pollack et al., Nat. Genet. 23:41-46 (1999), the disclosures of each of which are incorporated herein by reference in their entirety. Using nucleic acid microarrays, mRNA samples are reverse transcribed and labeled to generate cDNA. The probes can then hybridize to one or more complementary nucleic acids arrayed and immobilized on a solid support. The array can be configured, for example, such that the sequence and position of each member of the array is known. Hybridization of a labeled probe with a particular array member indicates that the sample from which the probe was derived expresses that gene. Expression level may be quantified according to the amount of signal detected from hybridized probe-sample complexes. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. One example of a microarray processor is the Affymetrix GENECHIP® system, which is commercially available and comprises arrays fabricated by direct synthesis of oligonucleotides on a glass surface. Other systems may be used as known to one skilled in the art.

Amplification-based assays also can be used to measure the expression level of the nORF or RNA in a target cell following delivery to a patient. In such assays, the nucleic acid sequences of the gene act as a template in an amplification reaction (for example, PCR, such as qPCR). In a quantitative amplification, the amount of amplification product is proportional to the amount of template in the original sample. Comparison to appropriate controls provides a measure of the expression level of the gene, corresponding to the specific probe used, according to the principles described herein. Methods of real-time qPCR using TaqMan probes are well known in the art. Detailed protocols for real-time qPCR are provided, for example, in Gibson et al., Genome Res. 6:995-1001 (1996), and in Heid et al., Genome Res. 6:986-994 (1996), the disclosures of each of which are incorporated herein by reference in their entirety. Levels of gene expression as described herein can be determined by RT-PCR technology. Probes used for PCR may be labeled with a detectable marker, such as, for example, a radioisotope, fluorescent compound, bioluminescent compound, a chemiluminescent compound, metal chelator, or enzyme.

### Protein Detection

Expression of the nORF can additionally be determined by measuring the concentration or relative abundance of a corresponding protein product (e.g., as compared to the nORF in a subject without schizophrenia or bipolar disorder or the dysregulated nORF). Protein levels can be assessed using standard detection techniques known in the art. Protein expression assays suitable for use with the compositions and methods described herein include proteomics approaches, immunohistochemical and/or western blot analysis, immunoprecipitation, molecular binding assays, ELISA, enzyme-linked immunofiltration assay (ELIFA), mass spectrometry, mass spectrometric immunoassay, and biochemical enzymatic activity assays. Proteomics methods can be used to generate large-scale protein expression datasets in multiplex. Proteomics methods may utilize mass spectrometry to detect and quantify polypeptides (e.g., proteins) and/or peptide microarrays utilizing capture reagents (e.g., antibodies) specific to a panel of target proteins to identify and measure expression levels of proteins expressed in a sample (e.g., a single cell sample or a multi-cell population).

Exemplary peptide microarrays have a substrate-bound plurality of polypeptides, the binding of an oligonucleotide, a peptide, or a protein to each of the plurality of bound polypeptides being separately detectable. Alternatively, the peptide microarray may include a plurality of binders, including, but not limited to, monoclonal antibodies, polyclonal antibodies, phage display binders, yeast two-hybrid binders, aptamers, which can specifically detect the binding of specific oligonucleotides, peptides, or proteins. Examples of peptide arrays may be found in U.S. Pat. Nos. 6,268,210, 5,766,960, and 5,143,854, the disclosures of each of which are incorporated herein by reference in their entirety.

Mass spectrometry (MS) may be used in conjunction with the methods described herein to identify and characterize expression of the nORF in a cell from a patient (e.g., a human patient) following delivery of the transgene encoding the nORF. Any method of MS known in the art may be used to determine, detect, and/or measure a protein or peptide fragment of interest, e.g., LC-MS, ESI-MS, ESI-MS/MS, MALDI-TOF-MS, MALDI-TOF/TOF-MS, tandem MS, and the like. Mass spectrometers generally contain an ion source and optics, mass analyzer, and data processing electronics. Mass analyzers include scanning and ion-beam mass spectrometers, such as time-of-flight (TOF) and quadruple (Q), and trapping mass spectrometers, such as ion trap (IT), Orbitrap, and Fourier transform ion cyclotron resonance (FT-ICR), may be used in the methods described herein. Details of various MS methods can be found in the literature. See, for example, Yates et al., Annu. Rev. Biomed. Eng. 11:49-79, 2009, the disclosure of which is incorporated herein by reference in its entirety.

Prior to MS analysis, proteins in a sample obtained from the patient can be first digested into smaller peptides by chemical (e.g., via cyanogen bromide cleavage) or enzymatic (e.g., trypsin) digestion. Complex peptide samples also benefit from the use of front-end separation techniques, e.g., 2D-PAGE, HPLC, RPLC, and affinity chromatography. The digested, and optionally separated, sample is then ionized using an ion source to create charged molecules for further analysis. Ionization of the sample may be performed, e.g., by electrospray ionization (ESI), atmospheric pressure chemical ionization (APCI), photoionization, electron ionization, fast atom bombardment (FAB)/liquid secondary ionization (LSIMS), matrix assisted laser desorption/ionization (MALDI), field ionization, field desorption, thermospray/plasmaspray ionization, and particle beam ionization. Additional information relating to the choice of ionization method is known to those of skill in the art.

After ionization, digested peptides may then be fragmented to generate signature MS/MS spectra. Tandem MS, also known as MS/MS, may be particularly useful for analyzing complex mixtures. Tandem MS involves multiple steps of MS selection, with some form of ion fragmentation occurring in between the stages, which may be accomplished with individual mass spectrometer elements separated in space or using a single mass spectrometer with the MS steps separated in time. In spatially separated tandem MS, the elements are physically separated and distinct, with a physical connection between the elements to maintain high vacuum. In temporally separated tandem MS, separation is accomplished with ions trapped in the same place, with multiple separation steps taking place over time. Signature MS/MS spectra may then be compared against a peptide sequence database (e.g., SEQUEST). Post-translational modifications to peptides may also be determined, for example, by searching spectra against a database while allowing for specific peptide modifications.

### Schizophrenia and Bipolar Disorder

The present invention contemplates treatment of schizophrenia or bipolar disorder in which a nORF exhibits increased or decreased expression, e.g., relative to a subject without schizophrenia or bipolar disorder. Schizophrenia is a mental illness that affects how a person thinks, feels, and behaves. Bipolar disorder, also known as manic depression, is a mental illness that brings server high and low moods and changes in sleep, energy, thinking, and behavior.

The method may decrease or slow (e.g., by at least 5%, 10%, 15%, 20%, 25%, 30%, 40%, 50%, 60%, 70%, 80%, 90%, 95%, 97%, or 99%) the progression of schizophrenia or bipolar disorder. The method may decrease (e.g., by at least 5%, 10%, 15%, 20%, 25%, 30%, 40%, 50%, 60%, 70%, 80%, 90%, 95%, 97%, or 99%) the risk of developing schizophrenia or bipolar disorder. The method may decrease (e.g., by at least 5%, 10%, 15%, 20%, 25%, 30%, 40%, 50%, 60%, 70%, 80%, 90%, 95%, 97%, or 99%) the risk of developing schizophrenia or bipolar disorder.

In some embodiments, the nORF is selected from Table 4.

In some embodiments, the nORF has at least 85% (e.g., at least 90%, 95%, 97%, 98%, 99%, or 100%) sequence identity to any one of SEQ ID NOs: 1-21 or a fragment thereof.

In some embodiments, the nORF has the sequence of any one of SEQ ID NOs: 1-21.

In some embodiments, the disease is bipolar disorder, and the nORF is selected from Table 7. In some embodiments, the nORF has at least 85% (e.g., at least 90%, 95%, 97%, 98%, 99%, or 100%) sequence identity to any one of SEQ ID NOs: 124-163 or a fragment thereof. In some embodiments, the nORF has the sequence of any one of SEQ ID NOs: 124-163.

In some embodiments, the disease is bipolar disorder, and the nORF is selected from Table 8. In some embodiments, the nORF has at least 85% (e.g., at least 90%, 95%, 97%, 98%, 99%, or 100%) sequence identity to any one of SEQ ID NOs: 164-207 or a fragment thereof. In some embodiments, the nORF has the sequence of any one of SEQ ID NOs: 164-207.

In some embodiments, the disease is schizophrenia, and the nORF is selected from Table 9. In some embodiments, the nORF has at least 85% (e.g., at least 90%, 95%, 97%, 98%, 99%, or 100%) sequence identity to any one of SEQ ID NOs: 208-263 or a fragment thereof. In some embodiments, the nORF has the sequence of any one of SEQ ID NOs: 208-263.

In some embodiments, the disease is schizophrenia, and the nORF is selected from Table 10. In some embodiments, the nORF has at least 85% (e.g., at least 90%, 95%, 97%, 98%, 99%, or 100%) sequence identity to any one of SEQ ID NOs: 264-324 or a fragment thereof. In some embodiments, the nORF has the sequence of any one of SEQ ID NOs: 264-324.

In some embodiments, the nORF is associated with a transposable element. For example, the nORF may have a positive or negative correlation with a transposable element.

In some embodiments, the nORF is associated with a human accelerated region (HAR). For example, the nORF may have a positive or negative correlation with the HAR.

### EXAMPLES

The following examples further illustrate the invention but should not be construed as in any way limiting its scope.

### Example 1. Novel Open Reading Frames in Human Accelerated Regions and Transposable Elements Reveal New Leads to Understand Schizophrenia and Bipolar Disorder

### Introduction

Although the heritability of both Schizophrenia (SCZ) and bipolar disorder (BD) is approximately 70%—placing them among the most heritable mental health disorders, the corresponding polygenic risk scores explain only a fraction of genetic disease liability, for example 7% in SCZ relative to 64-81% heritability derived from family and twin studies. Moreover, putative individual genome-wide association studies (GWAS) risk alleles account only for a marginal increase in disease risk with odds ratios typically under 1.1 and differences in allele frequencies between cases and controls are often less than 2%. SCZ and BD, therefore, pose an evolutionary-genetic paradox because they exhibit strong negative fitness effects and high heritability, yet they persist at a prevalence of approximately 1% across all human cultures.

We set out to investigate whether novel open reading frames (nORFs) that have recently evolved or have been associated with Human Accelerated Regions (HARs) could cast clues on the disease mechanism. HARs are genomic segments that are highly conserved among nonhuman species but experienced accelerated substitutions in the human genome. Many HARs are found in the introns of, and adjacent to, genes annotated with gene ontology (GO) terms related to transcription and DNA binding. We curated a list of 4,481 unique HARs split into three groups based on the extent of their conservation and verified that they are present in all chromosomes (FIG. 6). “vHARs” are HARs conserved in vertebrates, “mHARs” are HARs conserved in mammals, and “pHARs” are HARs conserved in non-human primates. Of the 4,481 unique HARs, 45.4% are vHARs, 11.0% are mHARs and 43.6% are pHARs.

The human centric nature of HARs led to investigations into their link with SCZ. pHARs were found to be enriched in SCZ-associated loci and pHAR-associated SCZ genes were found to be under stronger selection pressure than other SCZ genes. Additionally, mutations in HARs have been found to contribute to altered cognitive behavior, suggesting importance in neural function. However, HARs have not been systematically examined in any of the psychiatric diseases. The PGC meta-analysis provide a novel opportunity to investigate systematically the role of HARs in SCZ.

Another group of genomic features that regulate gene expression are transposable elements (TEs). TEs come in two classes. Class I are retrotransposons, consisting of long terminal repeats (LTRs), which include human endogenous retroviruses, and non-LTRs, which include long interspersed nuclear elements (LINEs), short interspersed nuclear elements (SINEs) and SINE/VNTR/Alu elements (SVAs). Enhancers can arise through the insertion of TEs; therefore, it is feasible that some HARs arose through TE insertion. TEs can be a source of non-coding RNAs and can act as insulator or boundary elements, splitting the genome into 100 kb-1 Mb domains of active and inactive transcription by preventing the spread of heterochromatin. Indeed, many TEs (especially SINEs) harbor binding sites for factors (CTCF, TFIIIC) that confer insulator activity and organize nuclear architecture. Furthermore, chromatin-based repression of TEs impacts expression of nearby loci; when said repression fails, neighboring loci may be expressed together with the corresponding TEs.

HARs and TEs are two classes of genomic regions that can play a role in the regulation of nearby genes. However, little attention has been placed on non-coding regions, especially nORFs and their transcriptional and translational end products in relation to SCZ and BD. nORFs are present in both coding and non-coding regions of the genome and may be biologically regulated.

We hypothesize that components of the genetic architecture of SCZ and BD are attributable to human lineage-specific evolution which were not previously discovered because of the usage of a conservative definition of a gene and because of analyzing genomic, transcriptomic, and proteomic data in silos. To investigate this, we performed a genome-wide evolutionary assessment of the overlap between nORFs present in HARs and in SCZ and BD-associated loci.

We systematically mined SCZ and BD datasets from the PsychENCODE consortium to detect the expression of nORFs with evidence of translation, which to make a nORF database. Following that, we assessed the relationship and association between differentially expressed (DE) nORFs (DE nORFs) and HARs and TEs, and their enrichment in SCZ and BD associated loci, with the goal to identify differentially expressed nORFs present in pHARs associated with SCZ and BD loci. We also investigated the correlation of HARs or TE transcript expression and nORF transcript expression to identify any potential regulation. In addition, for a smaller subset of samples, we were able to show evidence of translation of nORFs. Finally, we predicted structures for some of the nORFs implicated in both the disorders to demonstrate that they may serve as novel drug targets. Thus, this work highlights interesting molecular mechanisms that have been previously missed and we anticipate that this will lead to novel treatments.

### Methods

Creation of nORF dataset

We used nORFs obtained from two sources—nORFs.org and RPFdbv2.0; however, nORFs from RPFdbv2.0 were further processed. Briefly, expression of nORFs were compared to canonical ORFs (cORFs) from 53 studies (with 353 samples), downloaded from RPFdbv2.0 across 11 human cell lines. The 353 samples were divided into 11 groups based on cell types. Actively translated ORFs with clear sub-codon phasing or triplet periodicity footprints were detected using the RibORF tool for each study. Further, each ORF entry was appended with its corresponding annotations: genomic position, strand, ORF category (one of: canonical, truncated, extended, uORF, overlapping uORF, internal, external, polycistronic, readthrough, non-coding transcripts), length of encoded amino acid, ribosome profiling abundance (RPKMs, raw read counts) and the transcript to which the ORF maps (probable transcript from which ORF is translated). Raw read count abundance for each ORF was then converted to Transcript per million (TPM) values for downstream analysis.

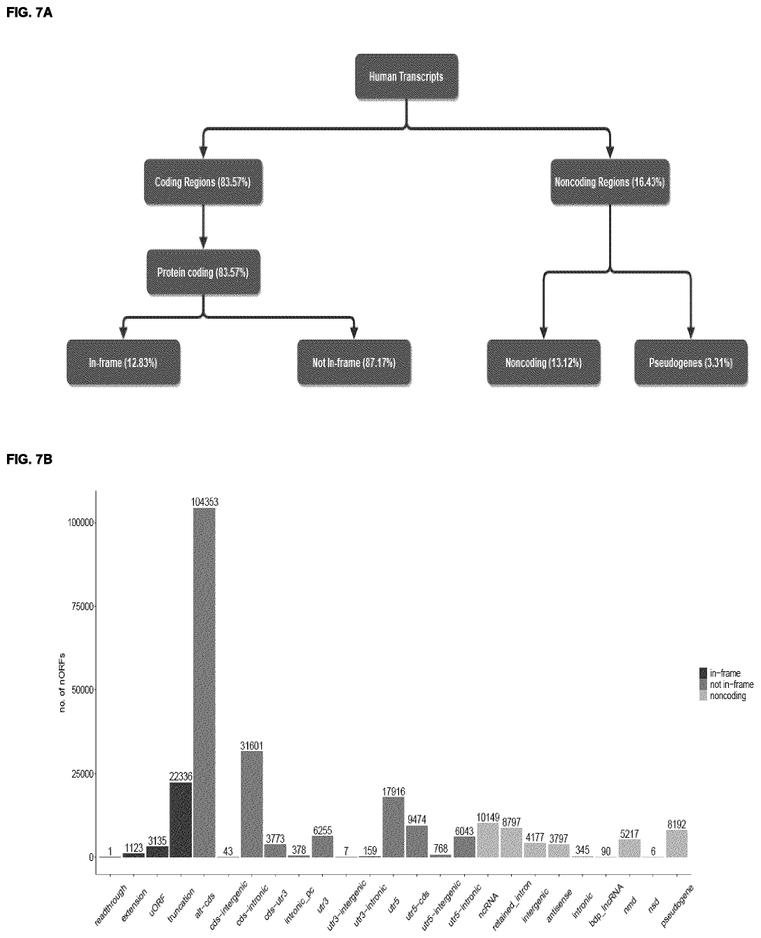

Mean and standard deviation (SD) of Ribo-seq expression TPMs for all 353 samples in each of the 11 groups were compared between the canonical and the ‘non’-canonical ORFs. Mean values were divided into exactly 4000 quantiles with every quantile containing the same number of ORFs. Within each quantile, the SDs were compared between nORFs and cORFs of consequently similar means. ORFs with SDs less than the median SD of cORFs were termed low noise ORFs. 101,797 such low noise nORF entries were added to nORFdbV1, and further, duplicates were removed, and classification was performed. Any nORF classified as in-frame to the CDS of a cORF was removed except for when an annotation such as readthrough, extension, uORF or truncation was determined using the RibORF tool, leading to a final of 248,135 nORF entries. Bedtools getfasta was used to extract the corresponding nucleotide sequence for the new nORF entries using GRCh38 DNA primary assembly (ftp://ftp.ensembl.org/pub/release-96/fasta/homo_sapiens/dna/) with parameters “name”, “s” and “tab” specified. nORF sequences identified using RibORF were translated using Biostrings package in R, which was appended to the curated amino acid sequences of nORFs. Results of this analysis is illustrated in FIG. 7.

Identification of nORF Transcripts in PsychENCODE Dataset

We chose three out of the eight studies, namely BrainGVEX, CMC and CMC_HBCC, which are part of the PsychENCODE consortium, for analysis. These three studies were selected based on availability of total RNA-seq data from SCZ, BD and control (CNT) adult post-mortem brain samples. The total number of samples used in the analysis were 1,340 patient samples—731 CNT, 428 SCZ, and 188 BD. The processed BAM files and RNA-Seq by Expectation-Maximization (RSEM) count files are available under freeze 1 and freeze 2 of the PsychENCODE Consortium. Briefly, CNT, SCZ and BD samples were isolated from the DLPFC, primarily BA9 and BA46, as part of 8 different studies. For analysis, RNA-Seq results of three studies: CMC_HBCC, CommonMind and BrainGVEX, with samples from CNT, SCZ and BD brain samples were used. RNA-Seq reads were aligned to hg19 reference genome using STAR 2.4.2a. Gene- and isoform-level quantifications were performed using RSEM v1.2.29.

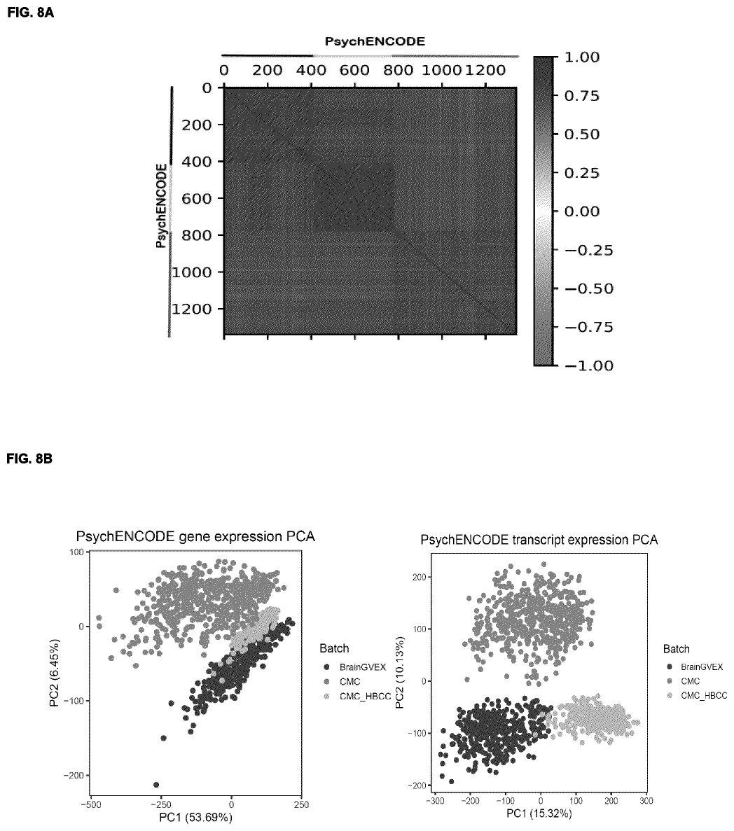

Correlation analysis of gene expression between samples showed higher correlation between samples from the same study group than between samples from different study groups (FIG. 8A). Moreover, Principal Component Analysis (PCA) of both gene and transcript expression for these samples were performed to identify batches/clusters corresponding to the different study groups (FIG. 8B). This analysis revealed considerable batch effect that were accounted for in downstream analyses.

Identification of Transcribed nORFs in PsychENCODE Dataset

GRCh37-based transcript and gene coordinates for 1,340 neuropsychiatric samples from the BrainGVEX, CMC and CMC_HBCC studies were obtained from the PsychENCODE consortium. Transcript expressions were filtered to retain those with TPM>0.1 in at least 10% of the samples. Additionally, transcripts from the Y-chromosome pseudo autosomal regions (PAR) were removed. GffCompare (v0.11.5) mapping was performed between the nORF and sample transcript coordinate. The results file was further filtered as specified in github.com/PrabakaranGroup/norfs_in_neuropsychiatric_disorders. Transcripts containing nORFs with biotype not equal to ‘protein-coding’ were retained.

Identification of Differentially Expressed nORFs

To identify underlying covariates that could affect the DE analysis between SCZ, BD, and CNT, we used multivariate adaptive regression spline (MARS) and surrogate variable analysis (SVA) using the earth and sva package, respectively, in R. Sample transcript count values generated using (RSEM) were normalized using trimmed mean of M-values (TMM) method with edgeR. Earth model with linpreds set to true was run 1000 times and covariates identified at least half of the time were retained. seqPC1-3, seqPC5-7, seqPC10⁻¹⁴, seqPC16, seqPC18-25, seqPC27-29, RIN, RIN.squared, age, batch and individualIDSouce were identified as covariates which were then accounted for during differential expression (DE) analysis.

DE analysis was performed through a linear mixed effects model using nlme package in R, with the above set as fixed effects and individual id as random effect. EdgeR TMM normalized and log₂ (CPM (expression)+0.5) counts were analyzed for DE between CNT and BD and CNT and SCZ. Transcripts which were identified as differentially expressed at an FDR<0.05 after Benjamini-Hochberg correction of the associated p-values, were further evaluated for nORF presence using the GffCompare workflow, as described.

Potential Functional Inferences of nORFs from Amino Acid Sequence

For the 248,135 curated nORFs, GO terms were obtained from equivalent InterPro IDs generated using InterProScan5 run on the galaxy server. Of the total input, 27,430 nORFs with a total of 62,700 corresponding GO terms were identified. Further analysis revealed that of the 3,103 nORFs identified as transcribed in SCZ and BD samples, 49 nORFs had associated GO terms. Similarly, 2 out of 44 and 13 out of 61 DE nORFs in BD and SCZ, respectively, had corresponding GO terms. For the translated nORFs, 17 out of 21 had GO terms. GO term enrichment for each of these nORF categories was conducted using the GOEnrichment tool on the galaxy server. The required .OBO file for this run was obtained from www.obofoundry.org/ontology/go.html_Analysis was conducted at a p-value cut-off of 0.01 with Benjamini-Hochberg multiple testing correction enabled.

Enrichment Analysis of DE nORFs within SCZ and BD Loci

We evaluated the presence and enrichment of DE transcribed nORFs within SCZ and BD associated loci using an annotation and enrichment tool GLANET, which uses random sampling to calculate enrichment of genomic elements within the input query. In addition, we investigated enrichment of certain DNase I hypersensitive site (DHS1), histone modifications and transcription factors (TFs) within the transcribed and DE nORF cohort. SCZ associated high confidence regions were obtained from PsychENCODE resource (resource.psychencode.org/). For BD, associated loci coordinates were taken from www.nature.com/articles/s41588-019-0397-8 #Sec2. SCZ CNVs were curated from pubmed.ncbi.nlm.nih.gov/29687944/.

### Identification of Unique HARs

4,481 unique HARs were compiled. The genomic coordinates of HARs were mapped to hg19/GRCh37 genome assembly where required, using the LiftOver tool (FIG. 6). The seven coordinate files for HARs and the merged list are available in: norfs_in_neuropsychiatric_disorders/supplementary_data/on GitHub

Association of nORFs with HARs

3,103 nORFs were identified to be DE in the BrainGVEX, CMC and CMC_HBCC neuropsychiatric samples. These nORFs are defined to be associated with a HAR if the HAR overlapped the nORF or regions extending 100 kb upstream or downstream of the nORF. This association distance is in accordance with previous work, although a previous study looked at association within 1 kb and another study found that 52% of non-coding HARS examined in the study are located within 1 MB of a developmental gene and 59% are within 1 Mb of a gene DE between humans and chimpanzees. A nORF associated with a HAR is referred to as a nORF-HAR.

### Stratification of SCZ and BD-Associated SNP Loci

SCZ-associated SNPs and BD-associated SNPs were stratified by p-value (p<10⁻²; P<10⁻³; P<104; P<10⁻⁵; P<10⁻⁶; P<10⁻⁷), as shown in Table 1, below.

TABLE 1
The number of disorder-associated SNP loci for each disorder and p-value cut-off

| | P < 10⁻² | P < 10⁻³ | P < 10⁻⁴ | P < 10⁻⁵ | P < 10⁻⁶ | P < 10⁻⁷ |
|---|---|---|---|---|---|---|
| SCZ | 1407 | 1407 | 1404 | 672 | 347 | 181 |
| BD | 455 | 455 | 455 | 151 | 62 | 24 |

To summarize linkage-disequilibrium (LD)-dependent associations between SNPs, these sets of SNPs were clumped in PLINK 1.9 using LD-based clumping and data from 1000 Genome's EUR population (The 1000 Genomes Project Consortium, 2015). Clumping produces LD-independent sets (‘clumps’) of SNPs, which comprise of an index SNP with the highest association and SNPs in high LD with that index SNP. Parameters were chosen to retain SNPs in association with index SNPs with p<0.0001 and r²<0.1 within 3 Mb windows. Due to very high LD within the MHC region, only the most median index SNP and its associated clump were kept from the MHC region. The MHC region was defined as chr6:28,477,797-33,448,354 on the hg19 genome assembly.

The genomic coordinates for disorder-associated loci were found using the index SNPs and the ‘LD-calculations’ procedure on PLINK 1.9. Data from 1000 Genome's EUR population (The 1000 Genomes Project Consortium, 2015) was used to remove index SNPs not in Hardy-Weinberg equilibrium (p<0.0001) or those with a minor allele frequency less than 0.05. Disorder-associated SNP loci were then defined such that SNPs within loci were associated with index SNPs with r²>0.5 and were within 250 kb of an index SNP. The number of SCZ-associated SNP loci was markedly great than that of BD-associated SNP loci. In both disorders, the number of disorder-associated SNP loci is relatively constant for higher p-value stratifications, decreasing after p<10⁻⁵.

Enrichment of nORF-HARs with Disorder-Associated SNP Loci

To determine whether nORFs associated with HARs, especially those differentially expressed, are enriched within disorder-associated loci, an enrichment test was performed using INRICH. This was used as it accounts for SNP density as well as overlapping genes (nORFs in this case). The sets of loci used were those generated in the previous section.

The analysis was carried out for the full set of nORF-HARs, as well as the subsets of nORFs associated with vHARs, mHARs or pHARs. Although INRICH is usually used for analysis of genes, it can also be used for analysis of nORFs. INRICH requires four files: an interval file, which contained the loci-defining genomic coordinates for disorder-associated SNP loci and the ‘rs’ IDs of the loci's index SNPs; an interval map file, which contained the genomic coordinates for and the ‘rs’ IDs of the loci's index SNPs; a target set file, which contained the genomic coordinates and IDs of the nORF-HARs; and a reference gene file, which contained the genomic coordinates of the 3,103 nORFs expressed in the neuropsychiatric samples. Since no SNPs from the genome-wide association studies (GWAS) were present on the sex chromosomes, all nORFs on the sex chromosomes were removed before analysis. INRICH merges any overlapping nORFs before processing. Empirical p-values for enrichment are then calculated through a first round of 5000 permutations. A second round of 5000 permutations corrects for multiple testing and accounts for gene length to give corrected p-values.

### Identification of Unique TEs

3,987,910 TEs throughout the human genome were identified using RepeatMasker (repeatmasker.org/). All coordinates were already based on hg19 genome assembly. TEs that overlapped were merged and resulting in 3,863,891 unique TEs.

Association of nORFs with TEs

nORFs are defined to be associated with a TE if TE overlapped 2 kb region upstream of the nORF, but not the nORF itself. Association between DE nORF and TEs was investigated to gain insight into the impact of TEs on nearby nORF expression via correlation analysis of expression.

### Identification of DE HARs and DE TEs

A set of transcripts DE between SCZ and BD and controls in the PsychENCODE datasets was identified as mentioned above. HARs and TEs that were included in or overlapped with these differentially expressed transcripts were designated DE HARs (differentially expressed HARs) and DE TEs (differentially expressed TEs), respectively. DE TE expression was normalized using the TMM normalization procedure as provided in edgeR v3.30.3.

Correlation of Expression Between DE nORFs and their Associated DE TEs

Spearman and Pearson correlation coefficients and their corresponding p-values were calculated for the normalized counts for each DE nORF-DE TE combination (each DE nORF may be associated with many DE TEs). Expression of a DE nORF and its associated DE TE within a DE nORF-DE TE combination was defined to be significantly correlated if the absolute Spearman and Pearson correlation coefficients were above 0.5 and significant (p<0.05) for the DE nORF-DE TE combination.

Proteogenomic Analysis to Demonstrate Translation of Transcribed nORFs

Proteogenomic analysis to demonstrate evidence of translation of the transcribed nORFs was performed using the amino acid sequence of all the 248,135 nORFs or transcripts assembled from a subset of PsychENCODE samples, which are part of Stanley Medical Research Institute (SMRI) Array Collection. For this subset, we had matching raw transcriptomic and proteomic data; however, from different (adjacent) regions of prefrontal cortex (BA46 and BA10 respectively).

Analysis of Transcripts from SMRI Array Collection Samples

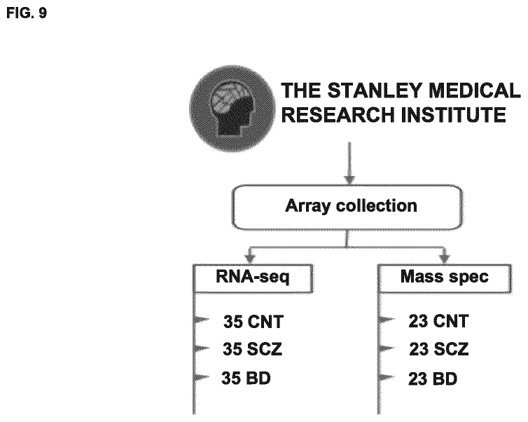

RNA-Seq data from BA46 of post-mortem brain samples, classified as Array Collection by SMRI, was obtained upon request. This comprised of 23 SCZ, 23 CNT and 16 BD samples—after matching with proteomic samples and removing any outliers (FIG. 9).

In brief, the RNA extraction was performed as follows. 1 μg of total RNA was poly-A selected using oligo-dT Dynabeads, libraries were prepared using Illumina's TruSeq v1 (Illumina, Hayward, CA) and sequencing was performed using Illumina HiSeq 2000 giving ˜3 Mb of 90 bp paired-end reads for each library. The resultant.FASTQ/.FQ files were processed as described below.

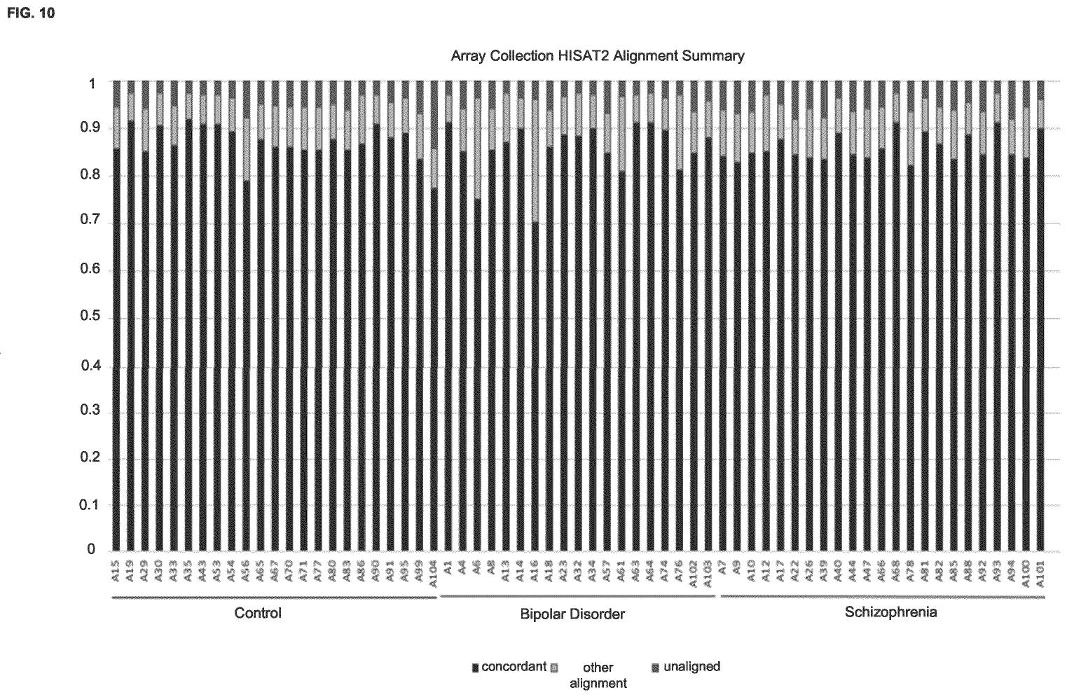

The .FASTQ/.FQ were assessed using FastQC for quality control. Read alignment was carried out using HISAT2 v2.1.0 with default parameters except ‘--add-chrname’, ‘-dta’ and ‘--summary-file’ were set to TRUE. Additionally, either Phred+33 or Phred+64 encoding was set to TRUE based on the sample being analyzed. Reads were aligned using the index for GRCh38 genome available at ccb.jhu.edu/software/hisat2/manual.shtml. The resultant summary file was used to generate counts of percentage read alignment (FIG. 10).

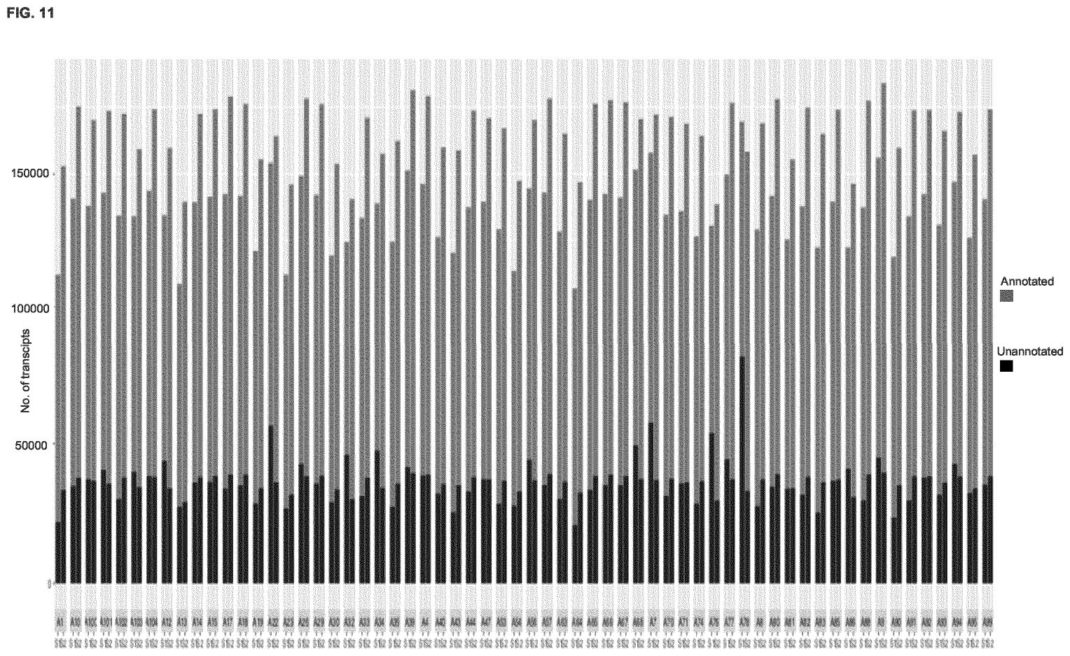

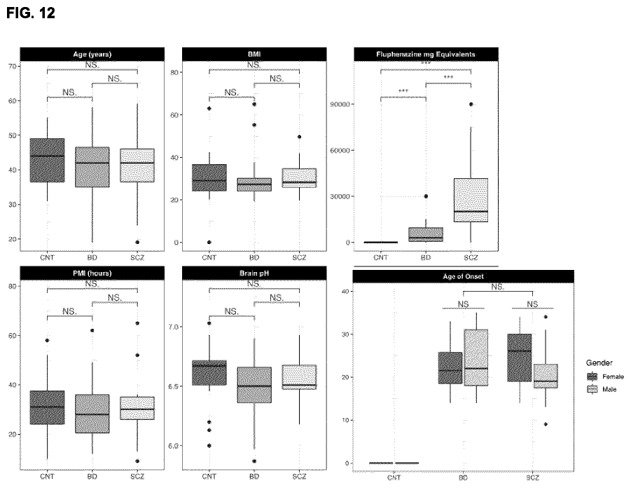

Following alignment, transcripts were assembled using StringTie v1.3.3 (FIG. 11). First, StringTie was run with default parameters and ‘-A’ set to TRUE to assemble sample-specific transcripts from the aligned reads (.BAM files), using gencode V30 primary comprehensive gene annotation (www.gencodegenes.org/human/) as reference. Second, all the .GTF files generated in the previous step were merged using StringTie-merge to create a union transcript dataset. Third, StringTie was rerun on the aligned reads with StringTie merged file as the reference and parameters ‘-B’, ‘-e’ and ‘-A’ set to TRUE, allowing us to calculate sample-specific transcript abundances for the union transcript dataset. Transcripts were filtered to retain only those from chromosomes 1-22, X and Y, with TPM >0.1 in at least 25% samples and no PAR (pseudoautosomal regions) suffix to the transcript IDs. Once again nORFs within this subset of samples were identified using the GffCompare workflow, as described. HISAT2 and StringTie runs were conducted on the cloud server platform provided by Seven Bridges Genomics. Sample metadata was analyzed for potential confounders using Mann-Whitney U pair-wise test for continuous data and Fisher's test or Chi-square test for categorical data in R. Significance was assigned as ‘*’ for p-value <0.05, ‘**’ for p-value <0.01, ‘***’ for p-value <0.001 and N.S. for non-significant p-values (FIG. 12).

Analysis of Mass Spectra from SMRI Array Collection Samples

Post-mortem anterior prefrontal cortex (BA10) samples were obtained from 23 SCZ, 23 BD and 23 control samples (after matching with RNA-seq data this led to the use of 23 SCZ, 16 BD and 23 CNT samples). 50 mg of tissue slices per sample were collected and processed. Protein samples were analyzed using Waters Q-TOF premier mass spectrometer. The output .RAW files were processed on PLGS and converted to .MGF files. The .MGF files were searched against the human UniProt database using Mascot to identify known proteins that are translated.

### Proteogenomics Analysis

Unmapped mass spectra were searched against two databases using Mascot. The first search was carried out against nORF amino acid database that was constructed using 248,135 nORFs that we curated.

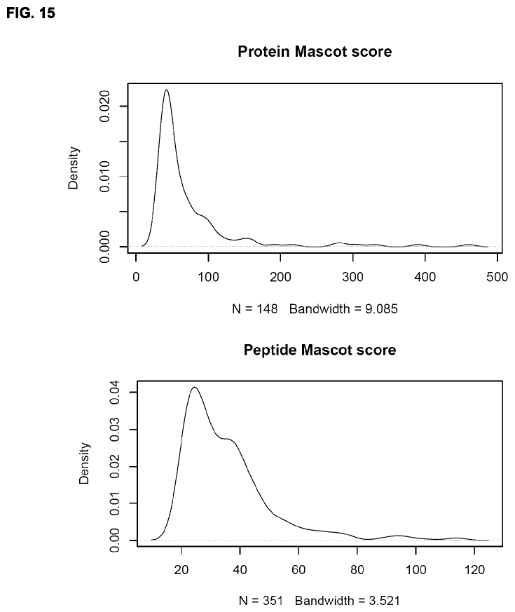

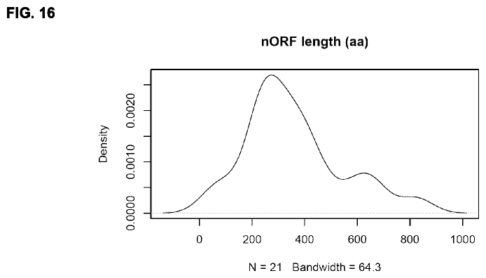

The results of mapping unmatched sample spectra to nORF amino acid database were filtered by protein and peptide score >50 and expectation value <0.05. Furthermore, only peptides expressed in at least 30% of each disorder group were evaluated (FIGS. 14A-16). Expression of the identified nORF proteins were evaluated across different metadata sets namely, gender, psychosis and suicide. Significant difference in the presence of a nORF protein between the metadata categories were determined using Chi-square goodness of fit test. Significance was determined as * for p-value <0.05, ** for p-value <0.01 and *** for p-value <0.001. Similar analysis was performed for additional novel peptides identified after spectra matching to the transcriptomic database. Additionally, to confirm that the identified peptides are novel, a protein environment was manually curated from the genes in the vicinity of the identified peptide. Finally, each peptide was matched to the curated protein sequences to retain unmapped and unique novel peptides.

Enrichment Analysis to Identify Potential Functions of nORFs

InterProScan was used to identify descriptive GO terms for the nORFs used in this study, and GO enrichment was performed using GOEnrichment tool available via usegalaxy.org. Next, using the GLANET tool for annotation and enrichment analysis, DHS1, TFs and histone modification enrichment was evaluated for nORFs. Default parameters were used, and 10,000 samples were processed across 30 core processors.

Potential Structures of Identified nORFs

Structures for 21 nORFs that were identified using proteogenomic analysis, and DE nORFs identified in BD or SCZ, were generated using 1-TASSER and RaptorX. Default parameters were used for the structure prediction run. For 1-TASSER, the model with the highest confidence score was chosen as the nORF structure. Models were visualized using Avogadro or Jena3D viewer.

Correlation Analysis of the Translated nORFs with Psychosis, Suicide and Gender

Expression of the 21 translated nORFs were compared for differences (presence/absence evaluated as Yes/No) between gender, incidence of psychosis and suicide. Significance was evaluated using a Chi-squared test for each disorder or inter-disorder. P-value significances were evaluated at 3 levels: ***<0.001; **<0.01; *<0.05 C. Similarly, the three new nORFs identified using transcriptomic data were compared for differences between gender, incidence of psychosis and suicide.

### Results

Creation of nORF Database and Classification of nORF Entries

We added ‘low-noise’ nORFs, as defined and identified across 353 samples from the RPFdbv2.0 using the RibORF tool to˜194,407 nORF entries from nORFs.org. Briefly, low-noise nORFs were identified as those with lower standard deviation of their RPKM read counts to that of the median deviation of canonical ORFs or cORFs (the main ORFs within protein-coding genes). This resulted in 248,135 nORF entries (GRCh38; 247,404 entries in nORF hg19) after removal of nORFs that were in-frame with the cORFs as determined by a classification scheme (FIG. 7A). These nORF coordinates were then extensively pre-processed to remove duplicates and re-classified based on their genomic locations with respect to known genes.

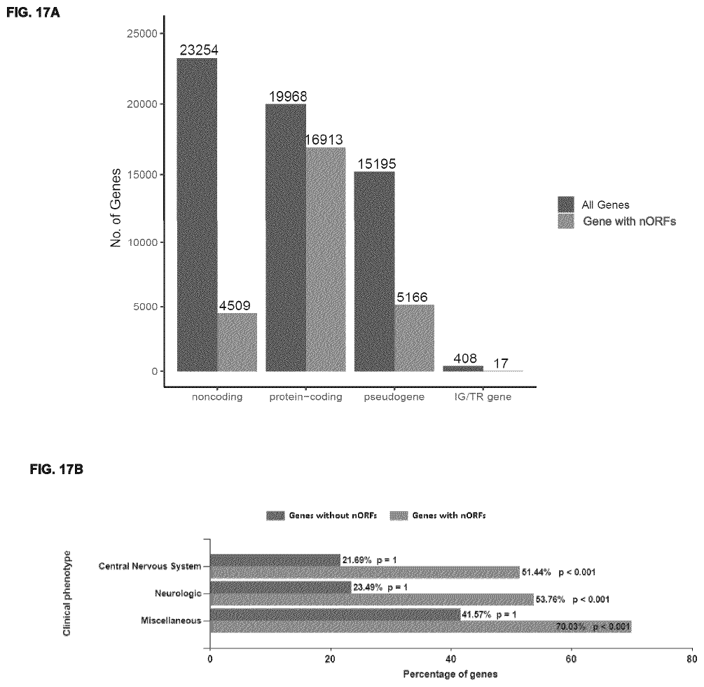

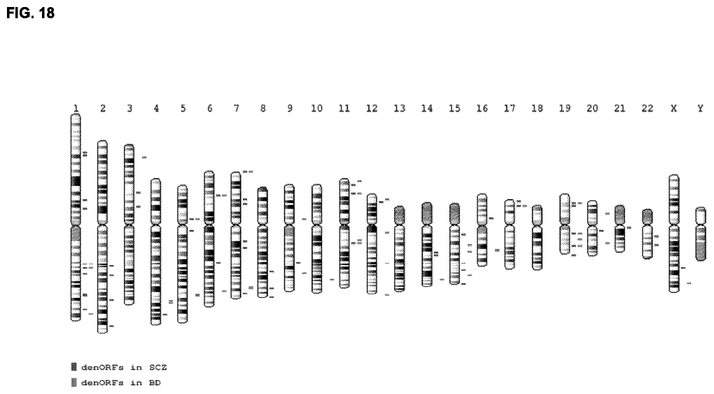

Classification of the 248,135 nORF entries with respect to known genes (FIG. 7B) was based on whether they are in-frame or in alternative frame to its corresponding known protein coding genes. Approximately, 42% of the nORFs in the dataset were identified to be within the coding sequence (CDS) of a protein-coding gene, but in an alternative frame. FIG. 17A displays the number of nORFs localized within known genes classified based on biotypes. Furthermore, we evaluated the potential function of protein-coding genes using FunRich v3.1.3 and genes with nORFs were identified to be associated significantly more with neurological disorders as shown in FIG. 17B. This analysis indicates that disruption of putative nORF functions could be involved in neuropsychiatric disorders, such as SCZ and BD, which may lead to new diagnostic and therapeutic opportunities.

Identification of DE nORFs in PsychENCODE Dataset

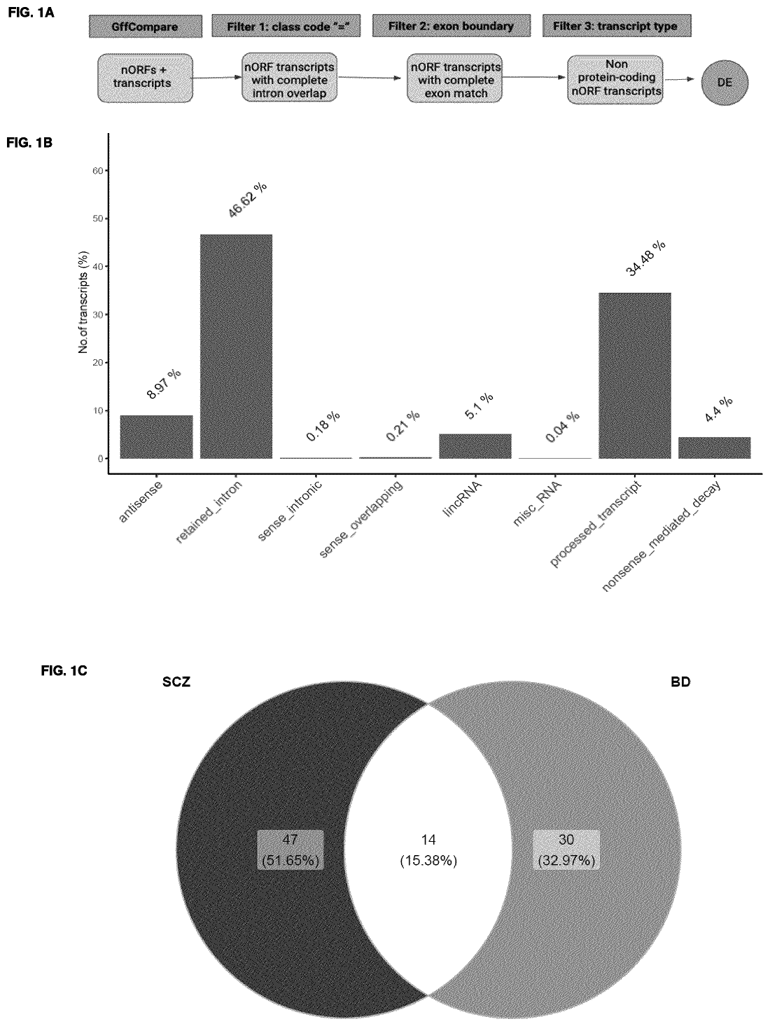

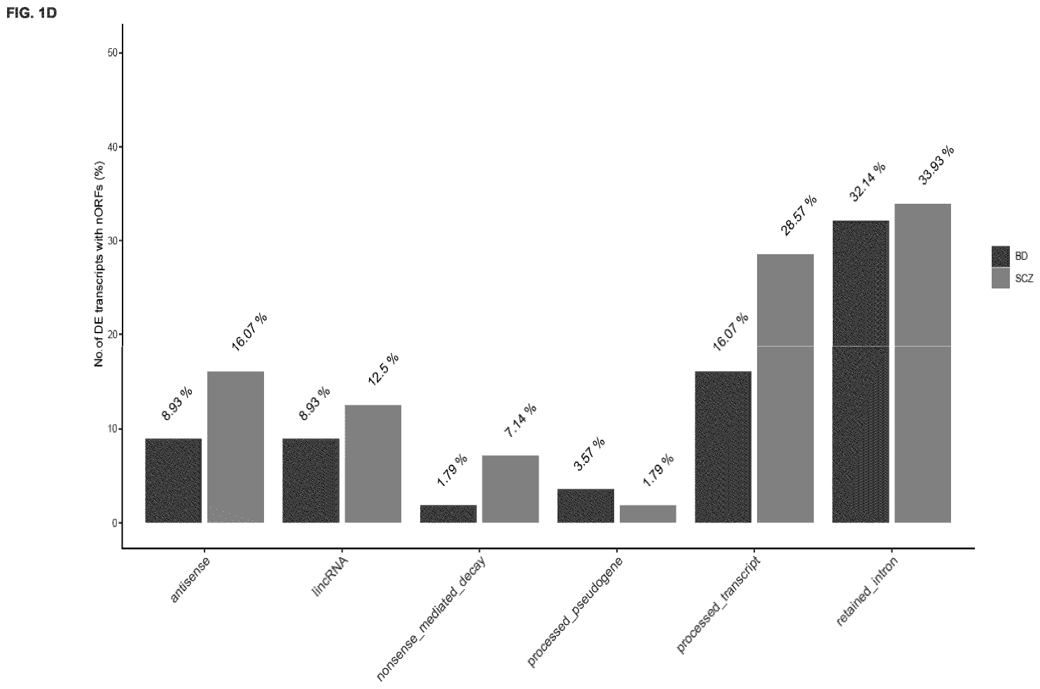

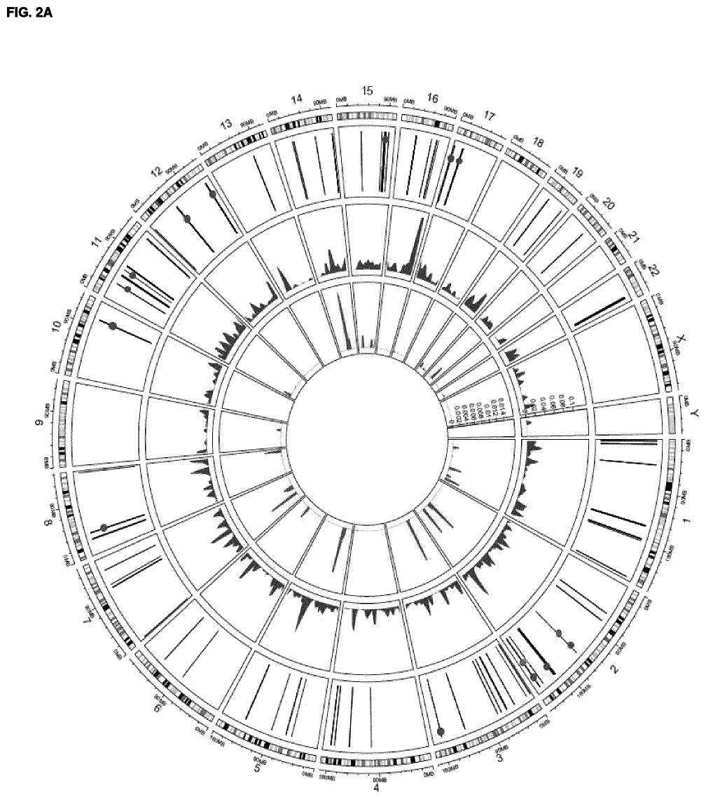

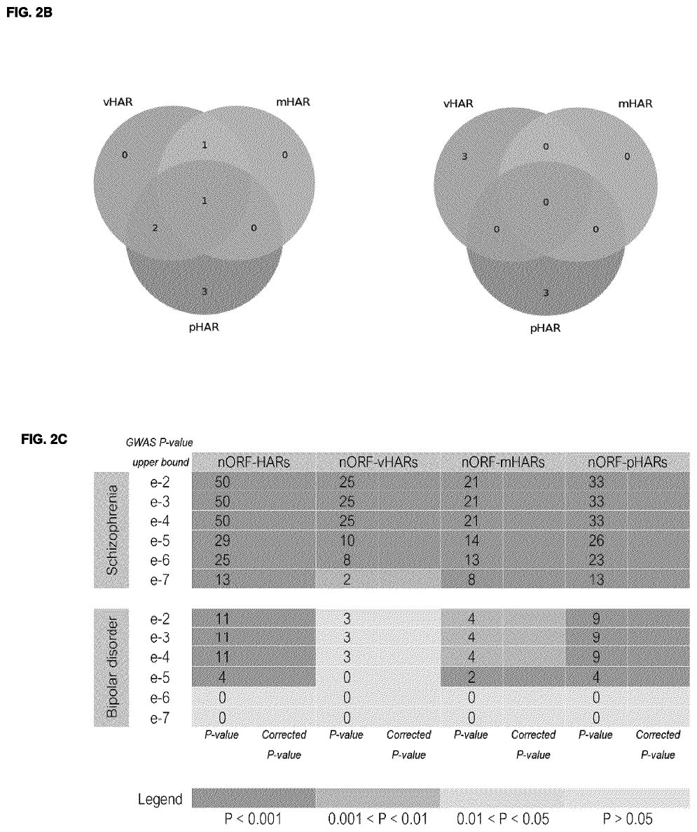

To investigate whether the 248,135 nORFs that we curated are transcribed in PsychENCODE samples, and whether they are up- or down-regulated compared to the control samples, we performed the following set of analyses. Transcripts from the three sample groups were pre-processed, as described, and their abundance was obtained and filtered to retain those with transcripts per million (TPM) >0.1 in at least 10% of the samples, resulting in 110,003 transcripts. We identified 3,103 nORFs using the workflow illustrated in FIG. 1A, with ˜46% within retained introns and ˜34% within processed transcripts (FIG. 1B). To identify differentially expressed nORFs we used linear mixed-effects models as it accounts for random effects. This analysis revealed that 2,935 and 1,689 transcripts containing 56 SCZ and 40 BD nORFs, respectively, are differentially expressed, as outlined in Tables 2 and 3, below. As indicated in Table 2, 1689 and 2935 transcripts were DE in BD and SCZ, respectively. As indicated in Table 3, 40/1689 and 56/2935 transcripts identified as DE in BD and SCZ, respectively, contain nORFs. 14 DE nORFs were common to both the disorders (FIG. 1C), indicating the overlap of the pathophysiology of the two disorders, and ˜30% of the DE nORFs are ‘retained’ introns (FIG. 1D). FIG. 18 illustrates the location of DE nORFs on all chromosomes.

TABLE 2
Results of the differential expression analysis for SCZ and
BD against CNT samples at 0.05% FDR with the corresponding
number of upregulated and downregulated transcripts

| | DE transcripts | Upregulated | Downregulated |
|---|---|---|---|
| Condition | (FDR <0.05) | transcripts | transcripts |
| BD/CNT | 1689 | 843 | 846 |
| SCZ/CNT | 2935 | 1263 | 1672 |

TABLE 3
Number of nORFs contained within DE transcripts
identified in neuropsychiatric samples

| | DE transcripts | Upregulated | Downregulated |
|---|---|---|---|
| Condition | containing nORFs | transcripts | transcripts |
| BD/CNT | 40/1689 | 21 | 19 |
| SCZ/CNT | 56/2935 | 25 | 31 |

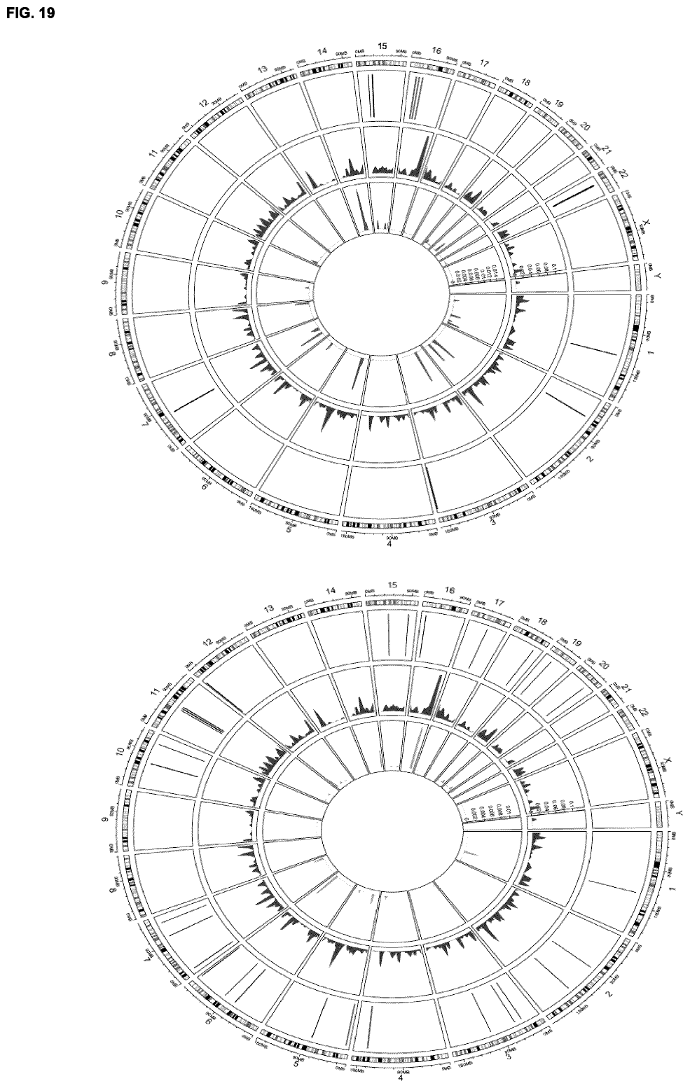

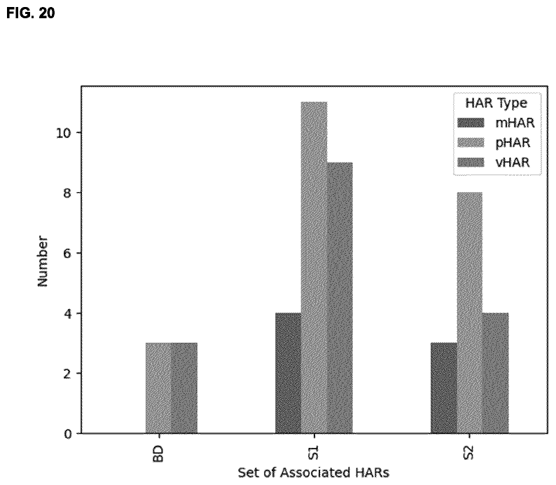

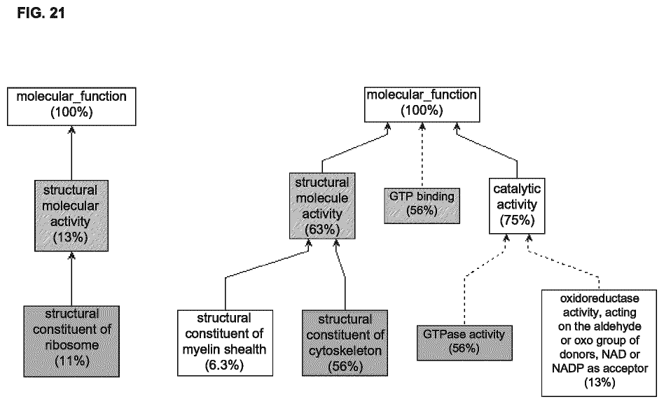

Next, we intended to investigate similar relationships between differential expression of nORFs in SCZ and BD and their association with the respective disease pathology. Because there is no equivalent metric to patient survival, we explored whether the identified DE nORFs, in the respective disorders, are associated with already identified genomic ‘hot-spots’ for the respective disorders. To do this, we used GLANET, a program that associates nORFs with genomic loci that are implicated in SCZ and BD and tests for the statistical significance of the enrichments. FIGS. 2A and 19 display the results of this analysis as circular plots. If nORF enrichment was identified, the corresponding loci (vertical line) is marked with circular dots—enrichment for nORFs that are transcribed is depicted as blue circles and enrichment for nORFs that are differentially expressed is depicted as red circles. It is interesting to note that two SCZ loci within chromosome 2 (FIG. 2A) are enriched for nORFs DE in SCZ. Similar analyses conducted for BD-specific loci and SCZ-specific CNVs (copy-number variations) showed no nORF enrichment (FIG. 19).

nORFs-HARs and their Enrichments within Disorder-Associated SNP Loci

Having demonstrated that some nORFs are indeed associated with SCZ hot spots, we performed the following analysis to investigate whether the nORFs constitute recently evolved vHARs, mHARs, and pHARs genomic regions. Out of 3,103 nORFs, 431 nORFs overlapped with 4,481 unique HARs. Seven nORFs DE in SCZ (3 over-expressed and 4 under-expressed) were found to be associated with HARs (7 DE nORF-HARs); most associated HARs resided within the same characterized region as their nORF, but some were found in intergenic regions or in different genes. Six nORFs DE in BD (4 over-expressed and 2 under-expressed in BD) were found to be associated with HARs (6 DE nORF-HARs); again, most associated HARs resided within the same characterized region as their nORF, but some were found in intergenic regions or in different genes.

The transcript types of the 7 DE nORF-HARs in SCZ are—2 ‘antisense’, 2 ‘processed transcripts’, 1 ‘nonsense mediated decay’, 1 ‘retained intron’ and 1 ‘lincRNA.’ 2 DE nORFs contained HARs within them: tracer_65443 and fs1rH2. The transcript types of the 6 DE nORF-HARs in BD are—3 ‘retained intron’, 1 ‘lincRNA’, 1 ‘processed pseudogene’ and 1 ‘antisense’. No nORFs contained HARs within their lengths. The HAR types associated with DE nORFs in SCZ and The HAR types associated with DE nORFs in BD are displayed in FIG. 2B (left and right panel respectively).

INRICH analysis revealed that out of the 431 nORF-HARs, 50 are associated with SCZ loci with a GWAS p-value upper bound of 10⁻²; 13 nORF-pHARs were associated with SCZ loci with a GWAS p-value upper bound of 10⁻⁷. Furthermore 11 nORF-HARs are associated with BD loci with a GWAS p-value upper bound of 10⁻², and only 4 nORF-pHARs were associated with BD loci with a GWAS p-value upper bound of 10⁻⁵ (FIG. 2C). The DE nORF tracer_65443 and its parent gene ZEB2 were both within a SCZ-associated locus (SNP locus that involved SNPs with p-value 10⁻⁷<P<10⁻⁶). The parent gene (SCL7A6OS) of one DE nORF (tracer_42939) was within a SCZ-associated locus p<10⁻⁷ and was also associated with a BD-associated SNP locus that involved SNPs with 10⁻⁵<P<10⁻⁴. This is consistent with phenotypic overlap between the two disorders, suggesting some commonality in the causes behind the two disorders. The association of DE nORF-pHARs enriched in SCZ loci suggests that these DE nORFs and their functions may have arisen in primates and then been subject to increased evolution in the human lineage, only to result in SCZ susceptibility in modern humans when dysfunctional.

### DE HARs and DE TEs

HARs and TEs that were included in or overlapped with these DE transcripts were designated as differentially expressed HARs or DE HARs and differentially expressed TEs, or DE TEs, respectively. 160 DE transcripts in SCZ contained HARs resulting in 305 DE HARs in SCZ; 59 DE transcripts in BD contained HARs resulting in 90 DE HARs in BD; 2,638 DE transcripts in SCZ contained TEs resulting in 193,111 DE TEs in SCZ; and 1,522 DE transcripts in BD contained TEs, giving 100,831 DE TEs in BD.

Association of DE nORFs with Differentially Expressed HARs (DE HARs)

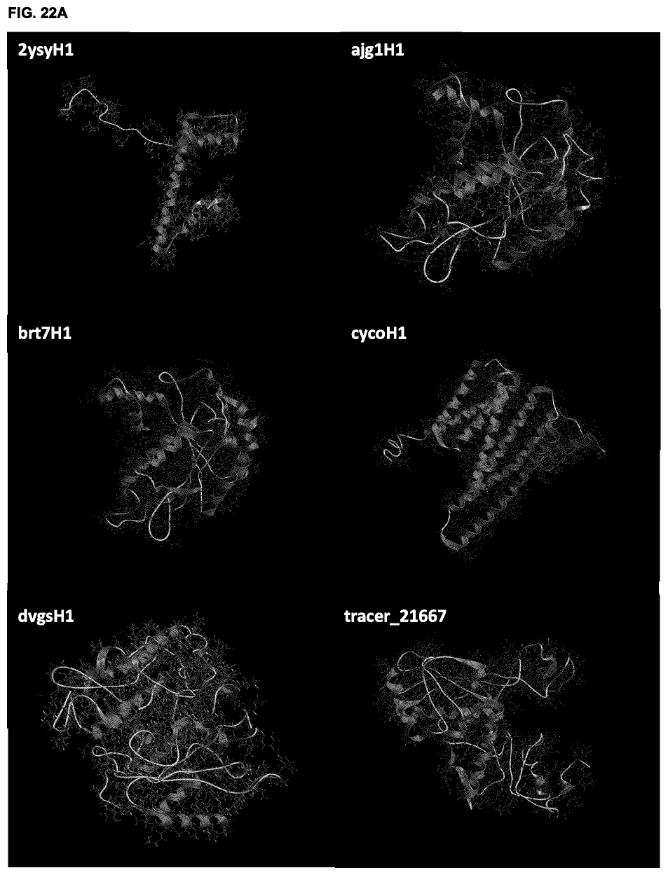

While most HARs are considered non-coding genomic regions, they do demonstrate evidence of transcription. RNAs containing HARs fall under various classifications of non-coding RNA—small RNA (sRNA), microRNA (miRNA), long non-coding RNA (lncRNA) or enhancer RNA (eRNA)—or may simply be a part of a known protein-coding region. If a DE HAR associated with a DE nORF is within a known protein-coding region, that could indicate a potential connection between that protein-coding region and the DE nORF. Three DE nORFs were found to be associated with DE HARs in SCZ (3 DE nORF-DE HARs); none were found in BD (FIG. 20). As the set of transcripts used to identify DE HARs was also used to identify the DE nORFs, the two DE nORFs that contain HARs within their lengths were identified as DE nORF-DE HARs. The third DE nORF-DE HAR had its DE HAR in a gene different to the DE nORF (tracer_87517). None of the three DE nORF-DE HAR were within SCZ-associated loci.

Association of DE nORFs with Differentially Expressed TEs (DE TEs)

Presence of a TE in the 2 kb region upstream of a DE nORF could indicate the presence of an alternative promoter. Therefore, DE nORFs were investigated for association with DE TEs based on the condition that DE nORFs are associated with a DE TE if the TE is within the 2 Kb region upstream of the nORF. 11 DE nORFs were found to be associated with DE TEs in SCZ (11 DE nORF-DE TEs); and 8 DE nORFs were found to be associated with DE TEs in BD (8 DE nORF-DE TEs). Of the 8 DE nORFs associated with DE TEs in BD, 2 are also associated with HARs: cp2xH1 and eveeH1. DE TEs could allow for different expression of nORFs under different conditions, leading to phenotypes of SCZ or BD. Besides differential expression-based regulation we also investigated whether there could be other unknown correlations between expression of TEs and nORFs. To understand this, we performed Spearman and Pearson correlation analysis of the expression of nORFs and each of their associated DE TEs.

This analysis revealed that 5 DE nORF-DE TE combinations had significantly correlated expression levels in SCZ. Notably, the DE nORF 2vnjH1 had its expression significantly correlated with two DE TEs: one LINE and one SINE. One DE nORF was overexpressed in SCZ; 4 DE nORFs were under-expressed in SCZ. The DE nORFs' biotypes were split into 2 ‘lincRNA’, 2 ‘processed transcripts’ and 1 ‘antisense’. None of the DE nORFs were within SCZ-associated loci. The 5 DE TEs were all unique and were comprised of 2 3′-end-of-a-L2 LINEs, 2 L2-end SINEs and 1 3′-end-of-a-L1 LINE. For BD, 4 DE nORF-DE TE combinations were found to have significantly correlated expressions. Of the 4 DE nORFs, 2 were found to be associated with HARs as well. The 4 DE nORFs' biotypes were split evenly between ‘retained intron’ and ‘lincRNA’. None of the DE nORFs were within BD-associated loci. The 4 DE TEs were also all unique and were comprised of 2 ERV1 LTRs, 1 Alu SINE and 1 3′-end-of-a-L1 LINE. 3 DE nORFs were upregulated in BD; 1 DE nORF was down regulated. The DE nORFs included cp2xH1 and eveeH1, which were also associated with HARs, suggesting that those DE nORFs were under HAR-related selection pressure as well as being regulated by TEs. This association is perhaps most significant for eveeH1 and is interesting given the parent gene of eveeH1 is ZNF84, a zinc finger protein that contains a KRAB/FPB domain that may regulate gene expression through TE regulation. As such, eveeH1 may serve as an initial regulation point from which other TE-associated genes and nORFs may be regulated. Its associated DE TE with correlated expression is an endogenous retrovirus sequence ERV1 conserved in primates; its insertion may have conferred an added layer of regulation that was later selected for along with the associated HAR, perhaps in part due to its far-reaching effects. 1 DE nORF-DE TE combination with significantly correlated expression was shared between the SCZ and BD datasets: tracer_18675 with its L1MC2 TE. The expression of the DE TE was correlated with the expression of the DE nORF. This was the only DE nORF-DE TE combination with significant overlap between the DE TE and the DE nORF.

Translation Evidence of nORFs in Brain Samples

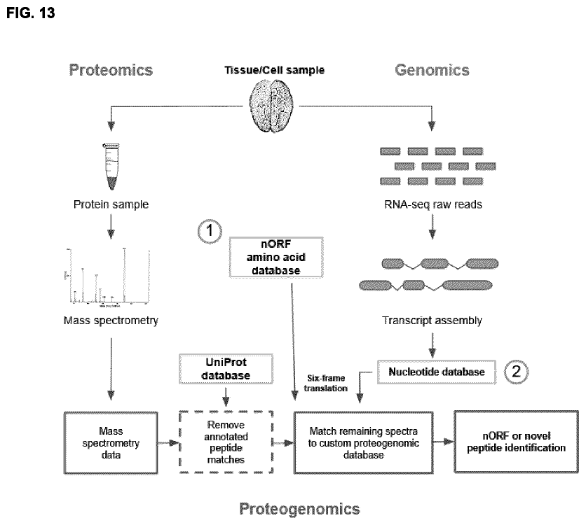

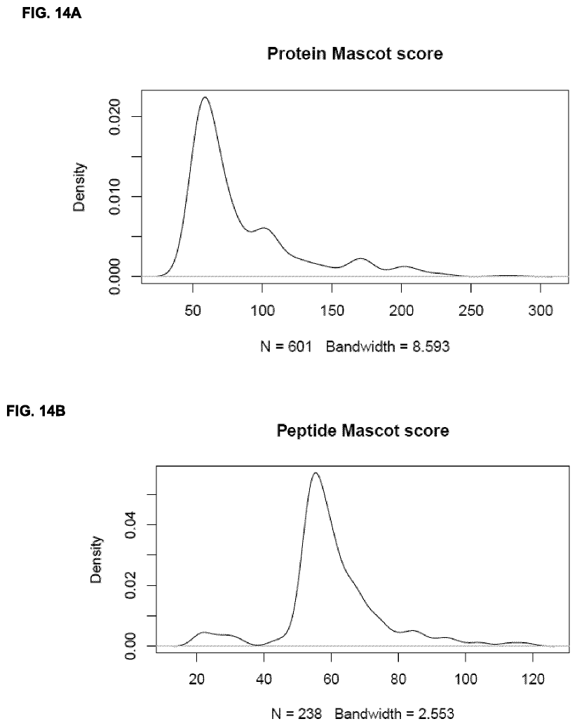

We aimed to obtain direct evidence of translation of these nORFs in SCZ and BD brain samples. To this end, we used a proteogenomic approach that combines both transcriptomic and proteomic data. We performed the proteogenomics analysis on a subset of samples for which both transcriptomic and proteomic data were available, which were suitable for investigating potential translation of nORFs. Transcriptomic and proteomic data from this subset of 62 samples from the SMRI Array cohort was analyzed using the proteogenomic framework as described in the methods and displayed in FIG. 13.

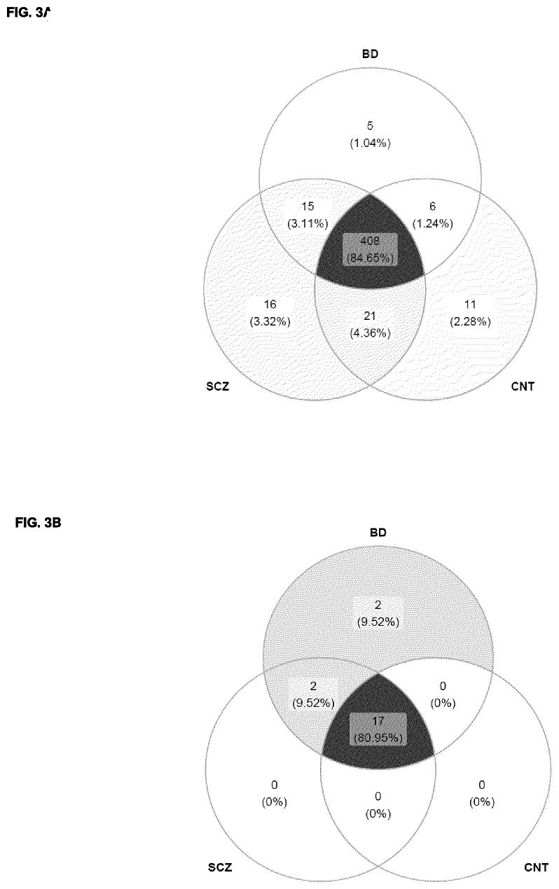

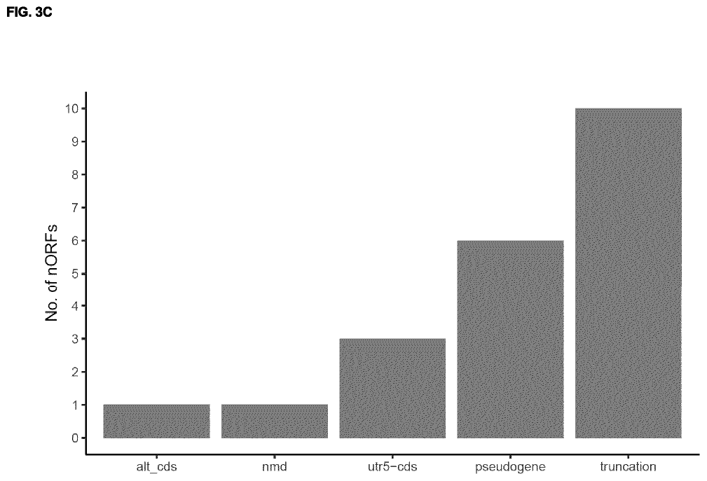

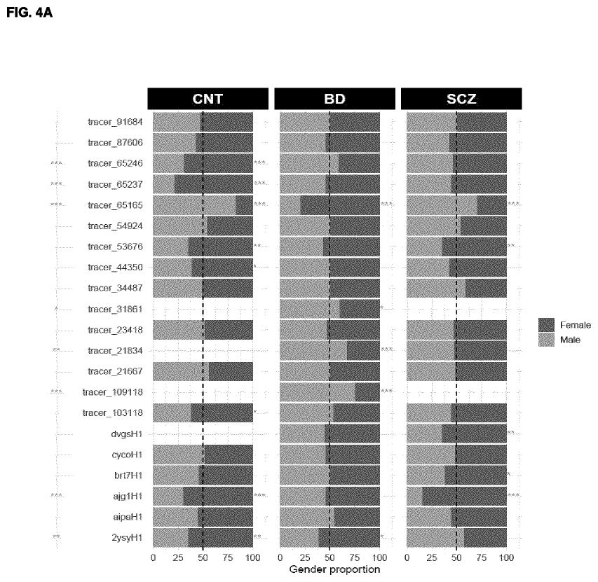

The proteogenomic analysis identified 446, 460 and 434 known proteins that were translated in CNT, SCZ and BD, respectively, among these 408 are common between all three sample sets (FIG. 3A). The results were filtered to retain entries with a peptide expectation score <0.05 and a peptide score >50, which were expressed in at least 30% of samples from each of the three groups. Additionally, each peptide entry that passed the filtration criteria was evaluated manually for novelty by matching against all known protein fragments. As a result, 21 nORFs from the list of 248,135 nORFs were identified as translated along with three novel ones, which were identified from the transcriptomic data. However, these three novel peptides were identified within four of the 21 nORFs. The nORFs and peptides that mapped to the nORFs are listed in Table 4.

TABLE 4
nORF Sequences

| | | SEQ ID | |
|---|---|---|---|
| nORF | nORF Sequence | NO: | Peptides identified |
| tracer_ | MDSVRSGPFGQIFRPDNFVFGQSG | 1 | LHFFMPGFAPLTSR (SEQ ID NO: |
| 91684 | AGNNWAKGHYTEGAELVDSVLDVV | | 23), LTTPTYGDLNHLVSATMSGVTTCLR |
| | RKESESCDCLQGFQLTHSLGGGTG | | (SEQ ID NO: 24), |
| | SGMGTLLISKIREEYPDRIMNTFS | | SGPFGQIFRPDNFVFGQSGAGNNWAK |
| | VMPSPKVSDTVVEPYNATLSVHQL | | (SEQ ID NO: 25), |
| | VENTDETYSIDNEALYDICFRTLK | | NSSYFVEWIPNNVK (SEQ ID NO: |
| | LTTPTYGDLNHLVSATMSGVTTCL | | 26), MSATFIGNSTAIQELFK (SEQ ID |
| | RFPGQLNADLRKLAVNMVPFPRLH | | NO: 27), GHYTEGAELVDSVLDVVR |
| | FFMPGFAPLTSRGSQQYRALTVPE | | (SEQ ID NO: 28), EVDEQMLNVQNK |
| | LTQQMFDSKNMMAACDPRHGRYLT | | (SEQ ID NO: 29), |
| | VAAIFRGRMSMKEVDEQMLNVQNK | | ALTVPELTQQMEDSK (SEQ ID NO: |
| | NSSYFVEWIPNNVKTAVCDIPPRG | | 30), LAVNMVPFPR (SEQ ID NO: |
| | LKMSATFIGNSTAIQELFKRISEQ | | 31), IMNTFSVMPSPK (SEQ ID NO: |
| | FTAMFRRKAFLHWYTGEGMDEMEF | | 32), ISEQFTAMFR (SEQ ID NO: |
| | TEAESNMNDLVSEYQQYQDATADE | | 33) |
| | QGEFEEEEGEDEA | | |
| tracer_ | LPSLRLLHRRRPLQVLVMRECISI | 2 | LISQIVSSITASLR (SEQ ID NO: |
| 23418 | HVGQAGVQIGNACWELYCLEHGIQ | | 34), AVFVDLEPTVIDEVR (SEQ ID |
| | PDGQMPSDKTIGGGDDSFNTFFSE | | NO: 35), TIGGGDDSFNTFFSETGAGK |
| | TGAGKHVPRAVFVDLEPTVIDEVR | | (SEQ ID NO: 36), DVNAAIATIK |
| | TGTYRQLFHPEQLITGKEDAANNY | | (SEQ ID NO: 37), TIQFVDWCPTGFK |
| | ARGHYTIGKEIIDLVLDRIRKLAD | | (SEQ ID NO: 38), |
| | QCTGLQGFLVFHSFGGGTGSGFTS | | FDGALNVDLTEFQTNLVPYPR (SEQ ID |
| | LLMERLSVDYGKKSKLEFSIYPAP | | NO: 39), NLDIERPTYTNLNR (SEQ |
| | QVSTAVVEPYNSILTTHTTLEHSD | | ID NO: 40), EIIDLVLDR (SEQ ID |
| | CAFMVDNEAIYDICRRNLDIERPT | | NO: 41), EDAANNYAR (SEQ ID NO: |
| | YTNLNRLISQIVSSITASLRFDGA | | 42), IHFPLATYAPVISAEK (SEQ ID |
| | LNVDLTEFQTNLVPYPRIHFPLAT | | NO: 43) |
| | YAPVISAEKAYHEQLTVAEITNAC | | |
| | FEPANQMVKCDPRHGKYMACCLLY | | |
| | RGDVVPKDVNAAIATIKTKRTIQF | | |
| | VDWCPTGFKVGINYQPPTVVPGGD | | |
| | LAKVQRAVCMLSNTTAVAEAWARL | | |
| | DHKFDLMYAKRAFVHWYVGEGMEE | | |
| | GEFSEAREDMAALEKDYEEVGADS | | |
| | ADGEDEGEEY | | |
| tracer_ | MPSDKTIGGGDDSFNTFFSETGAG | 3 | AVCMLSNTTAIAEAWAR (SEQ ID |
| 65246 | KHVPRAVFVDLEPTVVDEVRTGTY | | NO: 44), |
| | RQLFHPEQLITGKEDAANNYARGH | | TIGGGDDSFNTFFSETGAGK (SEQ ID |
| | YTIGKEIVDLVLDRIRKLADLCTG | | NO: 45), DVNAAIATIK (SEQ ID |
| | LQGFLIFHSFGGGTGSGFASLLME | | NO: 46), TIQFVDWCPTGFK (SEQ |
| | RLSVDYGKKSKLEFAIYPAPQVST | | ID NO: 47), |
| | AVVEPYNSILTTHTTLEHSDCAFM | | AYHEQLSVAEITNACFEPANQMVK |
| | VDNEAIYDICRRNLDIERPTYTNL | | (SEQ ID NO: 48), |
| | NRLIGQIVSSITASLRFDGALNVD | | FDGALNVDLTEFQTNLVPYPR (SEQ |
| | LTEFQTNLVPYPRIHFPLATYAPV | | ID NO: 49), NLDIERPTYTNLNR |
| | ISAEKAYHEQLSVAEITNACFEPA | | (SEQ ID NO: 50), EDAANNYAR |
| | NQMVKCDPRHGKYMACCMLYRGDV | | (SEQ ID NO: 51), |
| | VPKDVNAAIATIKTKRTIQFVDWC | | IHFPLATYAPVISAEK (SEQ ID NO: |
| | PTGFKVGINYQPPTVVPGGDLAKV | | 52) |
| | QRAVCMLSNTTAIAEAWARLDHKF | | |
| | DLMYAKRAFVHWYVGEGMEEGEFS | | |
| | EAREDLAALEKDYEEVGVDSVEAE | | |
| | AEEGEEY | | |
| tracer_ | MFCPVEGSSENKTIDFDSLSVGRG | 4 | FQLTDCQIYEVLSVIR (SEQ ID NO: |
| 103118 | SGQVVAQQRDVAHLGPDPQPPYSR | | 53), GTVVYGEPITASLGTDGSHYWSK |
| | QGRRAGGEPSVESGRKVEIRRASG | | (SEQ ID NO: 54), |
| | KEALQNINDQSDRLLIKGGKIVND | | ILDLGITGPEGHVLSRPEEVEAEAVNR |
| | DQSFYADIYMEDGLIKQIGENLIV | | (SEQ ID NO: 55), |
| | PGGVKTIEAHSRMVIPGGIDVHTR | | AALAGGTTMIIDHVVPEPGTSLLAAFDQWR |
| | FQMPDQGMTSADDFFQGTKAALAG | | (SEQ ID NO: 56), |
| | GTTMIIDHVVPEPGTSLLAAFDQW | | FQMPDQGMTSADDFFQGTK (SEQ ID |
| | REWADSKSCCDYSLHVDISEWHKG | | NO: 57), AITIANQTNCPLYITK (SEQ |
| | IQEEMEALVKDHGVNSFLVYMAFK | | ID NO: 58), THNSSLEYNIFEGMECR |
| | DRFQLTDCQIYEVLSVIRDIGAIA | | (SEQ ID NO: 59), |
| | QVHAENGDIIAEEQQRILDLGITG | | AAAFVTSPPLSPDPTTPDFLNSLLSCGDLQ |
| | PEGHVLSRPEEVEAEAVNRAITIA | | VTGSAHCTENTAQK (SEQ ID NO: |
| | NQTNCPLYITKVMSKSSAEVIAQA | | 60), IVLEDGTLHVTEGSGR (SEQ ID |
| | RKKGTVVYGEPITASLGTDGSHYW | | NO: 61), GLYDGPVCEVSVTPK (SEQ |
| | SKNWAKAAAFVTSPPLSPDPTTPD | | ID NO: 62), |
| | FLNSLLSCGDLQVTGSAHCTFNTA | | NLHQSGFSLSGAQIDDNIPR (SEQ ID |
| | QKAVGKDNFTLIPEGTNGTEERMS | | NO: 63) |
| | VIWDKAVVTGKMDENQFVAVTSTN | | |
| | AAKVFNLYPRKGRIAVGSDADLVI | | |
| | WDPDSVKTISAKTHNSSLEYNIFE | | |
| | GMECRGSPLVVISQGKIVLEDGTL | | |
| | HVTEGSGRYIPRKPFPDFVYKRIK | | |
| | ARSRLAELRGVPRGLYDGPVCEVS | | |
| | VTPKTVTPASSAKTSPAKQQAPPV | | |
| | RNLHQSGFSLSGAQIDDNIPRRTT | | |
| | QRIVAPPGGRANITSLG | | |
| tracer_ | MNTFSVVPSPKVSDTVVEPYNATL | 5 | LHFFMPGFAPLTSR (SEQ ID NO: |
| 54924 | SVHQLVENTDETYCIDNEALYDIC | | 64), LTTPTYGDLNHLVSATMSGVTTCLR |
| | FRTLKLTTPTYGDLNHLVSATMSG | | (SEQ ID NO: 65), |
| | VTTCLRFPGQLNADLRKLAVNMVP | | ALTVPELTQQMEDAK (SEQ ID NO: |
| | FPRLHFFMPGFAPLTSRGSQQYRA | | 66), NSSYFVEWIPNNVK (SEQ ID |
| | LTVPELTQQMFDAKNMMAACDPRH | | NO: 67), MAATFIGNSTAIQELFK |
| | GRYLTVAAVFRGRMSMKEVDEQML | | (SEQ ID NO: 68), LAVNMVPFPR |
| | SVQSKNSSYFVEWIPNNVKTAVCD | | (SEQ ID NO: 69), ISEQFTAMER |
| | IPPRGLKMAATFIGNSTAIQELFK | | (SEQ ID NO: 70) |
| | RISEQFTAMFRRKAFLHWYTGEGM | | |
| | DEMEFTEAESNMNDLVSEYQQYQD | | |
| | ATAEEGEFEEEAEEEVA | | |
| brt7H1 | MREIVHIQAGQRGNQIGAKFWEVI | 6 | SGPFGQIFRPDNFVFGQSGAGNNWAK |
| | SDEHGIDPTGTYHGDSDLQLDRIS | | (SEQ ID NO: 71), IMNTFSVVPSPK |
| | VYYSEATDGKYVPRAILVDLEPGT | | (SEQ ID NO: 72), |
| | MDSVRSGPFGQIFRPDNFVFGQSG | | GHYTEGAELVDSVLDVVR (SEQ ID NO: |
| | AGNNWAKGHYTEGAELVDSVLDVV | | 73), AILVDLEPGTMDSVR (SEQ ID |
| | RKEAESCDCLQGFQLTHSLGGGTG | | NO: 74), |
| | SGMGTLLISKIREEYPDRIMNTFS | | EAESCDCLQGFQLTHSLGGGTGSGMGTLLI |
| | VVPSPKVSDTVIESYNATLSVHQL | | SK (SEQ ID NO: 75), |
| | VENTDETYCIDNEALYDICFRTLR | | FWEVISDEHGIDPTGTYHGDSDLQLDR |
| | VTTPTYGDLNHLL | | (SEQ ID NO: 76) |
| tracer_ | MRDPSKIKWGDAGAEYVVESTGVF | 7 | VPTANVSVVDLTCR (SEQ ID NO: |
| 21667 | TTMEKAGAHLQGGAKRVIISAPSA | | 77), LISWYDNEFGYSNR (SEQ ID |
| | DAPMFVMGVNHEKYDNSLKIISNA | | NO: 78), |
| | SCTTNCLAPLAKVIHDNFGIVEGL | | VIHDNFGIVEGLMTTVHAITATQK (SEQ |
| | MTTVHAITATQKTVDGPSGKLWRD | | ID NO: 79), |
| | GRGALQNIIPASTGAAKAVGKVIP | | VIISAPSADAPMFVMGVNHEK (SEQ ID |
| | ELNGKLTGMAFRVPTANVSVVDLT | | NO: 80), IISNASCTTNCLAPLAK |
| | CRLEKPAKYDDIKKVVKQASEGPL | | (SEQ ID NO: 81), |
| | KGILGYTEHQVVSSDFNSDTHSST | | WGDAGAEYVVESTGVFTTMEK (SEQ ID |
| | FDAGAGIALNDHFVKLISWYDNEF | | NO: 82) |
| | GYSNRVVDLMAHMASKE | | |
| cycoH1 | MDKNELVQKAKLAEQAERYDDMAA | 8 | TAFDEAIAELDTLSEESYK (SEQ ID |
| | CMKSVTEQGAELSNEERNLLSVAY | | NO: 83), GIVDQSQQAYQEAFEISK |
| | KNVVGAHRSSWRVVSSIEQKTEGA | | (SEQ ID NO: 84), |
| | EKKQQMAREYREKIETELRDICND | | SVTEQGAELSNEER (SEQ ID NO: |
| | VLSLLEKFLIPSASQAESKVFYLK | | 85), DICNDVLSLLEK (SEQ ID NO: |
| | MEGDYYRYLAEVAAGDDKKGIVDQ | | 86) |
| | SQQAYQEAFEISKKEMQPTHPIRL | | |
| | GLALNFSVFYYEILNSPEKACSLA | | |
| | KTAFDEAIAELDTLSEESYKDSTL | | |
| | IMQLLRDNLTLWTSDTQADEGEAG | | |
| | EGVEN | | |
| tracer_ | MGQKDSYVGDEAQSKRGILTLKYP | 9 | SYELPDGQVITIGNER (SEQ ID NO: |
| 34487 | IEHGIITNWDDMEKIWHHTFYNEL | | 87), YPIEHGIITNWDDMEK (SEQ ID |
| | RVAPEEHPTLLTEAPLNPKANREK | | NO: 88) |
| | MTQIMFETFNVPAMYVAIQAVLSL | | |
| | YASGRTTGIVLDSGDGVTHNVPIY | | |
| | EGYALPHAIMRLDLAGRDLTDYLM | | |
| | KILTERGYSFVTTAEREIVRDIKE | | |
| | KLCYVALDFENEMATAASSSSLEK | | |
| | SYELPDGQVITIGNERFRCPETLF | | |
| | QPSFIGMESAGIHETTYNSIMKCD | | |
| | IDIRKDLYANNVLSGGTTMYPGIA | | |
| | DRMQKEITALAPSTMKIKIIAPPE | | |
| | RKYSVWIGGSILASLSTFQQMWIS | | |
| | KQEYDEAGPSIVHRKCF | | |
| 2ysyH1 | MTKAQKKDGKKRKRGRKESYSIYV | 10 | AMGIMNSEVNDIFER (SEQ ID NO: |
| | YKVLKQVHPDTGISSKAMGIMNSF | | 89) |
| | VNDIFERIASEASRLAHYKQALHH | | |
| | HVPRSADGRAPAAARRAGQARRVR | | |
| | GH | | |
| dvgsH1 | MRECVSIHVGQAGVQIGNVCWELYC | 11 | SIQFVDWCPTGFK (SEQ ID NO: |
| | LEHGIQPDGQMPSDKTIGGGDDSFN | | 90), EDAANNYAR (SEQ ID NO: |
| | TFSETGAGKHVPRAVFVDLEPMVID | | 91) |
| | EVCTGTYRQLFHPEQLITGKEDAAN | | |
| | NYARGHYTIGKEIIDLVLDRIRKLA | | |
| | NQCTGFQGFLVFHSFGGGTGSGFTS | | |
| | LLIERLLVDYGKKSKLEFSNYPAPQ | | |
| | VSTAVVEPYNSILTTHTTLEHSDCA | | |
| | FMVDNEAICDICCRNLNIERPTYTN | | |
| | LNHLISQIVSSITASLRFDGALNVD | | |
| | LTEFQTNLVPYSHIHFPLATYAPVI | | |
| | SAEKAYHEQLSVAEITNASFEPANQ | | |
| | MVKCDPRHGKYMACCLLCHGDVVPK | | |
| | DANAAIATIKTKRSIQFVDWCPTGF | | |
| | KVGINYQSPTVVPGGDLAMVQSAC | | |
| tracer_ | MNTFSVVPSPKVSDTVVEPYNATL | 12 | ALTVPELTQQMFDAK (SEQ ID NO: |
| 44350 | SIHQLVENTDETYCIDNEALYDIC | | 92), NSSYFVEWIPNNVK (SEQ ID |
| | FRTLKLATPTYGDLNHLVSATMSG | | NO: 93), LAVNMVPFPR (SEQ ID |
| | VTTSLRFPGQLNADLRKLAVNMVP | | NO: 94), |
| | FPRLHFFMPGFAPLTARGSQQYRA | | LATPTYGDLNHLVSATMSGVTTSLR (SEQ |
| | LTVPELTQQMFDAKNMMAACDPRH | | ID NO: 95), ISEQFTAMFR (SEQ ID |
| | GRYLTVATVFRGRMSMKEVDEQML | | NO: 96) |
| | AIQSKNSSYFVEWIPNNVKVAVCD | | |
| | IPPRGLKMSSTFIGNSTAIQELFK | | |
| | RISEQFTAMFRRKAFLHWYTGEGM | | |
| | DEMEFTEAESNMNDLVSEYQQYQD | | |
| | ATAEEEGEMYEDDEEESEAQGPK | | |
| tracer_ | MAKALLLYGADIESKNKHGLTPLLL | 13 | QEYDESGPSIVHR (SEQ ID NO: 97), |
| 65237 | GVHEQKQQVVKFLIKKKANLNALDR | | SYELPDGQVITIGNER (SEQ ID NO: |
| | YGRTALILAVCCGSASIVSLLLEQN | | 98), AGFAGDDAPR (SEQ ID NO: |
| | IDVSSQDLSGQTAREYAVSSHHHVI | | 99) |
| | CQLLSDYKEKQMLKISSENSNPEQE | | |
| | LKLTSEEESQRFKGSENSQPEKMSQ | | |
| | ELEINKDGDREVEEEMKKHESNNVG | | |
| | LLENLTNGVTAGNGDNGLIPQRKSR | | |
| | TPENQQFPDNESEEYHRICELLSDY | | |
| | KEKQMPKYSSENSNPEQDLKLTSEE | | |
| | ESQRLKGSENGQPEKRSQEPEINKD | | |
| | GDRELENFMAIEEMKKHGSTHVGFP | | |
| | ENLTNGATAGNGDDGLIPPRKSRTP | | |
| | ESQQFPDTENEEYHSDEQNDTQKQF | | |
| | CEEQNTGILHDEILIHEEKQIEVVE | | |
| | KMNSELSLSCKKEKDVLHENSTLRE | | |
| | EIAMLRLELDTMKHQSQLREKKYLE | | |
| | DIESVKKKNDNLLKALQLNELTMDD | | |
| | DTAVLVIDNGSGMCKAGFAGDDAPR | | |
| | AVFPSIVGRPRQQGMMGGMHQKESY | | |
| | VGKEAQSKRGILTLKYPMEHGIITN | | |
| | WDDMEKIWHHTFYNELRVAPEEHPI | | |
| | LLTEAPLNPKANREKMTQIMFETFN | | |
| | TPAMYVAIQAVPSLYTSGRTTGIVM | | |
| | DSGDGVTHTVPIYEGNALPHATLRL | | |
| | DLAGRELPDYLMKILTERGYRFTTM | | |
| | AEREIVRDIKEKLCYVALDFEQEMA | | |
| | TAASSSSLEKSYELPDGQVITIGNE | | |
| | RFRCPEALFQPCFLGMESCGIHETT | | |
| | FNSIMKSDVDIRKDLYTNTVLSGGT | | |
| | TMYPGMAHRMQKEIAALAPSMMKIR | | |
| | IIAPPKRKYSVWVGGSILASLSTFQ | | |
| | QMWISKQEYDESGPSIVHRKCF | | |
| tracer_ | LDVMASQKRPSQRHGSKYLATAST | 14 | YLATASTMDHAR (SEQ ID NO: 100), |
| 53676 | MDHARHGFLPRHRDTGILDSIGRF | 15 | TQDENPVVHFFK (SEQ ID NO: 101), |
| | FGGDRGAPKRGSGKDSHHPARTAH | | GVDAQGTLSK (SEQ ID NO: 102) |
| | YGSLPQKSHGRTQDENPVVHFFKN | | |
| | IVTPRTPPPSQGKGRGLSLSRFSW | | |
| | VGDERTSIGFLFRPSPHSPATFVF | | |
| | CSVSVASWPPFLSSRDLSAHSWDR | | |
| | AALSSTGEMKRGLPFRKGAEGQRP | | |
| | GFGYGGRASDYKSAHKGFKGVDAQ | | |
| | GTLSKIFKLGGRDSRSGSPMARR | | |
| tracer_ | LTPRSSSPWFSRPNVARLSVRMSA | | FEELNADLER (SEQ ID NO: 103), |
| 31861 | RGPAIGIDLGTTYSCVGVFQHGKV | | STAGDTHLGGEDFDNR (SEQ ID NO: |
| | EIIANDQGNRTTPSYVAFTDTERL | | 104), TTPSYVAFTDTER (SEQ ID |
| | IGDAAKNQVAMNPTNTIFDAKRLI | | NO: 105) |
| | GRKFEDATVQSDMKHWPFRVVSEG | | |
| | GKPKVQVEYKGETKTFFPEEISSM | | |
| | VLTKMKEIAEAYLGGKVHSAVITV | | |
| | PAYFNDSQRQATKDAGTITGLNVL | | |
| | RIINEPTAAAIAYGLDKKGCAGGE | | |
| | KNVLIFDLGGGTFDVSILTIEDGI | | |
| | FEVKSTAGDTHLGGEDFDNRMVSH | | |
| | LAEEFKRKHKKDIGPNKRAVRRLR | | |
| | TACERAKRTLSSSTQASIEIDSLY | | |
| | EGVDFYTSITRARFEELNADLFRG | | |
| | TLEPVEKALRDAKLDKGQIQEIVL | | |
| | VGGSTRIPKIQKLLQDFFNGKELN | | |
| | KSINPDEAVAYGAAVQAAILIGDK | | |
| | SENVQDLLLLDVTPLSLGIETAGG | | |
| | VMTPLIKRNTTIPTKQTQTFTTYS | | |
| | DNQSSVLVQVYEGERAMTKDNNLL | | |
| | GKFDLTGIPPAPRGVPQIEVTEDI | | |
| | DANGILNVTAADKSTGKENKITIT | | |
| | NDKGRLSKDDIDRMVQEAERYKSE | | |
| | DEANRDRVAAKNALESYTYNIKQT | | |
| | VEDEKLRGKISEQDKNKILDKCQE | | |
| | VINWLDRNQMAEKDEYEHKQKELE | | |
| | RVCNPIISKLYQGGPGGGSGGGGS | | |
| | GASGGPTIEEVD | | |
| aipaH1 | MVYMFLYDSTHGKFHGTIKAENGK | 16 | LVINGNPITIFQER (SEQ ID NO: |
| | LVINGNPITIFQERDPSKIKWGTA | | 106), VLHDNFGIVKGLMTTVHAITATQK |
| | GAEYIVEPTGTFTTMEKTGAHLQG | | (SEQ ID NO: 107) |
| | GAKRVIISAPSADASVFIMGVNNE | | |
| | KYDNSLKIISNASCTTNCLAPRAK | | |
| | VLHDNFGIVKGLMTTVHAITATQK | | |
| | TVDGPSRKLWHDGGGTLQNIIPAS | | |
| | TGTAKAVGKVIPELNGKLTGMAFC | | |
| | VPTANVSVVDLIYHLEKPTKYHGI | | |
| | KKVVKEASEGSL | | |
| tracer_ | MEGAGGGSVKHEDAVLLLQDDRHH | 17 | AAHVFFTDSCPDALFNELVK (SEQ ID |
| 109118 | DRGHNESRIAGSHVVEDINKRREP | | NO: 108), |
| | LPSLEAVYLITPSEKSVHSLISDF | | LIQHAQIPPEDSEIITNMAHLGVPIVTDST |
| | KDPPTAKYRAAHVFFTDSCPDALF | | LR (SEQ ID NO: 109) |
| | NELVKSRAAKVIKTLTEINIAFLP | | |
| | YESQVYSLDSADSFQSFYSPHKAQ | | |
| | MKNPILERLAEQIATLCATLKEYP | | |
| | AVRYRGEYKDNALLAQLIQDKLDA | | |
| | YKADDPTMGEGPDKARSQLLILDR | | |
| | GFDPSSPVLHELTFQAMSYDLLPI | | |
| | ENDVYKYETSGIGEARVKEVLLDE | | |
| | DDDLWIALRHKHIAEVSQEVTRSL | | |
| | KDFSSSKRMNTGEKTTMRDLSQML | | |
| | KKMPQYQKELSKYSTHLHLAEDCM | | |
| | KHYQGTVDKLCRVEQDLAMGTDAE | | |
| | GEKIKDPMRAIVPILLDANVSTYD | | |
| | KIRIILLYIFLKNGITEENLNKLI | | |
| | QHAQIPPEDSEIITNMAHLGVPIV | | |
| | TDSTLRRRSKPERKERISEQTYQL | | |
| | SRWTPIIKDIMEDTIEDKLDTKHY | | |
| | PYISTRSSASFSTTAVSARYGHWH | | |
| | KNKAPGEYRSGPRLIIFILGGVSL | | |
| | NEMRCAYEVTQANGKWEVLIGSTH | | |
| | ILTPQKLLDTLKKLNKTDEEISS | | |
| ajg1H1 | MQSHGVVLFSFQFWEVISDEHGID | 18 | INVYYNEAAGNK (SEQ ID NO: 110), |
| | PTGSYHGDSDLQLERINVYYNEAA | | GHYTEGAELVDSVLDVVR (SEQ ID NO: |
| | GNKYVPWAILVDLEPGTTDSIRSG | | 111) |
| | PFGQIYRPDNFVFGQSGAGNNWAK | | |
| | GHYTEGAELVDSVLDVVRKESESC | | |
| | DCLQGFQLTHSLGGGTGSGMGTLL | | |
| | ISKIREEYPDRIMNSFSVMPSPKV | | |
| | SDTVVEPYNATLSVHQLVENTDET | | |
| | YCIDNEALYDICFRTLKLTTPTYG | | |
| | TSTTWCRPP | | |
| tracer_ | MDVAASEFYRDGKYDLDFKSPTDP | 19 | YITGDQLGALYQDFVR (SEQ ID NO: |
| 21834 | SRYITGDQLGALYQDFVRDYPGNK | | 112) |
| | GACSCLM | | |
| tracer_ | MIGRPRHQGVMVGMGQKDCYVGDE | 20 | SYELPDGQVITIGNER (SEQ ID NO: |
| 87606 | AQSKRGVLTLKYPIEHGVVTNWDD | | 113), HQGVMVGMGQK (SEQ ID |
| | MEKIWYHTFYNELRVAPDEHPILL | | NO: 114), VAPDEHPILLTEAPLNPK |
| | TEAPLNPKINREKMTQIMFEAFNT | | (SEQ ID NO: 115) |
| | PAMYVAIQAVLSLYASGRTTGIVM | | |
| | DSGDGVTHIVPIYEGYALPHAILR | | |
| | LDLAGRDLTDYLMKILTERGYNFT | | |
| | TTAEREIVRDVKEKLCYVALDFEQ | | |
| | EMVRAAASSSPERSYELPDGQVIT | | |
| | IGNERFRCPEAIFQPSFLGIESSG | | |
| | IHETTFNSIMKCDVDIRKDLYANT | | |
| | VLSGGSTMYPGIADRMQKEIITLA | | |
| | PSTMKIKIIAPPERKYSVWIGGSI | | |
| | LASLSTFQQMWISKQEYDEAGPPI | | |
| | VHRKCF | | |
| tracer_ | MPSDKTIGGGDDSFNTFFSETGAG | 21 | AVCMLSNTTAIAEAWAR (SEQ ID NO: |
| 65165 | KHVPRAVFVDLEPTVVDEVRTGTY | | 116), TIGGGDDSFNTFFSETGAGK |
| | RQLFHPEQLITGKEDAASNYARGH | | (SEQ ID NO: 117), |
| | YTIGKEIVDLVLDRIRKLADLCTG | | TIQFVDWCPTGFK (SEQ ID NO: |
| | LQGFLIFHSFGGGTGSGFASLLME | | 118), NLDIERPTYTNLNR (SEQ ID |
| | RLSVDYSKKSKLEFAIYPAPQVST | | NO: 119), |
| | AVVEPYNSILTTHTTLEHSDCAFM | | AYHEQLSVAEITNACFEPANQMVK (SEQ |
| | VDNEAIYDICRRNLDIERPTYTNL | | ID NO: 120), LIGQIVSSITASLR |
| | NRLIGQIVSSITASLRFDGALNVD | | (SEQ ID NO: 121), |
| | LTEFQTNLVPYPRIHFPLATYAPV | | IHFPLATYAPVISAEK (SEQ ID NO: |
| | ISAEKAYHEQLSVAEITNACFEPA | | 122), DVNAAIATIK (SEQ ID NO: |
| | NQMVKCDPRHGKYMACCMLYRGDV | | 123) |
| | VPKDVNAAIATIKTKRTIQFVDWC | | |
| | PTGFKVGINYQPPTVVPGGDLAKV | | |
| | QRAVCMLSNTTAIAEAWARLVHKF | | |
| | DLMYAKWAFVHWYVGEGMEEGEFS | | |
| | EAREDLAALEKDCEEVGVDSVEAE | | |
| | AEEGEAY | | |

17 of the 21 nORDs identified as translated were common between CNT, SCZ and B whereas 2 were unique to B8 and 2 to SCZ and B (FIG. 3B and as shown below in Table 5). Ten out of the 21 nORF proteins were annotated as truncations of known proteins and 6 as pseudogenes (FIG. 3C). nORs uniquely expressed in SCZ or BD (2 common to SCZ and Be and 2 unique to BD) were present within genes such as syntaxin (a presynaptic membrane protein) binding protein (STXBP1), heat shock protein (HSPA2), and DISC1 fusion partner (DISC1IFP1), some of them are associated with SCZ and BD. We found 1 nORF-ajg1H1, that had evidence of both transcription and translation and contained a tubulin domain as determined using InterProScan.

TABLE 5
Characterization of disorder-specific translated nORFs.

| nORF ID | Disorder | Classification | Chromosome | Nearby gene |
|---|---|---|---|---|
| tracer_109118 | BD | nmd | 9 | STXBP1, syntaxin binding |
| | | | | protein |
| tracer_31861 | BD | utr5-cds | 14 | HSPA2: Heat shock protein |
| | | | | family A (Hsp70) member 2 |
| tracer_21834 | BD, SCZ | nmd (truncation) | 12 | ENO2: enolase 2 |
| dvgsH1 | BD, SCZ | pseudogene | 11 | TUBAP2: tubulin alpha |
| | | | | pseudogene 2 and larger |
| | | | | DISC1FP1: DISC 1 Fusion |
| | | | | partner 1 |

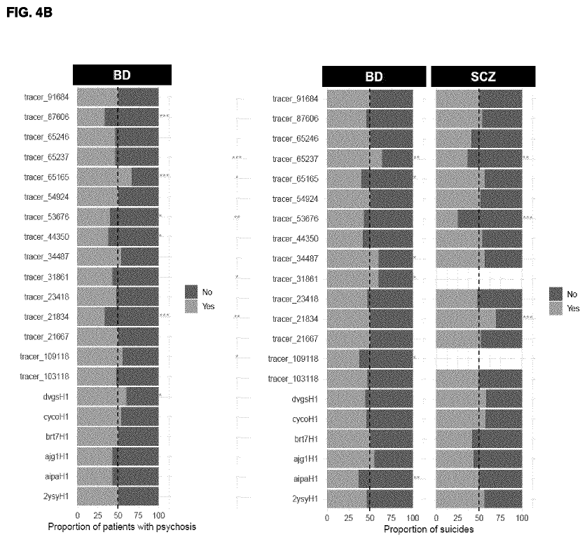

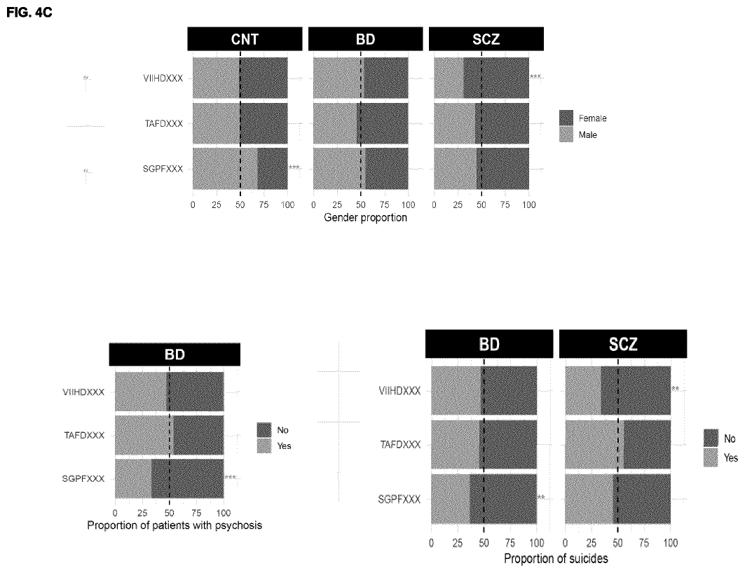

We further evaluated the expression differences of these novel peptides between disorders for metadata categories such as suicide, psychosis and gender and identified significant expression differences as determined using Chi-squared tests (FIG. 4). We found that eight of the 21 nORFs were significantly associated with gender, six of the 21 nORF were significantly associated with psychosis in BD, and six of the 21 nORFs were significantly associated with suicide in SCZ and BD. Among the three additional nORFs peptides, two were significantly different between the genders, one was significantly associated with psychosis, and two were significantly associated with suicide. This analysis revealed that nORFs expression can be used to stratify or diagnose patients who might develop psychosis or who might be prone to suicide based on their expression.

Gene Ontology Enrichment Analysis for Potential Functional Inferences of nORFs

To infer functions of the translated nORFs from their amino acid sequence we performed gene ontology (GO) analysis. For all the 248,135 nORFs used in this study, GO terms were obtained using InterProScan and GO term enrichment was performed using GOEnrichment tool via the galaxy server (FIG. 21). For the 3,103 nORFs with evidence of transcription, structural molecular activity within ribosomes and therefore, potential involvement in translation was found. For nORFs that were DE, no enrichment was found. nORFs identified as translated within samples showed enrichment for structural molecular activity as part of the myelin sheath and cytoskeleton, GTP binding, GTPase and other oxidoreductase activity.

The GLANET analysis, in addition to associating nORFs with the SCZ and BD disorder associated loci, also identified enrichment of certain DNase I hypersensitive site (DHS1), histone modifications and transcription factors (TFs) within the transcribed and DE nORFs, as shown in Table 6 below.

TABLE 6
Enrichment of genomic elements within nORFs.

| nORF | DNase I | Histone | Transcription |
|---|---|---|---|
| Category | Hypersensitive Site | Modification | Factors |
| All transcribed (3103) | 82 | 140 | 322 |
| DE BD (44) | 3 | 94 | 57 |
| DE SCZ (61) | 63 | 106 | 73 |

The nucleotide sequence (transcript) for the 40 transcripts, which contain 44 differentially expressed nORFS (amino acid sequences) mapped to these transcripts from BD are shown in Table 7 and Table 8, respectively.

The nucleotide sequence (transcript) for the 56 transcripts, which contain 61 differentially expressed nORFS (amino acid sequences) mapped to these transcripts from SCZ are shown in Table 9 and Table 10, respectively.

The nucleotide sequence (transcript) for all 3022 transcripts contained 3103 differentially expressed nORFS (amino acid sequences).

TABLE 7
DE BD nORF transcript sequences

| | SEQ | |
|---|---|---|
| | ID | |
| nORF | NO: | nucleotide sequence |
| >ENST00000495397.1:: | 124 | AAGGTGGTCTACAAGCTTCACTACTACCACGACGGCCAGGCCGTGCGCTACTTCCACTCCAGCGCCAACTACACTGTG |
| chr1: 161695690- | | TTACAGGCGCGTGCCAGCGACAGCGGGCGCTACCAGTGCTCGGGCACCATGCGCATCCCGGTGGAGAGCGCGCCCA |
| 161697932(+) | | TGTTCTCCGCTAAGGTGGCTGTGACAGTGCAAGGTGGGAGAGACCAGGGGCCCCGGGAGGGAGGCAAATGAGCATTG |
| | | AGAAATTCTGGGACAGGAGCTCGGCGAGAAAAGAAGGGGCGGAAGTTCAAATAGCTGCCACTTGGAGGGTTTCTCTTA |
| | | GACTATGGACGCTGTCTCCTCTCCTTTGCCGTGAAGCAGCGCTTGATCCGCCGCCTGCTTGGAGGCTGGTCCCTTTCC |
| | | CCGACGCACATCCTGGCTTCTCACTCTGGCTGGGGACTCCCACAGAGAGGGCAGCTCTGGCAGGGGCAGGGGCCACT |
| | | GTGGGACTGGGACGAAGGAGCGACTCCCTAGCGTCCTGCAAGGCAGGCGCAGCGTCTCCTATTCTAGGCTGCAGAAC |
| | | CCGAGCTGAGTGTCAGTCGGGATGTGACATGAAGCGTCTGGCCTGGTCCCTCTTCCTTTCAAGCTTTCCCCGTCCCTC |
| | | GTGGACTCGGTCCCCCTGCCCCACATTTCAGAAGGCTCCCCTTCCCCCTCCACGTGGACACACGGCCTCCTCCCCTCC |
| | | CCCCTTGGTCTGTGGGTCTGCAAGGAGCCCTCGCGGGAAGCAGGAAGGAGCGGGGTCGCGGAGCGGTGGACAAGCC |
| | | GGCGCCGTTGCTCCCCGCCCTCTCCGTAGAGCTGTTCCGGGCGCCGGTGCTGAGGGTGATGGGTCCGCGGGAGGCC |
| | | CGCGGCGCGGCGCTGGGTGGGGTGGTGCTGCGCTGCGACACGCGCCTGCACCCGCAGAAGCGCGACACGCCGCTG |
| | | CAGTTCGCGTTTTACAAGTACAGCCGCGCGGTGCGCCGCTTCGACTGGGGCGCCGAGTACACAGTCCCGGAGCCCGA |
| | | GGTCGAGGAGCTCGAATCGTACTGGTGCGAGGCGGCTACCGCCACCCGCAGTGTCCGGAAACGCAGTCCGTGGCTG |
| | | CAGCTCCCGGGGCCGGGTTCTCCCCTGGACCCGGCCTCCACCACCGCCCCAGCTCCATGGGCCGCAGCCTTGGCTC |
| | | CTGGTAATAGGCCGCTTTCCTTCAGAAAGCCCCCGGTGTCCAGATCGGTCCCGTTGGTCACCTCCGTCCGGAACACCA |
| | | CCTCCACCGGGCTGCAGTTCCCGGCGAGCGGCGCCCCGACTGCGGGGCCACCCGCCTGCGCTCCGCCGACGCCCTT |
| | | GGAACAATCGGCTGGAGCCCTGAAACCCGACGTGGACCTTCTGCTCCGAGAAATGCAGCTGCTCAAAGGCCTTCTGAG |
| | | CCGGGTGGTCCTGGAATTAAAGGAGCCACAGGCCCTCCGGGAGCTCAGGGGAACGCCCGAGACCCCCACCTCTCACT |
| | | TTGCTGTGAGCCCGGGAACCCCAGAGACCACTCCTGTGGAGAGCTGAGGGGGGGGCTACCGTCCCCTCTGCAGGCTC |
| | | ATTCCTCCTTGGTCTCCTGCTTCCCCTCACGCGAATTTCTTTCAAAGCCATCTGTTTGCATCCTTGTGTTTTGCTGTGGTT |
| | | TTTAAAGGAGCGCCCACGAAGTGTAGTGGCTGACGATTTCAACCTCACACAGCAGTTTGTAACCGCAAGCATTCTCTTT |
| | | GAATTCTCACAGAATTCAGCAAGAAGTAGAAACCTGTTATTTACTACATTGTGATTTAACTTTGGATGTGAATTTAGTCAC |
| | | CCTTAGCCCTTCAGATAAGCCTAGCCAGTACATATTTCAGCACAGGCAGTTTTTTTGGTATTTAAGTACATTGAGGTAAC |
| | | TGAGCACTTGAGAATATTTTAGGGTCAAAGTGTAATTATTCATAATGAATTTACTCTGTTGATATTAAAAAGACGTTCAGT |
| | | CCTATTACTGATGAGTTTACATCTTCAAATAAATCCTGGGTTCTATTT |
| >ENST00000472038.1:: | 125 | AGTCCCGCAGCCGAGCGCAGCCGGGCGCGCGCCACCGCCCACTCGCCCTGTGCCCGCCGCAGCCCGAAACTGGCCA |
| chr1: 167599497- | | CGGCCGGGAGCGGAGGGGACAGCGGGGATCGTGAGCTCCGGCCCGGGCGAGCGGGTGCGTCTGCCGCAGAGTCGG |
| 167634277(+) | | CACCTGAAGGACATGGAGCCTGGAGACATCCAAATGACCCCAGCACACAAATGGCCCTTCTCTGAGAGAGTGTCCTCA |
| | | TGGCTGGAGGCCAGAATCACACTCCAGCTTGCTCTTGTGGTTCCAAAGACTCCCATTTGTAAATTCCTCCGACATGAAG |
| | | ATGCAATGCCAGCCCATCTGCTCTGCCAACCTATTCTGCAAGATGGGAATGAAAATGACTTGGCAACAGCAGATCCCTG |
| | | CCACCCCCTTAGAATTCTTCAGGGCCAAACTGGCAACAGCCTGGTATCTCTACGAAGAGTGCAAGGGAGCCCACATTTT |
| | | GACTAACACCTACTGTGTGCCAGTTGGTATCTTAGGTGACAACAACAATAGCTAAAATGAGGGGAGAGCTTATTATGTG |
| | | CCAGGCACTGTGCTGGGGCTTTATATGCAATATCTCATTTGGGCTTTATCACAATTCTAAAAAATAAGTGCTATTATAAAT |
| | | GTTTATTTTACAAGTGGGGAAACTAAGGCTCAATAAGGTCAAGTGTCTTGCCTAAGATCACACATCTAGTAAAAGGCAAG |
| | | GTCTGGATGTGAACCCAAACTGTCTAATCAAACACCATCTCCTAGTATCCCCATGACCTTGTAGGGGGTGTTATTATCCT |
| | | CATTTTACAGATGAGAGTCTGAACAGTTAAGTCAATTGCCTAGGATCCCACAACCCGTCAATTACAGAGTGGGATCCAAA |
| | | CCTGGTCTTGCCAAGCTCCAAATTCTGTGCTGTCTCCACTATCTTGCTGCATGAGTCCTGGCAGCTGCCCTGGGCTGGA |
| | | CAGGAGTGGCTGGATGTGGCCTACTCCCAGCAGCTACCAGAGGCTGCCAGCCTCCTGCAGCCACAGCGCATTCCTGC |
| | | TCACGCCCTGAGGCACACAGAGCTGGCATCCTGAGACCAAGCCAGGTTCCTAGCCCTAGGGCAAATGAAAGACCTCCT |
| | | GCAGCCTGAGTGCACCTCAACTTTTGTATTTGAACTATCATGAGCACTTTAAAATGTTTTAATCACCTTTTTGAGGTAAAA |
| | | TATATCTACAATAAAATTCACCCCTTTTAAGTGTATACATTGATGAGTGTTGACAAATGTATACAGTCATATAGCCACCAC |
| | | CACGAGATGTAGACCATGATCAAGGTATAGAGCATTTCTGGCTGGCTGTTATGGCTCACGCCTGTAATCCTTGTGCTTT |
| | | GGGAGGCTGAGGCTGGCAGATCACTTGAGGTCAGGAGTTTGAGACCAGCCTGGCCAACATGGCAAAACCCTGTCTCTA |
| | | CTACAAATACAAAAATTGGCCCAGTGCAGTGGCTCGCGCCTAGAATCCCAGCACTTTGGGAGGCCAAGGCGGGTGGAT |
| | | CAACTGAGGTCAAGAGTTCAAGACCAGCCGGACCAACCTGGAGAAACCTTGTCTCTACTAAAAATACAAATTAGCCAGG |
| | | CGTGGTGGCGCATGCCTGTAATCCGAGCTACTCAGAAGACTGAGGCACGAGAATCTCTTGAACTCAGGAGGTGAAGGT |
| | | TGCAGTGAGCCGAGATCGTGCCACTGCACTCCAGCGTGGGTGATGAAGTGAGACTCTGTGTCACC |
| >ENST00000466626.1:: | 126 | GGTGGGGTCAGCCTCACCGAAACAAACAGCATGGCTGCCACCTACGGACTGAACTCCATTGATGTGAAGTATCAGATG |
| chr1: 237963328- | | TGGAAACTAGGAGTCGTTTTCACTGACAACGTAAGCCTACTTCATTATCACAAAAGAAAATGCACTCTGATTAGATAAGA |
| 237965675(+) | | CTGTGGGAGTTCTGCAGATGGAAGGCTGGTTATTTAGAAAATGTTTCGAATACCAATTCATTTCATACAGACCAGATCTG |
| | | GAGAGAGACCCACAGAATGATATGATTAATGAGCCCATCTCTCATTCCATTTCTTTCAATAGCTGAAATAGTAAAATGAAA |
| | | ATATATGAAAAACTGAGATGCAGATTTGTGAGTACGTTCATTAAGGTTTTAATTGGACAATGGCATACTTCAGTTTTATAG |
| | | ATGTTAAAAAGAAACTCCTAAATAATTTTAAAAGAACATTAATTGTGCTAAATTTAGGTTTTCCCCAAATTAAGTACATTAA |
| | | CTTATGGAGTTTTATTTTATTTTTTTTATTCTCCTTTTACCAAAGAGCCTTTGGAGACTAAATATAGGGCCGTAATATATAT |
| | | CCA |
| >ENST00000426200.1:: | 127 | GTCGCTCACGAGGTCGCGCCTCGCACCCGCCTTCCTCCTTTTCTTTTACCCTCCCCTTCAGAAAAAACGGCGGTTGGG |
| chr3: 14986199- | | CTGCGACGGCCGCCAAAGGCGGACTAGAAGCGGAGGGGTGAAAATCCCGGCAGAGAAGGAAGAAGGGACTGCAGGC |
| 14989931(−) | | GGGAGGAGGAGGAGGATAAGGAGGAAGGGAGCCCGCCCAGCCGGAGCCATCTCCGTCGAGAACAAAATGGCGGCGC |
| | | TGGCGGAGGGCCAACTATAAGGCGGGGCCGCGGCCATTCCCCCTCCACCCCCACCCTTGCGCCGGCCGGGCCGGTC |
| | | AGGGGGAACCCGCTCGCTTCGCCCGGCCGCGGGGGGGGAGGGGAGGGGAGCGGCCCGGCCCACTATGCAAAGCGC |
| | | CGGGCGCCGCCGCCGCCACCCCGTGGCAAAGAATATGCTTTTTGTCTAATGATGTTAGATAAAAAGCAAATTTGGAGCA |
| | | GTTTTCTTATTCGAGTTCAAAATGGTTCATAAAGCAGCGAAGACAACTCAAAACATCAGTAACACATTTGGCCCAGGAAC |
| | | TGCTAACAAACATACAGTGCAGTGGTGGCTTAAGAAGTTTTGCAAGGAAGAGAGCCTTGAAGATGAGGAACGTGATATG |
| | | GGCCATGGGAAGTTGACAACGACCAATTGAGAGCAATCATTGAACTACATGAAAAATTACAGAAGAACTCAGCGTTGAC |
| | | TATTCTACGGTCATTAGGGTCGTTCAGCATTCGAAGCAAATTGGAAAGGTGAAAAAGCTTGATAAGTGGGTGCCTCATG |
| | | AGCTGAGCGGAAATCAAAACTATCGTTTTGAAGTGTAGTCTTCTTTTATGCTACGCAACAACAAACCATTTCTCAATCGG |
| | | ATTGTGACATGTAATGAAAAGTGAATTTTATACAACAACCAGCTCAGTGGTTGGACCAAGAGGCAGCTCCAAAGCAGTT |
| | | CCCAAAGCCAAACTTGCACCAAAAAAAAGGTCATGGTCACTGTTCGGTGGTCTGCTGCCCATCTGATCCACTACAGTTT |
| | | TCTGAATCCTGGTGAAACCATTACATCTGAGAAGTATGCTCAGCAAATTGGTAAGATGCACCGAAAACTGCAATACCTGC |
| | | TGCCTGCATTGGTCAACAGAAAGGGCCCAATTCTTCTCCACGACAACACCTGACTGCGTGTCGCACAACCAGTGCTTCA |
| | | AAAGTTGAAGGAATTGGGCTACAAAGTTTTGCCTCATCCACCATATTCACCTGACCTTTCGCCAACTGACTACCACTTCT |
| | | GCTAGCATCTTGACAACTTTTTGCAGGGAAAACACTTCAGCATGGTGCAGAAAATGCTTTCCAAGAGTTCGTCAAATCCT |
| | | GAAGCACGGATTTTTATGCTACAGGAATAAACAAACATTTCTCATTGGCAAAAATGTGTTGATTGTAATGGTTCCTGTTTT |
| | | GATTAATAGAATATGTTTTAGCGTAGTTACAATGATTTAAAATTCGCAGTCTGGAAATTTAATTTTTGCATCAACCTAATAT |
| | | TTCTATGGTAAATCCTTGCAAACATGGAAACAATGCATTTGGCCCAGTGCTTTGTGGTTGTGTACTCTTTTTCTTTGTTTT |
| | | TTTAATAGATGGCATTGGCCGGGCATGGTGGCTCATGCCTGTAATCCCAGCATTTTGGGAAGGTGAGGTGGGTGGATC |
| | | ACCTGAGGTCAGGAGTTCAAGACCAGCCTGACTAACATGGTAAAACCCCATCTCTACTAAAAATACAAAAAAATTAGCTA |
| | | GGCGGGGTGGCGGGCATCTAATTCCAGCTACTTCATGAGGCTGCGACAGGAGAATCATTTGAACTCGGGAGGCAGAG |
| | | GTTGCAGTGAGCCGAGATCACACCATTGCACTCCAGCCTGGGCGATGAGCGAAACTGTCTC |
| >ENST00000462257.1:: | 128 | AAACATAGAAACCTCAGGACTGCCCAAGAAACCAGAAATTACTCCACGTTCACTTCCTCCAAAGCCTACTGTTTCCTCAG |
| chr4: 152091684- | | GGAAACCTTCTGTAGCTCCCAAACCAGCTGCTAACAGAGCTTCTGGAGAGTGGGACTCTGGGACTGAGAACAGACTCA |
| 152096444(−) | | AGGTGACCTCCAAGGAAGGACTCACCCCATACCCTCCCCTGCAAGAAGCGGGAAGCATCCCAGTAACCAAACCTGAAT |
| | | TGCCAAAGAAACCAAACCCTGGCCTTATACGAAGTGTTAATCCTGAGATTCCGGGAAGAGGGCCCCTGGCTGAGAGCT |
| | | CTGATAGTGGGAAGAAAGTGCCAACTCCTGCCCCGCGGCCTTTGCTGCTGAAGAAATCTGTTTCCTCAGAAAACCCCAC |
| | | CTACCCTTCAGCTCCACTGAAACCTGTCACTGTTCCTCCCCGACTCGCAGGGGCATCACAAGCCAAAGCATACAAGTCA |
| | | CTGGGAGAAGGGCCCCCAGCCAACCCCCCAGTTCCAGTTCTGCAGAGCAAGCCCTTGGTGGACATCGATCTCATCAGC |
| | | TTTGATGATGATGTTTTGCCCACCCCATCGGGGAACCTGGCTGAAGAATCTGTTGGTTCAGAGATGGTTCTAGACCATG |
| | | TGGAGGCATTACCTTTAACAAGAGAGTTAATCAATAATCTTTTTAAAAGAGCTGAACATAAGCATAATTAGGCTGAAAGAA |
| | | ACTGGGAGAATTATCTCTTGTCGTCTTTGCCACTTCTGAAGAACTGCTTCCCCCAATGACCCTCAGATTCTGATAAGTAA |
| | | AAATTTTTAAAATAATAAAATAGTGATTTATCTAAATGGTTATATTATTCAAGGTTACAAATAGAAGCTACTTGTTATGAACT |
| | | AAATATGCC |
| >ENST00000510733.1:: | 129 | GAGCACCGCGCGCGGCCCTGCCCCCGGCACGGCCCCCAGGTGCGCTCCTTCTCCGGCTGCTTGTAGCACTGGTCTCA |
| chr4: 155665151- | | CTGTCCCCGCCGTCAGCCACCGGTTCCTTATCCGTCTCATTCCCCATTGTGGCTTGGCTGAGCCGGTCGCCAGGCCTC |
| 155674270(+) | | GCTGTCCTCCTTTGCCTTCCTCTCTCCTCAGCGGCCGTACTTTGCGCCGTACCTCACCTGGCCTGCAGGTGAGCAGCA |
| | | GCGCAGCACCCCTGCCCGGCGAGCTTAACTTGCCCAGCCCGGCCCCTGCCGGAGTGGCACCGGCACCTCTCCAAGA |
| | | CGCCCTCTTCCCTGCAGGATGAAGAACCCCATGCTGGAGGTGGTGTCTTTACTACTGGAGAAGCTGCTCCTCATCTCCA |
| | | ACTTCACGCTCTTTAGTTCGGGCGCCGCGGGCGAAGACAAAGGGAGGAACAGTTTTTATGAAACCAGCTCTTTCCACC |
| | | GAGGCGACGTGCTGGAGGTGCCCCGGACCCACCTGACCCACTATGGCATCTACCTAGGAGACAACCGTGTTGCCCAC |
| | | ATGATGCCCGACATCCTGTTGGCCCTGACAGACGACATGGGGCGCACGCAGAAGGTGGTCTCCAACAAGCGTCTCATC |
| | | CTGGGCGTTATTGTCAAAGTGGCCAGCATCCGCGTGGACACAGTGGAGGACTTCGCCTACGGAGCTAACATCCTGGTC |
| | | AATCACCTGGACGAGTCCCTCCAGAAAAAGGCACTGCTCAACGAGGAGGTGGCGCGGAGGGCTGAAAAGCTGCTGGG |
| | | CTTTACCCCCTACAGCCTGCTGTGGAACAACTGCGAGCACTTCGTGACCTACTGCAGATATGGCACCCCGATCAGTCC |
| | | CCAGTCCGACAAGTTTTGTGAGACTGTGAAGATAATTATTCGTGATCAGAGAAGTGTTCTTGCTTCAGCAGTCTTGGGAT |
| | | TGGCGTCTATAGTCTGTACGGGCTTGGTATCATACACTACCCTTCCTGCAATTTTTATTCCATTCTTCCTATGGATGGCT |
| | | GGCTAACTTCATACCCCCATGTCAGTGTGTGTATTCTGTATGTAAATATGTTTATATTTATAGAGCATCAATCAATATAAG |
| | | CATTATTGAGAAAAATGTGACCCGTAACACTGTGTTCTGGATAAAAATGTGATTAGGAATCACGCAAAGTGCTTACTGTG |
| | | TAAGCCCAAGAACAAAGGCTTTCTGAATCTTCTCAGGCAGTTCAGATTTAAAGCACCATCCAAACCTTGGAAATACGACA |
| | | GGGTGTGGTAGAATTCAGCAATATGAGAAAACCAGCCCCTAAAATGATAGCCACAAGAGATTAATTGTGTTTTTTTTTCT |
| | | CCTGTAATCCTTGTACTGTTCGGCTGAATTTGAAGATTGGAAGACTTATATTGAGACCAGTAACTTTACTGTAAATTTACT |
| | | TTGTTTCATTGAAAAAACAAATTGATAAACATATTAAACTGGAAGAATTTTCTTTATTCAAATGAAAACATGTTTGATGACT |
| | | GGTCAAAAAATAAGCTCATAATCTATTTTTTTCATGTAGTATATAAGTCAAGAATGTTTTATTGTCATTATGTGAAACCAAT |
| | | ATTGGCAAATAGTACTTTAATGATGAAGTAAATGACCAGAAATTATAGAAATCTGTGTTTTCCTGTAAAAATAGCACTATA |
| | | GTATCACTTGAACAATTTGATTTGGCTTTACTTACTAGGAAGCCTGGAATTCATTATTTTTTTCCTTTTATGTGCCACTGTG |
| | | GCTACTTTAAATCACTCTGAGAGGTAAATGGATATAGGATTGAAGTTATGTGGGTATTTGGCATGTGTGTGTGAAATAAA |
| | | TATGTAGATAGTCACATATACACAGACTGAGAGATAAATTGTTCTTGATTGCTTTATTATCATCATACTAGTGTGTTCATTA |
| | | TAGAGTATCTGTAGAGGTGAATGTAAAAGTAAGTCCAATCTATTTTCTTATGTCATTGAATTTGTAGTGTTAACTTGCATAT |
| | | ATGTTATTGGATGGGTTGTCTTTTAAAGCATTTACTAATGTACTCTGAAATTTTTAAAAGCCTTCAGATTTGTTTTCTAGTC |
| | | ACTTTTTTCCATATCATTTCTAATTATAGTTTATATCCTTAAAAGAAGGATGCCACAGTAGTATGTAAAACCCAAACAAGTA |
| | | GAACCCAAGCAAATAAAATTATTTAAAATAATTTTAAAGTGGCTTAGTACTGCCAGTCATGTAAATTGATTCTGCTGAGGG |
| | | TCTTATAAGAATTGAGATATAACAATGGTAAAACAAGCATTCAAGCACTTTTACAAAATTACCAAATTCTTAAAATGAAGC |
| | | CACAGCTAGACTTGCATTTCAGGTATTAAAATTGCTTTCTTAACTGTCAAGAATCACAAAATAACAAATCATATTATGAGT |
| | | GAATATGGGGAGGGCGGGGCCAATCAGTCAATGATAATCTGAACAAATTTTAAGAGCAGATTTTAGATTAATAATGTTTT |
| | | ATCACCACTAATTTGCCCACAACAAACTCAGTATTTAATTTTTCAAATTAAATATTAAATTATTTAAGTATTTTAAATAATTA |
| | | AAACATTAAATGGCAACACCATAGAATATAGGTGTTCTCTGGACCTATTCTAACCACTTAAAATTATCTTAAGTATGCATA |
| | | CATAAAAGCAACCACTATGAGAACTACCGTGTTAGTGGTTTTTCACTTACTGTATATTACCCTTGTAGGAATAGTTTAAGG |
| | | AAATTCATTTCTTAAAAATATAGTGTCCTCAAATAATTAATTTTTTTGCAAACTTTAGTTATTACAGGCAGCAAAAACCACT |
| | | GTCTGAAACTAAATCTGTGTTCAAAGATGAAGACCCCTCATTAAAAGCCAAGGACGTTCTTAAGATTGGAACTGACATAA |
| | | TTAGTCTTGACTTACTTCATTAAAGCAAGATTCAATTCCTTGTTCATTTGAGTGTTCATTATATGCCAGGCATTGACCAGT |
| | | TGCAATTTTATGGTCCAGAATTAATATTATTATTCGTAAACTAGCTATAATAGAGTATATACAGTGCTGTGGGAATACAGG |
| | | GGAAGAAGCAATTAAATCTGCTAGAAGGGTAAGAGAAATCTGTATGCAGGAGGCAATGCTAGCATTAAGCCAAGAAATA |
| | | GTGTGAGGTGATCTTATCACACTGATGTGTTCAACTGTTTATGGGAAAAGGAAACTAGGATGGTTTTATTTGTGAAATGT |
| | | TCTACAACGCTAGAACTGTTGAACTTCTTGGGAGAAAAGGAGAATCTTTGTTTTGTTGTGCTGAGTTTGATTAGTTGCTTA |
| | | ACCAAGTAGAAAGAAAAGCACATCTTCCTGATCACAGTTATAAAAGAAACCCTGAGGCCGGGCGCGGTGGCTCAAGCC |
| | | TGTAACCCCAGCACTTTGGGAGGCCAAGGAGGGAGGATCACAAGGTCAGGAGATCGAGACCATCCTGGCTAACACGG |
| | | TGAAACCCCATCTCTACTAAAAATACAAAATTAGCTGGGCGTGGTGGCCGGCGCCTGTAGTCCCAGCTACTCATGAGGT |
| | | TGAGGCAGGAGAATGGCGTGAACCGAGGAGGCGGAGCTTGCAGTGAGTGGAAATTGCGCCACTGCACTCCAGCCTGG |
| | | GCAACAAAGTGAGACTCCATCTCAAAAAAAAAAAAAAAAAAAAAAAAAAAAAAAGAAGAAGAAACCCTGAGCCATTAATG |
| | | TGTCTCGTAACTTGAGGAAGTTCATGAATTCTCTGGGCTTCATCCATTAAGATTCATCCTTATGCTCCTGCAAGTTTCTTA |
| | | TATTTATGTATTTCTATTCTGATTTCTGAAATGATAACCTAGTAGTGTCTGATGAAGTTTTTTCATGCTTATTTTATTCTTTG |
| | | TCACAGATGAAAGAAGAATAGGACTAATTGGGGCTAAGATGAATTAATGTAAGTGTCAGATACTAGGCAGGTGTTAGGG |
| | | ATGAAATGATGAATGAGGCATGGACCTGTTCCTCAGAGTTTATGGCCCAGTAGACATAATTGTATTAGGACTTTCTGCAG |
| | | TTGACATTTGCATAAAGCTGACTGACTGAAGGAACAGAAAGTGAGACAGACAGATATGACCAAATTTATACAAACCATGT |
| | | ACCAACACTTGAAAGATGGGTGGATATTTTCCAGGAAACAGATTTTGGGCAGGACATTCCATGCACTGGAAATAGCATG |
| | | AACAAATGCATGAAAATTGCCTACAACATGCCTGGCACATAGCTGTGAGGTACCCTATGAGTTGTTGACTTGAAAACATG |
| | | GAACTGCACAGGGCATTCAGGCAAGTGCAAGTGGTTCAGGGGCCAGCGCAGAGCAGAGTGCTGAGTAAAGATGTCAG |
| | | AAAAGGAAAGCAATGGTCTGGTACAAGTCTTGTATAGAACAAGAAGTTTAGACTTTTGAACAGGCAATCGAGATTTGGG |
| | | GTTCAGAAAGATAAATCCCAAAAGTATGGAGAACAAAGGAACTCATTTTAAAGGACTAGCTAAGATGCTCAATCTCTAAA |
| | | AAAAAAAGGATCTGTATTCGTGAGAGTAGAGAGAAAGGTTGTTTAGGAAGAGTCAACAGACTTTAGCAAAATCCTTTTAT |
| | | TTGATTCATGCATAACTCCTGATGGAGTGTCAAGGAAGACTCATTCACTTTTCTTTCCTGCCAGAAAGTTGGTTCTTGCA |
| | | AAATAGATTAACTTGATGACTATGTGTATATTGGATACTCCTAATTTTAGCTCTGTTAGATATGTTTCTTTCTAAGGACAAG |
| | | CAACTATGTATAGACTTGTTGTTTGAATTTTTTTTCAGTATATATATGTTTCTTGTTTTTCTATTTTTCTACTTCATACTGTTA |
| | | ATGTCAAATATCAAAAATGTTTTCCAGCAATATCAAGTTATGGCAATGGCAAAAGTCCTTGTTGTAGATGAGAAGCATATA |
| | | AGAAATGGTTGCCAATCTTTGTTTTGTATGATCTATTTAGAGTTTTAACCAACCTAGATCTGTCAGCTAAGCTAATTTTATA |
| | | TTTGGTATACATGCATGTCAAATAAAGTTAATTTTACATAC |
| >ENST00000513560.2:: | 130 | AGCTTTCTTCCCGCCAGGCCCCCTCCACCCGATCGCCGCGCGCTCTCCGAACCAAAAGGCGACCTCACGAAATGCCC |
| chr5: 43571695- | | CTTTGAGCTCAAAGGCTAGTTACCCCCAGGGGCCCTTCCACTCTCGGGGACAGGCGAAACCTCTTTGTCTCTGCCTCG |
| 43603206(−) | | GCCTGCGGCCCCCAGCCCAGCCTCCGCGCTTTCCCTCCGCCAGTCCTTGTCAATCAAACCTGGTGCCAAACGCGGCA |
| | | GCTGAAGTTTTCAGGGACACATTTGCTTCTCCCCTTGAAGAACCAGTTACAAAGCGTGATGTCCTCTCTGGGGTCCCAT |
| | | CAGAACAAAGAAACAGGTCTAAAGACCCTCATTCCAGAGAGCATCCTGCCCCATATCCAGAATGAAATCCATGCTCAGA |
| | | GATGCCAAGAAGAATCTAGACAGACAGGCCTTTCTAGGCTGTACAAGAAGCATGATGCCAGCATCTGCTTCTGGTGACA |
| | | ACCTCAAGAAGTTTACAATTGGCCGCGTGCGGTGGCTCATGCCTGTAATGCCAGCACTTCGGGAGGCGGAGACAGATG |
| | | GATCATTTGAGGTCAGGAGTTCGAGACCAGGCTGGCCAACATGGTGAAACCCCATCTCTACTAAAAATACAAAAATTAG |
| | | CTGGGCATGGTGGCATGTGTCTGGAATCCCTGCTACTCAGGAGGCTGAGGCAAGAGGATCACTTGGAACTTGGGAGG |
| | | CAGAGGTTGCTGTGAGCTGAGATCACACCACTGTACTCCAGCCTGGGCAACAGAGTGATACTCTATCTCAAAAAAAAAA |
| | | GAAAGAAAGAAAGAAAAGAAAAAGAAGCTTACAATCATGGTGGAAGGCGAAGAGGAGGAGCAGGCATATCACATGGCC |
| | | AAAAAGGGAGCAAGAGAAAGGTGAGAAAGACACTAGACTCTTTTTAAAACCAGCTCTCACATGAGCTAATGGAATAAGA |
| | | ACTCACTCATTACCACAAGGACAGAACCCAGCCATTCATGAGGGACCTGCCACCATGACCCAAAAACCTCCTACTTGAC |
| | | CCAACCTCCAACACTGGGGATAACATTTCAACATGAGATTTGAAGAGGACAAATACTCAAACTATATCACTTGTTGTCCA |
| | | TTTTTAATCTTTCTCTAACATACCCTAACTTCTTCTTGAAAATGCTTAATTTTCTCTGTTTTGAGATGTAAATTTGCTATCCT |
| | | GTTTTTCTTAAAACTTGGTAAGATCTTCAGCCATGAAGGACAGACAAACTGTAACCTTTCCATTTACAAAGCTGCATTTGA |
| | | ATTCAACTGTCCTTTTAAACTAAGGAGTTTTACTGGTCTCGTGGCTAAAATTTTAAAGATGTAGGCCAGGTGCAGTGGCT |
| | | CATGCCTGTAATCCTATCACTTTTGGAGACCAAGGCGGGGATGGATCACCTGAGGTCAGGAGTTCAAGACCAGCCTGG |
| | | CCAACATGATGAAACTCCATCTCTACTAAAAATACAAAAATTAGTCAGGCATGGTGGCACACACCTGTAATCCCAGCTAC |
| | | TCGGGAGGCTGAGGCAAGAGAATGGCTTGAACCCGGGCGACGGAAGTTGCAGTGAGGCGAGATTGCACCATTGCACT |
| | | CCAGCCTGGGCAATAAGAGTGAAACTCTGTCTCAAAAAAAGAAAAAATGATATAAAGTATTTATTTGTGTTTATATTTTAT |
| | | GGATACGTGTCTATGTTTATAGATTGCCTGTATGGTACCAACTTGACATAAATAAATTAGTACTCATAAATTAAGTAAATA |
| | | AGCCCAAACGCTTTTCAAGTTCACAGGGCATTAGTAATCTGTGGTAAATGAAGATAGTTTAAAATTTGGGGTAAAATAAA |
| | | ATAGAAACATTTTCTGAATTTAATTTAGATATTTTTCTGGGTCTATTTGTTAGACAGGTTTACACTATCTCTGCTATTTTTTT |
| | | AAGGACCTAAAACTGAATGGACATAGAAAATTGAGGAAAGAAATAAAACCGTAACAATTATAAGAGGTTATAAAAGGTTC |
| | | ATGGAACCTTTTTATGTTTTTTCTTGTGCAGTCAAAACCAATTGCAGGCTGGATGCAGTAGCTCACACTTGTAATCCCAG |
| | | CACTTTGGGAGGCTGAGATGGGCAGATCACTTGAGCTCAGGAGTTTGAGACCATCCTGGGCAACATAGTGAAACTCTC |
| | | TCACTACCAAAGTACAAAAGGAATTAGCTGGGCATGATGGTACACACTTGTAGTCCCAGCTACTTGGGGGACTGAGATG |
| | | GGAAAATTGCTTGAGCCCGGGAGTCAAAGGTTGCAGTAAGCCAAGATTGCACCACTACACTCCAGCCTGGGTGACAAA |
| | | GTGAGACCCTGTCTCAAAAAAAAACAAAAAAAGGAAAAGAAAACAATTGCAATTGGATTGATCTGTTTATAAGGCTTTATT |
| | | AAAATTAGCTTTAGCATTTATAATACACTGGCACAAAGTTAGAATTTGGATTTCTCTTTTGAACAAGATTTTTGTATACATT |
| | | AATAAGAAATAGTAAAAGATTTTTGTTTACCTTTTGAGTAAACTGCAAAAAATATTAGTAATAAGAGGAGAGAATTTCCCTA |
| | | ATGCTGTTTTTATTATGTCTTTTGATTGTTTGGAAAACTGAATCTCCCCTCTGTCAAAGAGTAAAATTTTTGCTTTTTGAAA |
| | | TTTTTGAATTATCAGTTTGGCTAAATGAATAACTATTATCTTACAGTGACCTGTAATCTTATTTTGATCAAGTGTTTTAAAC |
| | | CTTTGATATTTGACAAACTTCCCAAAATCAAATGTAAAATTCTAACTTAAATGTTTTCGACCTCAAGCTAACTTTTGGGCAT |
| | | TAAAGCCCTTGGAAGTCCAAATAGGGCCCCTGGAAGTCCAAAAGAAACATATTAGGCTAATTTGGTATATTAAAATCATA |
| | | CAGGAAGCTGATCAAATAAAAAATGTTGTTTAATTTTCTTTGAGTTGTATTTGTATAAATCTGTTATTAATATGCATCCCAA |
| | | AATTGTATCATATTGCTAAAATGTTGATAGATCTTGTTATATGTTATTGGTAATAATTGTTATCCTGTTAAATTATTGTATAC |
| | | CACAGAAATAATGAAATTTCTTTGTCAATTGCATCAGTAATCATGGCTCTTCTAAGTCTTTTGTCATTCACAGATGATTATT |
| | | GTTTTACTTGGATTCTTTTCAAAAGTGGTTTGTAATTGGCTACAGCCTAAAATCTGCTTGTTCAAAAAAAAAAAAAAAAAAT |
| | | CCATGGAAAGGGCCCTGACAGCACACACACTTAAACACAGTTTTCTGATAACTTTGGAATTCACACCGTTGGACTAGTTA |
| | | AAAACTTCTAAAATAATTTTTTAAAATCTAATAAATTAATGAAGATTGCTAATCCAACATCAAGCAGAATAAGTTAATTACAT |
| | | GGGGCTGAACTGATAAAATGCCAAAATAATATTTTCATAACCTTTTTTTGGTTTGAGACATTGCTGATACTTTTTATGTTTT |
| | | GTTTTCCAGAGTCAAGAAAATTGTTTTTTTCTTTTGAGCTGTACATAGCTTACAGTAATTAGATAAATTACACTTTTGTGAG |
| | | AACAATTGAAACACTAACCTTTCTCTCTACCTGATGTCTCCAGAATTTGGAAAGTATTTGTGAGTATTCTTCACTTTTGGC |
| | | AATATAGTTATTTGTACAAATTCGATAGGAATCTGTTTTCTTTTGTAACAGAACACAGTTGGATACACTGATTATTTTGCCA |
| | | AGGCTTTCATTGGAATGGCATAATTTTTTAATGACCAGACTGCTTTGAGGATTTGAAGTTGACTTTATAGAGCCTATAAAA |
| | | AGCCTGTTGGAAAAATTAGCCTGATACCTTGTCTACACAGTTTCCTTACAAGGTTCCTGACCTTGCGGTAGTAAAGAATG |
| | | TCACTCTCTGGCAGGCCCAGGAGCCTCAGGATATTTTGGGAACCTTGACAAGAGAGGAGTGTATCCAATTTATACAGGA |
| | | ATTACAAGTGCAGTCTGATTGTGAATCCTTGTCTTGGCTTCTTAGCCTTGAGAGTTTTTAAAAGTTGAATGTGAAATTCCT |
| | | TATGAAAAAGTTCCAACAAAGCCAAACTTTAAAAGAGCCTATATGTGGTCAATCACTATTTTTGCTGTACTTTATGCAAAT |
| | | AATCAGGCCAAATATAATAAAACTAAAACTTATTTTGCAAATAAATTGGTCCTGTTATGATTTGCCTTTAATAGAAAAGGG |
| | | GGACTGGAGAGAGAAGAATTATGTTTCAGAAGAAAATGATAGCATACCTGTTGTTAGATTCTAGCTTTGTCCATTGTTTTT |
| | | AAGTTGTAATTATTTGCCTACATTTGAACTAAATCTTGAATTCTTTCCTGGCTACAAGTCTCCAAGCTAACATTTAAATTTT |
| | | TTTCTCCTATGTTTCTGACTTGGAATAAGTAGAAGTTAAAACTATGCTTTTCTTGAAGCCCTGCAGACTGGAGCAAGACA |
| | | ACTTGAATAAACTATGGGAAAAATCACTACAGCAACTTATATATAAACAGCTTTTATGCTTTGTTGATGTATGGAATACTC |
| | | AGAAAGTTCACTGCAACACCTGATTTAAACTACAACCAGGAGACTCTGTCAGATTAACACTACAATCTGAAGAACTACAG |
| | | AGACTCTCAAAAAACTAGTGTATAGTCTACAGTAGATATTAACCTTTGTTTTTCTTCTGTTTTCATAGAAACACCTTTTATT |
| | | AAAAATCTGTTTGCCGCTTCATATATAGAGTCCTAGTCCATCTGTAATGCCACCCCCTGGAATGAGACATAGCTGTTTAA |
| | | CTGAACTGATCTATTCTCGGGACTAAGAGACTG |
| >ENST00000509269.1:: | 131 | ATTTTTGTATTTCTAATAGAGACGGAGTTTCGCCATGTTGGCCAGGCTGGTCTCAAACTCCTGACCTCAGGTGATCCACC |
| chr5: 140022947- | | CACCTCAGCCTCCCAAAGTGCTGGGATTACAGGCATAAGCCACCACGCCTAGGCAACTGCTTCTCTTCTTAAAGGTGGA |
| 140023497(+) | | ATTGACTATATACCTAGGGACTGCTTGCTTCCTGTTGCCCAGCCAGGTCAGCAATCCTCTGCTCATTGGCCATGGGGCT |
| | | CTGTCTACTCTGGGGTTGCTGCTGTTGGACTTGGCTGGGGCTGTCCAGAAAACCGAGGATGCAGGACTGGAGCTGCTG |
| | | GCATGCCCCGTGCTTCGATGTCTAAGCAACCTGCTAACTGAGGCAGCAGTGGAGACTGTGGGAGGGCAAATGCAGCTC |
| | | AGAGATGAGCGTGTTGTGGCAGCCTTATTTATCCTTCTGCAGTTCTTTTTCC |
| >ENST00000503388.1:: | 132 | TTTGGATACACTTATGAGCATTTGTGCCCAATATTCGGGTCATTTTGGGAACTTCCCTTTTGAAGAGAACCAGTTTAATG |
| chr5: 141522542- | | GTGTTTTGTCTTGCAGAGCTCTGTGCAGAGCTCCCAAGCTGGCTGCTCTTGAGCTATAGTTAGCCTATAGTTTATTTGTT |
| 141533760(+) | | CCACAGTTATTTTTCAAGTTTTTTTAAATTAGTTGCCAACATTTTAAAAATCAAAATTATTTCACATAAAACGCCTGAATGTT |
| | | TTTACTTTTCTTGAAAGTTGGAATATCAAGAAAAGCTAGCCTACATTTCACATGGCACCCTTTGGCTGGAGCTGAGTTGT |
| | | TGCTGTCTCCTCCATACAGGTCATGTGCCTTCCAGTTCAGTAAGATCCCACCACCATACCCCAACTTAGTATTTGAAGGG |
| | | GTAACCCCTGCTCAATAGTCACTTGTTGCTTTGGGTCCCAAACAGAATGATGTTCCCCAAGCTCAGAGCTGACAGCTCT |
| | | TGGTTACTGCTAAAAATCAGTTCAACTGTCCAAGTACCATTTTCTACCACTGAGTATCCCAGTAAAAGTATTTAAAAAAAA |
| | | AAATGTGCTAAAGGCTCAGAGACCATCTCTGAAAGAGGAAGGCCAGGAGGAGTATCTGAGTTTCCACTGGAGGGGCAT |
| | | ATCACCTCTCTGCTGACAGTTTTAGGAGGTACAAACTTTTGTGAATGTATAGATTCTGGTGTAGTCACACAAAAAGAGTC |
| | | TTACTAAGAATAGAGAAATAGGCTGGGTACAGTGGTTGACACCTATAATCCCAACTCTTTGGGAGGCTGAGACAGGAGA |
| | | ATCACTTGAGCCTAGGAGTTGAAGGCCAGCCTGAGCAACATAGCGAGACCCCTATCTCTACAGAAAATTAAAAAATTAG |
| | | CTGGGCATGAACCTGTTATCTTAGCTACTTGGGAAGCAGAGGCAGGAGGATCATCTGGGCCCAGGAATTCAAGCTTGC |
| | | AGAGGCTGTGATCATACCGCTGCACTCCAGCCTGGGCAACAGAGCAAGACCCTGTCTGTTAATAATAATAATAATAATA |
| | | ATAATAATAATAATAGTAGACAAAGTTTCAAGTTGAGACGATGAGAATTGCTGTAATAGATAAGGGGATCCTGACATCTA |
| | | GTGGACAACATTATATACTGTCGACAGAAAAAACAGCCCATGACAAGGAAGGTTACATTTTTTCTTTTTACTTGAGAAAAA |
| | | CACAGTTATAACCTCTTTTTGTTGTTGTTGTTGTTGTTGTTGTTTGGAGTTCATTATCTTGGAATGCTTTTTTGGAACTCAA |
| | | TAGGTAGATTTTGATGTTTCTCAATGAGGCTTTCATATGTAAATGTTTGGCTCCATCTTATTTTTTATTTCCTTTTTAGACC |
| | | AATTCAATTCTGAATGTGCAAAAGACACTATTGACTCTTACATTATACCTTCTCATTGAAGGATGGATAATTTGGTATGAA |
| | | AATCCTCTGCAGATTCTGGAGGAAATTGCTAATGTTTTGATTACCTTTAGCAGAAAAATAACAATGTATCGCTATCAGGA |
| | | GAGGGATTTCTGGGGAGGGGACTTAACATGGAAATTATAAATTCATTCATGACTTTTCTTTTTTAAATTAGGCTTTCTCCT |
| | | GTTTCTCAGAGGATTTATCAATTATGCAAAAGTTCGGAAGATGCCAGAAACTTTCTCAAATCTCCCCAGGACCAGAGTTC |
| | | TCTTTATTTATTAAAGATGTTTTCTGGCAAAGGCCTTCCTGCATTTATGAATTCTCTCTCAAGAAGCAAGAGAACACCTGC |
| | | AGGAAGTGAATCAAGATGCAGAACACAGAGGAATAATCACCTGCTTTAAAAAAATAAAGTACTGTTGAAAAGATCATTTC |
| | | TCTCTATTTGTTCCTAGGTGTAAAATTTTAATAGTTAATGCAGAATTCTGTAATCATTGAATCATTAGTGGTTAATGTTTGA |
| | | AAAAGCTCTTGCAATCAAGTCTGTGATGTATTAATAATGCCTTATATATTGTTTGTAGTCATTTTAAGTAGCATGAGCCAT |
| | | GTCCCTGTAGTCGGTAGGGGGCAGTCTTGCTTTATTCATCCTCCATCTCAAAATGAACTTGGAATTAAATATTGTAAGAT |
| | | ATGTATAATGCTGGCCATTTTAAAGGGGTTTTCTCAAAAGTTAAACTTTTGCTATGACTGTGTTTTTGCACATAATCCATAT |
| | | TTGCTGTTCAAGTTAATCTAGAAATTTATTCAATTCTGTATGAACACCTGGAAGCAAAATCATAGTGCAAAAATACATTTAA |
| | | GGTGTGGTCAAAAATAAGTCTTTAATTGGTAAATAATAAGCATTAATTTTTTATAGCCTGTATTCACAATTCTGCGGTACC |
| | | TTATTGTACCTAAGGGATTCTAAAGGTGTTGTCACTGTATAAAACAGAAAGCACTAGGATACAAATGAAGCTTAATTACTA |
| | | AAATGTAATTCTTGACACTCTTTCTATAATTAGCGTTCTTCACCCCCACCCCCACCCCCACCCCCCTTATTTTCCTTTTGT |
| | | CTCCTGGTGATTAGGCCAAAGTCTGGGAGTAAGGAGAGGATTAGGTACTTAGGAGCAAAGAAAGAAGTAGCTTGGAAC |
| | | TTTTGAGATGATCCCTAACATACTGTACTACTTGCTTTTACAATGTGTTAGCAGAAACCAGTGGGTTATAATGTAGAATGA |
| | | TGTGCTTTCTGCCCAAGTGGTAATTCATCTTGGTTTGCTATGTTAAAACTGTAAATACAACAGAACATTAATAAATATCTC |
| | | TTGTGTAGCACCTTTTACTGTAGATTAGTGCTTAATTTCTTGGCTTGCATTTGTTGATTGCTAAGGCAATTTTTTCTAATCT |
| | | TAGGGAATCATTCAGTAGATGCGATTAAAAAACTAATGTTGGGTCAATTTTTTTCTTCATTTTCAGCACAAGAAGTCCTCT |
| | | TATATCCTACTAAATACATTCCTAAAAATGTATTTGAACATTGGTTCTGTAAAAGATAATGGACTAAAAAAGTAGAGAGGA |
| | | GTTGTAGAGATCTTAAATCATTCTGGAATTCCTAATTATGCTTCAATTTTTAGACATAATTTTAGATAATTTATTTCCAGTGT |
| | | TTTCTGCATGTTCTCATTTGTTCTTTTTCTCAGTTGAATGCACCAACTGGTTTGAGTCCTGTGAGCATTCAGTCAGTTGAA |
| | | ATTAAAGATTCCTCATTTCTCCTGATTTCTATTCTTGTCTCAATCTTAAATTTAGAGACCAGTTGTTTTTATGATATCAGCC |
| | | ATTTGATTTTTTTCATTTTCTATTTAAGAAATATGAAGAAAAAATACACCAAGATGGTCAAATTACTACACAAATCAGCACC |
| | | AGCACAGTCTGATAGCTGCAAATGTCCATTCATCTGCTGTGTATGTATATCCAGAATCAGCATAGGAAGTCGTTCAGGAT |
| | | ATCAGTATATAATGCACAGAAGTGTGGGTTGTTTGAAAGCCAAACAGGAAAATTAGGAGCCTCCTGGATTGACATTTCAA |
| | | TGATCCCTCTAACCAGTTTATGGATTATTATGAATAATAGTGTAGTGTGTTCTTTTTCAGAAGTTATATTTGATAATAGAGA |
| | | AGGGAGTTTTATGGAAGTTTCTTTGAAGATTTTTTTTTTTCCATTTCGAATCAGATTATAGCAACAATGGAGTTTGGAAGT |
| | | TTGTATGGCCTATAATGTTCTAAGTTCCAGAATGAAAAGATCTGTAACAATCTGAATAGATGTGGACACATATAGCAGAG |
| | | AGAACTATGTAAATTATCTTGCAGAACAAAATAGAAGGGTCCTAAATCACGTTAACTCAAACATTGTAGACTAGCTTTGTG |
| | | TTTATTCTTCAGGTCCTTGCGCCTTATTTGGTTTTGTATATTCAACGAACTGAAATATTTGGAATTCCTATTTCTACGTATT |
| | | TGGTGGTCCATAAGACTTTGTCAAATGTAAACCTACAGTTTGATACGCTTTAAAATACCTAGTTAAGAGGATGATTTCTCT |
| | | TTAATCGTTTAAATGTTCTGAAAATTAAAATCTTTTGAGGCACATGAAGTGGGCACCATATATCATCTAGAGTCCTTACTG |
| | | GTATTCAGGATGAAAATGTTCACGCTGCATTAATTGTCATTTTTCTCTCCCATGTTCTTTCTCACTTTGATACGTTAATACT |
| | | GATAATGGATAAAGAGTGAGTTTTTATAATAAATGGTTTTGGAAAGGT |
| >ENST00000464526.1:: | 133 | AGGCTGGGGGAGAAGTTAAAGCCAGAGGAGGGGCAGGAATGTCTGAGGTGGCAACACTTCTCTTCAGCCAGACAGCA |
| chr6: 31553970- | | CTGGCCAGTTTGGAGTCTGTCCATCCTGCAGGCCACAAGCTCTGGATGAGGAACTTGAGGCAAGTCACCAGCCCCTGA |
| 31556686(+) | | TCATTTCGCCTAAAAGAGCAAGGACTAGAGTTCCTGACCTCCAGGCCAGTCCCTGATCCCTGACCTAATGTTATCGCGG |
| | | AATGATGGTAAGTAAAGTGTCTCTTGCATCTGCATAGAGAGAGTCCTGGGAGCTTAGGAAGTGATGGGGAACAGTGATG |
| | | TATGCAGCTCATGACTAGGTGGACAGGCCTCTGGGGACAGCTGGTACAGGAGGGAAAGGGACCTCACGGGAGGCCCA |
| | | GAAACCTGGTAAGAGGTGAGGTATTAAGGTCTGGGATGGAGAAGCTCTGAGGGTATATTTTTCTGCCTCTAAAACTGTT |
| | | GGAGAGGGAATCTGAGAAAGCTGCAACCAACCAGGAGGCTGGGGTACGCTGGAGAAGGAATGGGCTTCCTAACCTTG |
| | | AGCCCTCTTCCCTGAAGATATATGTATCTACGGGGGCCTGGGGCTGGGCGGGCTCCTGCTTCTGGCAGTGGTCCTTCT |
| | | GTCCGCCTGCCTGTGTTGGCTGCATCGAAGAGGTGAGCGCTGCACTCCCTCCCTCCCCCTGCAGCAGTGCCCCCTGT |
| | | GCCCCCACCCCCACACGCTTTCCCACTGCTTTCCCAGAACACTGCCTGGCCCTGGAGCCACTGGGAAGCCAACAGGG |
| | | GAGTCCACGCCTGCTGGTGGGGGGAGCCCGGGAGGGCCCGGGAGAAGCACAAAGGGTGGGCTGTGTTGAGCTTCTT |
| | | CTTTTCTTCCAGTAAAGAGGCTGGAGAGGAGCTGGGTGAGTCTGGGGACAGGGAAGGGGGAGGGCAAGAGAGATCCT |
| | | GAGTGGGTGAGTGGGGAGAAGCATGGCTGAGCGCTGAGAGGAGGGTTGGGGACGGGAGACAAGGAGAGAGAAAGTA |
| | | GGAGCATGAGAGAGGCAGAGAAAATCGAGGCAAAAGAGAAAGAGAAAATGAGACAGAAACCAAGAGAAAAAGTGAGAC |
| | | AGAGGATAGGAGAGACAGGGAGAAAATGAGAGTGAGAGAGACACAAAGAGAAGAGCAATGAAAGAGAGAGAGAGAGA |
| | | GAGGCTCCAGAACCAGGCACAGTGGCTCACGTCTGTCATTCCAGCTATCGCAAGGCTGAGGCAGGAAGATAGCTTGAG |
| | | CTCAGGGGTTGAAGACAATCCTGGACAACATAGTGGGACTCTGTCTCCAAAGAAAAAAGAGAGAGAGAGAGAGAGAGA |
| | | GAGAGAGGGAGAGAGAGAGAGAGAGAGGGAGAGAAGTAAGAAAGGCTGGAGGTGGGAGCAGAACTCACAGGGAAGG |
| | | ATCTGACGGCATCGCCTCCCATCAGCACCTTCTGTCCTGGTCCCAGGCCCAGGGCTCCTCAGAGCAGGAACTCCACTA |
| | | TGCATCTCTGCAGAGGCTGCCAGTGCCCAGCAGTGAGGGACCTGACCTCAGGGGCAGAGACAAGAGAGGCACCAAGG |
| | | AGGATCCAAGAGCTGACTATGCCTGCATTGCTGAGAACAAACCCACCTGAGCACCCCAGACACCTTCCTCAACCCAGG |
| | | CGGGTGGACAGGGTCCCCCTGTGGTCCAGCCAGTAAAAACCATGGTCCCCCCACTTCTGTGTCTCAGTCCTCTCAGTC |
| | | CATCTCGAGCCTCCGTTCAAATTGATCATCATCAAAACTTATGTGGCTTTTTGACCTTTGAATAGGGAATTTTTTAAATTTT |
| | | TTAAAAATTAAAATAAAAAAAACACATGGCTCACCCTTCCACCCA |
| >ENST00000606388.1:: | 134 | GCTAGAGTAGGGTCTGGAGAGAGCTCCCACTGTGGCGAGTCCTGTCGTTGGATTCCCGCGGCGCCCCGTAAGGCTCC |
| chr6: 146136047- | | GCCCCGGCGGTGCTACCAAACCCTGTTCGATTTTAGAATCGTACATTCCCAGAGGGAATCCTAGACCCCTCATTTTACA |
| 146207721(+) | | GTTGAGGAAACAGGCCCAAGTAGCATGACTTGTTCGCGCCGCTAGTCGAAGGAGCAGCAGCCCCGATCCCTTGACTCT |
| | | GGGGCACTGCTTTCCCTTTAGCTCATTTTTGGAGGAAATGGGTGCGCCCTTGCTCGACCTTTGGTGATTGCCGCCACAG |
| | | GCCTTCCCGCCGGCTAACTCAGCGTGCCCAAGTCCCGTGACTTAGTGCCAGGACGGTGCTGTGACCCGTTATCCCCTG |
| | | TCGGGTTTTACCTCCCCCCGGGAGAGCTCAGCCCTGGATCCCGGCGGGCGGCGGAGCCTCTCTGGAGCCCGGACGG |
| | | GACAGCCCACCAGGGTCTGCTTCTGTGAGGGGCCGAGGCGTCATTCCGCGAAGCCTTCTCCTTGCTGTTGCCATGTGA |
| | | AGAAGGATGTGTTTGCTTCCCCTTCTGCCATGATTGAAAAGAAGCATAAGCTGCAATAAAAATTTGGTTTCAAGATAACA |
| | | AAGTCAGTCCCCAAAAATCAGTATTGAGAGTCACTGACATTAAATGATGAAGATACGAGGATGGTGTGGGGACCTTTAC |
| | | TCTTCGAAGATGAGCTATACATGCCCCTGAAAAGGCTGTCTGAAGACAGGGGAAGGCTGGAGATGGCAACTTTGGCCT |
| | | CTGCCAAGTACTCGTGCTATTTTCTGTATGTGATACTATCTGTTTTGTAACTCAGAGGCAACACCACATACCTAGCATAA |
| | | GGCTTGAACTCCCTTTGGGGTTATCAATTACTTACATAGCCTTCCTCTAAGGGAAGGCTTGCAAGTGGTTCATATTCGAC |
| | | CCTTCCATGTGATAAGCATTCATGTGGGCTTCACAATGTAACAGAGGTACACAGAGATTCTTGTAAACAGAAAATTTTAT |
| | | AAAACTGTTAACTGATCTCACTTTGAAGAAGTCTTACTGACTAGATGTCATTTATCAGAGTAGACAGGCTTAAAATGCATT |
| | | GCTTGTACTAGACTGTTGTAATGCATTTTTTTGTTTTTGCATTGGTTTTTAATTCATTTAATTGGTTTACACTCATTTTTTTT |
| | | TTTACATTCCATTTTTGCATTTGTATGCCAGTTAAAAATTATGAAATGGCACATTGTGGGATTTTGTTTAGCTAAATTCTAG |
| | | AAAAATTGAAAGATACCTTTTATTCGACTTAAGCCTGAGGTTATTCAGCACGGTGATCTCTGCATTTTTGAAAGGTTGTCT |
| | | CGTCGTTTAATAAACGTAAATAACTTAAGCACATTGGAAGAGCTATTAAATGAGAGCAGGAGATTAAAAAGACATGATTT |
| | | CTTACTTTATCCACCTGACAGGATAGCTTGCATTGGTGATTAACCTTGCATTGTTAAATTCATTTCCTAAATATTTAGGTAA |
| | | GTAGTTGGTTTGATCATTATTTGTAAAGTATTAGTAGTTGCATGTTGAATTATCATGTTAAGATTTTTAGTTTGTTGTTCAT |
| | | GTGATATGAAAGATAACAATTCCTAAAACTGAATGCATGCATGGCTTATTCAGCTCAGGTCACATACAAAGAGAAATGTG |
| | | ACGCTGCAATAAACATAGCAGAGCTATCTGGGTTTGAAGCAAAAAGGCTATGACGTTAGGTATATGCACTTTACATTGTA |
| | | TGTATCTTTTTAGTTCTAGTGAACTCATCTGGAATCATGTTACAGAAAATGATTCTCCAGGTCCAAACACATTTAAGTTTA |
| | | ATGACTGGGACTACTCTAACAATTGCGGCTATCTCATTTTGTCTCATTGGTTCATTAGCATAGCACCCATTTAAGAACAAC |
| | | TACTTCTAAACACAGTAATATTTCAGAGGCTATAAAATAATAGAGAAAGAAAACTTAAAAAATCCTTTGTTTGCAGCCAAG |
| | | AAAATGTGAGGATCAGATTTGTACTTGTTTTGAACAATTGGAAACTGTCACAATTGTTTTTAAGATGTAATAAATGTATATA |
| | | AAATATGGGATTCTATAGTATTAGCCATTTTGATATCTTTTGTTGAAAGAAAAAACCAAAAAACCCCATATGTTCTAAGTCT |
| | | CAGAAATCAAAGAATTCCACAATTTTCATTTCCACTAAAATATTTATTTAATGGATATCTTACAGAAATAAATAAAAACAGT |
| | | TTTTGTCTAATACAGATAGGTTTTTTTATATATATGTAATTTGATTTTACAAAATTGAAGCATATCAAATTGTTTTCTCTAAA |
| | | ACATAGCTCAGAAAGGAATAGTAGAAAACAAACACAAAGAAATACTGAAATAGTCTCATTTTTAAATTATAAGCATGGGC |
| | | CTTCTGAATCTCAGCATGACATGTTATTAGTAAAAGAATGTAAGATATGGAGTCAGGGGACCTGGGCTCCACTGTTTACT |
| | | TGCTACATGTCCTAAGAAAGTTTCAATATATCTGAACTTCAGTTCTCTACCTTTAAAACAGTAAAGATATTAATTATATCAA |
| | | CTTCACAAGATTGTTGGGTAAATTAAACACGTAAATGTACATGAAAGCTCTTTTTAATATGTGAAGAGCTATAAAAATAAC |
| | | AGTTTGATTTAATTTCATATAAAAGAATCAATGGATACCTCCTTTTAACTGAAGAATAAGCACCAAAAGCATGTGTGTGTT |
| | | TTCTTCAGTTGAATTACATCACTCTATAAACCTAGAGTAAGAAAATGCACCTTAACTAAGCACCAAAGCCTCTCGGACAA |
| | | ATGGGATCTAGGGAATGGTTCTATGAGTTTTCCAGGCAGCTAACTGGGAGCTAATAAGTCCTGAGGGTCAAAGAGGGT |
| | | CCTTTAAAACTCTGAAGGCTCAAAGAAAAGGAAGCCAAATAAGAACATTTGTATTACATCAAATGGCTAGGATGTAACAA |
| | | GCTACTCCTTATTCAGCAATTTCAAAGTCCATTAATTCCTCACAGAAGAAGAAACACTTCGTTATTATCACTTGCAACGAT |
| | | TTGCTTATGTGAACATGGCATTCTCTTGTTCAAAAGGTCAAAGACTCGTGCTTAAGGGAGGAACAACTGGAGTGAAATAA |
| | | CAACTCTACCATGAATTCAGATATCACTGGCTAAAAAGTCTAGTTACACAAGAACATGGGAAAGAAAAAGAATCCAAGAC |
| | | ATCAGTATAATAATGATTAAAAAAAAAACCCTGCCATATTTCCATGTTATAATAAAAAAAATCATAAAAACTGCAATATTT |
| | | ACAGATACATCAATCTTACAACATATAAAAACTTTAACAATAAGAAAAATTAAGAGTAGGGAAAGCAGATAATTTTAAGGT |
| | | AATCATTAATACATTAAAAAATCTATTTGGAGTTAACTGTTTACACTAACACCACTTTACACATAATAGAAATATGAATATTT |
| | | TCATTGTTTTTGTTTTCAGTAGCAAGATGCAGAAGAAACAAATAACTGTCTAACATTAACTTATTGTAAGGAGTTGTATTA |
| | | CTGCAAAATTACTAGGTGTAAAGTTATGGTATAAGCCTACTCAAAAAATGGGTTAATATAATTCACCATCTACTTAAAATA |
| | | TTACTAACAAGATATTGGTGAATCATGTGCTAAAGATACTGAAGGCCCTTCCAAAACTGAGATCTATGTTTGTTCAATATA |
| | | CCAGGTCATATAAAACTAGACTAGTTTACTTTCTTATTCCAACTGTTTTATGAGTGATCTTATCATAAGAAAAGTACACATT |
| | | ACCTCTCTTAATTCCCTTGCATCATCAGATATAGATACTATTTGGGACAAAAAATAAATGTTTTATTAGGAGTGTAAAATTA |
| | | AGAATCTTTATAGATCTTTTGGAAACAGTATATTGAAAAGGACTCAATAAGTACTAAGCCACTGTATAACCAGAACAATAA |
| | | ACAAATTCATTCAACTAGG |
| >ENST00000512382.1:: | 135 | GCAGAGGGCGTCCTTACTCCAGTATTTCCATGTGCTTCCCTGACCCGGGCCGGCCTGCCCACCAGGTCCCTCGAATCG |
| chr7: 296160- | | GGGCCTCTCAGCGTTTGAGCTCTGCTCTCGCCCCGTCCCTCTCCTCACTCCTGCGGGAGAAACGGCCCCTGTTCTTTC |
| 297419(+) | | CGCCCCACGTTGTCCTCGTGAGTGTGTAGTCCAGGTCCTGTTTCCCCACAGAGACTCTGCAAAAAACACGGGGCCCAG |
| | | AGGTGAAGGCAGCTCCAGGTGGGGTCACCCCGAGGCAGGGCAGAGCGGCTCCGTCCCCTCCCACACCCGTGCTCCC |
| | | GCTAATGCAGCCTCAGCGCCGCAGCCCGGGCGGGTCCATCTGCAGACGCCAAGGTCCCTGCCGCAGTGTTTCTCTTC |
| | | TGCTCCTCATGGCACGCGCCGGGCTCCCCAGAATCTGGCCTGGGCCCCCCGTCTCACGCTGGCTCCCCGCAGGTGG |
| | | GAGGTGGACCCTGACTACTGCGAGGAGGTGAAGCAGACACCGCCCTACGACAGCAGCCACCGCATCCTGGACGTCAT |
| | | GGACATGACGATCTTCGACTTCCTCATGGGAAACATGGACCGTCACCACTACGAGACTTTTGAGAAGTTTGGGAATGAA |
| | | ACGTTCATCATCCACTTAGACAATGGAAGAGGGTGAGCCTGTCCTCGCCCCTGCACACCCAGGGAAGGGCCGGCCAC |
| | | CTCCCAGCTACCTGCAGCCCACCTGAGACCCTGGGGACGGGGGGAGCAGACCCTCCAGTGGAGGGATGGGAATGTC |
| | | GCAAAGGCCCATCTCAGAGCCAGATGCAGAGGGCGGCCCCAGGCCCCAACCAGGAGGGAGGGCCGCCCCGGGAGT |
| | | GGGACCTCCGAGGCACAGGAATGCTGCAGACCAAGTCCCGGCAGAGCTGGTGTTACCTGGGAAACGGAGGCCAGGA |
| | | GAGGCTGCTGGTAGGAAGAGCAGGACCGTGCAGAATAGATGGGCCTCTGCCTGCACGCGGTACCTGGAGCCAGCCAG |
| | | CGGGGGATAGGCGGCCTC |
| >ENST00000496622.1:: | 136 | GTGCGCCGGAAGTGATCCCCTGCGTGGCTGGGCTGCTCGGGTTAGATCGTCAGGAAAAGCCTAAAGATTAGACTGTAA |
| chr7: 141438157- | | GAAAAGAAAATAGAAGCCATGTTTCGAAGACCTGTATTACAGGTAGTCACTTGTCTGTATTAATACTGAGATGTATTACTA |
| 141441490(+) | | TCAGCCACAGTGATCAGAAGACTTCTTTAGGCTTTTAACTACAGGGGCAAAAGACCTTTGAGCTCACCCACCCACCTCT |
| | | ATACTCATTTGATATGGAGGATTGGGGACGATTTTGCCAGTGTAAATTATGTTCACTAAAACAAAAAAAAAGTTCAGGTTT |
| | | ATTCACACGTAATAGGAATCATTAATTTTTAAAATGTGTGTGTGTTGCTTGAATTTAACCAGTGAGAGGACATTAAAGGTC |
| | | TTTAGTGACCTAAAGACTGTAGGAATCATTGTTCCAACTTTTTTCTTAAGGCATGTAGTTGCTTTTTTGACCATCTACCTC |
| | | CTGTGTTGCTAAACGTTCCTCTTCCCCCATCTTTCCACACTGAAGAGAGCATCTCAAGTCTCAGTGGTCACCAGAGATTT |
| | | CACTATGTAGAAAATGCCTTCTCATTCCACTTAGTATGGGTCCTCACTTTTCCAGACACTAGCCCACCTTGAGCCAGCTT |
| | | AGACTACCATTTTTCCTCACTTACCACATTCGGGCACCAGCTCCTGTAGATTCTTCTTTGGCTCTCCTACTCCCCATCCT |
| | | CCTGTGAAGACCAATGAAGAACCAGTTCCTCCATGACTCTGTCATTTTCCCCACCTTAATCCTATATTCTTGGATCCCCA |
| | | CTTTAACCTGTATGTATCTTTAATCTATTTTTTTCTGTATTGAAATGTGAAACATGAAAAAGTTATTGCTTCTTAACACTAAC |
| | | GTTACAGTATTAAAATGTCTTTAAATAAGAGTTAAAAGTTTAATTTAAAAAATTATCCAAGTTAAATAATCCTTTCCTAATCT |
| | | TCTCCAGTAGATTTTTGTGTGCCCCAAATGAGACATTGTTATTATAAATCTCTGTGTAAGATTTATGAGGTCTAGCTTGTC |
| | | TGAGCACTCAAATGACATTTCCAGAATCATGACACAGGTTCTCATTAGCAGTTTGATTCTATAGTATGTTGTACTCATATC |
| | | TTTATTTTCTTATCTGCCCTCTGCTCCTAAGTTTAGTGATAGGATTAGATAGCCAACAGCACCCTGGGGATTGGTGTTTAT |
| | | TTGATTGGTGGACTTCTGCTTTTTATATTAACAGTACTGCCACTTCAAAAATGTTTCTCTGATAGAATGGCTAGCTCTCTG |
| | | AGCTCTGAATTCAGTGATGTGTACTCCCATCTTTACAGGAGCTCTTGAAATTAAGGAGGCAGCAGAGGGCTTCGGGAAA |
| | | GCAGTAGCATTGGTGTCAAGGGCCCTGGGTCCTTGAGATCACTTGCCACATACTTCCTGCAGGAGCTTGGGCAGGTGA |
| | | TGCAGTCTCTCTGAGGTGTTTTCTCAGCCCTAAATGGGAATAATTTTACATAACTTGTAGATTTAGTTTTAGAATTGATAA |
| | | TAGATAAATCCCTTAGCTGATATTCAGTAATGATTGCTGCTATTATGTGGTAGTGACTACCTTTGCTGACAGTGATGTGA |
| | | CTTTGACTTGGACAGTCGAGTCTTTGTGTTTAATCAAAGAACCAAATTTATTAGACCTTTTTTGATGCACTAAAAACAGGA |
| | | AATTTCCTATGAAATATTTATAAAAACTGTATTAGGCTAAGCTCACTACTGAAGCTGTTTAATTTTTGCAATAGCCTTGTGA |
| | | AGTAGGTATTATGTATCATAATTTTTGCTTATTTTAAAATGAAACACTTTAGATTAGTCATATTAGGTATCCTAATTCCTTTT |
| | | AAGGTCTGAAGGAGCAACCAGGGGAAGGCAGTTTCATAGACACAGGGAATCTGACTTTGGAATATGTTCCTGTTGCTGT |
| | | GTCAGAAAGTCATTCATGTGATTAGTTTTAGAATGTATTGTTTTGTTGGAATGTAACCTATCTCTCGTTCTCTAAACCAGC |
| | | AGACCCCAACCTTTTTGGCACTGATAACTGGTTTCATGGAAGACAATTTTTCCATGGAGGAGGGGGGTGCCGTGGGGA |
| | | TGGTTTCAAGATGAAACTGTTCCACCTCAGATCATCAGGCATTAGTTAGATTCTCATAAGGAGTGCACACCCTAGATCCC |
| | | TCGCATGAGCGGTTTATAATGGGGTTCTGCTCCTGTGAGAATCTAATGCCACTGCTGATCTGACAGGAGGCGGATCTTG |
| | | GTCAGTAATGCTTGCTCGCTGCTTGCTGCTCACCTCCTGCTGTGCAGCCAGGTTCCTAACAGGCCACAGAACTCTACTA |
| | | GTCCTCAGCCCTGGAGGTTGGGGACTCTCCTCTAACTGGCTGTTCGTTATGCCTGAGAGTAAGGCTTTGCATTTATTCC |
| | | ACACCTCCTGACACTTGTTTGATCATTGCATATGTTATTAATGAATCTACTTATTAAATTATTAATTATAAAGCATCTCTTTT |
| | | AACTCTCCTTTGGTCTAATGATGGATTAAATCAGTACCCATGTTGGATCTAAATTTCTTAATTTCTAAAGTCTACTGTAACC |
| | | AAAATACAGTAGTAATATACCAGTTGTTTATACTAAAAAAAAAAA |
| >ENST00000481651.1:: | 137 | CTAGCCTCCAAGACAGCCCCGCCGCCAGAACAGCCTCTCCTCCAGGACCGGCCAGCCTACGAGAGAGCCCCGCCTAC |
| chr7: 143509060- | | GGTACAACCCGCCTCCGGGACCGCAGGACTCCACCTCCGAGACAGCTCAGTCCCCAGGACAGTCCCGCCTCCGAGAC |
| 143534026(−) | | GGCCCCGCCTCCAGGACGGCCCCTACCTCCGAGACCGCCCCGCCTCCGGGACCGCCCCGCCCCGCCTCGCCTCCGA |
| | | CACGTCCCCCGGGCGCCACTGCAGAGCCTGTCCGTCAGTCCCTAGGTATCCGCACTGCTCAGGGGAGGATTCCCTGG |
| | | GAGCACCCACCAGCTGAGATCTGCACATCAGCCACAATCCTCTCAGGACGGCGGAAAGGGAAGGGCTCAGCCGCCAG |
| | | CCTGGCCACAGCCTGCATCATCTCATCCCAGAGGCGGAGCACAGGCTCGGGGTCCTTCAGGGCCTGAAGGTTTGTGG |
| | | TTGGCACTGTCAGGATGATGTTGTCCGTGGCCAGCTCTCCCCAGGGAGCCAGGTTCTCCTGCATCTGCCTCTTCCACT |
| | | CCTCCAGCGATGTCTTACCTGGGAATAAATATCATGGAACCTACCACACCCACTTCTCCAACTTCCCTTGAGCTGAAAAA |
| | | TACCATTTGAACTCTGGAAGAACATTGCAATAATGAACTACTATCACAGGCGTCTATAGGCTGTAAAGTGAAGAAAAGGC |
| | | TTCCTGACTCTTCTCTTTGTCTGCCCTGAAGTCTCCATGGACACAGGGTATTCCGTATCTTCTGCCTCCATGTTCCCTCC |
| | | CAACAGTTCTTCCTCTTTCTCTCCACCAAAGCTCATCCCTCTCCCATTTAGCAACCACCCCACAGTTCCCACGGCATCTG |
| | | CCACTCTTCCTCCTCCCTAAATGTTCACTCCACTTACCCAGCTTGTAGTATGGGGCAGGCACAGCTCCCCTGATAGTGA |
| | | CAGGCACAGGGCCTAGTTGGCTGCCCTTGGGCACGATGACGTAGAGGAGGCCGCCCCAGAGGCAGGAGACTGACCG |
| | | CTCAGTCCTGTCCATCCAGCATTGGTGAGTCACCACGGGGGCTCGAGATAGCTTCCTGGCCTTGGTAAGGTCATCGGT |
| | | GTGGCAGCCAATCTGTACCTGGACAGGTGATTCCACCAAGCGTTGGAGAAATTAGAGTCTTTGTGATGCTTTGTGATGT |
| | | GCGTGTGAGGTTGTGTAGATGGAAGGTATTAGACAAACATGCCCATGAAACCCCAGCTTCCCTTTTCTATGTGCTAGGC |
| | | ATGGAAACTTATGAAATTTTAGCACTCCAAAGTCATTTGGACTTCAAGGCATTTAAAATCATTTTTCTAAGGATTTAAACA |
| | | GCTCCACTATAAGTCTTCACCTGACATGAATTGGTGAGAGACCAGGCTGATCCTGGCAAAGGTCTTGTGTCTTTCTGCC |
| | | AGGCAAAATCCTGGGTTCTTCTAGCAGGACCTAAGCCAGTCTGGGGACGCTGATATTGAGGATGAGCTGGGGGACTCT |
| | | GCTCTGTCCTCTGTGAACACACAGGAGGCCCATCCAGAGTGAGTGAGGTTGATTCTCTCTCCCTCTTTGCCCAGAGCTT |
| | | CCCTTTCTGGCCGCCAGATGGGTGGAGATCTGTTTTGTCTGGAGTCCTGGAGTTGCTTTTCTTAGGTTTGATATAAGCA |
| | | AGCTCCAGAAAGAATGCTGACAGAAAAGGGACCCTAGCTGTGGTAGGAAGTGGCCCTCAGAGTCAAGGAGGCAGGAT |
| | | GAATTTAAATTCTGCATGTAGGGCATATTTTGGGGAGTGATGGGATTATGCACACCCTTCAGGTGTCAAGATAAAGAGAT |
| | | AAAACCAGAGTTTGTGCAGAATGAGCTTGCTGACACACAGCCTAAATTTGTACCGCATGTTTCATACTAACTCCCTCTGA |
| | | GTTTGCACATGGGACCCATGAGGAGGCATGAAGAGGTAACTGCCCATGCCCGAGGATTTTCCAGCCCTTCCTTTTCTTT |
| | | CTATCAATCACCTACTAATCACAGAATCCACTCCCTACACCTTTTCTACTAAAATAACTCTTTAAAATAAGTACAATGGGA |
| | | CAGATTTGAGCTGGGCTCCTGTCTCCTTGTTAATCAAATTGCAATAAAATGTTTTCTTTTGTT |
| >ENST00000435586.1:: | 138 | ATGTAATTACAAGTGTGCTCCTGGTGGTCTTTAACTGAAGGGCACTGACTGGGTACTGGTGAAGTTCCCCGCAGGAACT |
| chr9: 44401765- | | GAGTCAGACCCCATCTCAGGGCCCTGCAGAAGATAGGTGCCTGCTCTAAGGCGTGGACCCTCGCGACAGCCCTGGCC |
| 44404440(+) | | CGTCTTGACGGGCGAGGGTTACTGTACTTGTCCCAACCGTACAGATGAGAAAGCTCAGACTCAGGGCCAGCAACCCCG |
| | | GTCCCAGCGGAGCGCCCGGCACGCGCCGACACTTCAGCACCAGTCGCGGTGGCCACCACTGTGCGCGGAGATGGCT |
| | | GCGACGCGTGCGCAGTCTAGTGCTTTTTTCCAGATCTCGATCCCAAACTCCCTCCTGCCAGAATCTGGACCCGAATCCA |
| | | CCCATTGCCCATTTTCCGCTGCCGCTGGAGAGAATCTCTGAGGTCCCCAGGACAGCCTGCCTGCACGGAAGAGATGCC |
| | | TCCTCAGTATGGCCGCCCCCGGAGAGGAGCGATTAAGTGCAGACCTCCATGTTGCTCTTGAGCCTGAGTGGCTTCAGG |
| | | GAGCCATGTTTGTTACTGGCGGGCGCCGGCCTCACTGAGCATGTGCAGCCCTGGCCGGGCGGCCTCAAAGTTCTGAC |
| | | ATCACAGGGCGGTTCCTGAAGTGGACGTAGTTGTAAGAGCTAGTTATTTTAGACAATGCCTCTGGGATCAGGGACTCTA |
| | | ATCTGGAAATAGGTAGTGGGAGAGGTCGGTGATGCTGTCTCGGGGTCAGAGACCTGAGCTATATGGGGTTAGAGAGG |
| | | GGCCCTGGGCAGGCGAGTCTCTGGGGAGTGTGGTGAGAATCCTTGTGTAAGATGCTGGGAGGAGGTGGGGTCAGGG |
| | | CTGGGGTCCGTGGGCCGATGGGTTGGGGGATGGCCAGCGTCAGGGATCAGTAGTAGAGATTCTATGTGCCCTGATCG |
| | | CCAGTAGAGGTTTTAAATACAGAGTATTCATGAGTTTAGCAATGTTATTCGCCTCTCATAATTTAAATAATTTGAAGTTTTC |
| | | CCCCAGTAACTAGTGTCTTAGAAGTCATGAAAATTTCAGAAAATGACAGGTCTTCTGAGTTCGTGATGGGAGTTGGGCA |
| | | GCAGTCGCATACGAGCACCTGGAGAGTCCTTGCCAGTTCTTTGGGGATGGGGAGCTCTTAAGACTGCCCTGAGACCGC |
| | | CCTTTGACCTCATTGTGGTCCTTTCTAGATTAAATGCTGTTTTCCATGACCGTCTCTGTTCTTCCCATACGCGAATGGCA |
| | | GGCATCCAGATCTCCAAGAATAGAGGATTAGGAGAGACTCCACCACCTATGTCCTCACAGTTAATTATCGATTTGTGTCA |
| | | CTTGCCCATTTTCTCACTTCCTGTTTGTGTGTCAAGGAGTATTAATAAATCTTTGCCTATTTAAAGATACTAGCCTTGGAT |
| | | TTTCAAATATTGCATATTTTGACATGTAAAAATGTGTTAGTTTTATTTTTTCAACCGATCTGACCTGTATAGTTGGGCTTGA |
| | | GGAAGCTTCCTCATTGTACATGTTACTATGGATTTTTTAATATTAACACAGAGTTGTATTTTTTCCATATAAATTTCTATTG |
| | | AATTTCTTTTTCCATATGATAGGTGGTGAAGGTTTAGCCCAGTAAAGCAGAGAGGTTAAGAGGTTAGACTGGGGGCTCT |
| | | GGAGCCAGACCTATGTAGATCTGAGTCCTGGCTCTGGCACTTGAAAGCCGTGTGACCTTGGTTAAGATACTTAGCCCCT |
| | | CTCTGCCAAATGGAGAAATAAGGGCACCTACCCCGTAGGGTAGTTGTGTGAGTACACAAGTTAATACACTTCAATCACT |
| | | AGCAAGAAAGTGAATGTCAAGCTGTATTTGTTCAGGCAGCCATATGGTAGCCCCACCTCATTAGTAAACTGAGGTATTG |
| | | AAGTATCTTCTTTTTCTTTCAGAAAATATTTGTCAAGCACCTTCTGTGTTCTAAGAACTGTTCTAGAGCTTTGCATGCTATG |
| | | GAGGTCTAGAAATTCACATTATGGTGGCAGGTGACAGACAATACAAAGAGATAAAGCAATTTCATATAGTGATAACCTAG |
| | | GGAGGCGTGCTAAAAGGAGCCAAGGTGTAGTGGTGACTGGAAGGTGACCAGGGAAACCACTGTAGGGTTTCTAACTGA |
| | | TGAGATCTGTGACAAATCCCTGTGGTCAGCAGCAGGCATTGGATATGTAACTTTATTCTGTCCCATTGATCTTGTAGTCA |
| | | ACCCTGGTCTGTTCTGTATTGTTTTAATTACATAATCTTTGTAATAATCTTTAATACCTGGTGGAGTGCTCTGTTTACTTAT |
| | | TTCTGCATAATTAACCACCCCTAAGCTTAACTTACTCATGGTTCTGCAGGATGACTGCTCTCCATGGTTATAGGGGTTTC |
| | | TGCATGGGGCCTCTCACATGTGCTACAGTCAAACATTAGCCGGGGCCACAGTCATCTGAAACTCACTTACACGGCTGAC |
| | | AATTGATGTTGGCTGTTGCCTGGAGAAGACATATGTGGCCTGGTGGAATGGAATGGTGACTAGATTCAAGAAAGAGCTT |
| | | CCCAGGAGCAAGGCTACCAAGAGACCGAAGTAGAGGCTTCAGTTTCTT |
| >ENST00000439875.1:: | 139 | GAGTCCAAGCCCAGCGGTGTTTGCGGCGGGTCCGGGGCCTGGAAGCGCGGGGGGGGCGCCGAGAGTGAGTAAGAA |
| chr9: 115865617- | | GTTCTGGAACAGCCTCAGTTCCAAAAAAATGAGTCGCTTTGTTGCGGCATTAAATTTTGCCCCTAACTCATGTGTCCCGG |
| 115873753(−) | | CGTGGCCCGGGAGCAGCGGGCCCTTCCTCTTCCAGGCTCCCTGGCCCCGGTCTTTGATGACTCAGAGCTCCTGCGGT |
| | | CACCGGGCCGCTCTGGAGACTCATCAGAGCAGCTCCCCCATCCCGTCAGCTCTGCGGTTACAGCCTCAGTTAATTTCC |
| | | AAAGTCGCCCACTTTCAAAACACGATCTTAGGGCGGGACGATATGTACGCATCTTCCTGTGGCCAGCAGCAGTGATGG |
| | | CAACGCTGTAGGCACCTCCTTCCCTTAGGAAGAAGCCAAACATTTCACGAAGTCCTTCTGTGCTTTTAGACGAAACTGG |
| | | AAAATGGCCTTCATTGGAAAAGCCCCAGTCCTCGGGGAGCAGATCCCCCATCTCTGGCAGTGCAGTGTGCCTGGGAGC |
| | | TGTCTGGAACATTCCGCAACGGTCACTCCTCTGCCCTGCATTGGCCTTGACCACATGACCCAGAGGTGCAGGGAGAAG |
| | | CCTCCCTTGGATGCTTGGAGTGACTTCAGCTGCTGAGAGTGTCTAAGGAGAGCCGGGAACACCGCACGCTTCCTGCAG |
| | | ATGGCGGGGCAGACCTGTGTGCAAGAGTGCCTGAAGGGGATCTCCCGTGGCGCTACCTGGTCCAGGGAGGTCACAGA |
| | | GAGCTCCTCCACCCTGAGCACAGGGGGTCCCACCAAGGACAAGGACAAGATTCAGCACCGTCGCACACAGAACAGCT |
| | | ACCTGGAGCCACAGGTCACTTTCCAAAAAGGGAGTTTCCAGGCCAGAAGCCATCCCTGCAGCATTGTCTTTGTGCTCCA |
| | | AGAGAAGCCCTGGCACACACTTCTGCTCAGATGGAACCACTGTTCCCTCAGTGTCCTTGTCAGTACCAGCTGGTGACTT |
| | | TCCTACTCAAACCCTTCCCTCACTCTCTGCCTCATTTTCTCAAGGTGCCGTGTTTGCCTGAAAGGACATTGGCAGCCAG |
| | | CAGGGGCGGCGGCTGGTCCTCTAGATGGGCACGAATGCCATTGGCAGGCAATGGGGCAGACTCCATGGAAAGATGCT |
| | | TCTCTTCCTCAAGGTATGCACTCAGCGGGGAGACCTCATTGTCAAGTTCAGAGTTACGCTGCAGACTGCACAGCACCTC |
| | | GCCTGCTTCTAGGGCCTGCTCCGGTCAGCCCAGGACCCACCACAGCAGGACCCCCCATCCTGTGCTCACCCGGGGCT |
| | | TGTCCATGGCACTGGAAACTCCTTCCTGTTCCTGATCCCCCTCTGGGCGAGGGGTGGGCAGGGACATGTGGCTCGTG |
| | | CCGAGGTCTAATACTGTGTTCCCAGCATGGAAGCAGGTGGGGACACTTCCTGTGGCATGCAGGATTCCGTGTAGAAAG |
| | | CTGTGACTGTCACCCCTCCTCCCCACTCAGTTTTGTTAGTGGACCTTTCCCTGGCCCTTCTCTCCTTGGCCCCCTCTTG |
| | | GCAGGAGAGAGGAGGAGAGGATCTCACTTTCCCCCTGTACCAGCCACACCCTCGGTCTGCGGGGTTCCCAGCAGCTG |
| | | GCCAGGGATGCTCCACACCTGGAGGTCAAGTAACCGTCCCCTCACTCTGGGCATCGGTGCCCTCTCTGGGGTTGGAA |
| | | CAGGAAAGAAAGCCAAGACCTGTATGTGGGACTTGAGTTGAGACTCAATTCTGTAGAGTCAGGGGTGAGGAGGAGCCA |
| | | GGCCTCCTGTGTGCTCTCCATACCCCAGAGCCGGGTGCCCAGTTCCATGGGTCCTGATGGACAGAAGGGAAGAACGG |
| | | GGGGGCTGCTGCACCGTGGGTGGTAATGCAGTGGGAGCCACCTCCAGCTGAATGCCCAGGGACTCCTGGGGCTGCT |
| | | GGCCCCGGGTCCCAGCAGTGGGTCCTGTTTCTTCTTTACTCTTGGAATGCAGGATCTGCCACACCAAAACGTCCCCTC |
| | | CACATTTGGTGAAATAGGAAGCTCCATGGCATGTCTCCATCTTTCAAGGTGACTGAGAGGTTTATTTTCATAGGAGCCTC |
| | | AGTGGCCTTGTCCGTACCCACCTCTGCACGTGGTTGCAGAAAGTGAAGGATTGTCAGGGAGCAGGGCAGACATTTGGT |
| | | TTATCTGTGTCATTGGTCAAAGTTTTGTTTTTCCTCCAGAAGAAAAAGGCTTGTGAACATGCCACCGTATTCTTCATTCCT |
| | | TCTTTTGGAAATGTATGAAGAACGGGTGGCATTTTGGAGTTATTGGCCTGTGAACAGCTGGCCCAGAGGAAGGGTTAGA |
| | | TGTGGGTGGGTGTGGCCGCCTCTGCCCCTCCCCAGCGCAGGTGTGCGTGGGCAGCCCAGGCAGGCGCTCAGGAAGG |
| | | GTGAGGTGGAGCCCTGCACGCTCTGAGCACAAAGTCCTGGGATCCCTTAAACCCAAGCACTGCCTTGACAGCAGCCAG |
| | | CATGGCTGATAGAAGAGACACAGAAGTCCAGCTGATGCCAGACAGAGGGCGTCACTAGAGAGCCGTGCTAGGTGGGG |
| | | GTCATTCAAGTTGTCTTGGCGTATGCAGACGTTGCTTCTAGAGAGTCAGAAGCTCTATATGCGTAGTGTTGGAGCAGAA |
| | | TTTCGGGATAGTGATGGCAAGCCTCCTCCCAAGCCAGCTGGGGAGTATGGGGAGGGGGTGGCCGGAGGAACCTGGC |
| | | ATCCCCGGGTTAGGACCACAGAGGTGGCTCTGCCTGCAGCTGGGCCTCTGCCTCATCCTGACTCCCCCTGCTTTGGCC |
| | | ATGGCTCTTCTTGTCCTTCCTCTTCTCAGTGCAGGAGGACCCTGAATCAAATGCCTCATCCTGGTTTGCATTTTACCCTC |
| | | GGATGAAGCTTGTGGCGAACCCCTGGACTCTGTGTCTCCTGAGTCTCTGCAGACCTCGGGTCCTGGACCCAGAGACTC |
| | | TTCTCCCTGGGACAGGAGGCACTGGGGTGGGTGGACAGGGGTGGCCTGGGGCACAGTAGCTGACGGGGGGACTTCC |
| | | TAGTTTTCTGGGCCTTTCCAACTCTGAGTGTGACCTTCCTATTCTTGATCACAGCCCCCAACTTCGGAGCCTGCTAGAG |
| | | CCTGCAGAATGTGGCTTCATCCTCTTCCTCTGTGGGAAAAGGCGGGGCCTGGCAGTCCCGCCAATCTTGTATATTTGCT |
| | | CCCCACCTCGGTGGTGAATACATTCTTGGGTGGTGAATAGGTTCTCTCCTTGCCTTCACTCTAGAAAAGTCCCCTGTTTT |
| | | GTGATGTAGGATGTGGTCAATGACTGACTCGTCCCTTTGGACCATAGACGCAGCTCTGATTTCTGGTGTTCCTGGGCTC |
| | | TGCACACAGCAGGAGCCACTCTGGGCTCTGAGAGGTGCATCTTCTGGCTCACTTTCTTCTTGTTGGCTCTTCTCCCTGC |
| | | GTCTTCTGCCACTGCCCTGTTCTGGGTGGGAGGCTGTCACACTGTGGTGGAGGGTCCCCTCTCCTGCCTCCCCTCTTT |
| | | GATCCTTTTCTGTGAGGGTCTGCTGGGGTCCTGTGTGTCGTGCATTGTTGATAGATTCCTGTCGTGCTTGCTGTCCTCC |
| | | CCATTCTAAGGGACCTCTGATTGCCTGTGAGCAGTTTACAGGGTCCTCTCCTGGCCCCTTCACCCGAGGAATTCCCGCA |
| | | ACGCATGAGTTTGTGAGGGGCGGATCCTGGGAGGATGTGACGTCAGGTGAGAAGGGAGGGCCCGTCCTAGTCCGGTG |
| | | GGCTCCTCTAACAAAGTGCATTAGCCTGGGGAGTTACTGACGCTCGAAATATGTCGTCAAAGTTCTGAAGGCTGAGAAG |
| | | TCCGAGATCAAGATGCCTGAGGATTCAGTGCCTGGTGAGGACCCATTCCTTAGGGCAGTGCCTCTAGCTGTGTCCTCA |
| | | TGTGGCCGAAGGGGACAAGGGAGGTTGCTGGAGCCTGTTTGATGAGAGCACTACCTTCCTTCAGGAGGGGATTATTCA |
| | | GAAAGTCTGTCTGGTTTCAGGGCATTCCCAAAGGCCCCACCTGTTCATTCTACCCCATTGGTAGAATTCCCATTGGGAA |
| | | TTAGGCTTCAACATACAAATTTGGGAGACACTGACATTTAGGCCATAGCGCGTCAAAGGCAAAGTGAGGCTGAGTGTGG |
| | | AAACCATCCCAGAATCTGGATGTGGCCCAGGCAGAAGGAAACTCAAAGTGATGGGGTGACCGATGGTCCCTGGTCTGT |
| | | GCAGGACTCAGCGGTGCGGATGGCCCTGGGCACGGTGGCAGTGGCTGTTGGAGGATGATAGGGAACATGTTGGTGG |
| | | GGGGAGGGTGGAGCCCACTATGAGTGGCAACTCCCTGGGGGCACAGGAAACAAAGCTAGAGGTGGCTCCCCTCTCCC |
| | | CTCCCTGCACACAGTGTCACCTCTGTGGTGGCCCTGTCGTCCGCCTCAGTTCACAGGTGTTGCTGTAACAACTCCTGAA |
| | | ATTACCTTCAAAAGGAGGCTCGTGCAGGTGCCTGCAGGAGCCAGGAGGCCGAAGTGCATCCTGAGTGTCCGGGGACA |
| | | GGCCAGCCTGGCGGGGTCCACGGCCCCACAGTCCCAACGACTGCCTCAGAAATGGGCAGAGAGCATGCCTGGTTCTA |
| | | GCTGGCAAGGCCCACATCTCAGAGCTGTGGACAGTCATGAAGGGTCCGTGAGCATGGAGAGGGGGCAAAAATGACCC |
| | | ATGAACCTCAGGCCACTGTGCTGCCATCCAGCCAAGTGCGGTACCAGCCCTTCGGGCCTCGAGATCCTCTACCCTTCT |
| | | CTGGGCCTTGCTGATTTCCTCCTCCAGCCCCTCTGTTTGTGTACGCACATACACACATGCACGCACGCACACGCACACC |
| | | CAGTGCAATTTCTCTCTGCTCCCTGTACTCCCCAAAATTCAATGGGTTCCTCTTTCCTCCTCAGAGATATAGTCCTGGGC |
| | | CCCATCTCAGCCTGCAGAGGCCTGAACGTTTCCCAGGATTGTCTTCTAATTCTCAGGTCCGGCTTTAAAACAAAACCTAA |
| | | ACTAAAACTAAAATGCTTTCTGCAAAGAATAATACACCAACATAAAGTCCGGGTATCCTAAGCATGTGGTGTGTATGGTG |
| | | TCTGAAATTGAATATGCTGTGAAACTGCCTGTGTCGAAATGCAGTGTTTCCCAAGACCTGGAGGCTCTCTCCAGCTGCT |
| | | GATTGTGATCACCTCTCCCGATCACCGCTGAGCCCTGCAGGTCACCTTCTCCTGACCTATTACACCACTGATTGCTTAT |
| | | GCTGGGCACAATTTTATACCCTTGCTGTTGTTCAGTCTAATCATCCCTATAACCCTATCGGATCATTCCCATATGACAGA |
| | | GGAGAAAACCTGGGGACACAGGGGCTGTGTCCTGGCCAAGGCCTTGCTTGCAGCAAAAGGCAGAGCTGGGGTCTAGC |
| | | CAGGAGCCCCAGCACAGTCCTGTTCTTCCCCCACTAAACTGAGCTGTGAAATAGATCATGGCAGTTTGGAAAACGGGCT |
| | | AACTGTATGCTGAGGGAGGTGGTGACGGATACCTACTTCTTTTCATGGGAAGGTGAGGTGTCTCCCTGCCCCCCTCAG |
| | | GTCTAGCCTCTCTCTGTAGCTCAGAACCTTGGGAAAGTGGGAGGCTCAGAGGAGGAAAGAGATTTACTTGCCCAAGGG |
| | | CCGCAGCTGCTAAGTGGCAGGTAAGGGACTGTTATTCTCCTGTCCCTTTCACAGAGGACAGGAGGAAAGTGAGGACAG |
| | | GTGGTGTACATGCATCTTTTCTGCAGCAGGCCCAGATCAGCTTTGCACATGGTGCTGACCTCAAATTCAGCCCTAGAGG |
| | | GGCCTGTCTCTGCCCTGAGTGAGTATTGAAGTGCAGGCTTCTTCCCCTTTGTTCTGTCCATTTCCAAAAATGTGAACACA |
| | | CACCTCCAACATGTTTCTGTGTATATTATTGTGTGCATAGATACTACCATTGTGAAATATTATGCAGATTAATAATGCACT |
| | | ATGCCATTGAAAATTTCAAAAGAAATGTGTAATGAACATTTAAAATTAATATTTTTGTTCTCAGTTAAGGGTTAAGGGATAT |
| | | AATCAGGTTCTCTGCAAAATACTATGGTTTAAAAATAACAAAGATAATATGGGTATTAATAACATTTCCTCAACAGGAATG |
| | | ATGGAGAATGCTTCAGTTTTTCTGTAGGGATTTAGTTTTTGAATTGGGATTTTGGGTAAGATATCTTTAGGATAAAATATG |
| | | TGCACACAAGAAAATAGCATATTTTTCCTAAAAATTTGTTTTTTATGCATTATTATATACACATAAAAATGGTCTTATTTTGG |
| | | CTGGGCACAGTGGCTCACGCGTGTAATCCAAGCACTTTGGGAGGCCGAGGCAGGTAGATCACCTGAGTTCAGGAGTT |
| | | GGAGAACAGCCTGGTCAACATGGTGAAACCCCATCTTTATTGAAAATACAAAAAACATTAGATGAGCATGGGGGGGGCA |
| | | CCCATAATCCCAGCTACTTGGGAGGCTGAGGCAGGAGAATCGCTTGAACCTGGGAGGTGGAGGTTGCAGTGAGCCAA |
| | | GATAGCGCTGTTGCACTCCAGCCTGGGCAATAAGAGCGAAATTCCATCTTAAAAAAAAAAGAAAGAAGTAGTTTTATTTC |
| | | AAAATTAGAAGCAAAAAGAAAATAAATATATCCTGCTATAGAGACTTGTCTTCGGATGCTTATGACCTCCTGTAAATGCAG |
| | | TTCCTCCCTCATCCCCGTCTAAGCATCCTTCCTCAGCCTCCTCAAATTGGAGCCTGCGTCATCAGAACCACGTGGAAGG |
| | | CTCTAAAAACATTGACGGCATAGAGGGAGACCCTATCTCCAAAAAACAAAACAAAAAAACCCTTTATTTTTAATGGGATA |
| | | AAAAGTTAAATGGTAGTTAGATATTCAAGTTTTGTATGCTCCATGAACAAACAAACAAACAAACAAAAAAACATGACACTT |
| | | GATGGAGACTGCAGGCCTGCGTTCTGTACCTGGCTTTGAGGGAGTGATTGGATGGGATGGTGTGCTTCCTGTGCCTGT |
| | | CCTGTCTGCTCTGGGTTTAATTTACTCTCAATTGGCCATTGTTCTTTCCATGATCTTATTTTCCAGGCATTGTCCTAGGAC |
| | | CCAAGGTCTCTTACATTCTTACATGTAACAGTAAGAATTCTTACAATAAGAATTGAGAGAGTAGAAGCTATTTTTTCCTAT |
| | | GACTTAGAACTTTGGGGGAAGTTTAAAGGGAGATACTGCATGTATTCTGTGTCATCATCTAATTTCTACAGACCCTTCTT |
| | | AAGGAACTGACATTAATGTTGTACTTAATTCTTCAGGCAGTGTGGGCCAAGAATAAAGCCAGATATACATTTAGTCTAAA |
| | | AGTACAAGTCATATTATTTCCATCAGTGGATTCCTATAATCCTATGAATAAAATCTTATACATTGGCTTATAA |
| >ENST00000412744.1:: | 140 | TCTATAGAACAGGATGTAGCAAGTGCAAAGGAAATGCCTGGTCAGGGAACATCAGATTCAGCTAACTCCCAAGGAAATT |
| chr10: 116261755- | | CAACACCACATCCTCAGGTCCCTGGTTACCCTCCAGACTGTGTTGTATTCCTGAATAACTTACCAGAAGAGACTAATGAG |
| 116262106(−) | | AGGATGATGTTTCATGCTATCATATCAGTTCCCGCTTTCAACAAAGTATGTTTGGTACCTGGGAGACATGACATTGCTGT |
| | | TGTTGAATTTGAAAATGATGGACAGGCCAAAGCTGTCAGGGATGCTTTGCAAGAATTCAGGATCACAGCATCCCATACC |
| | | ATGGGGGTCACCTATGTCAATAAATAATACTGA |
| >ENST00000488828.1:: | 141 | TTTTTATTTTTTAAATAGAGATGGGGTCTGTATGTTGACCAGGCTGGTCTCAAACTCCTGGCGTCAAGCAGCCCTCCTGC |
| chr11: 3716700- | | CTCAGCCTCCCAAAGTGCTGGGATTAGAGGCGTGAGCCACCACATCCAGCCTCCGTCTGGTACTATTTATGGTCTTTGT |
| 3718490(−) | | GCATTTGTACAGTGAGAATTTATCTTTGGGATTGATCTTGTATGAACTTATTATGAATCATTAAAAAAAGGTTATGCTCAC |
| | | CAGGACACCAACATGTAGTAGGAGAGGATCTTAAGGCATGCCAGAGTCAGATTCTTAGGGCTGTTGATTCATAAACTCC |
| | | ACTTTCTTCCTGTTTATTTTGACTGTAAGGCTAAACCTCTGGTAAAGGTACTGCCTTTTAGGTTACTAAAAGCTTTTGCTT |
| | | TACTACTCCCCCCCTTTTTTTTTGTTTTTTGTTTTGTTTTTGTTTTTGTTTTCGAGACAGTCTCGCTCTGTACCCCAGGATG |
| | | GAGTGCAGTGGCATAATCTTGGCTCACTACAACGTTTGCCTCCTGGGTTCAAGCGATTCTCCTGCCTCAGCCTCCTGAG |
| | | TAGCTGGGATTACAGGGGATCATCGTCTTGCTCTTCTTTTATCTCAGTTTGTGGGTAGCCAGTCAGTCCGGGAGCTGCT |
| | | CACCATGCAGTTGGTGGACTGGCATCAGCTCCAAGCAGACTCCTTCATCCAGGATGAGAGACTGCGCATCTT |
| >ENST00000469976.2:: | 142 | GAGTGCTGCAGCCGCTGCCGCCGATTCCGGATCTCATTGCCACGCGCCCCCGACGACCGCCCGACGTGCATTCCCGA |
| chr11: 18416107- | | TTCCTTTTGGTTCCAAGTCCAATATGGCAACTCTAAAGGATCAGCTGATTTATAATCTTCTAAAGGAAGAACAGACCCCC |
| 18418945(+) | | CAGAATAAGATTACAGTTGTTGGGGTTGGTGCTGTTGGCATGGCCTGTGCCATCAGTATCTTAATGAAGGTAAGTGAGA |
| | | GTCTACCACACTGGAAGCCCATACCTTGACCCCATCCTCTACCCCCACTCCTACCCCTAGAACTGTATTATTACATTTCA |
| | | TGTAACAGTATTTAGATTTATGCACTCATTCGGATAACTTTCTGTGAAACAAACTTTTGAAATATGATAATACACCAAAAGT |
| | | GTATCTGAAATTAAAAAGAATCAAAGGTTGTCAGGCTGGAGACCCAGTTCCTAAAATTCATTATTCTGTATTAACATGCAT |
| | | GGATTGACTACCAATGAAAAGGAAGGGTCCATGATTTTAAATGAGCCAAAATTCTTTTAAAGTGATTTTTGAATTGAAAAT |
| | | GACAATTCAAAAATTGTCATTTATTGGTAAAATTATATGGGAAATCATAAGTTCTCCCACTCAAATCTCATTGCCCCTGTG |
| | | CCTTGGATAGCAATT |
| >ENST00000524838.1:: | 143 | AGTTCCTCAGCCTCAGTGCTATGAAGGTGACAGCGTGAGGTGACCCATCTGGCCCGCCGCGATGCTGGCAACACGGC |
| chr11: 67374414- | | GGCTGCTCGGCTGGTCGCTTCCCGCGCGGGTATCTGTGCGTTTCAGCGGCGACACGCACCCAAGAAAACCTCATTTG |
| 67377739(+) | | GCTCGCTGAAGGATGAAGACCGGATTTTCACCAACCTGTACGGCCGCCATGACTGGAGGCTGAAAGGTTCCCTGAGTC |
| | | GAGGTGACTGGTACAAGACAAAGGAGATCCTGCTGAAGGGGCCCGACTGGATCCTGGGCGAGATCAAGACATCGGGT |
| | | TTGAGGGGCCGTGGAGGCGCTGGCTTCCCCACTGGCCTCAAGTGGAGCTTCATGAATAAGCCCTCAGATGGCAGGCC |
| | | CAAGTATCTGGTGGTGAACGCAGACGAGGGGGAGCCGGGCACCTGCAAGGACCGGGAGATCTTACGCCATGATCCTC |
| | | ACAAGCTGCTGGAAGGCTGCCTGGTGGGGGGCCGGGCCATGGGCGCCCGCGCTGCCTATATCTACATCCGAGGGGA |
| | | ATTCTACAATGAGGCCTCCAATCTGCAGGTGGGTAGGGAGAGATGTAGACAGATGAGAAGGTGTTCAGTGTGCACTCA |
| | | CACACCCCTCACCCAGCACAGTTGTTCTGAGGTGTTAGTACCTGGTCTGTCAGTGGTTGAACTGGGGGAGTGGTGGCC |
| | | AGTCCTGCATGCATCCCAAATGGGACTTGGCTCCCAAAGCCTCCTTCCAGGGCTCCTAATGCTGCTGGTGTAAATTTTT |
| | | TTTTTTTGCTTCAAAAATATAGTATTTTTTATAAAACAAGTAGCAAGAAGAAAAAACCTAAGTGGAACTATGTGTTTAGCAA |
| | | GAGAGCGGAATAATTATGAATATAAACAATTCCTATTTGGAGAAAATACATTATGTTCATTGCTTGGAACTATGAATAATA |
| | | CTTATATCTGTAGGAAATATAGAAAAATAAAATAGGTTAAGGCAATAAGGATATAGGCAATTACATTTTTATTTTAATTTTA |
| | | TAAGGACTTTTATTTTTCCTTTACAGACTAGGGTAGTTAGGAGACCTGATAGTAGCTACTTCGTTTTTATTTTCCTGGCAG |
| | | CAAAGCAGCTTACTTATGTGTTCCTTCTTGCTTATCTGTTCCAGATCATAGTCAAGTTTTCCAATTCGTTTTACTAAGCTA |
| | | GCAAAATTTTTTATACCAG |
| >ENST00000545202.1:: | 144 | ACAGGAAGCTGAAATGCATATTGCTAGATAGAAGAACCAATCCGAAAAGGCTACTTTCTCTATGATTCCAACTACGTGAC |
| chr11: 69240457- | | TTTCTGGAAAAGGCAAAACTATGGAGATGGTAAAAAGAGAAAAGGATTTCTGCTTTCAAATCCGCTGTTTCCTGAGGCTG |
| 69244389(+) | | GGTGCTGAGTTCAGCTGCCCCTGGAGGAGCAGCAGCTCTGGGCCGCTGGCAGGGCCGCATCCTCTTCAGTCTGGACA |
| | | AATTCCCTGCAGCCAGGGGAAGTGGCCCATGCCTGCTGTCCTGGTATGCAACATCGGACCTGCATGCTGGAATTTGAC |
| | | CCTTTTCCTGCCTGACACCCAGCAAAATAAACCCCAGGGAAGGGATCAATGCCTGCAACCGGAATCCAGAAGAAAGAG |
| | | GAGTGACAGATAAGCCCTGGGTCAGAGGCCAGTGAGGTCAGGACCTCCCAGGGACGGACACACAGCCCAAGGTTTGG |
| | | GTGTGAGATTTCAGAGCCCCAGGTAATAAAAATAAAAGAGTTAAGGTGCCTAGCAGCTCAGTCCCTCACAGTGCTACCT |
| | | GGGGCAAGGATGGAAATTCACCTCAGTTTGCTGTGGTTGCTATTGGCTGAATCATGTCCTCCCAAAATTCATATGTTGG |
| | | AGTGTACCTCGGGATGTGGTATCTCAGGATACCTCAGTACCTCGGGATGTGGCGTTGTTTGGAGAAAGGGTCGCTAGA |
| | | GAGGTAATTAAGTTAAAATGAGGCCATGAGGGTGGGCCCAAATCCAATTTAATTGGCATCCTTCTAAAAAGGGGAAATTT |
| | | GGACACAGAGAGAGACACAAACAGGGAGGGCGCCCTGGGAACAGGAAGGCGGCCACCTCCAAGCCAAGGAGGTCCT |
| | | GGAGCAGATCCTCGTCTCAGAGCTCCAGCGGGAACCACCCCTGCCCAACCTTCATTTTGAACTTCTGGCCTCCAGAGC |
| | | TGTGAGACGATACACTTCTGTGGTTTAAGCCGTGTAGCCTGTGGCACTTGGTTATAGCAGCCCCAGGAAGCCCAACACA |
| | | GTGGCCAAAACAGAGAGGACAGCTGGCTTGCTAACGTCACACAGCACACTGACGAGCAGGGTCTTCAACTCACTACTC |
| | | AGGAACCCAGTAGTCAGGGGTGAGGGGGAATGCCCTTCCAATATGGCTAGAGCCAGACTGTCTGGGCCCAATCCCCA |
| | | GCTTTGTATGTATTACCTAACCTCTCTGTGCCTCAGTTTACCAAGCTATAAAATGGGCATAATGACAGCACCTGCCTGGT |
| | | GATGCTCTTGGAGGGATGAAAGGAGGCAACGGTGTGGGCATCTGGACGGCACCAGGCATTGCCCTGAGAGCTGGCCA |
| | | TGCGGGGAATGCTCCATCCTTACGCCAGCCAGGTGTTGCCACCCCTCTCTTGCTGATGGAGACCAGGAGGGTCGGGA |
| | | CAGAAGGCCTTGCCCAAGGTCTCACAGCCAGGAGCCTGCCTCCACTGGTCCCTGTGTGGCCAGTGCTGCCCTGGGGG |
| | | CTGCTGCTGTGCACTGGAGGGTCAGCTGCATGGAGGAGCCAGAGGAGATGGTGCCGAGGACAGAGCTTGCGGCCCT |
| | | GTACCTGGCTCCCAGAGGAATGGGTCTTGGAGCTGCTCTGCACAGACACTGACCTTGAGGAGAGACAGGGGCGCCAC |
| | | TCCCTCCTCCACTCGCTCAGGTGAGCATTCAAAGCGTCATAGGTAACCAGGAGCACTAGTTGGCATGGCGATTTCAGA |
| | | GGTGAGTCCAATGTGGACCCTGGCCCTGGGAAGGTCACAGTTTGATGGAAGATGCCAGACTCACACATTCACTCACTC |
| | | ACTCATTCCCTCGTGTATTCATTCCGCAAAGCTTCACTCATTACCTACCTGGGGCCAGGACTCAGTCTCAGAGCCAAGG |
| | | AGAGCGTGGTTGGGAGGAGACTCAGACGCTTTCCAGCATCCCTGCCCCCAGGTGTCTACACACCTGTGTACTCCCCAA |
| | | CTTGGAGTGTGGCAGGGCTGCAAAGAGGGTGGATGTGCTCCCGTGGTGAGGTTACTTTATATCGCAAAGGTGCCGGAC |
| | | TTCTGCAGGTGTAATTAAGGTCCTAACCAGCTGACCTTGAGTTAATCAAAATGGAGATGATCCTGGGTGGGCCCAAGCT |
| | | AATCAGGTGAGCCCTTTCAAAATGGCCTTGAAGTCAGAGACTCTCCTGCAGGATTTGGAGATCCAGCCCCATGAGGAAC |
| | | AGGGGCAAGGAAATGCAATCTTCCACAACCGAGAGAGCTTGGAAGAGGGTCCCAAACCTCAGAGGAGACCCCAGCTC |
| | | CGGTTGGCACCTTGACTGCAGCCCCATGAGACAGAAGACCCAGCTATGCCATGTCCTGGCTCCTGACCTGCCCAAACT |
| | | TTAAGACAATAGATAAATGCCATTTTAAGCTTTT |
| >ENST00000544344.1:: | 145 | ATTTAAGCACGACTCTGCAGAAGGAACAAAGCACCCTCCCCACTGGGCTCCTGGTTGCAGAGCTCCAAGTCCTCACAC |
| chr12: 6898701- | | AGATACGCCTGTTTGAGAAGCAGCGGGCAAGAAAGACGCAAGCCCAGAGGTCCATCCAAGCTGAATGATCGCGCTGAC |
| 6924236(+) | | TCAAGAAGAAGCCTTTGGGACCAAGGAAACTTTCCCCTGATCATCAAGAATCTTAAGATAGAAGACTCAGATACTTACAT |
| | | CTGTGAAGTGGAGGACCAGAAGGAGGAGGTGCAATTGCTAGTGTTCGGATGTGAGTGGGGCAGGTGGGGATGAGGAT |
| | | ACCTCCTGCCTGGTTCCCTTCCCCACTACTCCCACCCCTGCACCAAATCCAGCCTGAGCTGGTGATACCGCAGCAGCC |
| | | CCAAGAGGACCAGGCTGTCAAACTGGCCTCCAAATGTCTTAAAACCCTTCTTGATCAGGTGAGGGATGCTGGTGGGCG |
| | | GAGGAGGGAAGAGGCCTTGGGAAAAGGAAAGAAAAGGGAAGGAGGCAAGGGAAGGAGGGAGAGAGACTGGGGAAGA |
| | | GAGGATGAGGGGAGAGGAGGAAAGAAGAGAGAGAGGAGGGGAGAGGGAAACCCTATCTTGGCTGGGGGTGCGCAGC |
| | | TGGGTGCTGGGAGGAAGGAGATGTTGGGACGGCGATAATGGAGAGATGTTGTTGGTTTCCTGTTGTCTGCCCTTCTCC |
| | | TTGGGGATGGTATGTGTGTGACACAGCTGGCCTTTCCCTCCACAGTGACTGCCAACTCTGACACCCACCTGCTTCAGG |
| | | GGCAGAGCCTGACCCTGACCTTGGAGAGCCCCCCTGGTAGTAGCCCCTCAGTGCAATGTAGGAGTCCAAGGGGTAAA |
| | | AACATACAGGGGGGGAAGACCCTCTCCGTGTCTCAGCTGGAGCTCCAGGATAGTGGCACCTGGACATGCACTGTCTTG |
| | | CAGAACCAGAAGAAGGTGGAGTTCAAAATAGACATCGTGGTGCTAGGTAAGGGAAGCCCCTCTTCGCGCAGTCTCCTC |
| | | CCTGCCCCAGGGGCTGACAGCCCCTCCCTCTGCTCTGACTGCCCTG |
| >ENST00000500365.2:: | 146 | AAGACTAATGACCGAAGCTTCAAGACTTTGACATGTGTGAAAACATGAAATGACCCTTGGCTCTTATTGTTCTTGCTGGT |
| chr12: 47602202- | | GTGGTATGTTCACGGCTGAAAAGATGGTCAGAAGGGGAAAGGAGGAAGTGAGAAGAAAGAAACAGGGAAATGACAGA |
| 47610218(−) | | GTGTTGCTCAGTTACCCAGGCTGGAGTGCAATGGCATGATCTTGGCTCACTGCAGCCTCCACCTCCTGGGTTCAAGCG |
| | | GTTCTCGTGCCTCAGTCTCCCAAGTAGCTGGGATTACAGGTATGCACCACCATGCCCAGCCAATTTTGTATTTTTAGTAG |
| | | AGGCAGGGTTTCACCATGTTGGCTAAGCTGGTCTTGAACTCCTGACCTCAGGTGATCTACCTGCTTCAGCCTCTCAGAG |
| | | TGCTGGGATTACAGGCATGAGCCACCAAGCCCGGCCTGAAATACATTTAAATAAATGAAGATGGAGAATCAGACATTAC |
| | | TTTCATTTATCTGAATCTGGTGTAGTTTAATTAATCTGTAATTGTAAGTCTTTGAATCATATGTCAATATTCTATTTTGTTAG |
| | | TCATGATTTCTAAGTGTTACATAACTGTGGCCCCAGCCACTTTATATTTTGGTATCATGACAGAAGACTCTGATTGTGGTT |
| | | ATTTTCTACCTATGCATTTATTCTTAGTGATATATGTTGGTTAAATTATGGCATAGTTTTAAAATACATGACAAAAATAGCT |
| | | AACAGTAAAGAAATGTCTTTGTTTCTGGTTGAATAAACTGACCACGCAAGGTTCCAATTTAAAATAAATTCTAAACGAATC |
| | | ATGTTATTTTCCAAAACTGTTCCTCCTCTCAGTAAATGAAATCAATATCCACCCGGTTACCCCAAGACAGAAATCTCAAGA |
| | | TCATCTGTGACTCCTTCCTCTTCATCTGCCACAAGAAGAGTAGCTTCTATCTCCAAAGCTAGTCTCTGATCAGTCTACTT |
| | | CTCTTCCTATAGCCTCTGCTTTAGTTCAAATCTAGAATCCTGCTGTGGTGGTGCTGTGGCTTCCAAGTTCATCTTCTTCC |
| | | AATCCGTTCTCTAAACTACAGTCTTGCTCTTTCTAAGACTCAGATCTTATAATTTTACTTCCCTTCATAGGTTGAATTTAAC |
| | | CTGCTAAATCACATAAAATTTTTCTGAAGATACCAGCCTTTCTGCCTTAATCAAACACATTAGCCAAATCCATAGCATTAC |
| | | TGGGATAAAGCCTGAAGATCCCAAATTGATAAGGCATCTGGATTCCCTTTGATAACAGTATTCAATAACAATGAACACCT |
| | | ACTGAGTATACTATTCCAAGCATTTAAAGTGTATCAACTCTTTTCACAAAATCAACAATAATTATTATTGCTACCATTTATT |
| | | GAGAGTCTACTTTAATCCAGGTGTTACATATTCATGTATTCATTATTATTACCTTTTTTTTTGAGACGAAATCTTGCTCTGT |
| | | TGCCCAGGCTGGAGTGCGATGGCATGATCTTGGCTCACGGCAAACTCCGCCTCCCTGGTTCAAGTGATTCTCCTGCCT |
| | | CAGCCTCCCGAGTAGCCGGGATTACAGGCATGTGCCACCATGCCCCGTTGGCCAGGCTGGTCTCGAACACCTTACCTC |
| | | AGGTGATCCTCCCACCTTTGGGATTACAGGTGAGCCCAGCCTATTGTTACCTTTAAAACCACCCTGCAGGGTAGCTCTT |
| | | ACTGTCTCCATGAGGAATATCAGAAAAGGGAAAGGACTTCCTGGAGGGCTGGTTCTGCCTCAAGCCAATCAGCTGACA |
| | | CTAAGCAGTGAAGCCGGAATCTGAGGTCCCTCTGCCTGCCCTGGTCTTTGCTCACCCTGACAACCAGACACCTCACTG |
| | | TGTCCTGAGCAGCATAGATGCCACACGCACAGTGCCACATAGAGAAGGGTGATGTAAATGAAAATTAAAACACTGTGAC |
| | | AAAGTCAAGCAGGGCATTTGTTTTTGTTGTTGTTAAGTTATTTTTGGTAGATACAGGGTCTCACTATGTTGACCAGGCTG |
| | | GTCTTGAACTCCTGGGCTCAAGCAATCTGCCTGCCTCAGCCTCTCAGAATGTTGGGATTATAGACATGAACCACTGCAC |
| | | CCGGCCAGTGTCAGTCTTTGTTCCCTACAAGTGAAATCAAAACAGCCTCACGCCTGTAAACCCAGCAGTTTAGGAGCCC |
| | | AAGGTGGAAGGATCGCTTGAGCCCAGGAGTTTGAAACCAGTCTAGGCAACATAGTGAGACCCTATCTATACAAAAATAA |
| | | AAATAGCCTGGGCAGGGTGACTCATGCCTATAATCCCAGTACTTTGGGAGGCCAAGGTGGGTGCATCACTTGAGGTCA |
| | | GGAGTTCGAGACCAGCCTGGCTAACATGGAGAAACCCTGTCTCTATTAAAAATACGCCTGTAGTCCCAGTTACTCGGGA |
| | | CGCTGAGGCAGGACTGCTTGAACCTGGGAGGTAGAGGTTGCAGTGAGCCGAGATCGCGATACTGCACTCTAGAGCCT |
| | | GAGTGACAGAGTGAGACTCCGTCTC |
| >ENST00000550589.1:: | 147 | GTGAGCTATTTGCGCTAAAAATAACAGGAAATGGGACCACGGGGGGTAAAAATGTAATTGGGGATGAAGGCAGAGACA |
| chr12: 82746617- | | CAAAAAGTTTACTACTTTTGGTAGGCTTGATCATTATATTGTTAAATAATCAGTATAAAGTTCAGGGAAAAGATTAGCAGT |
| 82749900(−) | | AGAAATGTAGATTTGAGTTATTCTCTGATAATTGAAGTAAGGAAGTTTAAATCATGAGAAAGGAAGGTAAAAAATCATGCT |
| | | TTTAAAGATTACGGTTTATTTTGGGGTGGAAACAGGGAAAATATTGTCATTGAGGAAGTTAAGCTTTTCAGGACTGGAGA |
| | | AAGAGAAATACAGTGACAGCTCAGTCCACAGAGAAAAGTTGTGAGGGATTAGTCAGCTATCAAATATTGTGGTCCCCCG |
| | | TTATCTGTTTGGAAGATGTTCCAAGACCCCCAGTGGATGCCCTGAAACTGCAAATGGTACCAAACCCAGTTTCTGTCAAT |
| | | TGGAATGTATTTCTGTTAATGTCTTTTACTTGGGAATTTAATGCCTTTTCCATCTTAAATCACGTACTGTGGCCATAACTTT |
| | | TGCAGTTTGAAGTATGACAGCAAAACTAGCATGAATTTATTTTTCCTTCACAATTTGATGTATAGAAGATTTTTTTTAAAAC |
| | | CATAGATCTTAGCAACTTCAACATACGATTTTTTTTCCTTCCTTATTAAGTCAAGAACTTTTACCTTTTCACTTAAAGCAAA |
| | | CACTCAGTGTGGTGTACAATTTAAAACATATGAATTGTTTATTTCTGGAATTTTTCAATTTAATATTTTTGGACCTCTGTTG |
| | | ACTGCAGGTAACTGAAACCGTGGAACAGGGAAACCATGGATAAGGGGTGTGCTACTGTACTATAAAGTTGAAGGTGAAT |
| | | AGGACTGAGGACAGGTCTCTGGATTTGTGGACTAGGCAGTCACTAGTCACTAGTCCAAAGTCGCTAGTGATTAGTAGCT |
| | | ATTAAAATATTATATTGTAGTTACAGAGTATAGTGAAAAGTTGAATGATAGAGGAAAGTAAAGTTATTACTGAATCAAGGA |
| | | TGCTGAGGAAATGAATGGAATTCCAAGTAGTTGGATTAGCCTTGACAAAAAGGACATCTTCAGAGAGGTGAGAAGTAGT |
| | | TAAAGATAGAACAGTTCAAAATGGTATCTAGATCCAAGGTATGGTGATTCATGACTAGGTAGGTAAAATGGAATAGAGAG |
| | | GAGGGGCTCAGAAAACTTCGAGATTAAAGTTCATATATACACATAATTTTCTTAACATCCTTAAACAACAAGTTTAATTCTT |
| | | TTTTCAGTCTGGCATTTCTATCATTCATATACTGGTTTCATTACTTTTAAAAGTTTTTTTTTTTAACATTTTAGGATTTTTCAT |
| | | AGCTTTCTATATTTTTTCCTAGTTGAAATATTTCTCTAAGAATCCCCATTTGTGCCTTTTATTTAATATATACTCTTAAAAGC |
| | | TACAACTGTGTTTTTAATACCTTTTTGGACATTTTACTAATAAAGTCCTGTTTCCTCATCCCCCAATTAAAAGCTCCTTTAC |
| | | AATTCCAAAGAAAAATAAAAAGAAAACATCAAATCAAAAAGCACAAGAAGAATATGAACAGATACAAGCCAAACGTGCTG |
| | | CTAAGAAACAAGAATTCGAGAGGAGAAAACAGGAGAGAGAAGAAGCCCAAAGGCAGTACAAAAAGAAGAAAATGGAAG |
| | | TGTTTAAAATACTGAACAAAAAGACTAAAAAGGGCCAACCAAACTTGAATGTACAAATGGAGTACCTTCTTCAAAAAATAC |
| | | AAGAAAAATGTTAAACATTTTGTTCCTACAGGTTAAAATATCTGCTGCCTATTAGGTTCTTCTGTGACATGTGCCTCCCAG |
| | | CAGTGAACTAAATTTGTCGACATAAACTGGATTGCTAAACTATGCTAAATATAAGATGTTCACATATTTTTATTATGGTAAA |
| | | AAATTTTCTAAATATGTTCTACATGTTTCTTATTTATTTGCCTCTGAAGGAAGGTTGGCCTGAAGAACTGAAAGAACCTCT |
| | | TATTTTGCAAGACAGGCCCAAGCATGTAATACTTTTGTACCATATGAGATTTATATGAAATAAATTTTTTAAAAATAAGGAA |
| >ENST00000543124.1:: | 148 | CCGTACTGGCTGTAGACACCTTCGTCTTTCTTTACCTCCCTGGATACACATGTAGTTTACTATGGCACCTGTCTTCTCAT |
| chr12: 133624206- | | GGCAGTACTCTGTTCCCAAACAAACATCATTTTCTTCTGGAGAGGCTCTCTTTAATCTTTAGGTTAACAGTTGTATAAATG |
| 133633918(+) | | GAAGGATGGAAGAATGCACAATGACATTTTATCACACAAAATATGCAAAGGTAGAGACATAGTAGGCATTTTGTATAACT |
| | | TGTTATGAGAATGAAGTGCTAAAGCTCCAATATTTTTTTCTCACATCATACGGTGTTAACTGCTTCAGATTTACCAGGATT |
| | | GTGCTCTTACAGGAGTCATTCTCATTTGACGATTTATCTGTGGACTTCACCCAAAAGGAGTGGCAGCTACTGGATCCCT |
| | | CTCAGAAGAATTTATACAAGGATGTGATGTTGGAGAACTATAGCAGCCTAGTGTCACTGGGTAATAAAAGCTTTCTTGAG |
| | | GACCTTGGACTATGCCCAATGCATTGCCTTTTATTTTTAGTTGCTGGGAAGTTTCAGAAGCCTATAGCTAGGCTTCTGAA |
| | | ATTTTTAATGATTTTAGACCTGAAACTTAGATCACAGTTGCTTGGACTATGACACCAAGAAAGTATTTGGGTCTCACCTTT |
| | | CAATAATGAAGTTGTAAGTTTGTCATGCAGCCTCTGAAACTTAAGTAGTAATAGTTTGAGCAGTAGTGATAGCACCTAAT |
| | | GCATATAACTTACTATATGCCACATCTTATACCAAGTGCTTTACGTATATCAACTCATTTAGTCATCAGAGCAATCCTATG |
| | | TGGCAAGTACAGTCATTATCCATTTTTCCTGATGAGAAGATCGAGGCCAAGAGAGGTTAAGTAACTTTCCCAAGAACTAA |
| | | CAGCTAGTAAATTAAAGAGCCTGGATGTAAACCCAGGCTTACTGGCTCCAGAGCCTCTGAAGTGTTCAGTGGCTTTGTA |
| | | CTAGACCCTATTAGCCACCAAGGGAGACCGTTACTGTAACTTTGAGAATTACTTACTCTGTTGGGGTACAGCTTCACCA |
| | | GGCCAACTTTGATGTGAAACCTTGCTCAACTTTAGAGGCCCTAAACCCCAAGCAGTTGGGCCCAAGGCCTTTGTGATTT |
| | | TTCCCTTAACAGGGTATGAAGTTATGAAACCAGATGTCATCTTCAAATTGGAGCAAGGAGAAGAGCCGTGGGTAGGAGA |
| | | TGGAGAAATTCCAAGTTCAGATTCTCCAGAAGTCTGGAAAGTAGATGGTAACATGATGTGGCACCAGGATAACCAAGAC |
| | | AAGCTTAAAATTATAAAAAGAGGTCATGAATGTGATGCATTTGGAAAAAATTTCAATCTGAACATGAACTTTGTTCCTTTA |
| | | AGGAAATCAAACAGTGAAGGTGACTTAGATGGATTGATTTTAAAACATCATTTAGATTTGCTTATTCCAAAAGGAGATTAT |
| | | GGAAAAGCAGAATCAGATGACTTTAATGTGTTTGATAATTTTTTTCTCCATTCCAAGCCTGAGGATACTGATACCTGGTTA |
| | | AAATACTATGACTGTGATAAATATAAAGAGAGCTATAAAAAGTCACAGATTATCATATATCATAGAAATCGTTTAGGGGAG |
| | | AAACT |
| >ENST00000503525.2:: | 149 | GTGCTGCTGGCGCCCGGCCCCCGCGGGGTGCAGCTCTGCGCGTTCTCATGCTGTCTCTCTCTCTTTCCCTCCGCGCT |
| chr14: 96343108- | | GCCTCTCCGAGGTCCTCCCGCCGAGCCCCGGCGCGGGGCATGAGGAGCCCCCGGGTGCCGCCCAGAGACCAGCAG |
| 96391908(+) | | GCTGCGCGCACACCTAGCCAGCGGCAGACGGGGACATGAGCAGCGCGCACGGGGTCCCGCGCCCGGCGGCCAGCC |
| | | CTATCCGGCGGCGGCCAGCGGGTCAACGCTGCCCGGGAGAATGAGGCAGGAGCCGGCGGCAGCCTCCTTTTTTTCCT |
| | | TCTCCTCGCCTTCCTGCGGCTCCGGCGCTCCGGGTCCGGGCCGGGCTGCGGCTCTGCTGCGTGCCCCGCGCGCCCC |
| | | TCAACCGCCTCCGGATGCGCTTCTCGGTTAGCCTGGCAAGGAAGATAAAGACATTTGCAACCAAGATGGTAATCACTAG |
| | | TGAAAATGATGAAGACAGAGGAGGTCAAGAAAAAGAAAGTAAAGAGGAGAGTGTCTTGGCAATGCTGGGGATTATCGG |
| | | GACCATTCTGAACCTGATTGTGATCATATTTGTCTACATATACACCACCCTGTGAATGGCCCAGAGCGTCCTCAGAGGC |
| | | CTCAGAATGGCCAAAGACGGAAGTCCTGCGTGTCGGCGCATCACTGACCAGACCCTGCGAGAACAAGCAGGCTTGAC |
| | | CCGCACATACCACCCAATCAAATGCACCTTCAAACTTTACAAAAGGTCACACAAATAGACCGATCCTGCTGCAGGGAGC |
| | | AGACACTAAAGCACAATGATTCCAACAAAACTCATTCACAGCACTAGGAACTCAACGTCTTTGGCAGGGGGCCCAGAAG |
| | | AATGCTTGGAAGACCAGCCTCTGACACCATCAGTGAGCGGATGGGTGCAGAAATTCATTATTCCAGATCGCTGACAGAT |
| | | ATCACATATTTGAAAAGATGAATAGGGCGGACATGGCTCAGATGTGTGTCTCCCAGGACAAGTGTTTCATCTTCACTTGA |
| | | CGAGCTATTTAGTGGAAAAACCACAGGCGCAGCCCTTTGACAGGCATCCCATTCATCAAAAGTGTCTAACTATTTGATAC |
| | | TGGGGAGATAACTTATTTTTCTTTTTTCATTGGCTTGACATGTGTATCTGTTCATGTCAAGGTTTATAAATATATATTTTTA |
| | | ATAAATGTGCTCTATTTTTTAGCATGAACCAAATACTTGGAGAGGCACTCCCAGATCCATAGAGCTTTCCTTAGTTTTATC |
| | | TGCTTTGTCCCCTCCTCCCCCAACTACAGATGTTCTGTTGTGGAGCCATTCTAGTCCTTTTGTCTCATCTTGAGTCTTTTA |
| | | CCTTGCGCTTTTGTTCTCTCTCTCTCCTCTCTCTCTGCCTCTTTGGTCTGAAGGACATTTTCCCATACTGTCAGCCATGGT |
| | | TTTGGGTGCATGTTTTAAGATTGTCCATTGAGTGGCTTTTTGTTGTTATCTCGGAGATATAAAATGATTGTGGGCATGCA |
| | | GACCTTAGATGCACCCTATCTTTACTGAGAATTATGCATGAATAAGGGCTGAGTGATAGATCAGCTTAAAATTAAAAGGA |
| | | CTACCTTTGAGGAAGAAGAGCGTGGCTATATTTGCAGATGAACTTTTGAACAGAATATTCAGCTTCTTACCGGCAGCGTT |
| | | ATTGTTTCATTCTTGTGACCATTCGTTTATCAGATTTTGATTTTAGCGGTCATGTACCGCGAGAGTTGGGAAGAACAAGG |
| | | GGGAAAGCTCGGGATTAGGTGCATTACTCCTTCCTTTGCAAGATACCTGGGATCCTCCTCAAAAGCGGGTGGGGTATAA |
| | | ATGACACAAGAACTCCCCCAGGAGATCTCATGGTGATTCAGGCTGTGAGGACAGCCCTGTGACAGGTGACTTTTCAGG |
| | | GACATGAGGAGGGGATTTAATGATTGCCCTAAAGGACTTCTGTATTTTTAAAGCCCCTGGTTTACACCCACATGAAGCTA |
| | | TTTCCTCTCTGGCAGGGATGGTTGCATAAAAACAAATTAGCTCCCTTCTGGCTCCCTGAAATGGGCCCTTGCCTGGCTA |
| | | CAGTGGCATGGCCTTAAAGAGAGGGTTAGTATTCCTTCTGCCATTGCCAGCTGTATTAGTCTGTTTTCACACTGCTGAAA |
| | | AAGACATCCCCAAGACTGGGCAATTTACAAAGAAAGAGGTTTATTGGACTTACAGTTCCACGTGGCTGAGGAGGCCTCA |
| | | CAATTACGGTGGAAGGTGAAAGGCACGTCTCACATGGTGGCAGACAAGAGAAGTGAACATGTGCAAGGAGACTCCCAT |
| | | TTTTAAAACAGATCTCGTGAGACTTTTTCACTATCATGCAAACAGCATGGGAAACCTGCCCCCGTGATTCAGTTACCTCC |
| | | CACCGGGTCCCTCCCACAACACATGGGTATTCAAGATGAGATTTGGGTGGGGTTACAGCCAAACTCTATCACCAGCCTT |
| | | GCCCCTGGGCAGAAGCAGCAGCAGTCTGCCTGGCTGGATTCAAATGATTCTGAGGCTTCTATAGTCTATGCCTGCAGA |
| | | TCTCTCCCTCACCCATGCTATAGTGTCTGAAATTCCACCATTAGAGAGTCATTTCTTGGGCTCTGTTAAATGGACCAGGC |
| | | TCTTTTATAAAGAAAATGCCCCTGAGCAGCTGGCTCTGGCATTGATTTATGATATCTTCTCTTCCCTGCCAGAAGGAAGG |
| | | AAGCTAAGGTGCATGTAGGGCGTACTGTGTGCCCAGGCACTGTGCCAGATGCTTTGGATACTTGGAGTCATTGAATTCT |
| | | TGTAGTAACCCTGTGAGAGAGGGAGTCTTTTCTCCACATTGTAGAAGAAGGAAAAAGGGCTCAGAGAGGTCAAGAAATG |
| | | TCCCTGAGATCACATGGCTTCTAGTGGAGTCAAGATCCAAACCCAATGTGTCTGATTCCTTAGCCCTTGGGGGTCCGGA |
| | | GGCTGCTGAACAAGAAAGGAGGTGGAGAGGAGAGAAAGCTGCAGGCATACCACCGCACACCCTTCTCCCTCCCCTGT |
| | | AAAAACAACCCTGGGAACTCCCTGGACACTAGCAGAATATCATACACTAAGGATAAGGGATGAGAGGAGGCTGGTTAG |
| | | AAATAAAGCAGTGTCAGGGGGAAGGAGCTACTCAGTAGGCTCTGTGTGATTCTAGAAAGACTGTATGAAAATTCTGAAC |
| | | AGTGAACAGAATAAACAATAAAGGTGCAATGGAAAAAAATA |
| >ENST00000560660.1:: | 150 | GTGCTCAGCAGCTTCTGTCACTAGCTCTGAATGGCCTGTCTCCTGGACAAAGAAGCTTTCACGGACTACTCTGCAGGGA |
| chr15: 40706604- | | GGTGACATTGGACCAGAGCTGACTCCACCTGGGGGAAAGAGTAATTCTCTGGTTTCTTTATAGGTGCCTGTGTAGAAAG |
| 40707660(+) | | AGTCAGGGAGGGATTGGGTGACTTCTAGACCAATTAACTTTTCTTCTTTCCCATCCAAGGGAGTCCAGAGAGGGAAGGG |
| | | AAAGATCTAACAACTCTAGCCTTTGTGGTTGGCCTCGTGGATGGGGTGGGAGTCCTGGCAGCTCCACTTCTGACTGGA |
| | | AGGGGTTCACTTGATCCTTTTCTTTCACCTGGAACCTTAGTTGAATAAAGGTCAAGTGACATATTTTAGTGCCTTACTTGA |
| | | GAGCCATGTTTCCAAATCTAGTACCTTTGATAAAAGTGAGGAGGCTGTGGACAGCTCTCTCCCTCTGACCAGCACTTAT |
| | | CCTGGCAGCTGCCAACATCCTGGGCCATGAGAATAAGGGTGTCTACGTGCTGATGAGTGGGCTGGACCTGGAGCGGC |
| | | TGGTGCTGGCCGGGGGGCCTCTTGGGCTCATGCAAGCGGTCCTGGACCACACCATTCCCTACCTGCACGTGAGGGAA |
| | | GCCTTTGGC |
| >ENST00000562107.1:: | 151 | GTTGAGCAGTTCAGCAGTGTACACAAATCACATTCTCAGGAATTTTTGGATGCTCAGCTGATGCAACCAGAAGATACTTG |
| chr15: 45118568- | | TGTGATGACAATATGTTATTGTAAGTGGGAAGTCAGTATTTCTTATCTTCTGAGCTTTCTTGTTTTTACCTCCTTTACAGG |
| 45120929(−) | | CCACTCGCTCTGGTGGGACCCTCGTGCTTGTGGGGCTGGGCTCTGAGATGACCACCGTACCCCTACTGCATGCAGCC |
| | | ATCCGGGAGGTGGATATCAAGGGCGTGTTTCGATACTGCAACACGTGGCCAGTGGCGATTTCGATGCTTGCGTCCAAG |
| | | TCTGTGAATGTAAAACCCCTCGTCACCCATAGGTTTCCTCTGGAGAAAGCTCTGGAGGCCTTTGAAACATTTAAAAAGG |
| | | GATTGGGGTTGAAAATCATGCTCAAGTGTGACCCCAGTGACCAGAATCCCTGATGTTAATGGGCTCTGCCCTCATCCCC |
| | | AGTCTCGGGATCTCAGGGCACAATGGCTGGACATGGGTGGGCTCTGATGCAGAACTTTCTCTTTTGAATGTTAAGAATA |
| | | ACTAATACAATTCATTGTGAACAGAAGTCCTTACGCAGAGGAATTGGTGTGCCTTAAAGATACAATCTGGGATAGTTTGG |
| | | GGGAACTTGTAGCCAGAATGCCCTGTTCATGCTGAGCAAAGTTCAGCAAGTAGAGCAGAGTTTGGCAGGCAGGTGCCA |
| | | GGAACTCCCCTTCTTCCTGGAGTGCCTTCATTGAGGAAGGAAATCTGGCCCTTGGGTTTCCTGGTTCCACTGCTACTGA |
| | | CCCAGAGGGGAATGAGGGCTGAGTTATGAAAAGATAACTTCATGAAGACTTAACTGGCCCAGAAGCTGATTTTCATGAA |
| | | AATCTGCCACTCAGGGTCTGGGATGAAGGCTTGTCAGCACTTCCAGTTTAGAACGCAATGTTTCTAGAGACATATTGGC |
| | | TGTTTGTTTTGATGATAAAAGGAGAATAAGAAAAGGCATCACTTTCCTGGATCCAGGATAATTTTTAAACCAATCAAATGA |
| | | AAAAAACAAACAAACAAAAAAGGAAATGTCATGTGAGGTTAAACCAGTTTGCATTCCCCTAATGTGGAAAAAGTAAGAGG |
| | | ACTACTCAGCACTGTTTGAAGATT |
| >ENST00000427525.2:: | 152 | CCGAATAGCCGTGTTTGGGACCTGGGCTCGGGCTTCTTGCGTCCCCGCTAAGAACATGTCACGGGGCCGAATCGTCC |
| chr15: 85174709- | | GTATTCTCTCAGCTTCAAGCTCCTCTACTTTTCAACCAGGTCACTAGCCCTTGACTCCTCTTATCAAACTTCCGGAACTG |
| 85178652(+) | | CCACCCCACCAGTGACTCCACAGGCACCAGGGCATGCAACAGGGCTGGGACAGGAAGGCTCTCTTCTTCACCTCAAG |
| | | CCTGCTGGGCTAACACTTGCGATTTTTACTAGAGTTAACTTTGTAATGTATGTCTCTGACTCTAGAATTTCAAGAGAAGTT |
| | | CCACTTAGTGACTCCTAAGTGGAAGTTCTAAGATGGCTTCCCAGTGAGGTGATGAAGAGGTTTGAGCTTTAGAGTGCGG |
| | | TTGCAAAGCTCTTCTCTGACCTGAACAATGGCTGTAGCTGTGGACCAACAAATCCAGACTCCTTCAGTACAAGATCTCCA |
| | | AATAGTTAAACTGGAAGAAGATTCCCACTGGGAGCAGGAAATTTCCCTTCAAGGGAATTACCCTGGACCAGAGACATCC |
| | | TGCCAGAGCTTTTGGCATTTCCGTTACCAAGAAGCATCACGACCCCGAGAGGCCCTCCTCCAGCTCCAGAAGCTCTGTT |
| | | GTCAGTGGCTAAGGCCAGAGAAGTGTACAAAAGAGCAGATCCTGGAGTTGCTGGTCCTAGAACAGTTCCCGACTGTCC |
| | | TTCTCCAGGAGATCCAGATCTGGGTCAGACAGCAGCATCCGGAGAGTGGAGAGGAGGCAGTGGCCCTGGTGGAAGAC |
| | | TTGCAGAAAGAACCTGGAAGACAGAGGCTGGAGCCTCGGGCGAGGCCGTCCGGCCGCACCCCTCCTGCTCAGCTGC |
| | | GGTCGCCATGGCCAATGACAGCTGCGGGCCCGGCGAGCCGAGCTCGAGCGAGCGAGACCGGCAGTACTGCGAGCTG |
| | | TGCGGGAAGATGGAGAACCTGCTGCGCTGCAGCCGCAGCTCCTTCTGCTGCAAGGAGCGCCAGCGCCAGGACTGGAA |
| | | GAAGCACAAGCTCGTGTGCCAGGGCAGCGAGGGCGCCCTCGGCCACGGAGGGGGCCCTCACCAGGACTCCGGCCCC |
| | | GCGCCGCCCGCTGCAGCGCCGCCGTCCAGGGACCGGGCCCTGGAGGCCAGGAAGGCAGCGAGGCGCCGGGACAGC |
| | | GCCTCCGGGGACGCAGCCAAGGCAAAGGCCAAGTCCGCGGCCGACCCCGCGGCGGCCGCGTCCCCGCCTCGCGCG |
| | | TCCCCGGGCCGGACAAAAGCCATGGCTGCTTGTTATCCGGTCAATGGAACGGGTTATGTACGTCATGTTGATAATCCAA |
| | | ATGGAGACGGAAGACGTGTGAAATGTATTACATTACGTTAAAGAACGGGATGCCAAGGTAAGTGGAGGTATACTTCGAA |
| | | TTTTTCTAGAAGGTAAAGCCTAGTTTGCTGACATTGAACCCAAATTTGATAGACTGCTGTTTTTCTGGTCTGACCATCGCA |
| | | ACCCTCATGAAGTACAACCAGCATATGCTACAAAGTACGCAATAACTGTTTGGTATTTGATGCAGATGAGAGAGCACGA |
| | | GCTAAAGTAAAATATCTAACAGGTGAAAAAGGTGTGAGGATTGAACTCAATAAACCTTCAGATTCAGTCAGTAAAGACGT |
| | | CTTATAGAGCCTTTGATCCAGCAATACCCCACTTCACCTACAATAATTGTTGACGCTATTTGTTAATTTGTGAATACGAAT |
| | | AAATGGGATAAAGAAAAATAGACAACCAGTTCGCATTTTAGTAAGGAAACAAACAACTTTGTGTGTTGCATCAAACAGAA |
| | | GATTCTGACTGCTGTGACTTTGTACCGCATGATCAACTTAGAATCTGTGATTGCTTACAGGAAGAAGATAAGCTACTAAT |
| | | AGAAAATGTTTTTACCTCTGGATATGAAATAAGTGCCCTGTGTAGAATTTTTTTCATTCTTATATTTTGCCAGATCTGTTAC |
| | | GTAGCTGAGTTAATTTCATCTCTACTTTTTTAATATATGTCAAGTTTGAATTGGAATAATTTTTCTATGATTAGGTACAATTT |
| | | ATCAAAACTGAATTGAGAAAAAATTACAGTATTTCTCAAAATAACGTCAATCTATTTTTGTAAACCTCTTCATACTATTAAA |
| | | TTTTGCCCTAAAAGACCTCTTAATAATGATTGTTGCCAGTGACTGATTAATTTTATTTTACTTAAAATAAGAAAAGGAGCAC |
| | | TTTAATTACAACTGAAAAATCAGATTGTTTTGCAGTCCTTCCTATCTTACACTAATTTGAACTCTTAAAGATTGCTGCTTTT |
| | | TTTTGATATTGTCAATAATGAAACCCAATTGTAAAACAGTCACCATTTACTACCAGTAACTTTTAGTTAATGTCTTACAAGG |
| | | AAAAAGACACAATAAGAAGAGTTTAATTTTTTTTTTTTTTTTGA |
| >ENST00000553077.1:: | 153 | CCGCCCCGCAGAGGGGGGCCTGCGGCGACCGCGAGCTCAGAGTCCGGACTTGTCGCGCAATCTGTCTCCTTCGGTT |
| chr16: 68118653- | | CCACCCCAACCTAGCTGGACTCTCGGTCCGGTCCCTGCCCTCTGGAGACTCAGCCATTAAAGTTGAGGTGGGGGGATG |
| 68226115(+) | | AGGATGTGGGCGGCGTCCAGGTAACTCTGAGGGGCCCTGATGCCTTCCCTAGAGCCAGCCATCAGGAGTAACACGAT |
| | | GCGCCCCCACATCACCGAACCCAGAACCTCAGTGCTTCAGCACCCCTTCCTCGACATGAGCTCCGCCCAATGAGGAGA |
| | | AGGGTCTTCTGCATTGGCCGGTGGTGGTCCCCTATCCAATTAGAGAGGCTAACGGCCTTCTCTCGTCTGGTTTAGAAG |
| | | GAATCGCCGGGGGAATACAGGCCAAACTACAATTCCCGAGAGATCTTGAGCCAGATGATTGTGCATCCATTTACATCTT |
| | | TAATGTAGATCCACCTCCATCTACTTTAACCACACCACTTTGCTTACCACATCATGGATTACCGTCTCACTCTTCTGTTTT |
| | | GTCACCATCGTTTCAGCTCCAAAGTCACAAAAACTATGAAGGAACTTGTGAGATTCCTGAATCTAAATATAGCCCATTAG |
| | | GTGGTCCCAAACCCTTTGAGTGCCCAAGTATTCAAATTACATCTATCTCTCCTAACTGTCATCAAGAATTAGATGCACAT |
| | | GAAGATGACCTACAGATAAATGACCCAGAACGGGAATTTTTGGAAAGGCCTTCTAGAGATCATCTCTATCTTCCTCTTGA |
| | | GCCATCCTACCGGGAGTCTTCTCTTAGTCCTAGTCCTGCCAGCAGCATCTCTTCTAGGAGTTGGTTCTCTGATGCATCTT |
| | | CTTGTGAATCGCTTTCACATATTTATGATGATGTGGACTCAGAGTTGAATGAAGCTGCAGCCCGATTTACCCTTGGATCC |
| | | CCTCTGACTTCTCCTGGTGGCTCTCCAGGGGGCTGCCCTGGAGAAGAAACTTGGCATCAACAGTATGGACTTGGACAC |
| | | TCATTATCACCCAGGCAATCTCCTTGCCACTCTCCTAGATCCAGTGTCACTGATGAGAATTGGCTGAGCCCCAGGCCAG |
| | | CCTCAGGACCCTCATCAAGGCCCACATCCCCCTGTGGGAAACGGAGGCACTCCAGTGCTGAAGTTTGTTATGCTGGGT |
| | | CCCTTTCACCCCATCACTCACCTGTTCCTTCACCTGGTCACTCCCCCAGGGGAAGTGTGACAGAAGATACGTGGCTCAA |
| | | TGCTTCTGTCCATGGTGGGTCAGGCCTTGGCCCTGCAGTTTTTCCATTTCAGTACTGTGTAGAGACTGACATCCCTCTC |
| | | AAAACAAGGAAAACTTCTGAAGATCAAGCTGCCATACTACCAGGAAAATTAGAGCTGTGTTCAGATGACCAAGGGAGTT |
| | | TATCACCAGCCCGGGAGACTTCAATAGATGATGGCCTTGGATCTCAGTATCCTTTAAAGAAAGATTCATGTGGTGATCA |
| | | GTTTCTTTCAGTTCCTTCACCCTTTACCTGGAGCAAACCAAAGCCTGGCCACACCCCTATATTTCGCACATCTTCATTAC |
| | | CTCCACTAGACTGGCCTTTACCAGCTCATTTTGGACAATGTGAACTGAAAATAGAAGTGCAACCTAAAACTCATCATCGA |
| | | GCCCATTATGAAACTGAAGGTAGCCGAGGGGCAGTAAAAGCATCTACTGGGGGACATCCTGTTGTGAAGCTCCTGGGC |
| | | TATAACGAAAAGCCAATAAATCTACAAATGTTTATTGGGACAGCAGATGATCGATATTTACGACCTCATGCATTTTACCAG |
| | | GTGCATCGAATCACTGGGAAGACAGTCGCTACTGCAAGCCAAGAGATAATAATTGCCAGTACAAAAGTTCTGGAAATTC |
| | | CACTTCTTCCTGAAAATAATATGTCAGCCAGTATTGATTGTGCAGGTATTTTGAAACTCCGCAATTCAGATATAGAACTTC |
| | | GAAAAGGAGAAACTGATATTGGCAGAAAGAATACTAGAGTACGACTTGTGTTTCGTGTACACATCCCACAGCCCAGTGG |
| | | AAAAGTCCTTTCTCTGCAGATAGCCTCTATACCCGTTGAGTGCTCCCAGCGGTCTGCTCAAGAACTTCCTCATATTGAGA |
| | | AGTACAGTATCAACAGTTGTTCTGTAAATGGAGGTCATGAAATGGTTGTGACTGGATCTAATTTTCTTCCAGAATCCAAA |
| | | ATCATTTTTCTTGAAAAAGGACAAGATGGACGACCTCAGTGGGAGGTAGAAGGGAAGATAATCAGGGAAAAATGTCAAG |
| | | GGGCTCACATTGTCCTTGAAGTTCCTCCATATCATAACCCAGCAGTTACAGCTGCAGTGCAGGTGCACTTTTATCTTTGC |
| | | AATGGCAAGAGGAAAAAAAGCCAGTCTCAACGTTTTACTTATACACCAGTTTTGATGAAGCAAGAACACAGAGAAGAGAT |
| | | TGATTTGTCTTCAGTTCCATCTTTGCCTGTGCCTCATCCTGCTCAGACCCAGAGGCCTTCCTCTGATTCAGGGTGTTCAC |
| | | ATGACAGTGTACTGTCAGGACAGAGAAGTTTGATTTGCTCCATCCCACAAACATATGCATCCATGGTGACCTCATCCCAT |
| | | CTGCCACAGTTGCAGTGTAGAGATGAGAGTGTTAGTAAAGAACAGCATATGATTCCTTCTCCAATTGTACACCAGCCTTT |
| | | TCAAGTCACACCAACACCTCCTGTGGGGTCTTCCTATCAGCCTATGCAAACTAATGTTGTGTACAATGGACCAACTTGTC |
| | | TTCCTATTAATGCTGCCTCTAGTCAAGAATTTGATTCAGTTTTGTTTCAGCAGGATGCAACTCTTTCTGGTTTAGTGAATC |
| | | TTGGCTGTCAACCACTGTCATCCATACCATTTCATTCTTCAAATTCAGGCTCAACAGGACATCTCTTAGCCCATACACCT |
| | | CATTCTGTGCATACCCTGCCTCATCTGCAATCAATGGGATATCATTGTTCAAATACAGGACAAAGATCTCTTTCTTCTCCA |
| | | GTGGCTGACCAGATTACAGGTCAGCCTTCGTCTCAGTTACAACCTATTACATATGGTCCTTCACATTCAGGGTCTGCTAC |
| | | AACAGCTTCCCCAGCAGCTTCTCATCCCTTGGCTAGTTCACCGCTTTCTGGGCCACCATCTCCTCAGCTTCAGCCTATG |
| | | CCTTACCAATCTCCTAGCTCAGGAACTGCCTCATCACCGTCTCCAGCCACCAGAATGCATTCTGGACAGCACTCAACTC |
| | | AAGCACAAAGTACGGGCCAGGGGGGTCTTTCTGCACCTTCATCCTTAATATGTCACAGTTTGTGTGATCCAGCGTCATT |
| | | TCCACCTGATGGGGCAACTGTGAGCATTAAACCTGAACCAGAAGATCGAGAGCCTAACTTTGCAACCATTGGTCTGCAG |
| | | GACATCACTTTAGATGATGGTAAGTTCATCTCTGATATGTTCTTGAAGTAGTGAAGATTCAGGGACTTTATTCTCCCAAGT |
| | | GTCATGAAAAAGTTTCTATGGATTGCTTATTGGCATATGGTTGGGCTTTTAAATAAGTTGTTATTAGAAATATATGTTAATA |
| | | TATAACTTTGCCAGGTACCACGGCTCACGCCTGTATCCCAGCACTTTGGAAGGCTGAGGCGGGTGGATCACAAGGTCA |
| | | GGAGTTCAAGACCAGCCTGGCCAACATGGTGTAACGCTGTCTCTACTAAAAATACAAAAAATTAGCCAGGCATGGTGGT |
| | | GTGTGACTATAATCCCAGCTACTCGGGAGGCTGAGACAGGAGAATCACTTGAACCCGGGAGGTGGCAGTTGCAGGGA |
| | | GCTAAGATCGCGCCATTGCACTCCAGCCTGGGCGGCAGAGCAAGACTCCGTCTCGGGAA |
| >ENST00000568538.1:: | 154 | CGAGTCAGCGGCACAGAAGACGTCGGAGGGTTTGGAGAGAGCGGCGGAGAATAATGTCTTCCACTTGGTGGCCACTG |
| chr16: 68337634- | | TGTGCTCCCAGGAGGAACCAGTCCAGCCTCTCCTGCGGGAAGTTCTGCGCCCGTCACGGGACAGCCAGCAGCGTGTC |
| 68344725(−) | | CGCCGTAATCTCCGCGCCTCGGCTCGGGAGGTCCGGCAGGAGGGCCGCTACCGGGTGCTTTCCAGCCGCCGATCCTT |
| | | GGGGACCACCTCGAGCGGCCAGGAGTCCGAGTACACGCCGGGGAACCCAGAAGCCGCCGGGAACTCGGGCTTTCAG |
| | | TTGTTAGACCTTGTCCACGAGGAGGGAGAACCTGAAGCCGCCTCTGCAGGCTCCTGCAAAACATCTGACCCAGATGTG |
| | | ATCCTCTGCAATTCTGTAGAGTTGATCCGTGAGCGATTGACTGTGTCTGAGGATGGACCAGGAGTCAGGCGCCAGGAA |
| | | GAACAAAAACACGATGACTATGTGTATGACATTTACTACTTGGAGACGGCCACTCCAGGCTGGATTGAGAACATCCTCT |
| | | CCGTGCAGCCCTACAGCCAAGAATGGGAGCTGGTAAGGGGGCCCATGAGCAGACGGGGCATGAGGCATCCTAAGCTC |
| | | TTAATCTAATCAGAATGCTTTGCTTCCCTGGGCTCCACTCCCGAACAACATTGTCCTTAGTTCTTTTCCTCCTGAAAAGG |
| | | AAGAAATTATTCTAAGTGCCTTTTAGGAACCTGACTTTATAGTACAGGGAATTTGGGGAAAGATCATCTTAGATTGATCAT |
| | | TTTTACATTAATGAATATTTTACAAGGTTTGCAACATCACCTTTATTGTTTATAGCTATCCATTCTTAGGGGGGAAATATGT |
| | | ATATGG |
| >ENST00000568641.1:: | 155 | GGCTGGGGGCGGAAGGAGGAGCCAGGCGAAGCGGCGCCTCAGCTGAGAGGACCGGCGGACCCTGCAGAGGCCCCC |
| chr17: 5402758- | | TGCCCCTCTGGCTCCGCCCCCACCCGGGTCGCTAGAAATACAGCCGTAGCCCCGCCCACCGCCCACTGCGCTCTGAC |
| 5404465(−) | | CCAGACCCGGCTGACCCACCTACCCGCGATCCTGCCCATGGCTGACGGGCTCTTTCGGCGCAGACCCTGGGGTCTCG |
| | | AGCAGATTCGCCCGGACCCCGAGTCCGAAGGCCTGTTTGACAAGCCTCCCCCGGAAGACCCTCCCGCTGCCCGCGGG |
| | | CCCAGGTCGGCGTCGGCCGCGGGCAAGAAGGCTGGTCGGCGCGCGGGCGGGAGGGCGCAGGGGGGCCGCGCCGG |
| | | GCAGCCCCCGAAGGCCGCATCGCGCCCCCCGCCCAAGAAGGAGGCGCCTCCACTGGACGAGGGCTGCTATCTCGAC |
| | | CATTTTCCGCACCTCTCCATCTTCATCTACGCAGCCATCGCCTTCTCCATCACCTCCTGCATCTTTACCTATATCCATTTA |
| | | CAGCTTGCCTGAGTGGCCAGCGCGGGACGGGGTGGGCGCAGGACCGAGCGGGGAGGGAAAGGGAAAACGGGGCTC |
| | | GGCATTTTGTGTTTTAGAACAGCGCTGCACCCCCTTCATGTAGCTTTCGATGCTTGTTTCTTTCCGTCTTTGTTGTCACTA |
| | | TCTTTGTCTATCAGTACGAAAGTACAAAGTAGCTGCCGGCAATGAAATAGGGGTGCTGTTTGCACCTGCAGGTTAGGGG |
| | | TGGAGGCGTTTAGAATTTTGGGGTGTGATTGAGCCCCGTTTATAATTAGAATGCCCCTGGACCCCTACCACTCTGTGAC |
| | | GTGGGGGCACGCGCAGGGATCCCATCATTTTGTGTTTGGGGAGCTCAGAGTGCGCCCAATCTTGGAATCTTTAAGGGA |
| | | TGAGCCAGACCCAGACCCGCGGCCTTCTAGAGAGGGTCCGGCAGGGAGGGTCGGCGCCCTGGCCCGGGGTGGGCC |
| | | GGAGCCCTGTGATGCTGCATCGCCCCCAGGAGGAGCCAGCTGTGCCCCAGAGTTGGCGCGGCCGAGAGAGGACAAG |
| | | AGCGCGCAGCAGGCGAAGCTGGAGGGCGGGACTCGACTTTGTTGTCGCTGCCCGGAGGAGTCGAGACTGGTACCCG |
| | | GAGGAGCTGTCTCACCAGGAGACCACGTCCTGGAAGTGTCCGGGACTCGCGGGACCTGTGGCTGCAGACCCCGCCG |
| | | GCACGCAGGCCCAGAGCTGGCGCACTCCTGAGGATGAGACTCTGGGGGCCCTAGCCGGGGTCCACGGGAGGGCTGT |
| | | CCTTGGGGACTCTAGGATGGCTTCGTTCTGGCCCGGCTCACTTCTGGAGCTGTGAGACCCAAGACAAAAGGGGCTGAG |
| | | GGATTTCTCATTGACAAGAGTTCGTGCGGGAAAACCACCTGATCCCTAGGGATTTGTCATCTTAAGACTCAAAAGGCTTA |
| | | ATACCAGGAACCACCTTGGCAAGATATTTACCCACCGGCCATCTCTGTTTACTCATGAATGTTAAATGTTAAAACGCAGC |
| | | GCTCTAACCCTGCATATTATTTACTTGCAAATGTCTGTAATCTGTAATTGTGATGCCTCTGATGGAATAAATTATCTTTTT |
| >ENST00000566166.1:: | 156 | GCCGTGGGGCGGCAGAGCGGGTGGGAAGGACGCCTGGGAGCTGGACCCAGTCTCAGCGTGGCACTTCCCACAGGGC |
| chr19: 7982512- | | CGCCCAAGAGTGTCCCCGGCCGTGCAGTGCGCCCTGAGCCTCCCGCGCCGGCCCCCGCGGCCCTGGAACCCGCGCC |
| 7983975(+) | | GGTGGTGGCGCTGGTGTTGGCAGCCTTCGTGCTGGGCGCCGCGCTGGCCGCCGGGCTGGGTCTCGTCTGTGCGCAC |
| | | TCAGCGCCCCACGCCCCTGGCCCGCCCGCGAGAGCCTCGCCCAGCGGTCCCCAGCCCAGGAGGTCCCAGTGAGGAA |
| | | GGGATGGTGCGCCCCCAACATGGTCCGGAGATACACCCAGCTACCAATTCGGGACCAGGACCAACAGGACCGGACCC |
| | | GCCTCCCTGGACCTCGGACCTGATGAGGCCACGACCCCTGCGCTTCTCTCCTCCCCCTGTCCCTCCCACCTGTGCTCA |
| | | AAATAAACCTCTGGACTGAC |
| >ENST00000587837.1:: | 157 | ACTTGCCTGGACGCTGCGCCACATCCCACCGGCCCTTACACTGTGGTGTCCAGCAGCATCCGGCTTCATGGGGGGAC |
| chr19: 36395302- | | TTGAACCCTGCAGCAGGCTCCTGCTCCTGCCTCTCCTGCTGGCTGTCTCCGTCCTGTCCAGGCCCAGGCCCAGAGCGA |
| 36399197(−) | | TTGCAGTTGCTCTACGGTGAGCCCGGGCGTGCTGGCAGGGATCGTGATGGGAGACCTGGTGCTGACAGTGCTCATTG |
| | | CCCTGGCCGTGTACTTCCTGGGCCGGCTGGTCCCTCGGGGGCGAGGGGCTGCGGAGGCAGCGACCCGGAAACAGCG |
| | | TATCACTGAGACCGAGTCGCCTTATCAGGAGCTCCAGGGTCAGAGGTCGGATGTCTACAGCGACCTCAACACACAGAG |
| | | GCCGTATTACAAATGAGCCCGAATCATGACAGTCAGCAACATGATACCTGGATCCAGCCATTCCTGAAGCCCACCCTGC |
| | | ACCTCATTCCAACTCCTACCGCGATACAGACCCACAGAGTGCCATCCCTGAGAGACCAGACCGCTCCCCAATACTCTCC |
| | | TAAAATAAACATGAAGCACAAAAACA |
| >ENST00000597336.1:: | 158 | ATGTTCCACTATGAGTCTTGGGAGGATTGTCTTCTGGATGAAGATGAAGATGAATTTCAGGGACTGAGAGAAGAAGATG |
| chr19: 40529290- | | AAGAGATTGATCAATTCAATGATGATATTTTTGGGTCAGGTGCAGTTGATGATGATTGGCAGGAAGCACATGAGTGCCT |
| 40531409(−) | | GGCTGAATTGGAAGAAAAGCTACCAGTGGCAGTTAATGAACAAACAGGCAATGGAGAAAGGGAAGAAATGGACTTGTT |
| | | GGGTGACCATGAGGAAAATCTGGCAGAAAGGCTCAGTAAGATGGTGATTGAAAATGAACTAGAAGATCCAGCTATCATG |
| | | AGGGCAGTGCAGACCAGGCCAGTTTTATAACCCCAACCAGGAAATCTGAATTCCAGTATCTGGGATGGATCCAAAGTTA |
| | | TGAGGCAAATCTGAGGACCACTGCTCACTTAGGAAATGTCTACAGTGTCTGTATTAGAATATGCCTTGCCTCAGAGGCC |
| | | CCCCAGGGTCCAGAAAATGATTGGGACCTTTCTGAACATGCATTACCAAGGTGGTCAACTTCACCTGTCATTGGCAGTC |
| | | CTCCTGTTAGAGCTGTCCCCATAGGCACCCCACCTAAGCAGATGGCCGTACCCAGCTTTACCCAACAGAGCCTGTGGC |
| | | CTGTCCATGTTCGGCCCCCAATGCCACCGTGTTATCCTGGTCCCTATGATGAGAGGATGCCTCCAAATCAGCTCTACAG |
| | | TGTCCTGAACTCTTCCCTCCTGGGTCACCCTTTTCCTCCTAGTGTTCCTCCTGTTCTCAGCCCCTTTCAGAGAGCACAGC |
| | | TTCTTGGAGGAGCACAGCTACAGCCTGGACAGATGTCTCCCAGCCAGTTTGCATGGGTCCCTGGATTTGTTGGTAGTCT |
| | | GCATGCTGCTATGAATCCCAAGTTGCTACAAGGGCAAGTTGGGCAGATGCTTCCCCCAGCACCAGGCTTCCATGCCTT |
| | | CTTTAGTGCTCCACCCTCCGCTACACCACCTCCACAGCAGCGCCCTCCTGGCCCAGGACCTCACTTGCAAAACTTAAGA |
| | | TCTCAAGTCCCAGTGTTTAGACTGGACGCAACTCCCCTCCATCCACAGCACCATTGACTCTTGCATCAGAGACAGCAAC |
| | | AGAATAGAAATCAGCATCAGAATCTCAGTGGTGCAGGAGATAGAGGAAGTCACTGGAGCAGTCATCAAGATCATCTCCG |
| | | AAAGGATCCATATGCCAATCTCATGTTGCTGTGGGAAAAGGATTGGGCCTCTAAAATTCAGATGATGCAACTGCAAAGC |
| | | AATGGTCCCCACCTGGATGATTTTTATTACCAGAATTACTTTGAAAAACTGGAGAAACCATCAGCTGCTGAAGAAATACG |
| | | AGGTGATGGCCCTAAAAAGGAGCATAACAAGCTTATTACCCCTCAAGTGGCCAAACTGGAGCGCACCTATAAGCCAGTA |
| | | CAATTTGTGGGCTCTTTGGGAAAGCTTACTGTTTCTAGTGTGAATAATCCCTGAAAAATGATTGATGCTGTTGTGACATC |
| | | TTGGAGTGAGGATGATGAGATAAAAGAAAAACAAATTCGAGACAAGAGGAGAAAAACCCGTCATAATTAAGAAAACCTA |
| | | CAGTTTACTCCTTGATGTGGAGGACTATGAAAGACATTATCTCCTAAGTCTAAGACAGCGACCTGCTCTAATGGATGAGC |
| | | GAAAGTACGGAATTTGTAGCATGTATGACAACTTAAGAGGGAAACAGCCTGGACAAGAGAGGCCTAGTGATGACCGCTT |
| | | TGTACAGATCATGTGTATCCAAAAAGGGAAGACAGTGGTTGCCCGTATTCTTCCTTTCCTCTCCACAGAGCAAGCAGCT |
| | | GACATTTTCATGACAACAGCCAGGGACCTCCCTTTCCTTATCAAGAAAGATGCACAAGATGAGGTGCTGCCATGCTTAC |
| | | TGAGTCCCTTCTCTCTCCTCCTCTATCATCTTCCAACAGTGACTATCACCAGCCTTTTGCAACAGCTAATGAACCTACCT |
| | | CAAAGTGCAGCTACACCAGCTCCCTCCAATCCTCACTTCACTGCTGTGCTCCAGAACAAATT |
| >ENST00000589503.1:: | 159 | AAGAGAGCTTGGAAGGGATTGTTCGAGGATGTGGGATTTGGTCTTAGAAGACCGAAGGATGAATGTTTCGAGACAAGG |
| chr19: 41937223- | | AGTGGATCAGGCCAGATAAGTATGGCCATTTCTCTCAGGAGTTCTGGAATTTCTGTGAAGTGCCTGTCGAAGCTGTGGA |
| 41945442(−) | | TGCCGGTGACTGTGACATCAACTACGAGGGCCTGGATAACCTCCTCCGCCTGAAGGAGCTCCAGTCCTTGTCGCTGCA |
| | | GCGCTGCTGCCACGTGGACGACTGGTGTCTCAGCCGCCTCTACCCACTGGCCGACTCGTTGCAGGAGCTCTCGCTGG |
| | | CCGGTTGCCCCCGCATCTCCGAACGGGGCCTCGCCTGCCTCCACCACCTCCAGTGAGACCTCAGCTCAGGCTGGGCC |
| | | ACATGCCCAGGCACCTCTCCCACCTAACCCAGATGCAGGAGAGGAAGTGGGGAGGGGCAATGTTAGGCAGTTCTCATA |
| | | TCCCCCTGCATCCATCACAAACCTAGAGTATTTATGGTAGATGAGCAGTCACAGTGAGTCTCTGGAAGAATTAAATGACT |
| | | CCTGGTTTTCTTCCTTTTTGTTTTAGTCAAAACTGTGTGATATCGACGCTGTTGCAGAACAGCAGGAGCTGGAGTTGCAT |
| | | ATTTGCAATTAACACAGTGGGCTTCATGTGCCTGGACAGCTATAAAGATTTTATTTTAGGAAGCTAAGGTTGAAATTTGG |
| | | GGCCAGTACCTCCCTATACACACACACATGCACGCATGCACGCACGCACACACACACACGCACGCACACACACTGTCC |
| | | TGTAAGGTTGAAATTTGGGGCCAGTACCTCCCTACACACACACACACACACACACACACACACACACGCACGCTGTCCT |
| | | GTACCTCCCTACACACACGCACACGCTGTCTGTACCTCCCTACACACACACACACACACACACACGCTGTCCTTATACT |
| | | GGCTTATCTCCCTATACACACACACACACACACACACACGCTGTCCTGTACCTCCCTACACACACACACACACATGCTG |
| | | TCCTTACACTGGCTTACCTCCCTATACACACACACACACACACACGCACACACTGTCCTGTACCTCCCTATACACACACA |
| | | CGCTGTCCTTACACTGGCTTACCTGCCTATACACACACACACACGCACACGCTGTCCTGTACCTCCCTACACACACACA |
| | | CATGCTGTCCTTACACTGGCTTCCTGTCCTTCTCACCCCTTTTCAGGAACCTCCGCAGGCTGGACATCTCGGACCTCCC |
| | | TGCCGTGTCCAACCCTGGCCTCACTCAGATATTGGTGGAGGAGATGCTGCCCAATTGCGAGGTTGTGGGAGTCGACTG |
| | | GGCTGAGGGCCTGAAGTCAGGGCCGGAGGAGCAGCCTCGGGACACAGCCAGCCCTGTCCCTGCCTAGCCTTTAGCC |
| | | CTGTCCCCACTCACGTGGCTTCTCAGCGGGCTGCATGGAATGTCTGGTAGCTCACCACACTTCTGGCTTCCATTTGTCT |
| | | TCACTCAACGTCAGGGTGGGGGAGTGGTGCTGGCCAATCACAGGAGAGAGCGTGAGTTCCCAGTATTTATTCCTGGCT |
| | | GCCCTTGGCTAAAGGTCACAGCTCCTGTCACCCTGTCAGGCAGCCCTTTCCATGCCCCTGTTCAGGCCTGGGGAGGTA |
| | | AAGGCTCAGGCTGTTAGTAGCCGCAGAGAGCCACACTCACCTTGTCAGGAGACTCTTCTCAAACTGTCCTTATGTGAGT |
| | | GCACTGCCATTTCTTGCAGGGACCCTGACTGACACAGGGGCTACTACTGACACTTTACAGGGATGGTTCTCCCCCGTG |
| | | CAGGGCCGCTGTGCCCACTGCAGGACATGCAGCATCCTTCGCCCCACTCCACTCACTAAAGGCCAGCGCACCCCAGG |
| | | CCCCATAGTATTGCTGGTTATGGATTTATTGACTTTATGTTCCAAATTCAGCTTTTTCAGTTGGCTGTTTTTTGAAAGGGG |
| | | ATAAGCTTTGTCAGTAGAGGGCACCAAACAGGCATTATAGGAGGAAAGGCGCCTCCTTCGTGGTTCTTGTTGGTTGTGC |
| | | TTCTGCCTCTGGACGCCGCAGTGCATGTGGCTTCCCCAGCACCCAGCTCCTGAAGCACCAGGCGGTCAGCAGCTGCC |
| | | CTTGGCACCCTCCAGCCCTCAGAAGTTGCGTAGGAGACACAGCGCCTCCACTGAGGCACCTCTCTGGGAATAACGTTC |
| | | CCCAGCACCCCAAATGGATTTCCAGTCAATTCAGAAGCATTTTACCAGTGAAGCCCTCATTATTCCAGTTCACTGTTAAA |
| | | GCCAGTAATTCTCTATATTAAACTTTCCCTGTTCAAGTT |
| >ENST00000602172.1:: | 160 | ACTTTGTCGTACTCCTTCCTTGCTGACTAAGAGGAACAGAACACAGAGCAGCCTGGCGGTGTCCTACCAACAAGCCTCC |
| chr19: 48758931- | | GTTTCTCCTTCCTGTACACTAGGGCTCCTGAAACTCACCTGATGAAGTCTCCGTCTGTCACCCAGGCTGGAGTGTAATG |
| 48761456(+) | | GAGCAATCTCGGCTCACTGCAACCTCTGCCTCCCAGGTTCAAGCGATTCTCTTGCCTCAGCCTCCCGAGTAGCTGGAAT |
| | | TATAGGTGCATGCCACCACGCTCGGCTAATTTTTTGTATTTTTAGTAAAGACGGAGTTTCACCATGTTGGTCAGGATGGT |
| | | CTCCGTCTCTTGACCTCGAGATCAGCCCGCCTCGGCCTCCCAAAGTGCTGGGATTACAGGCGTGAGCCACTGTGCCCG |
| | | GCCTTGGTTTCTAAATACTATTTTGAAGTATCTGTGGAGAAATGGTTACTTCGAGGGCTGGGGCAGGAATATATAAGATA |
| | | ACATTGGAACAAATTCTGACGCCAGAACATGAAGAAATATTTGAAAAAGGATGGGGCATATTGACTGAACACAGAAGAC |
| | | AAACATGCATGACCTCAGCCTAATCGTGAGAAAACATCAAAGAAATCCAAATTGATAAACATTCTGTAAAATAATTGGCC |
| | | ATGCTCAGCAAAAGTGTCACAATCACAAAAGACAAGGGAAGAATAAGAAACGGTCACAGCCTGGAGGAATCTAAGGACA |
| | | TAACAACTGAGTGCAATGTGAGATCCTCGATTCGATCTTGGAGAAGAAAGAGGACGTTAAGAGAAATATTGACAAAATTC |
| | | AAATAAGGTCTGCACATTAGGTAATGGTAAGATACGTTGACATCAATTTCCTGGTTGGATACTTGTATTACATATACTATT |
| | | ATGTATATAATTATTATATTACAATTACATATACAGTTGTATATTATATATAATACAATAAATGTATATATATGATTATAATAC |
| | | AATAAATGTATATATACAGTTTTATATATAACAACTATTATACATATAATACAATTATATATATAGCTAACATCAGGATAGGC |
| | | TGGTTGAGGATTTATATGAATCTTTGCTAATTTTGCATGTTTAAAATTATTTCAACATAAAAAAATTATATGGGTCATGTAG |
| | | TTTTTTGTGCAGTTTAATTCTAGGCACCATAGTTTCTGTTTTAATGGGTGTATTTTCTTCTTTCAAGGAATTGGGATAGTCT |
| | | GTTGTATTAAAAACCTATTCATTTTTGTACATTAAGATTCTTCTGGCCAGGCGCAATGGCTCAAGCCTATAATCCCAGCAC |
| | | TTGGGAACCTGAGGCAGGCAGATCACCTGAGGTCAGGAGTTCAAGACCAGCATGTCCAACATGGTGAAACCCCATCTC |
| | | TACTAAAAATACAAAAAAAATTAGCCAGGCATGGTGGCACATGCCTGTAATCCCAGCTACTTGGGAGGCTGAGGAGGAG |
| | | ACTCACTAGAACTCAGGAAGCAGAGGTTGCAGTGAGCCAAGACTGCGCCACTGCACTCTGGCCTAAGCAACAGAGGGA |
| | | GACTCCATCTCAAAAAAAAAAAAAGATTATTTCTCTAACTTACTGGTTTATCTTTTATAGCTTATTTCAGGTGATTTGTTGT |
| | | GTTTGTCTATAGAGTAAAATAACATGTCTAATGAGTTATAATTTAATGTCCTTCCATTCAATCACTGTATTTCAAATTTATTC |
| | | CAATATTTTGGAATAAGCTTCCAATATTTTAACTTAAATAGGGAAGGTCATACTTGGTATTTTCCTGACCTCAGCTGGAAT |
| | | GCCTTCATAGTGTTCACATGAACACATTTGGCCTGAGAGAGACTTATTATGCCGAGAATGTGTGTAACCCTTTTTACTTA |
| | | GGAATTGTGGGTGGAGTTTTTCCCCCCGCCCAAGAATGATTGTTAATTTCCGTCCATCATTTTCCTTTGATTGTTCTTCAT |
| | | CTCGAAAACAAATAATAAAATAATAAAATAAATAAATAAAA |
| >ENST00000435844.1:: | 161 | ACCGGAAGTCCCGCCTCTGCCGTGGGCCTGCGAGAATCGAGGCACTCGCTGGCGTACCCATGTATCGAAATGAGTTCA |
| chr20: 18548063- | | CGGCCTGGTACCGGCGGATGTCGGTGGTCTACGGGATCGGCACCTGGTCTGTGTTGGGCTCACTGCTTTACTATAGCC |
| 18550207(+) | | GGACAATGGCGAAGTCGTCAGACCAAAAGGATGGCTCAGCAAGTGAAGTACCCAGTGAACTCTCTGAACGCCCAAAAG |
| | | GATTTTATGTGGAAACAGTTGTCACATATAAAGAAGATTTTGTTCCAAATACAGAAAAGATCCTCAACTATTGGAAATCAT |
| | | GGACTGGTGGCCCTGGTACAGAACCATGACTGGCTGCTGAATTCTGAAAACCAGGACTTGGTTCAACATTTAAATTTGA |
| | | TAGTTGCCCTGATTCCCATTTTGGGTTTGTGAAAAGTGTATGTATTTAAATTTGCTGTAAAACATAATCACTAATAATATGC |
| | | AATAAATATTTTCTTGAAGGAAACTA |
| >ENST00000417721.1:: | 162 | CGGGGGCCCAGGGTGGAGAGCACGAGGGCCTGGCCCAGGCACGGCCGGCGCCTCCGCCCTCGAGGAGGGCGTCAC |
| chr20: 47894714- | | CTCAGCTCCCCCCGGCGGCGGAGCCGGCGGGCTCAGGCGGGCGCGGCTGAGGGGAGCGGACCGCGGGGGGGGG |
| 47905797(+) | | AGATGACTGCGCCCAAGGCCTTTGCGGGCCTCAGCCGGCCCCAGAGGAAGGGGAACCCGTCGAGCGGTTTGGTGCG |
| | | TGTGAAGCGCGACATGGCGAGGAAGCGGACAAGCCCGGGTGGCCCGGCGTGTAGAGGGAAGGGGGGGGGCTAGA |
| | | CGCGGCCTGGACAACTACTAGAGCGCCTCGGGCTGTGCTGCTCGAGACTACATTTCCCAGAGCGACGCGCGCGGAGC |
| | | GGGCGGGAAAGAGAGCGTTTCGGGTCCAGTGCGCAGGTGCGAAAGCCATCTTTGGTTATATAAGGGAGGTTCAGGAA |
| | | GCCATTCGTTCTTTCGCGTCTGCGGTGCCCGGAGTGTGGTACTTCTCCTAGTTGCAGTCAGGCTTCATACGCTATTGTC |
| | | CTGCCCGTTAGAGCAGCCAGCGGGTACAGAATGGATTTTGGAAGAGGGAGTCACCACTGGACCTCCAAGGAAGCCAC |
| | | GTGCAGACATCTACAACCTTCGATCTCCTGACGAAAACTGGCGATGGAATATGAGAGGAGCCCTCTGGAAAGAAAAGG |
| | | ACAGACCCTGTGCTTTCATGAAAGTGAAGATCTGGCTGAACCAGTTCCACAAGGTTACTGTATACATAGCCTGAGTTTAA |
| | | AAGGCTGTGCCCACTTCAAGAATGTCATTGTTAGACTTTGAAATTTCTAACTGCCTACCTGCATAAAGAAAATAAAATCTT |
| | | TTAAATCAAAA |
| >ENST00000460721.1:: | 163 | GGATGAGTTGGTGCTGCTGCTGCACGCGCTCCTGATGCGGCACCGCGCCCTGAGCATCGAGAACAGCCAGCTCATGG |
| chrX: 140269933- | | AACAGCTGCGGCTGCTGGTGTGCGAGAGGGCCAGCCTGCTGCGCCAGGAATAGCGCTCCAGTTACCTGCTGCTGGGG |
| 140271201(−) | | TCGGGGCTGGAGCCTCACTCACTCGGAAGTGCTTGGAAGTGTCATCTACCCTGGCCATCCCCGGGATCCCTCCCCTGC |
| | | TAATCTGAACTGTGTCCTGAACCCCAGCTATTGGCCAGGCCCCTGTTACAAACTGCGCAGACCACCCATAGCCCTGCTG |
| | | CTGCCACCTGCCCTGCTGCCAGGCTTACATCTTCCCAGAGAACTATGCTGCCACTTCACATCCACTCACCACATCCCAC |
| | | CTACAGACTACCACCTTTCGAGATCAGGACCAACCTGGAAAATGCCAGAGTTGGCTATGCCTTTTCAGACATTGATTTG |
| | | GACAGCTCACCAACCCCTGAAGGGGCCGTTCAGCCTGGTTAGTTTTCTACCTACTCCGAGTGTCCTCCCCTGCCCCAC |
| | | CAGATTGCTGCAGGGGCGCGGTGTGCCTGGCAGCCAAATTGTTGACACTTCTTTTTTCCTATGCACTGGTTTTACACAG |
| | | CTGTCATTTTTCTTTCAAAATTGCAGCAGTCCCACAGATGTGTGCATTTGGACAAATAGTACTTAAAAACAAAACAAACAA |
| | | GCACTCAGCCCAGCTCCTCAATACTACCTGGAAAAAGCATTGGCATTATTTTCAATAAATATCAAGCACTAAAAAAA |

TABLE 8
DE BD nORF peptide sequences

| | | | | n_ | SEQ | | |
|---|---|---|---|---|---|---|---|
| norf_id | n_chr | n_start | n_end | strand | ID NO: | V13 | de_dirn |
| 0zkdH6 | chr1 | 167599566 | 1.68E + 08 | + | 164 | MATAGSGGDSGDRELRPGRAGASAAE | down |
| | | | | | | SAPEGHGAWRHPNDPSTQMALL | |
| 1haeH1 | chr1 | 237963358 | 2.38E + 08 | + | 165 | MAATYGLNSIDVKYQMWKLGVVFTDNV | up |
| | | | | | | SLLHYHKRKCTLIR | |
| 718hH4 | chr4 | 155665986 | 1.56E + 08 | + | 166 | MAPRSVPSPTSFVRL | up |
| 95ivH6 | chr5 | 140023171 | 1.4E + 08 | + | 167 | MAMGLCLLWGCCCWTWLGLSRKPRM | down |
| | | | | | | QDWSCWHAPCFDV | |
| 96nxH6 | chr5 | 141524145 | 1.42E + 08 | + | 168 | MSQRIYQLCKSSEDARNFLKSPQDQSS | up |
| | | | | | | LYLLKMFSGKGLPAFMNSLSRSKRTPA | |
| | | | | | | GSESRCRTQRNNHLL | |
| 9izeH2 | chr5 | 43575997 | 43602916 | − | 169 | MSSLGSHQNKETGLKTLIPESILPHIQN | up |
| | | | | | | EIHAQRCQEESRQTGLSRLYKKHDASIC | |
| | | | | | | FW | |
| a87hH2 | chr6 | 31553973 | 31554980 | + | 170 | MGEKLKPEEGQECLRWQHFSSARQHW | down |
| | | | | | | PVWSLSILQATSSG | |
| a87hH3 | chr6 | 31554008 | 31555009 | + | 171 | MSEVATLLFSQTALASLESVHPAGHKL | down |
| | | | | | | WMRNLRQVTSP | |
| a87hH4 | chr6 | 31554088 | 31555065 | + | 172 | MDEELEASHQPLIISPKRARTRVPDLQA | down |
| | | | | | | SP | |
| axtlH1 | chr7 | 296640 | 297004 | + | 173 | MRGGEADTALRQQPPHPGRHGHDDLR | up |
| | | | | | | LPHGKHGPSPLRDF | |
| bc4xH2 | chr7 | 141438176 | 1.41E + 08 | + | 174 | MRGWAARVRSSGKA | up |
| cp2xH1 | chr9 | 44401965 | 44402364 | + | 175 | MRKLRLRASNPGPSGAPGTRRHFSTSR | up |
| | | | | | | GGHHCARRWLRRVRSLVLFSRSRSQT | |
| | | | | | | PSCQNLDPNPPIAHFPLPLERISEVPRT | |
| | | | | | | ACLHGRDASSVWPPPERSD | |
| cswfH1 | chr9 | 115872338 | 1.16E + 08 | − | 176 | MAFIGKAPVLGEQIPHLWQCSVPGSCLE | up |
| | | | | | | HSATVTPLPCIGLDHMTQRCREKPPLDA | |
| | | | | | | WSDFSC | |
| dkjeH1 | chr10 | 116261761 | 1.16E + 08 | − | 177 | MPGQGTSDSANSQGNSTPHPQVPGYP | down |
| | | | | | | PDCVVFLNNLPEETNERMMFHAIISVPA | |
| | | | | | | FNKVCLVPGRHDIAVVEFENDGQAKAV | |
| | | | | | | RDALQEFRITASHTMGVTYVNK | |
| dol2H3 | chr11 | 18416109 | 18418402 | + | 178 | MLQPLPPIPDLIATRPRRPPDVHSRFLL | up |
| | | | | | | VPSPIWQL | |
| ejkwH4 | chr12 | 6898743 | 6923489 | + | 179 | MGSWLQSSKSSHRYACLRSSGQERRK | down |
| | | | | | | PRGPSKLNDRADSRRSLWDQGNFPLIIK | |
| | | | | | | NLKIEDSDTYICEVEDQKEEVQLLVFGC | |
| | | | | | | EWGRWG | |
| eveeH1 | chr12 | 133625393 | 1.34E + 08 | + | 180 | MGRRWRNSKFRFSRSLESRW | up |
| ezgpH1 | chr12 | 47604500 | 47610169 | − | 181 | MTLGSYCSCWCGMFTAEKMVRRGKEE | down |
| | | | | | | VRRKKQGNDRVLLSYPGWSAMA | |
| f7tzH1 | chr12 | 82747009 | 82748311 | − | 182 | MNRYKPNVLLRNKNSRGENRREKKPK | up |
| | | | | | | GSTKRRKWKCLKY | |
| gcddH3 | chr15 | 40707135 | 40707646 | + | 183 | MGWTWSGWCWPGGLLGSCKRSWTTP | up |
| | | | | | | FPTCT | |
| hubwH1 | chr17 | 5403122 | 5403643 | − | 184 | MPLDPYHSVTWGHAQGSHHFVFGELR | up |
| | | | | | | VRPILESLRDEPDPDPRPSREGPAGRV | |
| | | | | | | GALARGGPEPCDAASPPGGASCAPELA | |
| | | | | | | RPREDKSAQQAKLEGGTRLCCRCPEES | |
| | | | | | | RLVPGGAVSPGDHVLEVSGTRGTCGC | |
| | | | | | | RPRRHAGPELAHS | |
| iu75H2 | chr19 | 48758950 | 48759608 | + | 185 | MLTKRNRTQSSLAVSYQQASVSPSCTL | down |
| | | | | | | GLLKLT | |
| iu75H4 | chr19 | 48758982 | 48759643 | + | 186 | MGGVLPTSLRFSFLYTRAPETHLMKSPS | down |
| | | | | | | VTQAGV | |
| jetaH6 | chr20 | 18548132 | 18550131 | + | 187 | MSSRPGTGGCRWSTGSAPGLCWAHCF | down |
| | | | | | | TIAGQWRSRQTKRMAQQVKYPVNSLNA | |
| | | | | | | QKDFMWKQLSHIKKILFQIQKRSSTIGN | |
| | | | | | | HGLVALVQNHDWLLNSENQDLVQHLNLI | |
| | | | | | | VALIPILGL | |
| ji52H8 | chr20 | 47895203 | 47905699 | + | 188 | MVLLLVAVRLHTLLSCPLEQPAGTEWIL | down |
| | | | | | | EEGVTTGPPRKPRADIYNLRSPDENWR | |
| | | | | | | WNMRGALWKEKDRPCAFMKVKIWLNQ | |
| | | | | | | FHKVTVYIA | |
| tracer_ | chr7 | 143510715 | 1.44E + 08 | − | 189 | TAPTSETAPPPGPPRPASPPTRPPGAT | up |
| 101813 | | | | | | AEPVRQSLGIRTAQGRIPWEHPPAEICT | |
| | | | | | | SATILSGRRKGKGSAASLATACIISSQR | |
| | | | | | | RSTGSGSFRA | |
| tracer_ | chrX | 140270477 | 1.4E + 08 | − | 190 | VREGQPAAPGIALQLPAAGVGAGASLT | down |
| 113051 | | | | | | RKCLEVSSTLAIPGIPPLLI | |
| tracer_ | chrX | 140270587 | 1.4E + 08 | − | 191 | MRHRALSIENSQLMEQLRLLVCERASLL | down |
| 113052 | | | | | | RQE | |
| tracer_ | chr11 | 3716793 | 3717934 | − | 192 | LSSWDYRGSSSCSSFISVCG | down |
| 15054 | | | | | | | |
| tracer_ | chr11 | 67374462 | 67377307 | + | 193 | LARRDAGNTAAARLVASRAGICAFQRR | up |
| 18449 | | | | | | HAPKKTSFGSLKDEDRIFTNLYGRHDW | |
| | | | | | | RLKGSLSRGDWYKTKEILLKGPDWILGE | |
| | | | | | | IKTSGLRGRGGAGFPTGLKWSFMNKPS | |
| | | | | | | DGRPKYLVVNADEGEPGTCKDREILRH | |
| | | | | | | DPHKLLEGCLVGGRAMGARAAYIYIRGE | |
| | | | | | | FYNEASNLQVGRERCRQMRRCSVCTH | |
| | | | | | | TPLTQHSCSEVLVPGLSVVELGEWWPV | |
| | | | | | | LHASQMGLGSQSLLPGLLMLLV | |
| tracer_ | chr11 | 69240563 | 69242419 | + | 194 | MVKREKDFCFQIRCFLRLGAEFSCPWR | down |
| 18675 | | | | | | SSSSGPLAGPHPLQSGQIPCSQGKWP | |
| | | | | | | MPAVLVCNIGPACWNLTLFLPDTQQNK | |
| | | | | | | PQGRDQCLQPESRRKRSDR | |
| tracer_ | chr14 | 96343363 | 96389122 | + | 195 | LPGRMRQEPAAASFFSFSSPSCGSGAP | down |
| 33416 | | | | | | GPGRAAALLRAPRAPQPPPDALLG | |
| tracer_ | chr15 | 45119253 | 45120790 | − | 196 | LFLPPLQATRSGGTLVLVGLGSEMTTVP | down |
| 35299 | | | | | | LLHAAIREVDIKGVFRYCNTWPVAISML | |
| | | | | | | ASKSVNVKPLVTHRFPLEKALEAFETFK | |
| | | | | | | KGLGLKIMLKCDPSDQNP | |
| tracer_ | chr15 | 85175273 | 85177596 | + | 197 | LAFPLPRSITTPRGPPPAPEALLSVAKA | up |
| 37550 | | | | | | REVYKRADPGVAGPRTVPDCPSPGDPDL | |
| | | | | | | GQTAASGEWRGGSGPGGRLAERTWKT | |
| | | | | | | EAGASGEAVRPHPSCSAAVAMANDSC | |
| | | | | | | GPGEPSSSERDRQYCELCGKMENLLR | |
| | | | | | | CSRSSFCCKERQRQDWKKHKLVCQGS | |
| | | | | | | EGALGHGGGPHQDSGPAPPAAAPPSR | |
| | | | | | | DRALEARKAARRRDSASGDAAKAKAKS | |
| | | | | | | AADPAAAASPPRASPGRTKAMAACYPV | |
| | | | | | | NGTGYVRHVDNPNGDGRRVKCITLR | |
| tracer_ | chr16 | 68118986 | 68225710 | + | 198 | VVPYPIREANGLLSSGLEGIAGGIQAKL | down |
| 42874 | | | | | | QFPRDLEPDDCASIYIFNVDPPPSTLTT | |
| | | | | | | PLCLPHHGLPSHSSVLSPSFQLQSHKNY | |
| | | | | | | EGTCEIPESKYSPLGGPKPFECPSIQIT | |
| | | | | | | SISPNCHQELDAHEDDLQINDPEREFLE | |
| | | | | | | RPSRDHLYLPLEPSYRESSLSPSPASSI | |
| | | | | | | SSRSWFSDASSCESLSHIYDDVDSELNE | |
| | | | | | | AAARFTLGSPLTSPGGSPGGCPGEETW | |
| | | | | | | HQQYGLGHSLSPRQSPCHSPRSSVTDE | |
| | | | | | | NWLSPRPASGPSSRPTSPCGKRRHSS | |
| | | | | | | AEVCYAGSLSPHHSPVPSPGHSPRGSV | |
| | | | | | | TEDTWLNASVHGGSGLGPAVFPFQYCV | |
| | | | | | | ETDIPLKTRKTSEDQAAILPGKLELCSD | |
| | | | | | | DQGSLSPARETSIDDGLGSQYPLKKDSC | |
| | | | | | | GDQFLSVPSPFTWSKPKPGHTPIFRTSS | |
| | | | | | | LPPLDWPLPAHFGQCELKIEVQPKTHHR | |
| | | | | | | AHYETEGSRGAVKASTGGHPVVKLLGY | |
| | | | | | | NEKPINLQMFIGTADDRYLRPHAFYQVH | |
| | | | | | | RITGKTVATASQEIIIASTKVLEIPLLP | |
| | | | | | | ENNMSASIDCAGILKLRNSDIELRKGET | |
| | | | | | | DIGRKNTRVRLVFRVHIPQPSGKVLSLQ | |
| | | | | | | IASIPVECSQRSAQELPHIEKYSINSCS | |
| | | | | | | VNGGHEMVVTGSNFLPESKIIFLEKGQD | |
| | | | | | | GRPQWEVEGKIIREKCQGAHIVLEVPPY | |
| | | | | | | HNPAVTAAVQVHFYLCNGKRKKSQSQRF | |
| | | | | | | TYTPVLMKQEHREEIDLSSVPSLPVPHP | |
| | | | | | | AQTQRPSSDSGCSHDSVLSGQRSLICSI | |
| | | | | | | PQTYASMVTSSHLPQLQCRDESVSKEQH | |
| | | | | | | MIPSPIVHQPFQVTPTPPVGSSYQPMQT | |
| | | | | | | NVVYNGPTCLPINAASSQEFDSVLFQQD | |
| | | | | | | ATLSGLVNLGCQPLSSIPFHSSNSGSTG | |
| | | | | | | HLLAHTPHSVHTLPHLQSMGYHCSNTGQ | |
| | | | | | | RSLSSPVADQITGQPSSQLQPITYGPSH | |
| | | | | | | SGSATTASPAASHPLASSPLSGPPSPQL | |
| | | | | | | QPMPYQSPSSGTASSPSPATRMHSGQHS | |
| | | | | | | TQAQSTGQGGLSAPSSLICHSLCDPASF | |
| | | | | | | PPDGATVSIKPEPEDREPNFATIGLQDI | |
| | | | | | | TLDDGKFISDMFLK | |
| tracer_ | chr16 | 68337874 | 68344706 | − | 199 | TSEGLERAAENNVFHLVATVCSQEEPV | up |
| 42939 | | | | | | QPLLREVLRPSRDSQQRVRRNLRASAR | |
| | | | | | | EVRQEGRYRVLSSRRSLGTTSSGQESE | |
| | | | | | | YTPGNPEAAGNSGFQLLDLVHEEGEPE | |
| | | | | | | AASAGSCKTSDPDVILCNSVELIRERLT | |
| | | | | | | VSEDGPGVRRQEEQKHDDYVYDIYYLET | |
| | | | | | | ATPGWIENILSVQPYSQEWELVRGPMS | |
| | | | | | | RRGMRHPKLLI | |
| tracer_ | chr19 | 7982552 | 7983974 | + | 200 | LDPVSAWHFPQGRPRVSPAVQCALSLP | down |
| 55120 | | | | | | RRPPRPWNPRRWWRWCWQPSCWAP | |
| | | | | | | RWPPGWVSSVRTQRPTPLARPREPRP | |
| | | | | | | AVPSPGGPSEEGMVRPQHGPEIHPATN | |
| | | | | | | SGPGPTGPDPPPWTSDLMRPRPLRFSP | |
| | | | | | | PPVPPTCAQNKPLD | |
| tracer_ | chr19 | 36395470 | 36399113 | − | 201 | LQQAPAPASPAGCLRPVQAQAQSDCS | down |
| 57824 | | | | | | CSTVSPGVLAGIVMGDLVLTVLIALAVY | |
| | | | | | | FLGRLVPRGRGAAEAATRKQRITETESP | |
| | | | | | | YQELQGQRSDVYSDLNTQRPYYK | |
| tracer_ | chr19 | 40530247 | 40530816 | − | 202 | MAVPSFTQQSLWPVHVRPPMPPCYPG | up |
| 58436 | | | | | | PYDERMPPNQLYSVLNSSLLGHPFPPS | |
| | | | | | | VPPVLSPFQRAQLLGGAQLQPGQMSPS | |
| | | | | | | QFAWVPGFVGSLHAAMNPKLLQGQVG | |
| | | | | | | QMLPPAPGFHAFFSAPPSATPPPQQRP | |
| | | | | | | PGPGPHLQNLRSQVPVFRLDATPLHPQ | |
| | | | | | | HH | |
| tracer_ | chr19 | 41939172 | 41945380 | − | 203 | MFRDKEWIRPDKYGHFSQEFWNFCEV | down |
| 58683 | | | | | | PVEAVDAGDCDINYEGLDNLLRLKELQS | |
| | | | | | | LSLQRCCHVDDWCLSRLYPLADSLQEL | |
| | | | | | | SLAGCPRISERGLACLHHLQ | |
| tracer_ | chr3 | 14987643 | 14989528 | − | 204 | VAKNMLFV | down |
| 76036 | | | | | | | |
| tracer_ | chr1 | 161696521 | 1.62E+08 | + | 205 | MGPREARGAALGGVVLRCDTRLHPQK | up |
| 7857 | | | | | | RDTPLQFAFYKYSRAVRRFDWGAEYTV | |
| | | | | | | PEPEVEELESYWCEAATATRSVRKRSP | |
| | | | | | | WLQLPGPGSPLDPASTTAPAPWAAALA | |
| | | | | | | PGNRPLSFRKPPVSRSVPLVTSVRNTTS | |
| | | | | | | TGLQFPASGAPTAGPPACAPPTPLEQS | |
| | | | | | | AGALKPDVDLLLREMQLLKGLLSRVVLE | |
| | | | | | | LKEPQALRELRGTPETPTSHFAVSPGTP | |
| | | | | | | ETTPVES | |
| tracer_ | chr4 | 152091866 | 1.52E + 08 | − | 206 | LPKKPEITPRSLPPKPTVSSGKPSVAPK | up |
| 85751 | | | | | | PAANRASGEWDSGTENRLKVTSKEGLT | |
| | | | | | | PYPPLQEAGSIPVTKPELPKKPNPGLIR | |
| | | | | | | SVNPEIPGRGPLAESSDSGKKVPTPAPR | |
| | | | | | | PLLLKKSVSSENPTYPSAPLKPVTVPPR | |
| | | | | | | LAGASQAKAYKSLGEGPPANPPVPVLQS | |
| | | | | | | KPLVDIDLISFDDDVLPTPSGNLAEESV | |
| | | | | | | GSEMVLDHVEALPLTRELINNLFKRAEH | |
| | | | | | | KHN | |
| tracer_ | chr6 | 146136420 | 1.46E + 08 | + | 207 | VTRYPLSGFTSPRESSALDPGGRRSLS | up |
| 96176 | | | | | | GARTGQPTRVCFCEGPRRHSAKPSPC | |
| | | | | | | CCHVKKDVFASPSAMIEKKHKLQ | |

TABLE 9
DE SCZ nORF transcript sequences

| | SEQ ID | |
|---|---|---|
| nORF | NO: | nucleotide sequence |
| >ENST00000496999.1:: | 208 | GCCCAGGAGTTCTCCAAACCCGCGCTGCGGAGTGAGTGACCAAGTTCCGGCCAGTTCGACCTCGAGGATCCAGAGGTGGA |
| chr1:46016517- | | GACGGTACTACCTCCCAGCTCTGTTTTCCATCCCCTTCAGGTCCTTCCTCGGGAGGCGGCGAAGGCGGTCCACCCTGCGC |
| 46032885(+) | | GTGATCCTTTATGCCCGGCCCCTGCCCCTCCCTCCGGGTGGAACTTCCCCCTCACCGCCAGACTTAAGCTGAGGATCGTTG |
| | | GATCTCTGGCGGGGTGCAGAACTGAGCCCAGGCCACAGTACCCTATTCACGCTCTGTGCTTGTGCCAAGGTAAAAGCAGCT |
| | | GTTAAGTATGCCCTTAGCGTAGGCTACCGCCACATTGATTGTGCTGCTATCTACGGCAATGAGCCTGAGATTGGGGAGGCC |
| | | CTGAAGGAGGACGTGGGACCAGGCAAGGCGGTGCCTCGGGAGGAGCTGTTTGTGACATCCAAGCTGTGGAACACCAAGCA |
| | | CCACCCCGAGGATGTGGAGCCTGCCCTCCGGAAGACTCTGGCTGACCTCCAGCTGGAGTATCTGGACCTGTACCTGATGC |
| | | ACTGGCCTTATGCCTTTGAGTGAGCCTTGCCAGAGCCTCATCTGGGGAATCAGGGGGTTGAGCAGGATGGTGTTAGTAACT |
| | | TATTGTAAGTCACAGCAGCAGAGCAGGATAGGAACACTCATTTGCATGCCAAGCTGAGGAGCTTGACATGGGATCTTAGCCT |
| | | CTTCTGCTACAGCAGCTTAGCTGTAGCTACAGGAGTTTAACTCTGGAAA |
| >ENST00000429328.2:: | 209 | GCCGCGCCTGAAGCTCAACTTCCGAGGCTCTCATCAATCTACGCTTCCTTTGGAGCGTGCAGCCACCAGGTGGCAGCCGAG |
| chr1:47644921- | | GCCACGGGCAGACAGAAAAGCTCCCGAAACTGAAGGACTGCCAGGCAGCAGAGTTCCGGAGGGCTCCCAGGACCTTCAAC |
| 47646011 | | CAGAAAGAGACAGAGCATATGCGCATATCCCTGCAAGGAATGTACTATAGAGCCAATTTTACCGGCAAAGGAATGGAAGATC |
| (+) | | TGAAAGGCTAGGCGATCCCACAAGCATCGAGGAGCAGGCTGGAATTTGAACCAATCGGCATGATTCCCAAGCTCAAGCTTT |
| | | TGGCCACCCCTGCACTGCCAGGGACAAAAAAAAAAAAAAAAAAAAACCCTGGGAGGAGAGCCAAGCTAGGGAGTCAAGACC |
| | | CTCCAGCCAGGTCTGGATGGGAGGCAGGTGCCTCCACCCTGGTCCCAGAGCAAGTTCTGCTGGCTTCAGCCACCTGACTT |
| | | CCCTGTGAAGGTGGGTAGAGGCCAGAGCAGACTGTGTGTGATTCTGGCCCCAGCCCCAGCACCTCTACCCCCACCTTTACT |
| | | ACCCCTAGGCTGCGGGAAGAGGCTTTGCCCAGCAATAGTCCTGATCTAATGGATCCTTGAAGCACAATAAACAGATAGTATT |
| | | TCTGCATGTGTCA |
| >ENST00000435559.1:: | 210 | CTGCTCACCATTTCTGAGCCGATGAGGACTCACCTGGGCTCCTCACTGGTCAGGGAACTGTGGCCCAGCCGCAGAGTGATT |
| chr1:95086706- | | GCTTTGTCAAGAAGGGAAACATTTACCTTTCCTTCTTCCCTCTGCTCTCTCCTAATTAACAATTAAATGGAAGGAGTTGAAAAG |
| 95089740(−) | | ACATGTCAGAGACTTCAATCTCCTCCCGGCCTGAGGATGAAGGGGGCTCGAACCTGAATGTTCTCTTCGCTGTGGCCAGAA |
| | | CCTAGGGGGCTGGGGTTGCTGCCAGCTCGACTGGAGGCCTGCTTCCAGCCCCAGGCTGTGGTGAGGGGCCTCACAGATTT |
| | | CACAAACAGGAGGGCGGCTGACCAAGCGCCAAACTATTTAGACAAACACACACCCTCCCTTCCCGCCAGAGTTTGGGCTGA |
| | | TTTCTCCCAATGCTAATGAACAGCTCATAAGTATTAATAGACTCCTTGGCACCTAGGAGTCAGGCAGAGTAACTTCCAGTGTG |
| | | AATTTTGGCCAAGGACCCTGATGCAGCCTCAGGATCTTCCAAGCCCCTGTTGAGTCCAGGGGGCATGAGACACACAGGGAG |
| | | GCTGGGGCTGCTCCCAGGAAGCAGGCTCACAAGAGCCAGACAGGCTCCCACAGACAGCTTTTAGGTCACAGAAACCTCTG |
| | | CCTGCCAGGTAGGCATGAACACAGAAACCCTCCCAAATCCTCAGACTGTGGAGCACACTCCAGGTGAGCCAGGATCAATCC |
| | | CTGGTCTCAGAGAACACTTAGATGGGGGTGAAAAGCAGGAACTGAGAGCTTCCCACCCAGCACCTGTCCCTGCTACCAGCT |
| | | AAACCTGTGTCGGTCAGTGCAGGAATTGTACAGGTGATGCTCTGAGCCTGTGCTGAGGCCATGTATGGACTGAGGAAACCT |
| | | CACTGTGTGTATGTAAAGTTAATTCAAACCTATTTCCCTGAGTGCTTACATATTTTCATAAGGAAAGTTCTGTTAGGAGAGCTG |
| | | CCTGAAACAACACCTCTGCCTGTGCTCCCACTTTCTCCCGCTGGTGTGGGGAGGAGAGGCTGACATCGCTGGGTCCACGTT |
| | | CCTCTGCCCTGGGTGGGGTATTAATGCTGCCATTCCCACGCTGCCTGCTGTTTCCCCTCATCCTCAGGAGGGCTGGTCTCT |
| | | GTGCTGCAGGCAGCTCCACCAGGCCTGGGAGCATGACACCGTCTGCCAATGCCTTGCCCAGGTCCACTGGCCTCACCCTT |
| | | CCGCAGGCCTGCGCCTTCTGTGACTCTCTGCCTCAGCAATTTCTCTGGATTTCTTTGTCCTCAGGAATGTGCTGGGGCCACA |
| | | GGCAGGTCAGGCCAGGCTTTCTGGGGAGTGAATGCTCTCTGCCAGTGGCGGATGGAACTGGTAGACACGTCCCCCGGATT |
| | | CCTCGCCTGCAAGTGGGATGGTGCCAGGAAAGTTCTACACCATCCTTCCCAGGTCCCAGGAGGACAAGCACAGTTGTCCAC |
| | | AAGGGAACCAAGAGGATGTTGAAAAATCCTGGTTGGATCTGAACTTGGAAACAAATTTGAGGAATCTATTTTACTTAATCCTA |
| | | CTGAAAAATTCCCTAATGAGTTAAAGAAGAACAAAGTAAAAGAAGATAAAAAAGCAGTTGGTACTTGAAATGAAGATCAAATC |
| | | ATCAAGTCCTATATGTCACCCACAGATCTGAGATCACTGACTCCCAGAGACACCCAGTTTCTCAGTGTCAGTACACACTGCC |
| | | TGGGTTAGTAAGCACAGGCTGAGACCTGGGAGGAATTGATGAACCAAATGTGTCTGGGCTTTTCACTTCCTCGGCATGAGTC |
| | | AGCCAAGAAAGCCCACCTCTCCTCCCCAGCAGCGTGCAGGGCCTCTGCAGATAAATGTGCAGGATAGCAGACCTGGGTGT |
| | | GGTGCTGCCTGGCACTGAGCCAGCCCCCAGATTCTTTGGCTGCAGAGTGGCCTGGCCAACTCACACAGGTAGCGCCCTTTT |
| | | ATTCTTGGAAAAGGAAGCACCAGGTGGAAAGGGAGGTTAAAGGAGGGAACACAACTTTTTTCCAGACCCCTCTGCCCAGGA |
| | | TGTTAAAGGTTGCTGGAAAATGCCTTTGGTGGCTTTAAATGTCACAGATGACATATTCCAGGATGGATCCAGCCTCCACTGG |
| | | ACTTGGCCCCTCTATTTTGATTCCTGCCTTTCCCCAGCCTCAGCCCTGAGCGGCATAATATCTACACGGGGTTAGCTGCAGG |
| | | TGGAACTCAGAACACAGCTCCTAGACAAGTAGAGTGAGGCCTATGGCTTCATCATCAAAATGTAAAATTTAAA |
| >ENST00000482384.1:: | 211 | ACAGGCTGAGTGCTGCGGCGCGATCCTTGCTTCCCTGAGCGTTGGCCCGGGAGGAAAGAAGATGGTGCTGGATCTGGATT |
| chr1:109756553- | | TGTTTCGGGTGGATAAAGGAGGGGACCCAGCCCTCATCCGAGAGACGCAGGAGAAGCGCTTCAAGGACCCGGGACTAGTG |
| 109757775(+) | | GACCAGCTGGTGAAGGCAGACAGCGAGTGGCGACGATGATCAAAGGCTTCTCCTGAGACTGCGTCACTTCTGCAACACAGT |
| | | AGATTCATTCCTTCGGTATCGGAAGCATTTCGGGGCGTTGGAGGCCGCTCTTGGCCAAAATAAATGACCCTGAAGCTTTTCG |
| | | GAAGGCCATCCCCCGACCTATGGGCATACCTTTAAGAATTAGAAAAGTCTTTTCCGTGGTAGATCAAACGCGAGGGGAGTGA |
| | | GGAGGCGAGCAAAAGATTGTAATGACTGAAGCTGTTTAATTGTGATTTGTGGACGTTTTCCTTTAAAGACATTGGCAGAGTTG |
| | | GGCTAGAACCTGGAACTGCCGGATCTTGGAGTCATATCGGGCATCTATCATGAAGCCGAATAAAACCATAGAACCATCTGCC |
| | | CATAAATCTCTGCCTGTATTAACTGGAGTGGTCGCTGGTTGGGCGGATGCTGTCAAGTACTTGTGATTTGATTTGTCTGATGC |
| | | AATAGGTCTGCAACCATCTGGGAGCTAGGTCGATGACAGTACACTGCAAAGGCAGCCACTATCCAAGAGTGTGATGGATTTC |
| | | TATTAGGAAAATTGATTTTCTCCTGTTTATAAGGATCTTGGATGTCAAAAGAATATTTGTTTAACTTGA |
| >ENST00000495397.1:: | 212 | AAGGTGGTCTACAAGCTTCACTACTACCACGACGGCCAGGCCGTGCGCTACTTCCACTCCAGCGCCAACTACACTGTGTTA |
| chr1:161695690- | | CAGGCGCGTGCCAGCGACAGCGGGCGCTACCAGTGCTCGGGCACCATGCGCATCCCGGTGGAGAGCGCGCCCATGTTCT |
| 161697932(+) | | CCGCTAAGGTGGCTGTGACAGTGCAAGGTGGGAGAGACCAGGGGCCCCGGGAGGGAGGCAAATGAGCATTGAGAAATTCT |
| | | GGGACAGGAGCTCGGCGAGAAAAGAAGGGGCGGAAGTTCAAATAGCTGCCACTTGGAGGGTTTCTCTTAGACTATGGACG |
| | | CTGTCTCCTCTCCTTTGCCGTGAAGCAGCGCTTGATCCGCCGCCTGCTTGGAGGCTGGTCCCTTTCCCCGACGCACATCCT |
| | | GGCTTCTCACTCTGGCTGGGGACTCCCACAGAGAGGGCAGCTCTGGCAGGGGCAGGGGCCACTGTGGGACTGGGACGAA |
| | | GGAGCGACTCCCTAGCGTCCTGCAAGGCAGGCGCAGCGTCTCCTATTCTAGGCTGCAGAACCCGAGCTGAGTGTCAGTCG |
| | | GGATGTGACATGAAGCGTCTGGCCTGGTCCCTCTTCCTTTCAAGCTTTCCCCGTCCCTCGTGGACTCGGTCCCCCTGCCCC |
| | | ACATTTCAGAAGGCTCCCCTTCCCCCTCCACGTGGACACACGGCCTCCTCCCCTCCCCCCTTGGTCTGTGGGTCTGCAAGG |
| | | AGCCCTCGCGGGAAGCAGGAAGGAGCGGGGTCGCGGAGCGGTGGACAAGCCGGCGCCGTTGCTCCCCGCCCTCTCCGTA |
| | | GAGCTGTTCCGGGCGCCGGTGCTGAGGGTGATGGGTCCGCGGGAGGCCCGCGGCGCGGCGCTGGGTGGGGTGGTGCTG |
| | | CGCTGCGACACGCGCCTGCACCCGCAGAAGCGCGACACGCCGCTGCAGTTCGCGTTTTACAAGTACAGCCGCGCGGTGC |
| | | GCCGCTTCGACTGGGGCGCCGAGTACACAGTCCCGGAGCCCGAGGTCGAGGAGCTCGAATCGTACTGGTGCGAGGCGGC |
| | | TACCGCCACCCGCAGTGTCCGGAAACGCAGTCCGTGGCTGCAGCTCCCGGGGCCGGGTTCTCCCCTGGACCCGGCCTCC |
| | | ACCACCGCCCCAGCTCCATGGGCCGCAGCCTTGGCTCCTGGTAATAGGCCGCTTTCCTTCAGAAAGCCCCCGGTGTCCAG |
| | | ATCGGTCCCGTTGGTCACCTCCGTCCGGAACACCACCTCCACCGGGCTGCAGTTCCCGGCGAGCGGCGCCCCGACTGCG |
| | | GGGCCACCCGCCTGCGCTCCGCCGACGCCCTTGGAACAATCGGCTGGAGCCCTGAAACCCGACGTGGACCTTCTGCTCCG |
| | | AGAAATGCAGCTGCTCAAAGGCCTTCTGAGCCGGGTGGTCCTGGAATTAAAGGAGCCACAGGCCCTCCGGGAGCTCAGGG |
| | | GAACGCCCGAGACCCCCACCTCTCACTTTGCTGTGAGCCCGGGAACCCCAGAGACCACTCCTGTGGAGAGCTGAGGGGGC |
| | | GGCTACCGTCCCCTCTGCAGGCTCATTCCTCCTTGGTCTCCTGCTTCCCCTCACGCGAATTTCTTTCAAAGCCATCTGTTTG |
| | | CATCCTTGTGTTTTGCTGTGGTTTTTAAAGGAGCGCCCACGAAGTGTAGTGGCTGACGATTTCAACCTCACACAGCAGTTTG |
| | | TAACCGCAAGCATTCTCTTTGAATTCTCACAGAATTCAGCAAGAAGTAGAAACCTGTTATTTACTACATTGTGATTTAACTTTG |
| | | GATGTGAATTTAGTCACCCTTAGCCCTTCAGATAAGCCTAGCCAGTACATATTTCAGCACAGGCAGTTTTTTTGGTATTTAAGT |
| | | ACATTGAGGTAACTGAGCACTTGAGAATATTTTAGGGTCAAAGTGTAATTATTCATAATGAATTTACTCTGTTGATATTAAAAA |
| | | GACGTTCAGTCCTATTACTGATGAGTTTACATCTTCAAATAAATCCTGGGTTCTATTT |
| >ENST00000472038.1:: | 213 | AGTCCCGCAGCCGAGCGCAGCCGGGCGCGCGCCACCGCCCACTCGCCCTGTGCCCGCCGCAGCCCGAAACTGGCCACG |
| chr1:167599497- | | GCCGGGAGCGGAGGGGACAGCGGGGATCGTGAGCTCCGGCCCGGGCGAGCGGGTGCGTCTGCCGCAGAGTCGGCACCT |
| 167634277(+) | | GAAGGACATGGAGCCTGGAGACATCCAAATGACCCCAGCACACAAATGGCCCTTCTCTGAGAGAGTGTCCTCATGGCTGGA |
| | | GGCCAGAATCACACTCCAGCTTGCTCTTGTGGTTCCAAAGACTCCCATTTGTAAATTCCTCCGACATGAAGATGCAATGCCA |
| | | GCCCATCTGCTCTGCCAACCTATTCTGCAAGATGGGAATGAAAATGACTTGGCAACAGCAGATCCCTGCCACCCCCTTAGAA |
| | | TTCTTCAGGGCCAAACTGGCAACAGCCTGGTATCTCTACGAAGAGTGCAAGGGAGCCCACATTTTGACTAACACCTACTGTG |
| | | TGCCAGTTGGTATCTTAGGTGACAACAACAATAGCTAAAATGAGGGGAGAGCTTATTATGTGCCAGGCACTGTGCTGGGGCT |
| | | TTATATGCAATATCTCATTTGGGCTTTATCACAATTCTAAAAAATAAGTGCTATTATAAATGTTTATTTTACAAGTGGGGAAACT |
| | | AAGGCTCAATAAGGTCAAGTGTCTTGCCTAAGATCACACATCTAGTAAAAGGCAAGGTCTGGATGTGAACCCAAACTGTCTA |
| | | ATCAAACACCATCTCCTAGTATCCCCATGACCTTGTAGGGGGTGTTATTATCCTCATTTTACAGATGAGAGTCTGAACAGTTA |
| | | AGTCAATTGCCTAGGATCCCACAACCCGTCAATTACAGAGTGGGATCCAAACCTGGTCTTGCCAAGCTCCAAATTCTGTGCT |
| | | GTCTCCACTATCTTGCTGCATGAGTCCTGGCAGCTGCCCTGGGCTGGACAGGAGTGGCTGGATGTGGCCTACTCCCAGCA |
| | | GCTACCAGAGGCTGCCAGCCTCCTGCAGCCACAGCGCATTCCTGCTCACGCCCTGAGGCACACAGAGCTGGCATCCTGAG |
| | | ACCAAGCCAGGTTCCTAGCCCTAGGGCAAATGAAAGACCTCCTGCAGCCTGAGTGCACCTCAACTTTTGTATTTGAACTATC |
| | | ATGAGCACTTTAAAATGTTTTAATCACCTTTTTGAGGTAAAATATATCTACAATAAAATTCACCCCTTTTAAGTGTATACATTGA |
| | | TGAGTGTTGACAAATGTATACAGTCATATAGCCACCACCACGAGATGTAGACCATGATCAAGGTATAGAGCATTTCTGGCTG |
| | | GCTGTTATGGCTCACGCCTGTAATCCTTGTGCTTTGGGAGGCTGAGGCTGGCAGATCACTTGAGGTCAGGAGTTTGAGACC |
| | | AGCCTGGCCAACATGGCAAAACCCTGTCTCTACTACAAATACAAAAATTGGCCCAGTGCAGTGGCTCGCGCCTAGAATCCCA |
| | | GCACTTTGGGAGGCCAAGGCGGGTGGATCAACTGAGGTCAAGAGTTCAAGACCAGCCGGACCAACCTGGAGAAACCTTGT |
| | | CTCTACTAAAAATACAAATTAGCCAGGCGTGGTGGCGCATGCCTGTAATCCGAGCTACTCAGAAGACTGAGGCACGAGAATC |
| | | TCTTGAACTCAGGAGGTGAAGGTTGCAGTGAGCCGAGATCGTGCCACTGCACTCCAGCGTGGGTGATGAAGTGAGACTCTG |
| | | TGTCACC |
| >ENST00000486075.1:: | 214 | TTTTTTTTTAACTTGGGACTGCCAAGAGCTGAAGGAGAGTGGTGAGCAAAGGAATGAAGGGAGAGAGAGAGAAACTAAACTG |
| chr1:176522623- | | GAAGCTTGAGTTTTGTGTAGCTTCTTGAAACCTTATGAATGGATTACTAGAAGCTGAGAGCCAGGAGAGACCTATAGGGGAT |
| 176524872(+) | | GCAAGATCCCTACACATTAAAAGGGGGAGAAAAGCAGGCGGAATTCTTCTTCCTGGCTGTGTTATCCCTCCTTCCTGAAATG |
| | | AATGGAACCCACAAGACTCCCAGAAGGTGAAGTTAAGAGCTCCCAGACTCATAAGGTTATTAGAACAGCAAACTGGCACCCC |
| | | AAAGAACTTTACGGAGACTTGCAACCTATCAACAAGTTGGATGAGGGATTAAAAGCCTTCAACAACCAACAACCCCAAGCAT |
| | | CAAACTGAAGGAAACATTCTAACCTTCACAGACAGACTGGAGGCTGGATGGGGACCTGGCTGAAGACATCTGGAGAATGAA |
| | | AGTTAAGTACCAGCTTGCATTTTTGTGCCCCTAGATTATTTTTGCATTTTAAAATAAGAAGCATCAAATTGCGTGTCTCTGTGT |
| | | AAAAG |
| >ENST00000437764.1:: | 215 | GAAGAATAAGAGCTATTATTATTCTTTATGTCTTATCCTTTTTTTTTTTTAAAAAAAAAACCAAGTTTCCTTCTAAGGGAAATAAA |
| chr1:210404800- | | CACCAAAGGTAGGATTCCAAGGAAATGACTCACAGCGGCTGGCAGGGCTCTGGCGGGGCTGGCCCGGGCCCCCGCGCTC |
| 210407389(−) | | CAGCCCGCAGCAGATGTCGGCCCGGCCTTCCCTCCCTCCCTTGCCGGCCAAACCCGCTGGAAGCCCGCGGTTGCGGGAG |
| | | CGCCCTGCGCCCGTGGGACCTGGCGGCGAGGACGCCTATTCCCTGCTTCTGCGACCACAGCTGGGCACTCGGAAAGTTGC |
| | | TAGGGCCGAAGAGGCGGGAGGGGAGGAAGGGAAGCGAGAGGCGGAGGCGTGGACCAGGCGGGCAGCAGCCTCAGCCC |
| | | GCCGAGGAGTGATCCTCCGACTGTGAAAAACCAGACCTCTGGCTGCCTGGCTCCCTAGGAATCAAGAAGACGCACAGACTG |
| | | AAAATCAAGGGAGGAAGGAACAAGATCAAGGATGAGGATTAGCATCTGCAAATTGAACTCTATCTAATTTGCATGTTTATTTC |
| | | TTAGAATTGTAATTAAATTTGTGTAATAAATGTTTTCCATTTCATTTGTACACTTAAAACTTGGGTGACTGACATGCAGTTAAGT |
| | | GAATTTCCTTTTTTTAAAAGCAAATGTAGATTATTCAATAAAGCAGCAAACCAATATTAAACATAATTAGAACTAA |
| >ENST00000478275.1:: | 216 | ATTAAATGTTTCTGCTGGGTTGGCAGGTGTGATCATGGTGATGAGTCCAGTTTAGAGTCAGCTCCCCGAGGGCAGTATGCAG |
| chr1:212859759- | | GCTGCTTTCTTGTGCTAGTGACTTTTTGATTACAGAGAGCAAAGAGGCATGAGAAGCACTATGCAGAATACATTTTTTTTTAAA |
| 212872097(−) | | GTAGCAACTTCAACAAAGGCAGGTAACTTCTTGAGGGCATACTGTATGCTGGGAGCTTTAGGACAAAATCTCATTTCATCGC |
| | | ATCCCAATCCTAAGCATTAGGCTGTTAGCACCAAGTCTGCAGAGTTCACTGACCCTTTGTTTAGGGCTTGTATCCCCAGTGTC |
| | | TAGAACAGTGTCTGGCACATGGTAGGTGCTCAGTTAATACATATTGAAAGAATGAATGAATTATGCTTACAATAAATGAGAAA |
| | | ACTGAAAATTTAGAGAATTTAAACCATTTACACAAGACCATGTTGACACTAAGAGACAAAACTAGGATCTGTGTGATTCTAAAG |
| | | CTAAAGAGCTGTCTTCTAACCACATGTTCCCCTGTAATGTTCTGGTTTTGTTTATCAAGACTTCTTTGCCTCTCTAGAAGACTG |
| | | TATTATCATACTTTGTTCCCTTAAGCTTCTATTTGCTATGTATGTTATTTATTCCTCTCCTTTTTATGCCTTTATATTCTTGCATAT |
| | | CTGAAACTTCTTACCTGCAGTAACTTGGGAAGATGTGTGTCTTATGTGGAATACAAGATGAGATCAAATGATAACTGAAAATA |
| | | TCCTTTTTTGTGTTATTAATATTTGCATTCCGTGAATATTAATAACTGAGTAGTTTTCCTTGTTTGTACCTCTTGGCTCTTAAACT |
| | | CTGTAAGTGTGGTATTTTTCCCGCTTGGTGGTGTCTTCTCATGGGGTTCTAGAAAGCAAATACTGCTCTCCTTTTTCATGTTTT |
| | | CTATAGCTGATTGAAGTGTTTTTACCATGGTCAGTGCATTTTTCCGCCTGCCTGTTTCATGATCACTATTGAGGGATTTGACT |
| | | GAATTTGGTTAAATCTTACATATAGCTAACAGCTTTCAAGACACATAAAAAATTTGCTGACTTCTGAAACAAAATGTCAGCCAA |
| | | GCATTGTTCAAATGAGTACACTGTCAGTGGGGTTTAAGATCATTTGGTGTGTTGGTAGAGTTATGTGGCTGCAATTAGTTGTA |
| | | AGTGTAATCATGATTTGCATGCAGTATCAAAAGTACCTTTCATGTGTGGTGGTATAAGGACTGGCAAACAGCAAGTTCCAGAT |
| | | AGCAGATCTCTCCACCGCCCCCCACCCCCACCCAGCTTTCCAGTCTCAGATGGACGACATTGACACCACAGCTCTTCCAGG |
| | | CACACGCCCTGTGGGCTAGAGGTTTATGAACGTTGATTTGGAGACCAACAGACCTGAGTTCCACTTCCCACTCACCCACTTA |
| | | GTGGCTGTAATACCTGGAGCAAGGTCCTTAACCTCTCTGAGTTTCTTATTCCTCATCTTTTAAACGGCGGTCACACCACACAC |
| | | TTCACACACTCACATGGTGAGTGCTCCTGGGCAAGGGGCTGTTGTGAGCCCATTGGTATAGCAACTAGTGGTGCTGTCTACT |
| | | GCAAAGCAAATGATGAAGCTCAGTGAACAACGCTGAGTGATGAGACAGGGCAGAAAAGGGTAAGGCGAGGGGCCACGACA |
| | | TCACACCCACCCAGTGTTTTTCCCCAGAGCCCTGAGGATGATGACAGGAAGGTCCGAAGGAGAGAAAAAAACCGAGTTGCT |
| | | GCTCAGAGAAGTCGGAAGAAGCAGACCCAGAAGGCTGACAAGCTCCATGAGGAATATGAGAGCCTGGAGCAAGAAAACAC |
| | | CATGCTGCGGAGAGAGATCGGGAAGCTGACAGAGGAGCTGAAGCACCTGACAGAGGCACTGAAGGAGCACGAGAAGATGT |
| | | GCCCGCTGCTGCTCTGCCCTATGAACTTTGTGCCAGTGCCTCCCCGGCCGGACCCTGTGGCCGGCTGCTTGCCCCGATGA |
| | | AGCCGGGGACACTCCTCTGCCCAGCAAGGAGCCTTGGTCATTTTCATACCTGGGAGGAAGGCTTTTCCTTCACAATTGTATA |
| | | CAGGGGGCACCTGTGGCCAGGCCTCCTCCTGGGAGCTCCAGGACCAGCCAGCTGTGTTCCCTGCAGACTGGGCTCAGCC |
| | | CGACATCCAACAGGCGCCAAACTCACAGAGCCCTTGTGCAGATCCAGCATGGAGGCCACCCTCAGGAGTGACTTCTCATCC |
| | | ACCCTGGCAGCTAGTAGGTTCTGCTGTTATGCAGAGCCATTTCCTCTAGAATTTGGATAATAAAGATGCTTATTGTCTCTCCC |
| | | TTCTCCAGTTCTGGGAATTTACAGGCACAATACACTTCCTTTTCCTGG |
| >ENST00000472190.1:: | 217 | GAGTCAGTAGAAAATGGGGAGCCTCTGTGGGCTCCTGGTCCTGCACCGTGATAAATAGCCTGTCCTGCAACCTGGCTTAAA |
| chr1:233765303- | | ACAGGGAAAGCCGGAGTTAGGCAACAGAGTGCCTAGCTTTCTCAGTCATTCAAACACAAGCACGGAAGAAGAGATTGCACT |
| 233807445(+) | | GACCAGAGGACCGCCAGGGTGTGATGCTCCGTAGTCGCAGGGATGAAGGAAAACCATGGGCGGAGCTTGTCATGGACAGC |
| | | TCCTAGGTGGACATAAATGGAAACTCATTGGTTCTGGGGCACGGCGGGAAGAGGGAGACGGCCCTGGACGGCCGCACCAA |
| | | GAATTTCACAACCCAAACCCGACTAGAAAGCCTGGGTTATGGCCACACCGTGCCCTTGTCAGATGGAGGTAAGGCCTTCTG |
| | | CATCATCTACTCCGTCATTGGCATTCCCTTCACCCTCCTGTTCCTGACGGCTGTGGTCCAGCGCATCACCGTGCACGTCACC |
| | | CGCAGGCCGGTCCTCTACTTCCACATCCGCTGGGGCTTCTCCAAGCAGGTGGTGGCCATCGTCCATGCCGTGCTCCTTGG |
| | | GTTTGTCACTGTGTCCTGCTTCTTCTTCATCCCGGCCGCTGTCTTCTCAGTCCTGGAGGATGACTGGAACTTCCTGGAATCC |
| | | TTTTATTTTTGTTTTATTTCCCTGAGCACCATTGGCCTGGGGGATTATGTGCCTGGGGAAGGCTACAATCAAAAATTCAGAGA |
| | | GCTCTATAAGATTGGGATCACGTGTTACCTGCTACTTGGCCTTATTGCCATGTTGGTAGTTCTGGAAACCTTCTGTGAACTCC |
| | | ATGAGCTGAAAAAATTCAGAAAAATGTTCTATGTGAAGAAGGACAAGGACGAGGATCAGGTGCACATCATAGAGCATGACCA |
| | | ACTGTCCTTCTCCTCGATCACAGACCAGGCAGCTGGCATGAAAGAGGACCAGAAGCAAAATGAGCCTTTTGTGGCCACCCA |
| | | GTCATCTGCCTGCGTGGATGGCCCTGCAAACCATTGAGCGTAGGATTTGTTGCATTATGCTAGAGCACCAGGGTCAGGGTG |
| | | CAAGGAAGAGGCTTAAGTATGTTCATTTTTATCAGAATGCAAAAGCGAAAATTATGTCACTTTAAGAAATAGCTACTGTTTGCA |
| | | ATGTCTTATTAAAAAACAACAAAAAAAGACAAATGGAACAA |
| >ENST00000472146.1:: | 218 | AGTTCTAACCTGCTCTGCAGGAATAACGGTCCTGCCTCCCGACACTCTTGGCGAGGTTTTTGTACAGTTTGCTCCGGGAGCT |
| chr2:145184371- | | GTTTCTTCGCTTCCACCTTTTTCTCCCCCACACTTCGCGGCTTCTTCATGCTTTTTCTTCTCACCATTTCTGGCCAAAACTACA |
| 145277686(−) | | AACAAGACTTCGCAGATCGAGCCTGCGTGCTGCCGAAGCAGGGCGCCGAGTCCATGCGAACTGCCATCTGATCCGCTCTTA |
| | | TCAATGAAGCAGCCGATCATGGCGGATGGCCCCCGGTGCAAGAGGCGCAAACAAGCCAATCCCAGGAGGAAAAACGTGGT |
| | | GAACTATGACAATGTAGTGGACACAGGTTCTGAAACAGATGAGGAAGACAAGCTTCATATTGCTGAGGATGACGGTATTGCC |
| | | AACCCTCTGGACCAGGAGACGAGTCCAGCTAGTGTGCCCAACCATGAGTCCTCCCCACACGTGAGCCAAGCTCTGTTGCCA |
| | | AGAGAGGAAGAGGAAGATGAAATAAGGGAGGGTGGAGTGGAACACCCCTGGCACAACAACGAGATTCTACAAGCCTCTGTA |
| | | GATGGTCCAGGTAAGTGTCTAAAATCACTTGGCCTCTTCCTGTACCATCTTCACATCTTCAACTAATCTGTTCCACCTAGCAG |
| | | ATCGCCCACAACCAAATAATTAAAATATTTCTTTCCATTGAGCCCTTGCAGAATAGAGATACCAAAGCTGGTGACAGATTATG |
| | | AAGTGATAGTAATGTTATCACATTGCGGCAGTTACATCTTAGGAAGGGATTATATTTCAGTTTTGATGCAAGTCAAACATGAAC |
| | | CACACTTTCAACACATAACATGAGGTATTTGCACCACATTTACAATGTGCAAATTATGTAACATGATCAAGATCTATACTTTCA |
| | | TGATAGGAAATGGAAGCAAATGTAAAAACTCACAACATATATATGTATATAAAAATATTTTGTGTGGAAATTATGTAGCATTTCA |
| | | AACCAAATAACTGTCAGTTTAATGTAGCTTCAGTGTCTGTCTGACACCTCCTATTGGAATTCAATCGTTTAATGATTCCCTTTT |
| | | GTTAAGTGTGAATGTGATTTCACACTTTTACCTGGTCAGGTAGCTTTCTTATTATTTATCAGCTAAGTACTTGATAATGATTCCT |
| | | GCTGTTTGGTGGGACTCTCAAGGGAAGTAAAAATGGTCCCAGGCCAACTATAATAAAACTAAGGTAAATTATATTCAAACTAA |
| | | AACTTCCGACAACACTGTGCAAACGGGAAATGTCTTGGTCTAAAATTCACTTATGACGGAGAATCGCTAAGGTATGAAATGTT |
| | | TAACAAAATACAAGAAATCTTATTCCTGTGAACTTTCTCTGCTCATCTTTACATGGTAAAGTGGCCTGGAAAATTTGTTTAAAT |
| | | GACCTCTCTAATTCAGACCTCATGTCACCTTCATCTCTATTCCCAGGACCCACTGAGAGTTATCATTGTTTACCCCACCTTGTT |
| | | TTTTCTTTGACAACATAAGCAACAAAAGCATTTTTATAAATAGGTGAAGTGGCTGGGAAGGAGTCTACCTATATGCTGTTGTG |
| | | ATGTCGCAGACTATAAACCATCAAACATAGGCAGAATAATTGTTTGGTATACTGGAAGGAGGATAATAAAAGCACTTCCACTA |
| | | CGAAAGAAAATTAAATGTGAGATGAAAGAAAAAGAAAAAAGACTGATGGCAATTTGAGTACAGTGTATTTGTTTTTCGTTTTCC |
| | | TAACATACTGTGCCAAGTTTTCTACTCTTTCCGTTTCCAATTTTGCTTTCGGTGAAGGTTTCTCGAATCTCAGCAATCCCACAC |
| | | ATAAACATTTATCGTTATATCCATTAAGCCTTAAGGTTTTAGATTTTCAACTATGAGGGAGAACTCAGCCAAAGATGCTTGAAA |
| | | GATAAATACGTTTAATTAGGGGCAGAGGATGATCATTAAAGGAAGTCTGACTGCTGCAGAAGTCATTAACAATCTTAAATGAA |
| | | ATTTTTATTTTTATGATAGCCATTTACATGTATAATAAGAGCCAATGATTCTTGGGAGCAGTGTGACAGAAACAGAGTGTGGC |
| | | GAGAACTCATGTAAGACATACTATTTAAATGAGCTGGTGTTAGCTATTCATATTTGTTAATGAACTAGATATGCAGGGCTATAA |
| | | GAATGAATGTTCTGATGCTTTAAATTTTAATATCTAGAAATCAAGGGTAAGGACCAATTACTAGAATTTCCACCCTTAGAAGAA |
| | | TAATTAATAACAATGTAACAAATGTACAGCCCCAAACCTCTTTTAAAAAAGTTTAATTTAAAAAATTTAATATCAATAGTTGAAAA |
| | | ACAGCCAAACAGCAATGTCAAGCTGTAGTAGTTTGTGATTGTGGCAGCTTTTTGTGTTGGTCATGGATCATAATTCTAAACGT |
| | | TTTAATATACAGTAATCCATATGAATGCATTGCAGACCCTGTTATCACCTGTAGTTAATGGTGCCTGTGATACAAGTGAACAG |
| | | GCTGTTCTTTGCTCTTACCTTTATGGTAAATAAACCAGCATTCTGGAGTCACAATAATTAAATAAAATGTAAATCCCTAATCTCA |
| | | TTTGGCTATTTGGACCAAGAAACTTACCCGGCTATTACCTGTCTAGTTCTAATGATTACTAATGATTTATTGTTCTTTTTTTTTA |
| | | TTGTTCTAAAAGTGCTCTGATGTGGTGCCTTGATGTCTAATGAAAAGAAATTGGTCTTAAAAAGCTACTCATTGTTTATCCTGA |
| | | CTCTGAGATAACACATTTTTCTTCTGATAAAATATTTTTCTTATTTATTGTTCAAAACACACAATATAGACAATAACTCTAATTCT |
| | | GGTGCAGTTTTATTTAGTAGTGTTTTATCCCCGGTGCCTGGCTGTCTACAGTCATACCTCAGCACATAGCTTTACTCTGAGAT |
| | | AAGCCCTCTCTCCTTTGCTCTTTCTCCCATGCTTTCTGGAGTTTGTCTGCAGGATGCAGGTTTTGAACTGAAGAGCGCAGTG |
| | | GAGTTCTTACTGTGCCTTTCCGGTTCCAGCACAATCAAAGTTTTGTGTTTGGAATTGTCCTTGACATTCTTTCGTCCCACAATG |
| | | GGGTGGACACAGTGTGCTGGAAACCATGACATCATTATCATTGACACAGACTTAAATAAATCACCTGCATCTTGGCCTCTTTC |
| | | TGCTTATTCAAGGATTCTTCATCTTAAGGAAGAAAGTGCTCTTTATTTTTACAAATCTTCATCCCAGGGGTGACTGGAAGACG |
| | | CAAACTTTTACAAGTGTTTTAAAAATAAATATACTATGTGGTTTTAGACACAGTATGCTGAGTCAGAGGTGTCTTAGAGAATGT |
| | | TTAAGGTTAGACATAAGGCAGAAAGACAGCCAGATTGCCTTTAATTCGGAGGCAATATTCCTAAACTGTGTATCATTATGTCC |
| | | TTCTGAATATGGATTCACTTGAAACTTAATTAAATAGGCATATACATAACATTTCAAATACATACCTGAAAAGGCTTAGTAGTGT |
| | | TTCATTTTCACACATTGAGCTTGATATTCAAAAAAGAAAAA |
| >ENST00000480171.3:: | 219 | TTGAACCAGGAGATCTGCAGATGAAATTGAGAGGAGTAAGCAAGTTGGAAAGGGAAGCTTGCTAGATAGACAAATGCTGAAC |
| chr2:159518753- | | TTGGTTTTAGACACTCAAACACTCAGTTAAATGACAGTTGGCCTCCCAATCAGAGGTGTGATGCAGTCTTTTAGGGAACAAAA |
| 159537938(+) | | GACCATGTACAGAAGGCAAATGTCAGCAGGGAAAGACACCGCTGAACATGGCTCAAAAGGAGGGTGGAGTTGGGAGATTG |
| | | AGAAGGAGGCAGACATGGGACTTGGGGATATAGTTGTCCCCAGGGAGGGAGTTGGGGCATCTGTTGTCACGCCAAGGATC |
| | | TGCAGTAGTGAGAAGTGAGGCTCAAGCATCATGGGTGCAGTCTGCACGGCAGCAGCCCTGAAGAGAAACACTGGGCCGGA |
| | | GCCTCAAGCATTTCGGTCGGCATGAAGTCAAATCCTCAGCAGGACTGAGAAGGGCTCTGCATCCTGTCATCTGGGTATTCCA |
| | | CAAACACTCTAGAATCATAATTCTCTCTTCAGATTGTTTCAAGTTCTCATAAATAAAAAGTAGTTCTTTCATATTTCCCATTTATT |
| | | TCCTGTCTGTAGTGTTCAGTACAGAAGTGTTTTGCTCTAATGTGCCGAGTTGTTTTTAATTATACGCCATGCTGGGTTTCAGA |
| | | CGGTGGAGAACTGCGTGTGCACCCTGAGGAACCTGTCCTATCGGCTGGAGCTGGAGGTGCCCCAGGCCCGGTTACTGGGA |
| | | CTGAACGAATTGGATGACTTACTAGGAAAAGAGTCTCCCAGCAAAGACTCTGAGCCAAGTTGCTGGGGGAAGAAGAAGAAA |
| | | AAGAAAAAGAGGACTCCGCAAGAAGATCAATGGGATGGAGTTGGTCCTATCCCAGGACTGTCGAAGTCCCCCAAAGGGGTT |
| | | GAGATGCTGTGGCACCCATCGGTGGTAAAACCATATCTGACTCTTCTAGCAGAAAGTTCCAACCCAGCCACCTTGGAAGGCT |
| | | CTGCAGGGTCTCTCCAGAACCTCTCTGCTGGCAACTGGAAGTTTGCAGCATATATCCGGGCGGCCGTCCGAAAAGAAAAGG |
| | | GGCTCCCCATCCTTGTGGAGCTTCTGAGAATGGATAACGATAGAGTTGTTTCTTCCGTGGCAACAGCCTTGAGGAATATGGC |
| | | ACTAGATGTTCGCAACAAGGAGCTCATAGGCAAATACGCCATGCGAGACCTGGTCAACCGGCTCCCCGGCGGCAATGGCC |
| | | CCAGTGTCTTGTCTGATGAGACCATGGCAGCCATCTGCTGTGCTCTGCACGAGGTCACCAGCAAAAACATGGAGAACGCAA |
| | | AAGCCCTGGCCGACTCAGGAGGCATAGAGAAGCTGGTGAACATAACCAAAGGCAGGGGCGACAGATCATCTCTGAAAGTG |
| | | GTGAAGGCAGCAGCCCAGGTCTTGAATACATTATGGCAATATCGGGACCTCCGGAGCATTTATAAAAAGGATGGGTGGAAT |
| | | CAGAACCATTTTATTACACCTGTGTCGACATTGGAGCGAGACCGATTCAAATCACATCCTTCCTTGTCTACCACCAACCAACA |
| | | GATGTCACCCATCATTCAGTCAGTCGGCAGCACCTCTTCCTCACCAGCACTGTTAGGAATCAGAGACCCTCGCTCTGAATAC |
| | | GATAGGACCCAGCCACCTATGCAGTATTACAATAGCCAAGGGGATGCCACACATAAAGGCCTGTACCCTGGCTCCAGCAAA |
| | | CCTTCACCAATTTACATCAGTTCCTATTCCTCACCAGCAAGAGAACAAAATAGACGGCTACAGCATCAACAGCTGTATTATAG |
| | | TCAAGATGACTCCAACAGAAAGAACTTTGATGCATACAGATTGTATTTGCAGTCTCCTCATAGCTATGAAGATCCTTATTTTGA |
| | | TGACCGAGTTCACTTTCCAGCTTCTACTGATTACTCAACACAGTATGGACTGAAATCGACCACAAATTATGTAGACTTTTATTC |
| | | CACTAAACGACCTTCTTATAGAGCAGAACAGTACCCAGGGTCCCCAGACTCATGGGTGTAGCATCAAGATGCCCAACAGAG |
| | | GAACTCTTTCTTTCTAACCTTGTTCAGATTGAGGTGAAAAGTCCATCTTGCTGATTTGATGATTGAAATGTGAAAGTGAAGTG |
| | | GAAGGAATGAATGAAGTGTGTTTTTTTTTTCTTTTTTGAGGAATTATCAGGGAAGTGAGGAAATGTTTGGGAGAGGACTTTCT |
| | | AAGCTCTATTTAGGTGTTAGATCTAATTACTTATAGATTCTGTAGTCTGGTGAAGGTGTGGGTGACGTGATGAGAGGTTTGAG |
| | | AAATGGGTGAAATGAAATGGGGGATATGTAGGTCAAATCAAATTAAAGATGATTTTTTTAATGTGAATAAAGTTATGTTCTGAT |
| | | AGTTTGTACAGAAAAAATAAAATGGATGCCCATGTTTTATTGCTATTACTAAATGTCAAGATTGTATGCTATTATGTCTTGTAAA |
| | | TTTCTTTTGTTGGTGTAAATATGGAAATGCCACATTGGTTAAGTGCCATCATTTGTAATGCAATGTGTCACTTGAAAAGAGATT |
| | | TGAAGAAACTGACAACTTCAAAAACAAATGAGAAGCCCAAGGAACTGTGAGCAATTAAAAGCAAACCGCGACACCCTTTGTC |
| | | TCCACCACACATAGTGTACTTTGGAAGCACAACGTCCAGGCTGGTACCGCAGCGCCATGCCCATTCCTCGCCTCATTCATAG |
| | | GACACTTCACTGCCATTTTCTATTCACATAAAAGAAAAATAAATGTGGAAATTTCATCCTTGG |
| >ENST000004 | 220 | GAGTGGACCTTGTACGCCGCAAGCGTAGCAGGGTGTCAGACGCGCCGGTTTCTGCGACGCAGTTAGCGCAGTCTGCTTTG |
| 30412.1:: | | GTGAATACACGATTTGGTGCAGCCGGGGTTTGGTACCGAGCGGAGAGGAGATGCACACGGCACTCGAGTGTGAGGAAAAA |
| chr2:196521851- | | TAGAAATGAAGAACGTAATGGTCATGATCCTGGTCGTGGACACCAAGATCTTGATCCTGATAATGAAGGTGAACTTCGACAT |
| 196602426(+) | | ACTAGAAAGAGAGAAGCACCACATGTTAAAAATAATGCAATAATTTCTTTGAGAAAAGATCTAAATGAAGATGACCATCATCAT |
| | | GAATGTTTGAACGTCACTCAGTTATTAAAATACTATGGTCATGGTGCCAACTCTCCCATCTCAACTGATTTATTTACATACCTT |
| | | TGCCCTGCATTGTTATATCAAATCGACAGCAGACTTTGTATTGAGCATTTTGACAAACTTTTAGTTGAAGATATAAATAAGGAT |
| | | AAAAACCTGGTTCCTGAAGATGAGGCAAATATAGGGGCATCAGCCTGGATTTGTGGTATCATTTCTATCACTGTCATTAGCCT |
| | | GCTTTCCTTGCTAGGCGTGATCTTGGTTCCTATCATTAACCAAGGATGCTTCAAATTCCTTCTTACATTCCTTGTTGCATTAGC |
| | | TGTAGGAACAATGAGTGGAGACGCCCTTCTTCATCTACTGCCCCATTCTCAGGGTGGACATGATCACAGTCACCAACATGCA |
| | | CATGGGCATGGACATTCTCATGGACATGAATCTAACAAGTTTTTGGAAGAATATGATGCTGTATTGAAAGGACTTGTTGCTCT |
| | | AGGAGGCATTTACTTGCTATTTATCATTGAACACTGCATTAGAATGTTTAAGCACTACAAACAACAAAGAGGAAAACAGAAAT |
| | | GGTTTATGAAACAGAACACAGAAGAATCAACTATTGGAAGAAAGCTTTCAGATCACAAGTTAAACAATACACCAGATTCTGAC |
| | | TGGCTTCAACTCAAGCCTCTTGCCGGAACTGATGACTCGGTTGTTTCTGAAGATCGACTTAATGAAACTGAACTGACAGATTT |
| | | AGAAGGCCAACAAGAATCCCCTCCTAAAAATTACCTTTGTATAGAAGAGGAGAAAATCATAGACCATTCTCACAGTGATGGAT |
| | | TACATACCATTCATGAGCATGATCTCCATGCTGCTGCACATAACCACCACGGCGAGAACAAAACTGTGCTGAGGAAGCATAA |
| | | TCACCAGTGGCACCACAAGCATTCTCATCATTCCCATGGCCCCTGTCATTCTGGATCCGATCTGAAAGAAACAGGAATAGCT |
| | | AATATAGCCTGGATGGTGATCATGGGGGATGGCATCCACAACTTCAGTGATGGGCTCGCAATTGGTGCAGCTTTCAGTGCT |
| | | GGATTGACAGGAGGAATCAGTACTTCTATAGCCGTCTTCTGTCATGAACTGCCACATGAATTAGGAGATTTTGCAGTTCTTCT |
| | | TAAAGCAGGCATGACTGTAAAGCAAGCAATTGTATACAACCTCCTCTCTGCCATGATGGCTTACATAGGCATGCTCATAGGC |
| | | ACAGCTGTTGGTCAGTATGCCAATAACATCACACTTTGGATCTTTGCAGTCACTGCAGGCATGTTCCTCTATGTAGCCTTGGT |
| | | GGATATGCTTCCAGAAATGTTGCATGGTGATGGTGACAATGAAGAACATGGCTTTTGTCCTGTGGGGCAATTCATCCTTCAG |
| | | AATTTAGGATTGCTCTTTGGATTTGCCATTATGCTGGTGATTGCCCTCTATGAAGATAAAATTGTGTTTGACATCCAGTTTTGA |
| | | CCTTTCCCAGTAATCACTGTTGATTACGAGAATGTTACCATGCAGCTTTGCATCTGTTCCTTGTACTGTATGCACATTGCTCAA |
| | | AGGAAAGTCAGTGGCTTGCACTACTTACAAGTTTCATAGATTTGAGCCTAACCACAAGAGGCTGGTGCTTAGTACTGTTTTCC |
| | | CTGCACGTAGGGGTCTTTTAAAAATATAAAGCTTGTGATAAAGAGAGGAGAATATGGGACTCCATGAACCAGTGTTGATATGT |
| | | TTGATTAAGACTTTTCACAAAATAATCATATAAAACACTAGTCTCTTTATTAGTAGAAACTTCTGTGGCTATGCAGAAATAGAGA |
| | | TCGAACCAAAAAAAATCATTTAAACTTTAAAAATATTTTAAATGGACTTTGGGGAGACATTTTTTGTGTGTTTTAAGAATGAATT |
| | | GTAGTGCTCTTTAATTCAGCTACATATATTCATGTGGTGATAGGGATCAACTTGACACAACTTTGAAACTGCATAAAGTAGACA |
| | | TAGGAACTAGAGGAAAGCTCAGGCTGCATTAGAGTATGAATTTAGCATTGGGAAAAGCCCTTATTCTTGAATCTAGAGTTACT |
| | | ATTTTTGTATATATTTGCATAGTGTTTAAACCTGCAGCCTAAACTACTGAAATTTGTGATTGTATGTTTGTGTGAGCTTCAGTTT |
| | | AATGAAAGATTCATAATGGTTCTTTGTATTATTATAATACTTGGTGTTGGGGTGTTCTTTCTGTTTTGTTTTTTACTTTAATTTTG |
| | | TTTTGATTTTTTTTTTTTTTTTTTGGCGGGGGTAGGTGAGGGTTTGGAGCATGTGGTCTTTTTAAAAAATTGTAACCCTCTAGA |
| | | AAATATCAAAGAAATGAACCAGACGTGGTTTAAATAGTTGATTTTCCTATTTTAACAGTACCAACTAGTTAATTGGGAAATGTA |
| | | AGTTCTGAATGTTCACATTGCTTTACCAGTTTGGCACTGGAACCAAGAGCACATGTCGTGGCTGGCTACAAGGTTGTAAAGC |
| | | AGAAAATCGAAGTTTACCATGTCTGTAATGTGTACATGAAGTGTCAATTTAGAACAGTTACTAGGATAAACTCCATTATTGCCA |
| | | TGGCTGTCATGGTACCCAAGTGACTTGGAAGATGCATTTAAATTACTCAGCTGAAATCACTTGATCATCTTGTGCCAAGATAT |
| | | GCTGTTGGTGCCTGATAGGGATTAGTCTTTTAGGTGCCCTGTTCTCCTACCATAATTGTGAATGATTTGTGAGAAGTGCAAGC |
| | | CATGTTTATCCTGAATTTTTACTTAATAATTTGTATTACTAGTCATATGCATGTAGCTTTCTGTTTACATCCTATGCCACATGGT |
| | | CTTCATTTATGCCAGGTAAACTGTATTTGAACTATGTGCAGCTAGCTTTGTTTTAATCTGCTTGGCAACCAGTGTAGCTGCTGT |
| | | AACAATCTATCTTATTGTTCAAATATATAAGAGCCAAACTCTTTTCCATTCCATCTAAAATGTTTTCATTTAGTACTCTTCTTTCC |
| | | TCCTACTCTATGAACTTCAAAACAAAAACAAAACTTTGAGAGCAGCACATGCATCCAGGTATTTATAGATTATTGCCAGTGTCT |
| | | TTTCTGTATGCTATAAGCAAGGGAGCTTAGGTGTTATTTCTTTAATTTATGCTTGAATCTGAAAAATTATTTCTGACTTACTCCA |
| | | TGGCCTCCTTATAATAAGTAGAAGTTTTATATATAATTAATTTTCAGCATTGGGCACTGAATTAGGACAGTCCTCATCTCATTG |
| | | CTTGGCCCTTCAAGCAACCTAGCTAAAAGGTGCTGATATTTTATTTAGTACTGCCAACTTCAAGTGATTTAGATATCTATCTAT |
| | | CTAGATTTCTGAACCAAGATATATTTATAGTTCACTTTTGGGTTTTTATACCCACGGTAGGATTCTGCATTCCAGCATTAAATC |
| | | TGCTTCATTTTAGAACCTTTATAAAAGCAATAGCTGGAATATACTCCCAGTTTTAAAATAAATGCCTGATTGATTTAAAGCAAGT |
| | | AGGTTATGCTGAAGTATATAAAGAAGTTTTATATTCTCTCAAAAATGGTATTATCTTTCTTTATTTGCTAGATTCTTACAAATCTT |
| | | TTAAGAGGGCTGTAACAGTTGCTGCTAGTATTAGGGTTCCACATCATTCTAATGTATAGTTTCAAGTCTTAATAGACAATCTGA |
| | | ATTCCACTACATTTCTTTTGGCTCCAACATTCCTTTTAGCTTGACCAGTCTAATTTAAAATGTGTTTGTTGGAGGTCATTAACGT |
| | | TACTTGTACAATGCTGTCACTGTGTGACATCCATATGAATTTTGGTATATATCAATCAATCAATCAATCACATTGCATTCAATCA |
| | | ATCAGCTGTGATTGATTGATTATGCTTAGAAATACTATAGTAACTAGATGCAGTGTGAATTTTTTCCATTAACAAACAAACAAG |
| | | TCAGTGGCTTAAATGTGATTATGGTCCTGCAAGGTGATTCTTGCTAAAATATCTAAACTTTTGTTTTGTTTTAACTGAATCATTT |
| | | TTTAACTTAAAAAGCTGGAAAATATCAAATGCTGTTTTTTTTTTTTCATTGTCAACAGTGGTGTGTCATTTTATGTATGTTCCTA |
| | | ATGCTTATGGAACTCCTCCAAAATAAAGTTACTCAAAGAGAGCAAATA |
| >ENST00000467665.1:: | 221 | GGGAAATGTGAGGGCTAGTGCAGCGCTGAGCCCAGACCCCTCACACAGCGCTACCGGCAACTTGCTTAACCTAGAGGTGG |
| chr2:233740508- | | GGGGTGACGTCCCTGTCGGCCCCGTCACCTGGGGCCCCAGCACCTCTCCTTGGCTTTGCAGACGATGTTCCACAGGAGCC |
| 233743414(+) | | CGTGCCCACGCTGTGGAACGAGCCGGCCGAGCTGCCGTCGGGAGAAGGCCCCGTGGAGAGCACCAGCCCCGGCCGGGA |
| | | GCCCGTGGACACCGGTCCCCCAGCCCCCACCGTCGCGCCAGGACCCGAGGACAGCACCGCGCAGGAGCGGCTGGACCA |
| | | GGGCGGCGGGTCGCTGGGGCCCGGCGCTATCGCGGCCATCGTGATCGCCGCCCTGCTGGCCACCTGCGTGGTGCTGGC |
| | | GCTCGTGGTCGTCGCGCTGAGAAAGTTTTCTGCCTCCTGAAGCGAATAAAGGGGCCGCGCCCGGCCGCGGCGCGACTCG |
| | | GCTGCACTCCTCACGCGCCTGTATGTCCGCGCGTGCGTGTCCGCGCATGCAGGTGTGCCAGAGCGTGAGCGCGCGCAGG |
| | | CGAGCGCTCAGGGGGGGGGTGCGCGCGGGGCCGAGGGTGGGTGCCGTGCACGCGCGCGGGTCCGGAGGCGCGCCCGA |
| | | ATTCCCCGCAGGGGCGCCGGGGCGTGCGTGAGCGCACAGCATGTCCGAGCCCGCCTGCGTGTGGCGTGCACCTGAGCGC |
| | | GCGCAGGGCCGCGCGGCTCGGCGTCCCCGTGCACCACGGCTGGAGTGCCTCAGGAGCGCGCCGCATGTGCGTGCCCGG |
| | | TGCGCGTGCGCCGGGGCCGCCAGGGCATCGGAGCGGGTGTGCGGCCAGCGGTCTTAGCCCGTCCGGGGGCACCGTCAC |
| | | GGTCAGGGCCACGCCACGGGCGGCCCCTTCTCCCGGCGCCGCCTCCTCTGCCTGGGCGCGGGCCGAGGCCCTCGCCGC |
| | | GCTCCCCGGGGCCTGCCTCCCATCCCGCTCCGCTGGGGTTCAGAGCGTTCCGTGAGGAGGTAAGCGCTAGGGCAGGAGA |
| | | GGCCGACAGAGACCCCGGGGCCCGCCGCTCCTTGAGGCCGGGGTTGGGGAGGGCAGGGGCCGTCGGCTCTCCCAGCCA |
| | | GAGGCCGCGTGGTTTTGGGGGACGTCTTGACTCTGAGTCCTGCAGCCAGGGCCCCATCCGCGGGCTCAGACCAGCAGCC |
| | | CCAAGCCCCCCTCTGCCCAGGGAGCCTCAGGAGCAGGATGAGTCGAGAGTCGACGCCGGACACGGCCCGCCCCTGCTTC |
| | | CTACTCTCACCCCCAGGCCTGGCTGGGGGCCGGCAGCCCCCACGCCCACGGTTGGTTTGTTCAGGGAGAGAAAACGGCTT |
| | | TCCCAGCTCTACTCACCTCACTTCACCCTCAGAGAGTCGGGGCGGAGTGAGGGTGAGGAGCCTGGTCCAGGGTCCCCACC |
| | | TCGGAAGCATTTAGAGATGTAAACTTTGATCCTTTGATCATTTAACCTGCTCAGCACAGCAGGAAGTGGGAACTTGGTCCCTA |
| | | CCCCAGTCCCCAGGGCCTTTGGAGGGAAGGGTTGGACAGTTTGGAGGCCAGCAGACCCCTTGGGGACAGCTTAATGTATC |
| | | TGAAGATTCCTGAGTGGGAATGTTGCTGGAGGGCAAGCTGCTTCTCTTTTTTAAAAATTCATTTTCTCTAGCAGCTGTTTTGG |
| | | CCCCTTTGGTGTCCCTGAGCTCCCCATGGCACTGTTTCCTGCCGTGACTTTCTCCTCCTGGACACTGGAGCCCAGGTGGTG |
| | | CCCGCGCACCTCTCACACACTGGGGGATAGGGTCAAGGCCTCATCCAAGTCTGTTAACAGCTTCCCACTCAACCAGTCTCC |
| | | CCAAAATACACTGTGAGTTCAATGATTCTGCTTATAAAGGAGTGCTATGAAACTGACCTATTTTCCTAATTGGTTAACTGACTT |
| | | CATCTCACATAACCTTGAGTAACCAAAGCCTTCATCAGAGCGGACGATCCAACCCAACATGCTGTTTCCCATCCTCAAAAGG |
| | | CACGGGGACCAATGGCCACCCAACTCCTTCCCAACCCTCCCCTCCAGGGACGAGAGATGGCCCCCTCTTTCCCTTTCCCCA |
| | | CTCTGCAAGCACTCCCCCACCAACGGGTGCATCACAGCCCCTCACTGGGACCAGGGGGCCTCCCTCTCCGGTGGAACCAG |
| | | TGTCACATGCCTGTGCATCTCTGTGATTCTCAGAGGGTCTGAGGTCCGAGCACCCTGCTGTGGCCTGTGGGAAGACGCTAC |
| | | AAAGTCCCACTGGATCTAAACCAGAGGCCTGTTCTGGCGAGCAGGGAAACTGTGTCCTGGCAGAGATCGTGGTCCTGGGCA |
| | | CACAGGACCCCTCAGCACACTGAGGTGGAGCTGGGGCGAGGGGAGGGGGTGCGCTCTGGGTAACTGAAGGTGTGAAGGG |
| | | CCCAGGGCCTGTTTCTGGGCAGTGCAGGAAGTCCCAGCCCCATGCCTGTGGTGAGATCCCCTGTAGGGCCCCCCCCACCA |
| | | TGGACACTTCGGGGCCTCTACGGTCTTCCAAAGCTGTGTCCTCATTTCCACTGCAGCAGAGGGGCGTCCCCAGCTCCGTCA |
| | | AACAGCCCTTTCTGTTTCTGGAGTCCTACAAGTGGAGGCCCAAATCCGTTCCCATGTTGAGGCAAGGCCCTGGCTGTTCCTT |
| | | CCTCTCTGGAAACCGCCTTGAACTCTTCCTTTGGGACATGCCTCCTCGACCAGCCTTGAAGGGGTGCTCCTCTCTCACTACC |
| | | TGGAACCAAACACCCCCTTCCTTTGTGTACAAGGGCAATAAAGAGTAGACCTTCATCTTCTTTA |
| >ENST000004 | 222 | CTTCGGGCCCCACAGTCCCTGCACCCAGGTTTCCATTGCGCGGCTCTCCTCAGCTCCTTCCCGCCGCCCAGTCTGGATCCT |
| 78619.1 :: chr3: | | GGGGGAGGCGCTGAAGTCGGGGCCCGCCCTGTGGCCCCGCCCGGCCCGCGCTTGCTAGCGCCCAAAGCCAGCGAAGCA |
| 50373807- | | CGGGCCCAACCGGGCCATGTCGGGGGAGCCTGAGCTCATTGAGCTGCGGGAGCTGGCACCCGCTGGGCGCGCTGGGAA |
| 50378411(−) | | GGGCCGCACCCGGCTGGAGCGTGCCAACGCGCTGCGCATCGCGCGGGGCACCGCGTGCAACCCCACACGGCAGCTGGT |
| | | CCCTGGCCGTGGCCACCGCTTCCAGCCCGCGGGGCCCGCCACGCACACGTGGTGCGACCTCTGTGGCGACTTCATCTGG |
| | | GGCGTCGTGCGCAAAGGCCTGCAGTGCGCGCATTGCAAGTTCACCTGCCACTACCGCTGCCGCGCGCTCGTCTGCCTGGA |
| | | CTGTTGCGGGCCCCGGGACCTGGGCTGGGAACCCGCGGTGGAGCGGGACACGAACGTGGTGAGCGCGGGGCCGAGGGC |
| | | GTATGGGAAGGGCGAGGATGGGCAGGCCACAGTGCAGGCATTCTCGAGGGCTGCCTGGGTGCCGCGCGCAAGGAGCGTT |
| | | CTAATTGCCGATTTCCCGGCGGCACAGAGAGGCTAATTCTGCGCGGGGGCTGGGAGGGGAGCCTGGATTGCCGGCTCCG |
| | | CAAGTACTCCACCCGCTGCAAGCGGACCCGGGCCCAGGCTGACCCAGGCTCCGCGCACGCGCACTTCCCGCACCTTCCC |
| | | GCCCTCGCCTCCGGCCAGAGGCCACTCTTGTGCGCTTGCCCGGACGCTGGCACCCGCCCCCGTTCCCTGTGGTAGGTGG |
| | | GGTCTGTGAGTGGAGCTCCGGAGCGATGAGGTCATTCCTGGGGGCGAAGCGTGCGTGTCCCCGCCCCGGCGTTCCTGCC |
| | | CCAATGAGACAAGAGCTAGATCCCGGCGATCTACGTTTCAGTCTTAACGGTTGCGGCGCGGCTCTGGCCCGGGCGCACGC |
| | | GCACACTGACACGCGTACACGCACGCACGCGACCGGGGCGGTGGTTGGCGGCTACGGACGCGCAGGACTGGGGGACGG |
| | | GCGGGTACGGCTATGGGCGAGGCGGAGGCGCCTTCTTTCGAAATGACCTGGAGCAGCACGACGAGCAGTGGCTACTGCA |
| | | GCCAAGAGGACTCGGACTCGGAGCTCGAGCAGTACTTCACCGCGCGAACCTCGCTAGCTCGCAGGCCGCGCCGGGACCA |
| | | GGTGGGAGCCAGGGGGTGCCGGCGGGCGGGAGGGGAAGCGGTCGCTGGAGCTCCGCCCTCCCCGGTCCGTTGCCGCGT |
| | | CCTGGGTCGGTGGGCAGCCCCACCCTCCTGGCTACGTGGCTCCCCGCGGGTCCTGGCCGGGGACCTGCCCGCGGAACCG |
| | | TGCGTAAGACCCCGATTCCACCGCCTAGATGCTGGGTGCCGGGGCCCCCTTGGTTTCTGTCACAGACAGGTTGAACACGGA |
| | | AAAAGCAGCTGTATGGCTTGTGGTAGACCTGAGCCGGGCATTATCCAGCTATGACTAAAGCCGACCGAGCAGTTTGGACTA |
| | | GCACCTCGATTTCCGCGTTCGAATGCTCCTGCTCCCTCCTTGGGGAGACTAGGGGAGGATGTGGAGAGGGAAGAGTCCTC |
| | | GCCAGGAATTGAGAAGTATGTTTAGGAAAACTTGAGAGGCAGAGAGAGATCCTGCTCCTCCATCTGCACTCCTGTATGGAGC |
| | | CAGCTGAGCCCTCACCTCTTCCCTGTTCTGGCCTGTCACCAGCTGCTGGAATGTGGAAGATTCTGTTCCCTTCCTCTAGGGT |
| | | GGATCTGGAGAAAGATTTGGGAATAGATAGGAAAGAAGTCTTGTTTTGGACCATAAGCATTCAGGAGCACTTTACCCACAGG |
| | | AAGGGGGAAAGCTAGATTATAAAATGCCTAAAGAGGTGGAAAAAGAGATCCAGGTTACTAACCCAGGACTGTAAGGTGTCTC |
| | | GGAACCTCCTAGGTATCCCCATTATCGGAGAACTGTGTGCCAGATGCCATTGGTGTGACCACCAGGCTCA |
| >ENST000004 | 223 | GGAATTGGGGGTTACTATCTTGGAATCTAGGGGCACTCCAGGCTCTGGGCTCAGACGGCTGGCTTCTGCCTACCCGAGCCT |
| 60833.1:: | | TAACCTTTCAAGGACCAGAAGGATTCCAGAGCTCTTGCCCTAGGTCCTGGGGCAGCGATGACTCACTGCAGCACCCCCTCC |
| chr3:64670584- | | CACTTCGCCAAGCTGCCGTCTCCGCCCACCCCCAAACAATCTCGACAGCGCATTTCGGGAGCCACGGCTCCGGGCGCTTT |
| 64788401(+) | | GCTGGGGGCTAAAGGGGTTTATCCCTTTCCTTGAATCCCAGCAGGCTAGAACTACCCCCTCCCAGTCTTCAGGCTTGCCAC |
| | | GCTCTCCACCCGATCCTTCCATTGAAAGGCAGAGAAGGAAGGATGTGCTTGGGAACTTTAAGACCCACGAACGACAGCGCA |
| | | CTGATGGAGCAGCCCAGTGTCTGGGGCAAAGTCCTCGAGGTTCATTCATTCAGGAAGCCTCTGACCAGCCTTTACCATGCG |
| | | CTGAGTGAGAAGCTGAGGATAAGGAAAGAGCAAGACCCCCAAGAAACCCTGATGTCTGGCTGAAAGCCGAAGCATGACGCA |
| | | ACTTTGCTATATTTCTCTCCAACAAGGATTTGTATATTTTCGCTTTCTCCTCAAGTAACACCTGGACCTGCTCCTTTCCCTTCA |
| | | AACGCTGAGGGCTCAGTCTCCAAGTTCCTTTATGAAACAGGGTGTACCATCAGAGACGCAGGTAAGAAACCTGAACTGCGT |
| | | GGCCTCACATAAACCCCACCACACCTCTGTCCTGGGCTCCTTCCCCGCAACAGGTGTAAAGACAAAAGGCTCATTGTCCTTT |
| | | GGAGCCTATAGGATAAACAGAAAGAACATGTCCTTGCCGGTTAATCTGCCGGTGATAAAGAGTATTGTTTTATCTCCTGCCAT |
| | | CTCCACGTTCCAAGAGATGAATTCTATTGTGACACAAAGTCTGCTTTGTCCTTCCAACCCACTGAGATGTTTAAATTGGCCTC |
| | | TCATATATTCCTTGGTTGTCACAATAAGAGCAATGAGTTGTTATAATGTGTTGAGAGGATCTTTCCCCCATGACAAGGAGACC |
| | | ATCATCTACAGAGATGCTATGATGCTGAGAAATAATGTGCTTCTCTTCTGCTCTAATTTTTTTTAATTAAGACTTTTATTTTTTG |
| | | AGCAGTTATAGGTTTAGAGAAAAATTGAAGGGAAAGTACAGAGAGTTCCCATCTTCTCTCACCCCTTTCATCTCTGTTTCCAT |
| | | ACGTTTGCTACAAATGATGAGCCAACAGCCATACGTTATTAACTAAAGTTCACAGTTTACATTAGGGTTCACTCTTTGCTTGTA |
| | | CATTCTATGGGTTTTGTCAAATATATGATAACCTGTATCCTCCATTATAGTACAATACAGAAGAATCTCACTGCCCTAAAAATC |
| | | TCCTGTGTTCCTCCTGTGCATCCCTCCCTCCCATCCCCTGGGGAACCACTGATCTTTTTGCTGGCTCCAGTTTTACCTTTTTC |
| | | AGAATGTGAGGTAGTTGGGAGTCATACAGTATATGTGTATATATATATGTGTATATATATGTGTATATACATATATATATACACA |
| | | TATATATACATATATATATACACATATATATACATATATATATATATAAAAAATAGCCTTTTTAGATGGATGTCTTTCCCATAGCA |
| | | ATATGCATTGAACTTTCCTCCATGACTTTGTGTGGCTCAATAGTTCATTTATTTTTGTTGCTGAATAGCTTCTGCTTAATTTTCA |
| | | AAGTGTTTAATGTAAGAAAGCGGTATAGTCCAGGCGCAGTGGCTCAACACCTGTAGTCCCAGCACTCTGGGAGGCGGAGGC |
| | | GGTGGATCACTTGAGCCCAGGAGTTTGAGACCAGCCTGGGTAACATGATAAAACCTTGTCTCCATAAAAAATACAAAAATTA |
| | | GCTGGAAATGGTAGTGTGCGCTTGTAGTCCCAGCTACTCAGGAGGCTGTGGTGGGAGGATTGCTTAAGCCCAGGAGCTAGA |
| | | GCCTATAGTGAGCCATGATTGTGCCACCGCACTCCAGCATGGGTGACAGAGTGAGATCCTGTCTCAGAAAAAAAAAAAAAAC |
| | | AAAAAAAAAAAAACCCAGAAACAACAACAACAAAACGGCAGTATATTTGTCCCCACCCTCATCTCTTCCTTTATGGGTATTTCC |
| | | CTAATTGTGAGAGATTACCTGTGTTTTAAAAGGTGGCTTACTGTATGCATTGAGAAAATTAAATGACATTTCAGACCAGTTCCA |
| | | AGTACCTTTTGCAAAGCTAGAGAAATAAAAGTGAATTCTAGTATCTCAATCACAGAGTAAAACTAGAGAAGGTCTGTTGGGCT |
| | | TCAGGCAACATTGGTGTTCTTGGCAATTTTACCCAGAAGAATTTATATTCCTATAATTAGTATTTCCTCCAAAATTAATGATCG |
| | | CCTGATTGATTTATTTCTTTCCTAGCAGCACTGGAGGTCCCACTTACCTAGTTAGTAATAACATTTGCTCTTAATACATGAGTC |
| | | AGTTTTCTTTTTTCCTTTTTTCTTTTTCTTTTTTTTTTTACATTTCCAAAACAGAATACCCTGCAGGAAGAATGGGATGGCAGTG |
| | | GAGGAGCTCTCACTCACATATCATGTAATGTGGGAGGCAGGGCTATGCGATAGTCATAAAGGGTGCAGGCTTCAGAATCAA |
| | | AGGAGTGAGGTTGGTGTCCCTGCTTACGCCCTACTAGCTGAGTGTCCTTCAGGTCATTTCTCCTCTCTCAGACTCAGTGGAC |
| | | TGTTTCCTAAAGCACAGCTCATAACAGGCTGTACGTGGTAAGACCGTCGGGAAGATGGAAGGAGATCAGTCATCCCTAGAG |
| | | AGCATTAAGCACAGTCTCAGCCACAAAACACTTCCTCAAACATGATGCAAGCCACACAGGGCATTTGGCCTGCGTATAAAGG |
| | | ACATACTGAGGACAGTGTAGATGCTCAAGAAATTAGTCCTGAATGATCAAGGGGGGAAAGAGAGGGTTAATCTTATCATATG |
| | | TGGGCTCTCAGAGAACACAGAGCTATAGAAGGAAGCTGACTTAATAAGGGTCAAGCATGATAGGGGAGATTAGGAACCGTG |
| | | TGCAATCTAATGGAACCACAGATAACACCTTCTAGTACTTTTTCCCTTTGGCCCTGCCCCTGCTGAGACAGGGCCACTGCTA |
| | | ATAACTCACATTCTTCTCTTCTCTCCTCTCAAAAAGCCCATTTCCTGAGCAAGTATATGCTTTGGTAAGGTCATTAGCGGTCAA |
| | | CATGGTTCCCAAGTTTAGTGAATACTTTCAGATAAAACTGAAGGGTACAAAATCACTCTTTCCTCTCTCCCTCTCCTTGTCCTA |
| | | CCTCTCACAATAAAACGACTTATCTACTAATTACAAAGACCTCAATAGTGGAAGAAATCCATATCCTACATAACATGGAGAGCT |
| | | AGCTATATACCCTTGGCATCAGTTTTTATCCTACTTTTACTTTTTACTGACTTTCATTTACTGAACAAAGCCTCCTTTGCCAAAG |
| | | GCAGGGTAAATAAACTCAGTCACATCCTGTCTTCCTTACATCTTTGTTTATCCATAAACTGTCGAAGATACAGATGAAAATCCA |
| | | CTAAAGCATGCTTGAAGCGGTTACACGAAGGCCAAATAAGATGGGAATGGAAATGCCTGATGTTAAGGGAAGATACTACCTA |
| | | AAATGATACTGAGCTGGCCAGTGTAATGACATGAAAACAAAACAAAAAAA |
| >ENST00000511316.1:: | 224 | CGTGGCCTAGCTCGTCAAGTTGCCGTGGCGCGGAGAACTCTGCAAAACAAGAGGCTGAGGATTGCGTTAGAGATAAACCAG |
| chr4:174252855- | | TTCACGCCGGAGCCCCGTGAGGGAAGCGTCTCCGTTGGGTCCGGCCGCTCTGCGGGACTCTGAGGAAAAGCTCGCACCA |
| 174255583(−) | | GGTGGACGCGGATCTGTCAACATGGGTAAAGGAGACCCCAACAAGCCGCGGGGCAAAATGTCCTCGTACGCCTTCTTCGT |
| | | GCAGACCTGCCGGGAAGAGCACAAGAAGAAACACCCGGACTCTTCCGTCAATTTCGCGGAATTCTCCAAGAAGTGTTCGGA |
| | | GAGATGGAAGACCATGTCTGCAAAGGAGAAGTCGAAGTTTGAAGATATGGCAAAAAGTGACAAAGCTCGCTATGACAGGGA |
| | | GATGAAAAATTACGTTCCTCCCAAAGGTGATAAGAAGGGGAAGAAAAAGGACCCCAATGCTCCTAAAAGGCCACCGTAAGTT |
| | | TAAAATAACCCAAATTGCTCCTTGGATTTTTCCTTCAGTTTATTAAACTCTGTTGCTTCCTTTCAGATCTGCCTTCTTCCTGTTT |
| | | TGCTCTGAACATCGCCCAAAGATCAAAAGTGAACACCCTGGCCTATCCATTGGGGATACTGCAAAGAAATTGGGTGAAATGT |
| | | GGTCTGAGCAGTCAGCCAAAGATAAACAACCATATGAACAGAAAGCAGCTAAGCTAAAGGAGAAATATGAAAAGGTACAGTG |
| | | TCATCTTTTTTAAAGCCGTGGATAAGACTAGGTATAGGTAATAACTGTAGAAAACCTGGGAAATTTAGTTAATCTTGTATTAAT |
| | | GGTTGTCAGCTATGTTTTGAAAAGGCCTAATGAAAATTGTACACTTCAACACAAGGTAATTGAAACCTTCCTTTTGACTGAAAC |
| | | CAGTGTTTGTAGCACTAGTATATTCCTGCAGACAGACTTGTAGTTACTTGTAGTTATTGTATAGTCTGTATAGTCTGTTATTTTT |
| | | TTTTTTAAGCAGAGAGTCAAAGAAATTGTTTTATGTAGATATATATAAAATGTGAAGGTACAGGAGGGACTATGGCACTGTGT |
| | | GTGATGTAAAAGGGTATTGGTAGTGAAAGTACTGATACTGCTGTATCGCAACCCTTGTCATTTTACGTCATTAACTTGTTAAA |
| | | GCCTAGTGGGATAAGTGCTCTAAAAACTTAGACTGGTTACCTTTTTAGACAGTTATTAGGGTTATTGGTCAATCATCTTAGATT |
| | | GTTTACAACTAAGTGGTTTTTCACAGTTGAGTAATGATACCGGATGCTTTATTTTTTTGACAATATTTCAGGATATTGCTGCATA |
| | | TCGTGCCAAGGGCAAAAGTGAAGCAGGAAAGAAGGGCCCTGGCAGGCCAACAGGCTCAAAGAAGAAGAACGAACCAGAAG |
| | | ATGAGGAGGAGGAGGAGGAAGAAGAAGATGAAGATGAGGAGGAAGAGGATGAAGATGAAGAATAAATGGCTATCCTTTAAT |
| | | GATGCGTGTGGAATGTGTGTGTGTGCTCAGGCAATTATTTTGCTAAGAATGTGAATTCAAGTGCAGCTCAATACTAGCTTCAG |
| | | TATAAAAACTGTACAGATTTTTGTATAGCTGATAAGATTCTCTGTAGAGAAAATACTTTTAAAAAATGCAGGTTGTAGCTTTTTG |
| | | ATGGGCTACTCATACAGTTAGATTTTACAGCTTCTGATGTTGAATGTTCCTAAATATTTAATGGTTTTTTTAATTTCTTGTGTAT |
| | | GGTAGCACAGCAAACTTGTAGGAATTAGTATCAATAGTAAATTTTGGGTTTTTTAGGATGTTGCATTTCGTTTTTTTAAAAAAAA |
| | | TTTTGTAATAAAATTATGTAT |
| >ENST000005 | 225 | AGCTTTCTTCCCGCCAGGCCCCCTCCACCCGATCGCCGCGCGCTCTCCGAACCAAAAGGCGACCTCACGAAATGCCCCTTT |
| 13560.2:: | | GAGCTCAAAGGCTAGTTACCCCCAGGGGCCCTTCCACTCTCGGGGACAGGCGAAACCTCTTTGTCTCTGCCTCGGCCTGCG |
| chr5:43571695- | | GCCCCCAGCCCAGCCTCCGCGCTTTCCCTCCGCCAGTCCTTGTCAATCAAACCTGGTGCCAAACGCGGCAGCTGAAGTTTT |
| 43603206(−) | | CAGGGACACATTTGCTTCTCCCCTTGAAGAACCAGTTACAAAGCGTGATGTCCTCTCTGGGGTCCCATCAGAACAAAGAAAC |
| | | AGGTCTAAAGACCCTCATTCCAGAGAGCATCCTGCCCCATATCCAGAATGAAATCCATGCTCAGAGATGCCAAGAAGAATCT |
| | | AGACAGACAGGCCTTTCTAGGCTGTACAAGAAGCATGATGCCAGCATCTGCTTCTGGTGACAACCTCAAGAAGTTTACAATT |
| | | GGCCGCGTGCGGTGGCTCATGCCTGTAATGCCAGCACTTCGGGAGGCGGAGACAGATGGATCATTTGAGGTCAGGAGTTC |
| | | GAGACCAGGCTGGCCAACATGGTGAAACCCCATCTCTACTAAAAATACAAAAATTAGCTGGGCATGGTGGCATGTGTCTGGA |
| | | ATCCCTGCTACTCAGGAGGCTGAGGCAAGAGGATCACTTGGAACTTGGGAGGCAGAGGTTGCTGTGAGCTGAGATCACACC |
| | | ACTGTACTCCAGCCTGGGCAACAGAGTGATACTCTATCTCAAAAAAAAAAGAAAGAAAGAAAGAAAAGAAAAAGAAGCTTACA |
| | | ATCATGGTGGAAGGCGAAGAGGAGGAGCAGGCATATCACATGGCCAAAAAGGGAGCAAGAGAAAGGTGAGAAAGACACTA |
| | | GACTCTTTTTAAAACCAGCTCTCACATGAGCTAATGGAATAAGAACTCACTCATTACCACAAGGACAGAACCCAGCCATTCAT |
| | | GAGGGACCTGCCACCATGACCCAAAAACCTCCTACTTGACCCAACCTCCAACACTGGGGATAACATTTCAACATGAGATTTG |
| | | AAGAGGACAAATACTCAAACTATATCACTTGTTGTCCATTTTTAATCTTTCTCTAACATACCCTAACTTCTTCTTGAAAATGCTT |
| | | AATTTTCTCTGTTTTGAGATGTAAATTTGCTATCCTGTTTTTCTTAAAACTTGGTAAGATCTTCAGCCATGAAGGACAGACAAA |
| | | CTGTAACCTTTCCATTTACAAAGCTGCATTTGAATTCAACTGTCCTTTTAAACTAAGGAGTTTTACTGGTCTCGTGGCTAAAAT |
| | | TTTAAAGATGTAGGCCAGGTGCAGTGGCTCATGCCTGTAATCCTATCACTTTTGGAGACCAAGGCGGGGATGGATCACCTGA |
| | | GGTCAGGAGTTCAAGACCAGCCTGGCCAACATGATGAAACTCCATCTCTACTAAAAATACAAAAATTAGTCAGGCATGGTGG |
| | | CACACACCTGTAATCCCAGCTACTCGGGAGGCTGAGGCAAGAGAATGGCTTGAACCCGGGCGACGGAAGTTGCAGTGAGG |
| | | CGAGATTGCACCATTGCACTCCAGCCTGGGCAATAAGAGTGAAACTCTGTCTCAAAAAAAGAAAAAATGATATAAAGTATTTA |
| | | TTTGTGTTTATATTTTATGGATACGTGTCTATGTTTATAGATTGCCTGTATGGTACCAACTTGACATAAATAAATTAGTACTCAT |
| | | AAATTAAGTAAATAAGCCCAAACGCTTTTCAAGTTCACAGGGCATTAGTAATCTGTGGTAAATGAAGATAGTTTAAAATTTGGG |
| | | GTAAAATAAAATAGAAACATTTTCTGAATTTAATTTAGATATTTTTCTGGGTCTATTTGTTAGACAGGTTTACACTATCTCTGCT |
| | | ATTTTTTTAAGGACCTAAAACTGAATGGACATAGAAAATTGAGGAAAGAAATAAAACCGTAACAATTATAAGAGGTTATAAAAG |
| | | GTTCATGGAACCTTTTTATGTTTTTTCTTGTGCAGTCAAAACCAATTGCAGGCTGGATGCAGTAGCTCACACTTGTAATCCCA |
| | | GCACTTTGGGAGGCTGAGATGGGCAGATCACTTGAGCTCAGGAGTTTGAGACCATCCTGGGCAACATAGTGAAACTCTCTC |
| | | ACTACCAAAGTACAAAAGGAATTAGCTGGGCATGATGGTACACACTTGTAGTCCCAGCTACTTGGGGGACTGAGATGGGAAA |
| | | ATTGCTTGAGCCCGGGAGTCAAAGGTTGCAGTAAGCCAAGATTGCACCACTACACTCCAGCCTGGGTGACAAAGTGAGACC |
| | | CTGTCTCAAAAAAAAACAAAAAAAGGAAAAGAAAACAATTGCAATTGGATTGATCTGTTTATAAGGCTTTATTAAAATTAGCTTT |
| | | AGCATTTATAATACACTGGCACAAAGTTAGAATTTGGATTTCTCTTTTGAACAAGATTTTTGTATACATTAATAAGAAATAGTAA |
| | | AAGATTTTTGTTTACCTTTTGAGTAAACTGCAAAAAATATTAGTAATAAGAGGAGAGAATTTCCCTAATGCTGTTTTTATTATGT |
| | | CTTTTGATTGTTTGGAAAACTGAATCTCCCCTCTGTCAAAGAGTAAAATTTTTGCTTTTTGAAATTTTTGAATTATCAGTTTGGC |
| | | TAAATGAATAACTATTATCTTACAGTGACCTGTAATCTTATTTTGATCAAGTGTTTTAAACCTTTGATATTTGACAAACTTCCCA |
| | | AAATCAAATGTAAAATTCTAACTTAAATGTTTTCGACCTCAAGCTAACTTTTGGGCATTAAAGCCCTTGGAAGTCCAAATAGGG |
| | | CCCCTGGAAGTCCAAAAGAAACATATTAGGCTAATTTGGTATATTAAAATCATACAGGAAGCTGATCAAATAAAAAATGTTGTT |
| | | TAATTTTCTTTGAGTTGTATTTGTATAAATCTGTTATTAATATGCATCCCAAAATTGTATCATATTGCTAAAATGTTGATAGATCT |
| | | TGTTATATGTTATTGGTAATAATTGTTATCCTGTTAAATTATTGTATACCACAGAAATAATGAAATTTCTTTGTCAATTGCATCAG |
| | | TAATCATGGCTCTTCTAAGTCTTTTGTCATTCACAGATGATTATTGTTTTACTTGGATTCTTTTCAAAAGTGGTTTGTAATTGGC |
| | | TACAGCCTAAAATCTGCTTGTTCAAAAAAAAAAAAAAAAAATCCATGGAAAGGGCCCTGACAGCACACACACTTAAACACAGT |
| | | TTTCTGATAACTTTGGAATTCACACCGTTGGACTAGTTAAAAACTTCTAAAATAATTTTTTAAAATCTAATAAATTAATGAAGATT |
| | | GCTAATCCAACATCAAGCAGAATAAGTTAATTACATGGGGCTGAACTGATAAAATGCCAAAATAATATTTTCATAACCTTTTTT |
| | | TGGTTTGAGACATTGCTGATACTTTTTATGTTTTGTTTTCCAGAGTCAAGAAAATTGTTTTTTTCTTTTGAGCTGTACATAGCTT |
| | | ACAGTAATTAGATAAATTACACTTTTGTGAGAACAATTGAAACACTAACCTTTCTCTCTACCTGATGTCTCCAGAATTTGGAAA |
| | | GTATTTGTGAGTATTCTTCACTTTTGGCAATATAGTTATTTGTACAAATTCGATAGGAATCTGTTTTCTTTTGTAACAGAACACA |
| | | GTTGGATACACTGATTATTTTGCCAAGGCTTTCATTGGAATGGCATAATTTTTTAATGACCAGACTGCTTTGAGGATTTGAAGT |
| | | TGACTTTATAGAGCCTATAAAAAGCCTGTTGGAAAAATTAGCCTGATACCTTGTCTACACAGTTTCCTTACAAGGTTCCTGAC |
| | | CTTGCGGTAGTAAAGAATGTCACTCTCTGGCAGGCCCAGGAGCCTCAGGATATTTTGGGAACCTTGACAAGAGAGGAGTGT |
| | | ATCCAATTTATACAGGAATTACAAGTGCAGTCTGATTGTGAATCCTTGTCTTGGCTTCTTAGCCTTGAGAGTTTTTAAAAGTTG |
| | | AATGTGAAATTCCTTATGAAAAAGTTCCAACAAAGCCAAACTTTAAAAGAGCCTATATGTGGTCAATCACTATTTTTGCTGTAC |
| | | TTTATGCAAATAATCAGGCCAAATATAATAAAACTAAAACTTATTTTGCAAATAAATTGGTCCTGTTATGATTTGCCTTTAATAG |
| | | AAAAGGGGGACTGGAGAGAGAAGAATTATGTTTCAGAAGAAAATGATAGCATACCTGTTGTTAGATTCTAGCTTTGTCCATTG |
| | | TTTTTAAGTTGTAATTATTTGCCTACATTTGAACTAAATCTTGAATTCTTTCCTGGCTACAAGTCTCCAAGCTAACATTTAAATTT |
| | | TTTTCTCCTATGTTTCTGACTTGGAATAAGTAGAAGTTAAAACTATGCTTTTCTTGAAGCCCTGCAGACTGGAGCAAGACAACT |
| | | TGAATAAACTATGGGAAAAATCACTACAGCAACTTATATATAAACAGCTTTTATGCTTTGTTGATGTATGGAATACTCAGAAAG |
| | | TTCACTGCAACACCTGATTTAAACTACAACCAGGAGACTCTGTCAGATTAACACTACAATCTGAAGAACTACAGAGACTCTCA |
| | | AAAAACTAGTGTATAGTCTACAGTAGATATTAACCTTTGTTTTTCTTCTGTTTTCATAGAAACACCTTTTATTAAAAATCTGTTTG |
| | | CCGCTTCATATATAGAGTCCTAGTCCATCTGTAATGCCACCCCCTGGAATGAGACATAGCTGTTTAACTGAACTGATCTATTC |
| | | TCGGGACTAAGAGACTG |
| >ENST000005 | 226 | ACTGCTCCCAAAAATGGCGGACGCATTCGGAGATGAGCTGTTCAGCGTGTTCGAGGGCGACTCGACCACTGCGGCGGGAA |
| 06750.1:: | | CCAAAAAAGACAAGGAAAAGGACAAGGGGAAATGGAAGGGGCCTCCAGGGTCTGCAGACAAGGCAGGGTTGCACTTCCTG |
| chr5:54603828- | | CAGAAGAGGATTATTTACCACTTAAACCACGAGTTGGAAAAGCTGCTAAGGAATACCCGTTCATTCTTGATGCTTTTCAAAGA |
| 54720934(+) | | GAGGCCATTCAGTGTGTTGACAATAATCAGTCTGTTCTAGTATCTGCACATACCTCAGCGGGAAAAACAGTATGCGCCGAGT |
| | | ATGCCATTGCATTGGCCTTAAGGGAAAAGCAGCGTGTAATATTTACCAGCCCAATTAAGGCTCTGAGTAACCAAAAATACCGT |
| | | GAAATGTATGAAGAATTTCAAGATGTTGGTTTGATGACTGGTGATGTTACTATTAATCCTACGGCATCTTGTCTTGTTATGACC |
| | | ACAGAGATTTTGAGAAGTATGCTTTACAGAGGTTCCGAAGTTATGAGAGAAGTTGCTTGGGTTATATTTGATGAAATTCATTAT |
| | | ATGAGAGATTCAGAACGTGGTGTAGTATGGGAAGAAACTATTATTTTGCTTCCTGATAACGTCCACTATGTCTTTCTTTCGGC |
| | | TACTATTCCAAATGCCCGACAGTTTGCTGAATGGATTTGCCATTTACATAAACAGCCTTGTCATGTTATTTACACAGATTATCG |
| | | GCCCACTCCATTGCAACACTACATTTTTCCAGCAGGGGGAGATGGCCTGCATCTTGTGGTTGATGAAAATGGTGACTTCAGA |
| | | GAAGATAATTTTAATACTGCAATGCAAGTGCTTCGAGATGCAGGTGATTTGGCCAAAGGAGACCAGAAAGGGCGGAAAGGA |
| | | GGAACAAAAGGACCATCAAATGTTTTCAAAATTGTGAAGATGATTATGGAAAGAAATTTCCAACCTGTGATTATTTTCAGTTTT |
| | | AGTAAGAAAGATTGTGAAGCCTATGCACTTCAAATGACCAAATTAGATTTCAACACAGATGAAGAAAAGAAGATGGTTGAAGA |
| | | AGTATTCAGTAATGCAATTGATTGCTTATCCGATGAAGATAAAAAACTCCCTCAGGTAGAACATGTACTTCCTCTTTTGAAGAG |
| | | GGGAATTGGTATTCACCATGGTGGTTTACTTCCTATTTTGAAAGAAACTATAGAAATTCTCTTTTCTGAAGGATTGATAAAGGC |
| | | CTTATTTGCCACGGAGACCTTTGCTATGGGAATTAACATGCCAGCTAGAACTGTTTTATTTACAAATGCCCGCAAATTTGATG |
| | | GGAAGGATTTCCGATGGATTTCTTCTGGTGAATACATTCAGATGTCTGGTCGTGCTGGAAGGAGAGGAATGGATGATAGAGG |
| | | AATTGTAATTCTTATGGTAGATGAAAAGATGAGCCCAACAATTGGAAAACAATTACTTAAGGGCTCCGCTGATCCTCTAAATA |
| | | GTGCTTTCCATTTGACCTACAACATGGTTTTGAACTTACTACGTGTAGAAGAAATTAATCCTGAGTACATGTTGGAAAAATCCT |
| | | TCTACCAGTTTCAGCATTATAGAGCAATTCCAGGAGTAGTAGAGAAGGTAAAGAATTCAGAAGAACAGTATAATAAAATAGTA |
| | | ATTCCCAATGAAGAAAGTGTGGTTATCTATTATAAGATTAGACAGCAGCTTGCCAAATTGGGTAAAGAAATTGAAGAATATATT |
| | | CACAAACCAAAATACTGCTTACCTTTTCTACAACCAGGTCGTTTGGTAAAGGTAAAGAATGAAGGAGATGACTTTGGCTGGG |
| | | GAGTAGTGGTGAATTTCTCAAAAAAGTCAAATGTTAAGCCTAACTCTGGTGAACTGGATCCTTTGTATGTAGTAGAAGTACTT |
| | | CTGCGCTGTAGCAAAGAGAGCTTGAAAAATTCAGCTACAGAAGCTGCAAAACCAGCTAAACCTGATGAGAAAGGAGAGATG |
| | | CAGGTTGTCCCAGTTTTGGTGCATCTCCTGTCTGCTATCAGCAGTGTTAGGCTTTACATTCCTAAAGACCTTCGGCCGGTGG |
| | | ACAATAGACAGAGTGTTTTAAAATCAATACAGGAAGTTCAGAAACGTTTTCCTGACGGCATCCCCTTATTAGACCCTATTGAT |
| | | GATATGGGCATTCAAGATCAAGGGCTGAAAAAAGTCATTCAGAAAGTAGAAGCTTTTGAGCATCGAATGTATTCTCATCCACT |
| | | TCACAATGATCCAAATTTGGAAACTGTGTATACGCTTTGTGAAAAAAAAGCACAGATTGCAATAGATATTAAATCTGCAAAGC |
| | | GAGAACTGAAGAAAGCAAGAACAGTCCTACAAATGGATGAACTCAAATGTCGCAAACGTGTTTTAAGAAGGTTGGGATTTGC |
| | | TACTTCTTCTGATGTAATAGAGATGAAAGGACGAGTGGCTTGTGAGATAAGCAGTGCTGATGAGCTCCTTCTAACTGAGATG |
| | | ATGTTTAATGGCCTTTTCAATGACCTTTCTGCAGAACAGGCAACAGCATTATTAAGCTGCTTTGTGTTTCAAGAGAATTCTAGT |
| | | GAGATGCCCAAATTAACAGAACAATTAGCAGGACCACTTCGTCAAATGCAGGAATGTGCTAAAAGAATTGCAAAAGTTTCAG |
| | | CAGAAGCCAAATTGGAAATTGATGAGGAAACTTATCTAAGCTCATTTAAACCTCACTTAATGGATGTAGTATATACCTGGGCA |
| | | ACTGGAGCTACATTTGCCCATATCTGCAAAATGACAGATGTCTTTGAAGGCAGCATAATTCGTTGTATGAGGCGCCTGGAAG |
| | | AATTGCTTCGACAAATGTGTCAAGCAGCAAAAGCCATTGGAAACACTGAGCTGGAAAATAAATTTGCAGAAGGAATCACCAA |
| | | AATCAAGAGAGATATTGTGTTTGCTGCCAGCCTCTACTTGTAGAGTCAGCTAAAGGAATGTGAGATTTTAAATTATTGACCAC |
| | | CTGTTTGATTACAGTTGACTACAAATGCCTGCAAGTGTGGATTTGGTTCTCCCATACATTTTAATATGTATTATATTTAAATCAA |
| | | ACATCATTCATAGAAAGCATATTACATACATGTTTATACATAAGCATTACATTTTTTTAATAAAAATGTATACAGGTGGGGCACT |
| | | GTTTTGGTGGAAGGCTTGGAGTTTTTTTAATGAGTTTAGAGCTATTAGATAACCACTGAGTTAAAGGTAACTATGTACACACAA |
| | | AGTGTGCATCCAAGAGGCATAGCAGCAGCAGAAGTCTTTA |
| >ENST00000464526.1:: | 227 | AGGCTGGGGGAGAAGTTAAAGCCAGAGGAGGGGCAGGAATGTCTGAGGTGGCAACACTTCTCTTCAGCCAGACAGCACTG |
| chr6:31553970- | | GCCAGTTTGGAGTCTGTCCATCCTGCAGGCCACAAGCTCTGGATGAGGAACTTGAGGCAAGTCACCAGCCCCTGATCATTT |
| 31556686(+) | | CGCCTAAAAGAGCAAGGACTAGAGTTCCTGACCTCCAGGCCAGTCCCTGATCCCTGACCTAATGTTATCGCGGAATGATGG |
| | | TAAGTAAAGTGTCTCTTGCATCTGCATAGAGAGAGTCCTGGGAGCTTAGGAAGTGATGGGGAACAGTGATGTATGCAGCTCA |
| | | TGACTAGGTGGACAGGCCTCTGGGGACAGCTGGTACAGGAGGGAAAGGGACCTCACGGGAGGCCCAGAAACCTGGTAAG |
| | | AGGTGAGGTATTAAGGTCTGGGATGGAGAAGCTCTGAGGGTATATTTTTCTGCCTCTAAAACTGTTGGAGAGGGAATCTGAG |
| | | AAAGCTGCAACCAACCAGGAGGCTGGGGTACGCTGGAGAAGGAATGGGCTTCCTAACCTTGAGCCCTCTTCCCTGAAGATA |
| | | TATGTATCTACGGGGGCCTGGGGCTGGGCGGGCTCCTGCTTCTGGCAGTGGTCCTTCTGTCCGCCTGCCTGTGTTGGCTG |
| | | CATCGAAGAGGTGAGCGCTGCACTCCCTCCCTCCCCCTGCAGCAGTGCCCCCTGTGCCCCCACCCCCACACGCTTTCCCA |
| | | CTGCTTTCCCAGAACACTGCCTGGCCCTGGAGCCACTGGGAAGCCAACAGGGGAGTCCACGCCTGCTGGTGGGGGGAGC |
| | | CCGGGAGGGCCCGGGAGAAGCACAAAGGGTGGGCTGTGTTGAGCTTCTTCTTTTCTTCCAGTAAAGAGGCTGGAGAGGAG |
| | | CTGGGTGAGTCTGGGGACAGGGAAGGGGGAGGGCAAGAGAGATCCTGAGTGGGTGAGTGGGGAGAAGCATGGCTGAGCG |
| | | CTGAGAGGAGGGTTGGGGACGGGAGACAAGGAGAGAGAAAGTAGGAGCATGAGAGAGGCAGAGAAAATCGAGGCAAAAG |
| | | AGAAAGAGAAAATGAGACAGAAACCAAGAGAAAAAGTGAGACAGAGGATAGGAGAGACAGGGAGAAAATGAGAGTGAGAGA |
| | | GACACAAAGAGAAGAGCAATGAAAGAGAGAGAGAGAGAGAGGCTCCAGAACCAGGCACAGTGGCTCACGTCTGTCATTCCA |
| | | GCTATCGCAAGGCTGAGGCAGGAAGATAGCTTGAGCTCAGGGGTTGAAGACAATCCTGGACAACATAGTGGGACTCTGTCT |
| | | CCAAAGAAAAAAGAGAGAGAGAGAGAGAGAGAGAGAGAGGGAGAGAGAGAGAGAGAGAGGGAGAGAAGTAAGAAAGGCT |
| | | GGAGGTGGGAGCAGAACTCACAGGGAAGGATCTGACGGCATCGCCTCCCATCAGCACCTTCTGTCCTGGTCCCAGGCCCA |
| | | GGGCTCCTCAGAGCAGGAACTCCACTATGCATCTCTGCAGAGGCTGCCAGTGCCCAGCAGTGAGGGACCTGACCTCAGGG |
| | | GCAGAGACAAGAGAGGCACCAAGGAGGATCCAAGAGCTGACTATGCCTGCATTGCTGAGAACAAACCCACCTGAGCACCC |
| | | CAGACACCTTCCTCAACCCAGGCGGGTGGACAGGGTCCCCCTGTGGTCCAGCCAGTAAAAACCATGGTCCCCCCACTTCTG |
| | | TGTCTCAGTCCTCTCAGTCCATCTCGAGCCTCCGTTCAAATTGATCATCATCAAAACTTATGTGGCTTTTTGACCTTTGAATAG |
| | | GGAATTTTTTAAATTTTTTAAAAATTAAAATAAAAAAAACACATGGCTCACCCTTCCACCCA |
| >ENST000004 | 228 | GGCAAGACGGGGGCCCACCGGCCTGCTGGCTCCTCACCAAACTCTACTCCGCATTGACTGTGGGCTCTTGTTCTAGAGGCA |
| 41184.1:: | | CCCGCATCCTCAGGATGAAATGTGAGTACAAGAGCAGAGACTGCCGGTGCTGGTGTTTGCAGCCTCTGATTCAACAGAAAT |
| chr6:108444836- | | CTGGAAGTGTTCACAGCTTGGCCCGGAGAAGATCGTTTTAAATCTACAGCTTTAAAATGTGGTTGAAAAGGAGCCCAGGTTG |
| 108480596(+) | | GATTCTGCCAGCAGGTCCGCTGGCCGCTGTTACCTCCCCACTGTCAACGCCTAGGTCTCTGCCTCCAGCCCTGTTTCAGCC |
| | | TGTCCTTGAGGATTTGCTTCACCTCATTTACTGAGCTTGCCACAGATTCATTACCTGGCATCAGAGCTCCCCTCTCACTGAGC |
| | | CTGCCCAGAGGGGTGCCAGTATACTAGCCAGATGCCGCTGGAGGGACTCCACAATAAAGCCTGGCTATTAAAGACTTCAAC |
| | | AGTCTCATAAAGTCACTTTGCCAGCTGTAGAGCACACAAGTGCCCACAGATCCAAGCCTCCTTCCTTCCCCAAAGAAACACC |
| | | TTGGATAAAGTCTTGGAATATCATCCATTAGCACATGGCTCCAGCCAACACATGAATTCCCTTACACCAGCACATGGTCTCTC |
| | | TTTCCTGTTCATTCACTTTGGAAGACAGTCCTTGAAATGACAAGGCCTGGAGTGTCTTGTACAACCACCGCCAATCCGGTTTC |
| | | CAGCAGAGGCTTGTGGGCTAGGGAGTGTATCGATTTGTATTGTTAAAGGAATTTCCTGCTCCTCTCAGTGTCAGCTGAAGAG |
| | | CCCTCTGCAGTCATTCTCCTATTTTGAGAGAAGAGAAACAAGAGGAGACGAACTCCTGGTTCCTTCCTACGGGGTGTATAGA |
| | | CCAGTGACAATGTTAGTCCTACAATTTGGATTTATTTGTTTGTTTTCTAATCACCGCCACTTCTCCAAGTACAAAGTCTGTGTC |
| | | TATAATTAGATATCTTCTACATGTTACACCGGCAAGTCCATCTCATCATTTTACAGAGGGATGGTGACTTCCTTTAGCGCCCC |
| | | ATACTGACCGCGGTCCCTCACCTGCTGACTGATTGACTTATAAATCTGCCCCCCACCCACTTGTCTGTCCATTGAAGGCAAA |
| | | TTCGCCATCGGGAACACAGTAGTTTCTCAATAACTATCTAATGCATGGCCACCCTTCCCAGGATTTAGAATATCAAAGGGACG |
| | | TGTTCTTTCCTCCCAAATCACCTGCCTGCGCTTCCCCCAGCCGGGTGCCCCAGCCTTCACCTCGGTCCGGCCACCACCTCA |
| | | TCCCGCTTCTTCGCGGCTTCCAGGCCTCGGGCTGGGATCTCAGCCCCTGCCTGTAGGTGCGGCCCAGAGGGTGCGACACG |
| | | CGTCGGAGCGGGGCAGCGGAGGAGGGGACAGGGACGCGGCAGCCACACCCGCATCTCTTTCCCCTCTCGGGCATCCCCG |
| | | AGGGCCCTTCTGCCTCCAGTTCCTTGAGCCCCCGAAGCCCCCCATGCGGCAAGAGCGAGTCGCAGGAACAAGTGGGGAAT |
| | | CTTGGCCGCGGCCCCCTCCCCCGCCTTATAAAAAAGAAATTGACTTTTGTTTGGAGTTTGTGAAAAGTGGGGCTCGGGGCC |
| | | CAGTCAATGGGGCGCCCCGCGGCGCGGGCTGAGTGGAGCTAGCGCGAACCGCTCAGCCGCGGCCCCAATTAATCCGCCC |
| | | TTTGTGCGGCCCGCCCGGCCGCCCCCGCCGCAGCCGCACCAGCGGCCCATTGTTCGGCCTCGCCGGGCCGCGGGATTTA |
| | | CCCTTTTCAAACAGCCGGTTTTGTCCAGGGCAGTTCGAGCGGAAGTTTCTCACTGACAATTTGCCCCAAATAGATAGATTTGT |
| | | CAGAAGCGACCTTCGGGAAGGAAGCAAAAAGCCACCGGCCCGAAGGTTGGGCCCAAAACAGGGACTCTGCGTCCCACCCG |
| | | CGGCCGCGCCGCCCCCCCGCGCCCCCGGCCCTGCAGTCCCGAAGCCCCGCGGGGGTCCAGACCACTGCACCTGCTGAG |
| | | CCAGACACTCCGCGGACCTGACCTCCGTTTTACCCATCTGTAAAATGGAGCAGCTGGGCGCTGGCAAGCCGTAAGTCCAAG |
| | | AGCTTTGGCTCCACCAGCGTGTACGACGCTCATTTCCTTTCTCTCTCCCCACACCCTTTCCAAAAACTGTAAGCCAGAAAACC |
| | | TGGCCAGGCCTCCGCGACGGCGAGAAACGGTCTTGAAGGTCGGTCGGTCGCCGTAATGCGGTCCGGATCGTGCACGTGCT |
| | | GTTTCGCCACTGCCCTCCGGCTCCTCCTTAACAAAGATGTCAACCGTGCTAAATCACAGGTGGTGCCTTTGTAAGACCAGCT |
| | | ATTGTCCCCTCATAAGTCAAGATATAAAACGCTTTGTACTGAATGGCCCATGATCTGGAGCGCTCAACTTGGGAGTTGAGCT |
| | | GTTAGATTTTCTTTTCCCCACCCACCAGAGCCACTCTTCTCCCAAATGACCTCACTTAATAAGCAAAATTTAAATGGGGTGGG |
| | | GGGGGACTCAACATGCTGTGGATGAAAAGAATAGTTACTGATTTCTTCACACTTAATGGCATGTAGGAGTCTTTTAAAATACA |
| | | GTCTCATACCAAACAGATCTGTGAATTTTAAGCAGAACTGAATTGTATTAATCAGTTGATGGGTTTGTTCATCTTCTATCACTA |
| | | GATGAGAAATACAAAGGTTGGGAATCCACAGCGTCTAATTCTACTGTACTTAAGCAATCTCTCCCTTTAGTCCAGAAAATTTC |
| | | CTACTAGTTGCACATGAGAGAAAAATTGAAGCAAAAAAAAAAAAATCTGTTTTTTGTTTATCCATCAGGTGAATACACACTTCA |
| | | GCATCATACAGAGGAAACCCTAGGTGGTCTTTAGCAAGATTTCAGTGGTTAAGAGCTACAATTTAGAAAAGATGACAATACAT |
| | | ACACAGTGTCAATAGTATTTAAACAAACATCCCAGACAACAGAATAATGGTTATTAACTATGATGCTTTGGGCTTCTGACTAAT |
| | | TTCCCATGTCTGTCTGCAGCCAAACAGCTGCAGGATTTTTCCCCCTTTAAAAACTGTTTGTGCATCATTTTAAAAATTGCATTT |
| | | AACAGCCATATTCTCCAGAGCAGTCCCTCTGTGTGGAACTGAAGTGAATGGTTTAGCAATAGCGATGAAGCCAGTAAGTAGT |
| | | AAGAATGTCTTTCATGCCAAAAGAACTCTAACCACTCTGCTAGAAATGGGCTACATTTTTACTCTCTGAAATGTAGAAGCTGT |
| | | CTGCTACTGTATGACCTTCAGTATGGTTTACATGTCTAAATAAATGGAATTATTTAACCC |
| >ENST00000512382.1:: | 229 | GCAGAGGGCGTCCTTACTCCAGTATTTCCATGTGCTTCCCTGACCCGGGCCGGCCTGCCCACCAGGTCCCTCGAATCGGG |
| chr7:296160- | | GCCTCTCAGCGTTTGAGCTCTGCTCTCGCCCCGTCCCTCTCCTCACTCCTGCGGGAGAAACGGCCCCTGTTCTTTCCGCCC |
| 297419(+) | | CACGTTGTCCTCGTGAGTGTGTAGTCCAGGTCCTGTTTCCCCACAGAGACTCTGCAAAAAACACGGGGCCCAGAGGTGAAG |
| | | GCAGCTCCAGGTGGGGTCACCCCGAGGCAGGGCAGAGCGGCTCCGTCCCCTCCCACACCCGTGCTCCCGCTAATGCAGC |
| | | CTCAGCGCCGCAGCCCGGGCGGGTCCATCTGCAGACGCCAAGGTCCCTGCCGCAGTGTTTCTCTTCTGCTCCTCATGGCA |
| | | CGCGCCGGGCTCCCCAGAATCTGGCCTGGGCCCCCCGTCTCACGCTGGCTCCCCGCAGGTGGGAGGTGGACCCTGACTA |
| | | CTGCGAGGAGGTGAAGCAGACACCGCCCTACGACAGCAGCCACCGCATCCTGGACGTCATGGACATGACGATCTTCGACT |
| | | TCCTCATGGGAAACATGGACCGTCACCACTACGAGACTTTTGAGAAGTTTGGGAATGAAACGTTCATCATCCACTTAGACAAT |
| | | GGAAGAGGGTGAGCCTGTCCTCGCCCCTGCACACCCAGGGAAGGGCCGGCCACCTCCCAGCTACCTGCAGCCCACCTGA |
| | | GACCCTGGGGACGGGGGGAGCAGACCCTCCAGTGGAGGGATGGGAATGTCGCAAAGGCCCATCTCAGAGCCAGATGCAG |
| | | AGGGCGGCCCCAGGCCCCAACCAGGAGGGAGGGCCGCCCCGGGAGTGGGACCTCCGAGGCACAGGAATGCTGCAGACC |
| | | AAGTCCCGGCAGAGCTGGTGTTACCTGGGAAACGGAGGCCAGGAGAGGCTGCTGGTAGGAAGAGCAGGACCGTGCAGAA |
| | | TAGATGGGCCTCTGCCTGCACGCGGTACCTGGAGCCAGCCAGCGGGGGATAGGCGGCCTC |
| >ENST00000470261.1:: | 230 | CTGGTCCCAGAAACCTGGCCGCATCAAGTCAGCGCTTCCCATGCTGGAAGAAGCAAGCAGCCCCAGGGTGGAATCTTAAAA |
| chr7:29186247- | | ATTAATGAAGAGCATCGGCGGGGTGCCATTCAGGACTTACTTGCAAGCCCAGGATTTACATTTGGAAAGCGAGTGGTGTTTG |
| 29186920(+) | | ATTCCCACTGTTTAAAAAGGCAACACACGTTCGCTGATGGTCTACATTCCAGCTGCACTAGCAGTAAGCTCGCACCAAGTGC |
| | | CACTGTCCTGCCAAGCACTCTCCTGAATCACTGCGCTGGAGTCTCAGAGCCACCTGATGTGTTCTGTACCATTATACCCATT |
| | | TTGCAATTGGGGGACACAAACTAAGTGAGGTTAAGTAACCCATCCAGGGTCACATTGCTTGTAAGTGACCTGAGTGTATATT |
| | | ATTAGCCACCACCAGGTGCTGGGCATCGAGCCAGACAGTACGGGAGCAAGAACTAAAAAAGGCAAGGAGGTTGCAATGGC |
| | | AAGTAGATAACACAAAATAAAGGAGGGTCATCATTATCAGCAACAGCAGCGTCAACTGCCAGACAGGTGGGAGAGGCCACG |
| >ENST00000484589.1:: | 231 | TGCCAAGGCTGCAAAAGCTTCAAACACTTCTACACCTACCAAGGGGAACACGGAAACTAGTGCCAGTGCATCACAAACAAAC |
| chr7:40027434- | | CATGTGAAGGATGTGAAGAAAATTAAAATTGAACATGCACCTTCTCCCTCAAGTGGTGGAACTTTAAAAAATGACAAAGCAAA |
| 40109687(+) | | AACAAAGCCACCTCTTCAGGTAACGAAGGTGGAAAATAATTTGATTGTAGATAAAGCCACCAAGAAAGCAGTCATAGTTGGA |
| | | AAGGAGAGTAAATCTGCTGCTACAAAGGAGGAATCAGTATCTCTTAAAGAGAAAACCAAACCACTTACACCAAGCATAGGAG |
| | | CCAAGGAGAAGGAGCAACATGTAGCTTTAGTCACCTCTACATTACCACCGTTACCTTTGCCTCCCATGCTGCCTGAAGATAA |
| | | AGAAGCTGATAGCTTACGAGGAAATATTTCAGTAAAAGCAGTTAAAAAAGAAGTAGAAAAGAAACTCCGATGTCTTCTTGCTG |
| | | ATTTACCGCTGCCCCCTGAGCTACCAGGAGGAGATGATCTTTCAAAGAGTCCAGAGGAAAAGAAAACAGCAACACAGTTACA |
| | | TAGTAAAAGGAGGCCTAAAATATGTGGGCCTCGCTATGGTGAAACCAAAGAAAAAGATATTGACTGGGGAAAACGCTGCGTG |
| | | GATAAATTTGATATCATCGGAATTATTGGAGAAGGTACTTACGGACAAGTTTACAAAGCCAGGGATAAAGACACTGGAGAAAT |
| | | GGTAGCCTTAAAAAAAGTACGTCTGGATAATGAAAAGGAAGGCTTTCCAATTACAGCAATTCGAGAAATTAAAATTCTCCGGC |
| | | AGCTTACCCATCAGAGTATTATCAATATGAAGGAAATAGTGACTGATAAAGAAGATGCTTTGGATTTCAAGAAGGACAAAGGT |
| | | GCATTTTATCTGGTGTTTGAATATATGGACCATGATCTGATGGGACTACTGGAATCAGGCTTGGTTCATTTTAATGAAAATCAC |
| | | ATAAAGTCATTTATGAGACAGCTCATGGAGGGTCTGGATTATTGTCATAAGAAGAACTTTTTGCATAGAGATATTAAATGTTCC |
| | | AATATCCTTCTAAATAATAGAGGGCAGATAAAACTTGCAGACTTTGGACTTGCTCGATTGTATAGCTCAGAAGAAAGTCGGCC |
| | | GTATACTAACAAGGTAATTACTTTATGGTACCGTCCACCTGAACTGCTACTGGGAGAAGAACGATACACACCAGCCATTGAT |
| | | GTATGGAGCTGTGGCTGTATCCTTGGCGAACTCTTCACTAAAAAACCTATATTTCAAGCAAATCAGGAACTTGCACAACTAGA |
| | | ATTAATAAGACATGAAGAAAATGAAGTTTCTGATAAACAAATCTGACTATAATGAGGCAACGGAGTATACCGAAAAGTGTGTG |
| | | GCCTTTAGAATCTTACAGGGTTGGGTTCTAATGCCAGCTCAGCTACTTCCTGCGCTTATGGTTTAGGGTCCCATATGCTCTTA |
| | | AGTTTTTTTCTTCAAAAGTAAAAGAGGACTGATAACATGCTTAGGGTTGTGAGAATTAAATGTAAATAAGTTTTCAGTACATATT |
| | | TCTTCCCTCACCTTATTTACAGTAATGCTAGGTTGTTTATAGGCTCAGGAATGATGAGACCTAGTGTCAGTATAGTACAGATA |
| | | CATGTAAAACTGATCAAGAGTGATAACACTAATTTGCTTTAAATGGCTTTACACCATAGTGAATGAGTTATTTTATTTATAATTT |
| | | GCTTTGTTCCAGAAAGTATTTTAGGCAGATTACAAATATTTTATAATAGAAGTAGCATAGATTAAAAAGTGATGGAGGGAGGC |
| | | CGGGCACGGTGGCTCATACCTATAATCCCAGCACTTCGGGAGGCCGAGGCAGGTAGATCATCTGAGGTCAGCAGTTTGAGA |
| | | CCAGCCTGGCCAACATAGTGAAACCCCGTCTCTGCTAAAATACAAAACAAGCCGGGCACAGTGGCTCATGCCTGTAATCCTA |
| | | GCACTTTGGGAGGCCGAGGCGGACAGGTCATGAGGTCATGAGGTCAGGAGATCAAGACCATCCGGGCTAACACGGTGAAA |
| | | CCCCGTCTCTACCAAAACTACAAAAAAAAAAAAAAAAAAAAAA |
| >ENST00000522342.1:: | 232 | AGGATGGAGTCTGGATTTGGCTTCAGAATCCTCGGGGGAGATGAGCCTGGACAGCCTATTTTGATTGGAGCTGTCATTGCC |
| chr7:77787523- | | ATGGGCTCAGCCGACAGAGATGGCCGCCTTCACCCAGGAGATGAGCTTGTGTATGTTGATGGGATTCCAGTAGCCGGCAAA |
| 77807384(−) | | ACCCACCGCTATGTCATCGACCTCATGCACCACGCAGCCCGCAATGGGCAGGTCAACCTCACTGTGAGAAGAAAGGTGCTA |
| | | TGTGGAGGGGAGCCCTGCCCAGAGAACGGGAGAAGTCCAGGCTCTGTATCCACCCACCACAGCTCTCCACGCAGTGACTA |
| | | CGCAACCTACACCAACAGCAACCACGCTGCCCCCAGTAGCAATGCCTCTCCCCCTGAAGGCTTCGCCTCCCACAGCCTGCA |
| | | GACCAGTGATGTGGTCATTCACCGCAAAGAGAATGAGGGCTTCGGCTTTGTCATCATCAGCTCCCTGAACAGGCCTGAGTC |
| | | TGGATCCACTATAATCTTCCAGAGCTCATTCAGCAGCCTGCAAGCAGTAGAACAGAAGAGAGGATAGGAAAATTTGTGCATT |
| | | TTTCATGTTGAAGTGATTTGGCCCAAAATGTTGGGTATGGAAACACGAAAATAAAGGAGCACTAAGAGAAAGTAAA |
| >ENST00000488737.2:: | 233 | GATATTCTACTTTCAGCATTCTGAAGTCATGGAAATTCTTACTGTAGAAACTCAATAAACTTACAAGTAGACCTTTACTTTTTAG |
| chr7:87133179- | | TTCATTACTGATAAAATAATGAATATAGTCTCATGAAGGCTATAGGTTCCAGGCTTGCTGTAATTACCCAGAATATAGCAAATC |
| 87161013(−) | | TTGGGACAGGAATAATTATATCCTTCATCTATGGTTGGCAACTAACACTGTTACTCTTAGCAATTGTACCCATCATTGCAATAG |
| | | CAGGAGTTGTTGAAATGAAAATGTTGTCTGGACAAGCACTGAAAGATAAGAAAGAACTAGAAGGTTCTGGGAAGATCGCTAC |
| | | TGAAGCAATAGAAAACTTCCGAACCGTTGTTTCTTTGACTCAGGAGCAGAAGTTTGAACATATGTATGCTCAGAGTTTGCAGG |
| | | TACCATACAGAAACTCTTTGAGGAAAGCACACATCTTTGGAATTACATTTTCCTTCACCCAGGCAATGATGTATTTTTCCTATG |
| | | CTGGATGTTTCCGGTTTGGAGCCTACTTGGTGGCACATAAACTCATGAGCTTTGAGGATGTTCTGTTAGTATTTTCAGCTGTT |
| | | GTCTTTGGTGCCATGGCCGTGGGGCAAGTCAGTTCATTTGCTCCTGACTATGCCAAAGCCAAAATATCAGCAGCCCACATCA |
| | | TCATGATCATTGAAAAAACCCCTTTGATTGACAGCTACAGCACGGAAGGCCTAATGCCGAACACATTGGAAGGAAATGTCAC |
| | | ATTTGGTGAAGTTGTATTCAACTATCCCACCCGACCGGACATCCCAGTGCTTCAGGGACTGAGCCTGGAGGTGAAGAAGGG |
| | | CCAGACGCTGGCTCTGGTGGGCAGCAGTGGCTGTGGGAAGAGCACAGTGGTCCAGCTCCTGGAGCGGTTCTACGACCCCT |
| | | TGGCAGGGAAAGTGCTGCTTGATGGCAAAGAAATAAAGCGACTGAATGTTCAGTGGCTCCGAGCACACCTGGGCATCGTGT |
| | | CCCAGGAGCCCATCCTGTTTGACTGCAGCATTGCTGAGAACATTGCCTATGGAGACAACAGCCGGGTGGTGTCACAGGAAG |
| | | AGATTGTGAGGGCAGCAAAGGAGGCCAACATACATGCCTTCATCGAGTCACTGCCTAATAAATATAGCACTAAAGTAGGAGA |
| | | CAAAGGAACTCAGCTCTCTGGTGGCCAGAAACAACGCATTGCCATAGCTCGTGCCCTTGTTAGACAGCCTCATATTTTGCTT |
| | | TTGGATGAAGCCACGTCAGCTCTGGATACAGAAAGTGAAAAGGTTGTCCAAGAAGCCCTGGACAAAGCCAGAGAAGGCCGC |
| | | ACCTGCATTGTGATTGCTCACCGCCTGTCCACCATCCAGAATGCAGACTTAATAGTGGTGTTTCAGAATGGCAGAGTCAAGG |
| | | AGCATGGCACGCATCAGCAGCTGCTGGCACAGAAAGGCATCTATTTTTCAATGGTCAGTGTCCAGGCTGGAACAAAGCGCC |
| | | AGTGAACTCTGACTGTATGAGATGTTAAATACTTTTTAATATTTGTTTAGATATGACATTTATTCAAAGTTAAAAGCAAACACTT |
| | | ACAGAATTATGAAGAGGTATCTGTTTAACATTTCCTCAGTCAAGTTCAGAGTCTTCAGAGACTTCGTAATTAAAGGAACAGAG |
| | | TGAGAGACATCATCAAGTGGAGAGAAATCATAGTTTAAACTGCATTATAAATTTTATAACAGAATTAAAGTAGATTTTAAAAGA |
| | | TAAAATGTGTAATTTTGTTTATATTTTCCCATTTGGACTGTAACTGACTGCCTTGCTAAAAGATTATAGAAGTAGCAAAAAGTAT |
| | | TGAAATGTTTGCATAAAGTGTCTATAATAAAACTAAACTTTCATGTG |
| >ENST00000467458.1:: | 234 | GTCCCGCTCCCCGTCGCCCCACGCCGCCCTCGTCGCCGCGCCCAGACCCCTGCGGCGGCCGCAGCCGCTTGGGACTCGC |
| chr7:151574549- | | TGCGAGCTGGTTTCGGATCCATCCCGCCTCCCGGCGTCTCACTGTGTGCCCTACCCTTTGAAACACGCCCCCGCGCCCGC |
| 151576299(+) | | CCTGCCGTAGACCAGGCAGCGAGGAAGCCCACAGTCTCCGGGGGCGCTGCGCGCGAAGTAGCACGTGCTTCTCGAAACA |
| | | CCGCAGCCCCCGGGTCCCGCCCCGCCCGGCGCGCGCACTCGAACCCGCCCAGAGAGCGGTGCGTGGCGCTGGGTGCGA |
| | | GCAGGGTCTAGCCACCCCCACCCTCACCCCACCCCACCCCACCCTGCTTTTTTCAGGTTCATCAAGGTTTGCGCAGTGGAT |
| | | CCGCGAATGAAGCCAGCCTGGAAGATCCCCAGTCTCGAGACAGAGCCTGACAGGGGCAGATGCACTGGAAGGACCCTGTC |
| | | TGGGTTTAGCAACCAAGCAGCCATCCTGGCCCCACAGGTGTGGGACTTCTGGGTCTTCTCCTGGCTGGCTTCTTGCTAGAG |
| | | GATTTCAAGAGACCCAGCAAGACTGTATTGTCCCACTGAATGCTCAAGATATTGGTTAGAAGTAGAAAAGGGGAGGGGGTAG |
| | | TATTTAGCCTCTGTCCCCACTAAAAATTATTCCCAATTGTCATTTGTGTCATCTGTTTAGCTTACAGTTTTAATCCTTGTCAAAA |
| | | TGATCATTTGCCAGGGTGCATAAAACACCCTATTTGATGAGAACTGGTTTTAAAGGAAAATGGGGCCATCAGAAGGAAGCCG |
| | | GTTGGTGTGAAGAGACGTCTGGGAAGAGCCCCTGGCGAGGGCCCTCATTCCACTGGGTGACCCAGAAGGAGAAAACCGCG |
| | | ATGGCGCTTCTCATTTGAAATGTTTTCTTTATGGACAGTGTTTAGCAATTGTTATAATGTGTACTTTTGTTGATTGATCTGTATA |
| | | CATATGTAAAATAATTTGTTCTCTGAGTTAATTTTGTTGGAACAAAGACTTTAGTGCTTCCTACATTAAAACATTTTACTGTA |
| >ENST00000521369.2:: | 235 | GTAGCTTCCACGCGGGCAGGTCCGGAAACTGAGCATGTCTGCAAGCGCTCAGCGGCGCCGGCAGCAGCGGGGCTAGAGC |
| chr8: | | TGGGCTGCGTCAGGCTGAGCCCATTCACCTCGCGGCCACAGGAGCTCAGCGCCGGCGCCGCGCCGCCCAGCCCCGCCGA |
| 107282371- | | GAGGGGCGCACTCGCCGCCGCGGGGCCCGCCGCCGCTCACCGCAGCCCCCTCCTGGCGACCCGCAAGAGGACTCAGCC |
| 107285127(+) | | GCCGGCTGCGACCACCGTGGACTCCCTCTATGGAAGGGAATCCTGGGAGTCCCGGCCGTCAGAGGGGTTCCCAAATCCAA |
| | | AGGGTCCATCTCCAAGGCGTCACTCCAGGGGTCCGCGGTTCACTTTGCTGCCCCTTGCAGGTGATGCTGTGATTGTTGCCT |
| | | GCGGACTGGGAAGCACGATGGCTCCAAAAGGCCAGCGGGGCCGAGAAGCAGGGACAGAGACCAGCATTTAAGGGATCTCT |
| | | GGTGGGGGCCTCCCTGGAGTTCTCCACATGTAAATATAGCGAAAAAAGAAAACACTACGTTACTAGAATAAACACGTCCTAA |
| | | AATTTCTGTGCAAAAACACCTTTTTGAACAGCAAAGATTTCACCCACCAAAACAAGGAAGCAAGAAGCTTCCACAGTGTGCAG |
| | | GATCCGAAACAGGACCGGCCCGGCGTTGGGTGAGAGTGGGGGAGGGGTTGTCGGCTTCTCCATCCAGCTTGCCATGTTCT |
| | | GTCGTCGGTTTCACATTTTCAGAGAGACTTTACCAGATGCAGGAAATGAGGATGAAGGCAACTCTGGAAGGATGCAAAGAAG |
| | | GGAGAGCAAGCACTGTACCTACTGTCTGCTGTCTAAGAGGGCTGGTCTCATAACTAAAAGGTCCAGTCCCTGAGATCTTGTG |
| | | TGCAACCGTCAGAAATAGTTCAGGACATCAAATTGCACAGAGAAGATTCAGATGAAGGGAAACAAGAACTCTGTGGAAAACT |
| | | GTTGGGGACAGTGGCACGAGTTGTCTTGCAAGGTGATGTCTGTCCCGTGGAGGGACACAAGAGCGAGGACAGCATGGTTC |
| | | TAATGGCCAGGGCATGTCTTCTTATTCCATGTGACAGTGGCTGGCTGAGTTGCTAGTACGTTTTTGAAATATACCCTTTGAAA |
| | | TATGTTGATTATTCTGGACATCTGTAAGGTGGGAGATTTGAATAAAAAGACTTCTGCTGTCATTATTGGCTTTTATGGCTATGA |
| | | TTTTTTTCAGTCATTATTATAAATCATGTGAAGGTAAAATAAATGATCATTTTAGCCATTCTGTTCTGCACAGAATGCTGTTCAA |
| | | ATAAAACATCATCTGATA |
| >ENST000005 | 236 | GAGCGTCCTCCTTTTGTGCGAGCGCCGGCGGCTCGGCTTCTCGAAGGAGAAGAAACTGAGGCCTGGAATTTGATTAACTCA |
| 23514.1 :: chr8: | | TTCAAGGTTACCCAGTTGGTAAGTAACAGAGCTGGAATTTGACCATGGTCATCTGGCTCCAGAGCCAGCACTCTTAACCACA |
| 130982754- | | CTTCCTATGAGGAAAAATTAAAGTGGATGGTGATGGCTACAAGATGGATTTCAGTTGCATAGAAATAAGAACCTCCTACCTCT |
| 131028898(−) | | GTTGAAATCAGAATACATATACACCCCTTCATTCACTCTATGAATATTCATTCATTCCATGAACCTACAAGGTTTTTAGGTCAA |
| | | ATTATTAGTTTAAATAAGAACTATAGGCTGGGCGCAGTGGCTCACACCTGTAATCCCAACACTTTGGGAGGCTGAGGTGGGT |
| | | GGATCACTTGAGGTCAGGAGTTCAAGACCAGCCTGGCCAACATGGTGAAATCCCGTCTCTACTAAAAATACAAAATTTACTAA |
| | | AAATACTAAAAATACAAAATACTCACTTGAACCCAGGA |
| >ENST00000531995.1:: | 237 | GCAGGCCTGTCTCCCATGGGCTGGGCCCCCAGTGCTCCTCACTGCCGCCTCTCTCAGGCCAAGAAGTACGCCATGGAGCA |
| chr8: | | GAGCATCAAGAGTGTGCTGGTGAAGCAGACCATCGCGCACCAGCAGCAGCAGCTCACCAACCTGCAGATGGCAGCAGTGA |
| 144902402- | | CAATGGGCTTTGGAGATCCTCTCTCACCTTTGCAATCGGTCAATAGAAATGCTCACTTCTTCCGGGGCTCATGTCCTTAGTCA |
| 144903913(−) | | GGGCTAGAGGGGGGCAGTGCAGCCCACGTGCTGGGGACCTTGCTCCCCTGGCCGGCCCTGCCCGCCAGCTCTGGACGAG |
| | | CGCAGCAGAGCGCCAGGCGTGGTGGGGCCAGTGGTGTGGGCACCCACCCGGGACCGCCCCTGGGGAGGCCTCCATCTG |
| | | CTCTGAGGGCCCACCTTAAAACTAATCGGCCAGGTAACTGCGGAGGGCAGCCGGCCAGGGGGGTAGCTGCCAGCACCA |
| | | GGCCCGTCTGCGCCCACGCCACCTGCACGCACGGGGTGTGCCCACGGCCTCCAGCCCCGTCAGTCTCCAGGCTGCCTGG |
| | | CTCTTGGGTTGGGGTCAGGCCCCTGAGGAGAGAGGGGTCCTAACTCATGGCCCCATCAGACCCTGGAGGGAGG |
| >ENST00000468206.1:: | 238 | TGAGCCTAGCACCTGATCAGTGGAGAGCTGTGGATTGCATGTTTGTTTGCCATTGCCCCCGCCACCCTGCAAGTTGCACCTT |
| chr9: | | CTAGAATCAGCAAGCCAAGCTCCTCTCACCCAGCGTAATGATGCGGAAATGCAAATGCACCATCATGTTGTGACCCATATTG |
| 95726387- | | CGAAAATTAGAAAAAAGGAAGTTGTGTTTCGCTATTGCACGAAGTTCAGCCCAGAGGAGAAACTCGCTCGCCTTCAGAAGAC |
| 95738991(+) | | AGTACCTCCTAAATGGCTCTACTTTGAACCTGCTGGGCAAGGAAGAGATTTTCAAGGAAACCATCTACCGTGTGCAAGCTCC |
| | | TGCCGGCCAACCCCAGACCCCAGCACGGAGCCAGGCGCCTGTGCCCGCCGACCCTCAGCATCCTCCTCAGAAAGGCTGGT |
| | | GGCATCAGGAAGCCCCTGGCCAGCCTCCACCTGAGCCCAGTGAGCTCAGCTTTAAGGATGGAGTCAGGCAGGGGGTCCTC |
| | | AACCCCTCCAGGACCCATTGCTGCCCTAGGGATGCCAGACACTGGGCCTGGCAGTTCCTCCCTAGGGAAGCTTCAGGCGC |
| | | TCCCTGTTGGGCCCAGAGCCCACTGTGGGGACCCTGTCAGCCTGGCTGCAGCAGGGGACGGCTCTCCAGACATAGGCCCC |
| | | ACGGGAGAGCTGAGTGGTAGCTTAAAGATCCCCAACCGGGACAGCGGGATCGACAGTCCCTCCTCCAGTGTGGCTGGAGA |
| | | GAACTTTCCCTGCGAGGAGGGCTTGGAGGCTGGCCCAAGCCCCACTGTACTGGGGGCGCACGCAGAGATGGCCCTGGAC |
| | | AGCCAGGTCCCGAAGGTCACCCCCCAGGAGGAGGCGGACAGCGACGTGGGTGAGGAACCTGACTCTGAGAACACCCCCC |
| | | AGAAGGCTGACAAGGATGCCGGCCTGGCCCAG |
| >ENST00000501079.1:: | 239 | AGGTGATAGCGGAGCGGCTGGGTAGGAAGCAATTGTTCTCAAACTTCACTAGCCCCGTCGGCGCGGACGCTTGTCGAGAAT |
| chr11: | | GCAGATTCCTGGGTACTGCCAGATACGAATTGAGCATACCACAAAAAAGTTCTCATTTTGTGTCCTCCCATCCCATTCTCCTC |
| 10879805- | | ACTAACCAAAGGCTAGGAATTATCTGTGAATGTAGGACCACTGGATTTGCAGTCTTCATCTGACACTGTGGAGAGTTTCTAG |
| 10900823(+) | | GAATGAAACAGATATATGGCCTTGGGTCCCCTTTTTTTTTCTTTTTTTTTTTTTTAATAGAGACGAGCATCTCACTATGTTGCCT |
| | | AGGGTAGTCTTGAACTCCTGGCCTCAAGCAATCCCCACCCGACTCCGCCTCTCGAAGTGATGGGATTACAGGCATAAACCA |
| | | CCACGCCTGGCCAGAAGGTGCTTTAACACCAAATCTGAAAATTGTTCAGAAGAGAAACATTGAGCATGAACACCATCTGTGC |
| | | GAGTCATTTACTTATTGCCCCTCACCTCTAAATCTACCTTCTGTACTCTTCTTCCCTGTAATGATGGGGCTAGTTGTCCTCAAA |
| | | CTGTTTCTCAGACTTCTTTTTAAGCTTGCTTCCTGTTCAGTTCTGCCAATAGGGGTCACTAGAGAGAGACTGGGAGGCAGAA |
| | | GGAGAGAATATGCTTCCTGTTTTTTCTGTTCTTGTTAATGTTGCTTACAGGACCAGCAATGCTTCTTCACCTAGAGACACTTCT |
| | | CCCAGCAGTGGCAGTGCCACTTCAGCTTCTTTCAGCACTACTGGAATCAGCCTCAGTGATTCCCCCTGTACCCGCTCAGAGA |
| | | TTATCCACAGCAGCCAGATGGTTCTACCTTCCACAAAGATTGTGGTTGCAATTCTGGGCTTCTAAGTTCTGGTTACTTCATAT |
| | | TTTTCCTTTTGTTCCTCCAGCCCTAGAGGTGGTAGCTGCTTTCTGAAGTTATTATTTCTAGATGACTTTTGGTTTTTCAGCCTT |
| | | TGTATTTTGCTTTTCAGCCCTCTAATGCCTGTATAACCAATTTCCCTGTAATAAATAAATTTCCTCCATTGAAATACC |
| >ENST00000469976.2:: | 240 | GAGTGCTGCAGCCGCTGCCGCCGATTCCGGATCTCATTGCCACGCGCCCCCGACGACCGCCCGACGTGCATTCCCGATTC |
| chr11: | | CTTTTGGTTCCAAGTCCAATATGGCAACTCTAAAGGATCAGCTGATTTATAATCTTCTAAAGGAAGAACAGACCCCCCAGAAT |
| 18416107- | | AAGATTACAGTTGTTGGGGTTGGTGCTGTTGGCATGGCCTGTGCCATCAGTATCTTAATGAAGGTAAGTGAGAGTCTACCAC |
| 18418945(+) | | ACTGGAAGCCCATACCTTGACCCCATCCTCTACCCCCACTCCTACCCCTAGAACTGTATTATTACATTTCATGTAACAGTATTT |
| | | AGATTTATGCACTCATTCGGATAACTTTCTGTGAAACAAACTTTTGAAATATGATAATACACCAAAAGTGTATCTGAAATTAAAA |
| | | AGAATCAAAGGTTGTCAGGCTGGAGACCCAGTTCCTAAAATTCATTATTCTGTATTAACATGCATGGATTGACTACCAATGAA |
| | | AAGGAAGGGTCCATGATTTTAAATGAGCCAAAATTCTTTTAAAGTGATTTTTGAATTGAAAATGACAATTCAAAAATTGTCATTT |
| | | ATTGGTAAAATTATATGGGAAATCATAAGTTCTCCCACTCAAATCTCATTGCCCCTGTGCCTTGGATAGCAATT |
| >ENST00000545202.1:: | 241 | ACAGGAAGCTGAAATGCATATTGCTAGATAGAAGAACCAATCCGAAAAGGCTACTTTCTCTATGATTCCAACTACGTGACTTT |
| chr11: | | CTGGAAAAGGCAAAACTATGGAGATGGTAAAAAGAGAAAAGGATTTCTGCTTTCAAATCCGCTGTTTCCTGAGGCTGGGTGC |
| 69240457- | | TGAGTTCAGCTGCCCCTGGAGGAGCAGCAGCTCTGGGCCGCTGGCAGGGCCGCATCCTCTTCAGTCTGGACAAATTCCCT |
| 69244389(+) | | GCAGCCAGGGGAAGTGGCCCATGCCTGCTGTCCTGGTATGCAACATCGGACCTGCATGCTGGAATTTGACCCTTTTCCTGC |
| | | CTGACACCCAGCAAAATAAACCCCAGGGAAGGGATCAATGCCTGCAACCGGAATCCAGAAGAAAGAGGAGTGACAGATAAG |
| | | CCCTGGGTCAGAGGCCAGTGAGGTCAGGACCTCCCAGGGACGGACACACAGCCCAAGGTTTGGGTGTGAGATTTCAGAGC |
| | | CCCAGGTAATAAAAATAAAAGAGTTAAGGTGCCTAGCAGCTCAGTCCCTCACAGTGCTACCTGGGGCAAGGATGGAAATTCA |
| | | CCTCAGTTTGCTGTGGTTGCTATTGGCTGAATCATGTCCTCCCAAAATTCATATGTTGGAGTGTACCTCGGGATGTGGTATCT |
| | | CAGGATACCTCAGTACCTCGGGATGTGGCGTTGTTTGGAGAAAGGGTCGCTAGAGAGGTAATTAAGTTAAAATGAGGCCAT |
| | | GAGGGTGGGCCCAAATCCAATTTAATTGGCATCCTTCTAAAAAGGGGAAATTTGGACACAGAGAGAGACACAAACAGGGAG |
| | | GGCGCCCTGGGAACAGGAAGGCGGCCACCTCCAAGCCAAGGAGGTCCTGGAGCAGATCCTCGTCTCAGAGCTCCAGCGG |
| | | GAACCACCCCTGCCCAACCTTCATTTTGAACTTCTGGCCTCCAGAGCTGTGAGACGATACACTTCTGTGGTTTAAGCCGTGT |
| | | AGCCTGTGGCACTTGGTTATAGCAGCCCCAGGAAGCCCAACACAGTGGCCAAAACAGAGAGGACAGCTGGCTTGCTAACGT |
| | | CACACAGCACACTGACGAGCAGGGTCTTCAACTCACTACTCAGGAACCCAGTAGTCAGGGGTGAGGGGGAATGCCCTTCCA |
| | | ATATGGCTAGAGCCAGACTGTCTGGGCCCAATCCCCAGCTTTGTATGTATTACCTAACCTCTCTGTGCCTCAGTTTACCAAG |
| | | CTATAAAATGGGCATAATGACAGCACCTGCCTGGTGATGCTCTTGGAGGGATGAAAGGAGGCAACGGTGTGGGCATCTGGA |
| | | CGGCACCAGGCATTGCCCTGAGAGCTGGCCATGCGGGGAATGCTCCATCCTTACGCCAGCCAGGTGTTGCCACCCCTCTC |
| | | TTGCTGATGGAGACCAGGAGGGTCGGGACAGAAGGCCTTGCCCAAGGTCTCACAGCCAGGAGCCTGCCTCCACTGGTCCC |
| | | TGTGTGGCCAGTGCTGCCCTGGGGGCTGCTGCTGTGCACTGGAGGGTCAGCTGCATGGAGGAGCCAGAGGAGATGGTGC |
| | | CGAGGACAGAGCTTGCGGCCCTGTACCTGGCTCCCAGAGGAATGGGTCTTGGAGCTGCTCTGCACAGACACTGACCTTGA |
| | | GGAGAGACAGGGGCGCCACTCCCTCCTCCACTCGCTCAGGTGAGCATTCAAAGCGTCATAGGTAACCAGGAGCACTAGTTG |
| | | GCATGGCGATTTCAGAGGTGAGTCCAATGTGGACCCTGGCCCTGGGAAGGTCACAGTTTGATGGAAGATGCCAGACTCACA |
| | | CATTCACTCACTCACTCATTCCCTCGTGTATTCATTCCGCAAAGCTTCACTCATTACCTACCTGGGGCCAGGACTCAGTCTCA |
| | | GAGCCAAGGAGAGCGTGGTTGGGAGGAGACTCAGACGCTTTCCAGCATCCCTGCCCCCAGGTGTCTACACACCTGTGTACT |
| | | CCCCAACTTGGAGTGTGGCAGGGCTGCAAAGAGGGTGGATGTGCTCCCGTGGTGAGGTTACTTTATATCGCAAAGGTGCCG |
| | | GACTTCTGCAGGTGTAATTAAGGTCCTAACCAGCTGACCTTGAGTTAATCAAAATGGAGATGATCCTGGGTGGGCCCAAGCT |
| | | AATCAGGTGAGCCCTTTCAAAATGGCCTTGAAGTCAGAGACTCTCCTGCAGGATTTGGAGATCCAGCCCCATGAGGAACAG |
| | | GGGCAAGGAAATGCAATCTTCCACAACCGAGAGAGCTTGGAAGAGGGTCCCAAACCTCAGAGGAGACCCCAGCTCCGGTT |
| | | GGCACCTTGACTGCAGCCCCATGAGACAGAAGACCCAGCTATGCCATGTCCTGGCTCCTGACCTGCCCAAACTTTAAGACA |
| | | ATAGATAAATGCCATTTTAAGCTTTT |
| >ENST00000495442.1:: | 242 | GTGTGCTGTAGGATAATAAAGCTTTTCCCCCAAAAAACAGGTGAATACTTAAACTAATTCAAAGAGAGAAGAAAGCTTCCTGA |
| chr12: | | AAGGTCATTTAATTGACTTTTGCTTTCCAGGTGTCAAATCAGACACTGAGCTTGTTCTTCACGGTTCTGCAAGATGTCCCAGT |
| 9220307- | | AAGAGATCTGAAACCAGCCATAGTGAAAGTCTATGATTACTACGAGACGGATGAGTTTGCAATTGCTGAGTACAATGCTCCTT |
| 9221551(−) | | GCAGCAAAGGTAAGCCACTCACACTCCTCCAAAAGGCAGTCAGAGCTCCTTCAGCTTGCCCCCCAAACCTTCTCCTTCATAA |
| | | AACGCTGGGTAAATATTTGTCAAAAACATCAAATTACTCACACTGCACATTATTATAGAAAAACACATTTATTGGAGAGGGCC |
| | | GCTGACTCTGTCAAACCTCAGAGAGTCCATAGGATTGCTTATGGGTAATGATTTGGAATAGATTTGGTTTCCCACTGTACTGA |
| | | TTAGGTTTCCTTGGGCACTATGCTACCCAGAACTAAGGGAAAGAATACTCTCTGCTCATGGAGACCCAAATCTGTCTTAATTT |
| | | TTTTTCTTTCCAATGTCACAGATCTTGGAAATGCTTGAAGACCACAAGGCTGAAAAGTGCTTTGCTGGAGTCCTGTTCTCAGA |
| | | GCTCCACAGAAGACACGTGTTTTTGTATCTTTAAAGACTTGATGAATAAACACTTTTTCTGGTCAA |
| >ENST00000557208.1:: | 243 | TACTCACAACGCTGCCGCCGCGCTCCGTGGGCAACTCCTACTACTGCTGGGCTGGGCTGGGCTGGGCTGGGCTGCGCCG |
| chr14: | | GAGCTCGCCTGCACAGATCAGCTCCGGAGAGGGGAAAACCACGCTCCTCGGACCAAGCCTCGGGAGCTAAGCCAGATCTG |
| 52327358- | | CCAGTGAGCCTCAGGCTTTAGGAACTGAAGAGGTAAGAATTCCAAAATATTTTCTTCTAGAATCTTGAATTTAACCAGAACTTA |
| 52344762(+) | | AAGAGGAAAAAAAATGTTAAGTGGTCTATAGATGAAAGCATGTGATGTTTTATTGAAAGAAACAGTCATAGAGTCCTTATCAAT |
| | | ATTGACATGACTTCAGAATTTAAAGAAAAAATGGCTGTCTTTAATGATAGTTAAAATTGATTTTCATCCAGAATTTTTTCATTTT |
| | | CTTCTAAAAACCTGAGTTGGAGAGCCTTTGAGGCTGCTGTCTTAACAGAATGTATCCTCACGGCACAAACATGATTAATAGGT |
| | | GGCAGACATTGAGGGCATACCCTGTGCAAGATAAAGCCTCAGATGAGGCTGATTAGGTCAGGGAAAGGTAAACCCTGTTCT |
| | | TA |
| >ENST00000555112.1:: | 244 | CCTTGTGGGGGATTTTTAAAAAGTCGTTTTTTTTTTTAAAGAAACATTTCCGTGCTACTGTCTGTCATCGTCTCTGGGGAAATC |
| chr14: | | AGCCAGAACCACCGGAACGTAACTGAAACCAGACAAGAGAGGCAGGAGCCCAGGCAGTACCTGCAGCGTCCGGGCACCAG |
| 55034349- | | AGCCACCTTGGAACAGGAACGCGTCTCCGGCCGCGGGGCTGCGGCTCCGCCAAACTTTGGGGGGGGCGGGGGGGGCTG |
| 55170585(+) | | GGGCGCCCAGGGGGCTCTGTAGACCGAGGGCGGCCCCCTAACCATGATGTTTCGCGACCAGGTCGGGGTGCTGGCGGGC |
| | | TGGTTTAAGGGCTGGAACGAGTGCGAGCAGACTGTTGCGCTGCTGTCGCTGCTCAAGCGCGTGAGCCAGACCCAGGCCCG |
| | | CTTCCTCCAGCTCTGCCTGGAGCACTCGCTGGCCGACTGCGCCGAGCTGCACGTCCTCGAACGCGAGGCCAACAGCCCCG |
| | | GAATCATTAACCAATGGCAACAGGAATCCAAGGATAAAGTGATTTCCCTCCTGTTAACTCATCTGCCTTTGCTGAAGCCAGGA |
| | | AACCTCGACGCGAAAGTAGAATATATGAAACTGCTGCCCAAAATCCTGGCTCACTCTATTGAACACAACCAGCACATTGAGG |
| | | AGAGCAGGCAGCTGCTGTCCTATGCTTTGATACATCCAGCCACTTCGTTAGAAGACCGTAGTGCTTTAGCCATGTGGCTGAA |
| | | TCACTTGGAGGACCGCACGTCGACCAGCTTTGGTGGCCAGAACCGAGGCCGCTCAGACTCTGTGGATTATGGACAGACACA |
| | | CTACTATCACCAAAGACAGAACTCTGATGACAAGCTCAATGGGTGGCAGAACTCTCGGGATTCTGGGATTTGCATCAATGCC |
| | | TCCAACTGGCAGGACAAAAGCATGGGGTGTGAGAATGGCCATGTGCCCCTCTACTCCTCCTCATCTGTCCCCACCACAATC |
| | | AATACGATTGGAACCAGCACAAGTACAAGTAAGTTCCCCGGAATCCCTTTAACGTAGTCTGGTTTGGCGATTTGCTGTGTAT |
| | | GTGGCATGTCCTGCTGACAGTGTCTGCTCCATGCACCCTGTGACCCTGGGAGAGAAGGACTGATTGGAATTGGCTGTGGGA |
| | | AAGGTCCTTTGGACCCCATATTTGTTGCTCTTTGACTCTGCAAAGATGAAAATACTTGGGAAGGTAAAAGTGACTGATGGTCC |
| | | AAATTTGGGTTATAAACAGACCAAAAATAAATAGACAATAAATGAAAGAAGGAAACTTGCCGTTTCCACAATCAGAAATGCATT |
| | | TCACCCATGTCTTTCACCCTGGGGTTTTGGATTTGCTGAGACACTGAGTTACTGTGTGATTCTCTAAACCTAATTCTAACCCA |
| | | TGGTTAACCAGGCAGCCTCATGGGCTTTGTGCTTTCTCTTTATTTTTTTTTTCCTCTTTCTTTCTCTTTATTTATTTATTTTTTCT |
| | | TAAATTTCACCCTGGAATGTTTCCTCCCTTTGCATTTTCTGGTTTTAATCCCCAATTGTTGGGGCACTTTTTTCCTGACCAGTG |
| | | GGAGCAGGTTTGCTAACTGAGAATGCTGCTGTGTGCTGTGGCCTGGTGAGAATGCCATGGACTTCTCTCGCTCAGTCCAAC |
| | | ACCTTAGGAGAGGTGGGTCAGGAACTGCTTTGTGCGTGGGGAGAGCGGAGGACAGCCCTCTGCTCTTTTGACCACTAAAGC |
| | | AAAGTATTGCACCCTCAGCTGGGCACTGGGGCTGGGAGTCCTCTCTGTGTCTCCCCACTGAGGCTGAGCAAAGTGACAAAC |
| | | ACACCATTGGTGTCTGTATAAAGTAGCGGTGTGGAAGTGCACCCTGTCTTCAAACAGCGTTCCTTAATGCTAGTTTGCACCT |
| | | GTCCATGAATGCAGTGGCAGTGCCAAGATAACTTTCTTGAAGGAATGCTTTTGCAATTAGACCTCGTGTGCCTGTCTTCATGC |
| | | AGGAACCCCCAGTTAAAAGATATGAGAGGATTAATAATTGGGGTGCCGGCACAGAGACTCAGGCCTGTGATCCCACCACTTT |
| | | GGGAGGCCTAGGCGGGCAGATCACTTGAGGTCAGGAATTCAAGACCAGCCTGGCCAACATGGTGAAACTCTATCTCTACCA |
| | | AAAAATACAAAAATTAGCCGGACATGGTGGTGTATGCCTGTAATCCCAGCTACTCGGGAGGCTGAGGTTAAGGAATCACTTG |
| | | AACCCAAGAGGCAGAGGTTGCAGTGAGATGAGATTGTGCCATTGCACTTCAGCCTGGGCAATAGCATGAGTCTCAGAAG |
| >ENST00000554208.1:: | 245 | ATTCTTTATCCGGAATTTCAAGGGCCGCCGGAGGGCTGTCGCTTCTGCAGTGCGTAGGAGCGGCCGGGGGGGGAGGCTCC |
| chr14: | | GCGGAGCCGAGGCGTGGAGAATAAGAAAGCCTTGAGTTTTGTGAAAAACCGGAGAAGAGAAACTAAAAGGACAGTAGAAAA |
| 67708151- | | GGCTTTTCCAGTTTGCATAATGTAAGTTCTTTTCACTTTTCTTGGCTTGAATTCTGCCTTAGCTGACAAAGCTGTTTCTATGGA |
| 67738776(+) | | AACAAATAGTTTAACCTCTTAGAATGTCAGAGGAATGCTGATCACTTAAAATGAGACTTCCCAGCTGCTGGTCAAAGGGCATT |
| | | ACTTGCTGGAGAAAGAGGGGAAGGATGAATTTAAAAAATTCTAGAGTTCTGGACACTAGTGTAGACTGAACATGTTTATGCGT |
| | | ATGAAGTATATCTCTTCTATTTTACTCTGCCTAAAAATTAAGATACTGGTAATATAGGAAATTGGATTTCTGCTGACCTGTCTTG |
| | | ATTCTCAGCTCTTAAACAAATGGTTCTATTCGTTGGTACCCTTTTCAGTGGGTCTGTACCCTTTAGGCTATTTCAGTCCCTCCA |
| | | GTCTCAATCATTTGCACTTGTTGTGAAGCTTATTTAGCTTCCCATTGCCCCCTTCTCCCGCAACTAAAACCCTTTCTCCCTTAC |
| | | TAGTTTGAGGTTAGCATTGAAACAGCTTCGGCATGCCAGCCTGAAAATTTTTCTTGCTTTGTGGACTATGTAAAGATTTTTGTA |
| | | TTTGTGTTCGTTTTCTGGCAGGGATGGGAGCAGGGATTCTGATGAGGATTTGAAATGTAGTCCTTTGAAGCTTTCTTTCTTTC |
| | | TTATTTTTTTTTTAAACTTATAATTGGCAAGTTGAGAAGAAAAGAATTAGGGCCAGTAACTGCCTCTGCTGTCTTCATAAGATG |
| | | GCCTGTTATTTACACACAATGAGGTAATTCTCTAGTTCAGTCATTTACCAGCTTCTGAATTGTCAAAGGACTTTTTTTTTTTTTT |
| | | TTAAAGAGAGGGAGAGTGCAATTGCTTTGGACTGGGAGGACTTTTTCACTTTTATGATGTATATAGACTACATGTATGGGCTA |
| | | GAGTATTTGTGGAGTGCCTGCTTAAAAATTATAATCTTAATGCTAAAACTATAGGATACAGTATAATGTTTCTTTAATTCTGGAA |
| | | GAACTTTAGGATCTTTCATTGCCTTCATTAAATTAACTTGTGAGCATTATAGAGTTTTTGGAGTGTTAAATGGTGAAGTAATTAT |
| | | TTAACAGGATAGTCTGGAAAGATTTCTTGTAGAATTAGGGACTTGAGGGAGGTCTTGAAGGTAGAAATCAATGGAAAAGTAG |
| | | TTCATCAGTCATTTGGGCAGAAAGATGGGATAGACACAAATGAGGAAGGTAAATTAGAAGGCTCATGCAGGTGCGTAGTAAA |
| | | AGATATAGCTAGAAATCTTGGATTCATTTTTTGAGTTTGAAGTTTATTCTCCAGGTAGTGTGAAAGCCATCAGAAGTTCTGAAC |
| | | AGAGGAGTGGTTTGCTCATATACTTAATTAAGCAAACACTTAGCAGGTATCACGGCTATAGAGGTCAACAAGACGGATCCCT |
| | | GCCAATTAGATAATTTACCTGTGTGATCTACAGTTACCTTGAAATTAATGTGTCTAAACCCATATTTCCCTTTACTTCTATCTGA |
| | | TTCTCCTATATTCTCCATCATAATTAGTGACATTGTCATCTACCTAATAAGCCAGGGATAGTGGTTAAGAGTGATGGTTCTGGA |
| | | GTTATACTGCCTAGGTTCATATTCTGAAGCCTTCTGTTGTTTGGCCTTCTGCTGGGCAATGTTACGGATTCAGAGATGGGTCA |
| | | GCCTGGTTCCTGACTTCAAGGAGCTTCTAGTCTGATGAGATAGAAAGGCAGAGATATAAATAGCAGTAGTCCAACTTGATCA |
| | | AGAATGTGACAGGGATTATAGGATTCCAGAGGACAGTGTATTTGAGCTCAGACTTAAAAGAGTAGGCAAAGATCAGATCCTA |
| | | GGCAGTCTGATTCTGGAGGCTTTTCTCTTAAACATGCTGTCTTGGGTAACTGGATGGATGTAGTGACATTAGTTAGAATTGAA |
| | | AACAGGGAGAGGATTAAAGAGAATAGGAGATTAAAGGGAAATATGAGTTTAATTTTGGACATATTTCTAGATTCTTGTGGGAA |
| | | ATTCTGATAGATAGAATAATGGTCAGCCGGGCACGGTGGCTCACGCCTGTAATCCCAGCACTTTGGGAGGCCGAGGCTGGT |
| | | GGATCACGAGGTCAGGAGTTTGAGACCAGCCTGGCCAACATGGTGAAGCCCCGTCTCTACTAAAAATACAAAAATTAGCTG |
| | | GGTATGGTGATTGGCGCCTGTAATCTCAGCTACTCAGGAGGCTGAGGCAAGAGAATCGTTTGAACCTGGGAGGTGGAGTTT |
| | | GCAGTGAGCCGGGATCGTGCCATTGCACTCAAACTGGACGACAGAGTCTC |
| >ENST00000382938.3:: | 246 | GAAAGAAGACGTCCACGCTGCTGAGTGAGACCTTCCTCTGTGCTGCTGAGTGAGACCTTCCATCTGACCAGGGGGTCATGC |
| chr15: | | TCTCACTGCTCCTGCTTGGAGTTCTGGTGCTGTAGCGGGTCTCGGCCGCCCCTTCTGAGCTGGGTGGAGGAAGAAGTCCCT |
| 29362860- | | GTTGAAATATCAGATGAGTAGGGATGATCGCCTCTTTTGAAAACAGGAGCCGTGAAGGGATTCCCAGAGAAGATTGTCATCT |
| 29394962(+) | | AACGGAGTCATTCGTCCGCCCAGGACTTCTCTGTCACAGGGTTACGTTTGGGAGAATTTTCACAGGCCACTGGGGATGGCT |
| | | GTGGCTAGCCTGGCTTTCCACTGATGCCCTCTATCCCTAACCTCAGCTCCTGACATGGCTGTCATTCCAGAGAGTGCTTGGA |
| | | AGCATCCTGACTATGTTGACGATGGCCTGAGCGGAGTTTGCAATGGTCTGGAGCAGCCAAGGAAGCAGCAGCGCTCTGATC |
| | | TCAATGGACCTGTTGACAATAACAACATTCCAGAGACAAAGAAGGTGGCATCATTTCCAAGTTTTGTGGCTGTTCCAGGGCC |
| | | CTGCGAACCAGAAGACCTCATCGACGGGATCATCTTTGCTGCCAATTACCTGGGGTCCACCCAGCTGCTATCAGAACGGAA |
| | | CCCTTCCAAAAACATCAGAATGATGCAAGCGCAGGAGGCCGTCAGCCGGGTCAAGAATTCTGAGGGGGATGCCCAGACGC |
| | | TGACGGAAGTGGACCTCTTCATTTCCACCCAGAGGATCAAGGTTTTAAATGCAGACACGCAGGAAACCATGATGGACCACG |
| | | CCTTGCGTACCATCTCCTACATCGCCGACATTGGGAACATTGTAGTGCTGATGGCCAGACGCCGCATGCCCCGGTCAGCCT |
| | | CTCAGGACTGCATCGAGACCACGCCCGGGGCCCAGGAAGGCAAGAAGCAGTATAAGATGATCTGCCATGTGTTCGAGTCG |
| | | GAGGATGTAAGTAAGCCCTTGCCAGGGCACTCCCCTCCCAAAGTTCACAGCCCAGGGCGGCTCCAGGATCCAGGCGCTGT |
| | | GGAAACCACCCTCAGGTGGAAAGCCTCCATGCTGTTACTGATGTTTCCAGTGGATCAGTGATCTTTTGCATACTCTTTGGGTT |
| | | TGCAAAGATAGTGAATACAGTTTTATTCTACTTCTTGAAATAGGTTCTTCAGGAGCTGTTTATAAATTGAGTTGTGGTTAAATAT |
| | | ATGAGGGAGCTATTTGAAGAAATCCCTTTACAAAACATTTTCTCTACTAAAAATGAAGTTAATCTTTGCATAACTTTTGTTATTA |
| | | AAATGCAAATTTTCGCATGGCCCTGGCATGCTGTATAAAGAAAGCACATCTGCACATGAGGCTTAGTTCTGCCTTTGCGTGT |
| | | GGTCTTCAGAGGAAGTAAAAAGTGATTCTGAAGTATAAGATACCAAAGACTCAGGAAAAGATCACAAGCCCTTTGGCTCCCT |
| | | CCTTGGCTGGAGAAGAGTGTTGTTTTTAGCCTGGAGGGGGACAGAGGGGCTGAGGAAGGAGCAGCAGGGCCAAGAGGGG |
| | | AGCTCAGAGAGGAACTGTCCTTCCTGGAGGCTGATCTTACTCACAGACCAGCAGGGGGCGCTGCTGGTGAGCCAGTTTTGT |
| | | GGCTGTTGCCAGAGTGAAATTTTAAAATATGATCTATGGCTGGGCACGGTAGTTCATGCCTGTAATCCCAACACTTTTGGGA |
| | | GGCTGAGGTGCGTGGATCACCTGAGGTCAGGAGTTCAAAACCAGCCTGGCCAACATTGCGAAACCCTAGTCTCTACTAAAG |
| | | ATACAAAAAAATTAGCCAAGCTTGGTGGTGCGTGCCTGTAATCCCAGCTACGTGGGAGGCTGAGGCAGGAGAATTGCTTGA |
| | | ACCTGGGAGGCGGAGATTGCAGTGAGCTGAGATCGTGCCATTGCACTCCAGCCTGGGTGACAAGAGTGAAACTCCGTCTC |
| >ENST00000560864.1:: | 247 | AGGAAATTCCACTCATTGATAAAGCTCGGCTTCGACGCCCCTGCTCACAATGAAATACCTTCCTTGCCAACCACATTGCTTTA |
| chr15: | | TGATCGTGCCCCTTTACCACATTGTACTTGAAATGCCTTGTCATTTAAGAAACCATAAAAACGACGTCTTTAAAAATGGGTTCA |
| 61056938- | | TCATTTAGCTGGAGATGGCTTCTGTGCCTGGAACCAGAAGCCTGTCCTCTCGGTGGTATGCCTCCACAAAAGGAAGCGAGC |
| 61057824(+) | | TACCACAAAGGACAGCATCATGTGTTGCCTTCATTGCTATTGAATCGGGAAGTTGGTGCCCAGTTTCAACATGGATGACAAA |
| | | GTAGTGGGAATCTGAAATGCCTACAGTAGAAGCTATTTTTGAACGAGAAGGACTTTGGCTCAAATCAACAGTTTGGTTTCTTA |
| | | CTGTTTACATCTATGAGTCAAAGTAGCTTGCTCCCAGGATCCCCCACCAAACCGAATGAAAATAAATTCAATGTGTGCCCACT |
| | | GAGCCTGGCCTGGGAGTTCTTGAGATCAACTCAGAATCATGGTCTTTCTGTCACTCCCAAAACAATCCTACCTAATTCTACTT |
| | | GTTCCTAAGTATTGCCTTAGGATACTGACAGTCTTTTCTCCTACTTACAGCTTTTGAATGAAATTAAAGCTAATCCTCTCTGGC |
| | | TTGGTCGGGTCTTGTGTAAGATTCCCACACCCCACAATCAAGGCCTTGCAATGTGGTGTTAGAAGAGATAGCCAAGTTACCA |
| | | CACCTGTATCACGATTCGGATAAGGATAGTATTTTTTGCCTTCCATGAGACAGCTTTGAAA |
| >ENST00000568207.1:: | 248 | CCGCGATATTTGGGAGCGGCCCCGAGACGCGCCTGGCGCGGATCCTAAATCCCGACAGCTTTATAGAGCCCAGGCCTGGC |
| chr15: | | AGGCTCCCAGAACTTGAAGCCACCAGACCCCACATGGAACCAAAGGCCTCCTGTCCAGCTGCTGCACCCTTGATGGAGAGA |
| 75315926- | | AAATTCCATGTTCTTGTGGGTGTCACGGGGAGTGTCGCAGCCCTGAAGTTGCCTCTTCTGGTGTCAAAGCTTTTGGACATTC |
| 75337509(+) | | CTGGGCTGGAAGTAGCAGTGGTCACAACTGAGAGAGCCAAACATTTCTACAGCCCCCAGGACATTCCTGTCACCCTCTACA |
| | | GCGACGCTGATGAATGGGAGATATGGAAGAGCCGCTCTGACCCAGTTCTGCACATTGACCTGCGGAGGTGGGCAGACCTC |
| | | CTGCTGGTGGCTCCTCTTGATGCCAACACTCTGGGGAAGGTGGCCAGTGGCATCTGTGACAACTTGCTTGTGAGTGATGTC |
| | | CTGGTGCCCTCGTCCGTCCCTGGGCCTCACACCCAGTTTGCTGAGCTGCAGACATCCTTGTACAAGGAGACCTGCTGCTGT |
| | | GGGGCCCCGGTCTGCCCAGGGAGATCGTGCTCCGCCATTCCCCACATCGCCCCCAGTGCTCTCACCTTCTCTGTAGCACAT |
| | | GGGCACAGCCCTGGTCCTGATGGGTCAGTGTGACCTGCCACTGCTCCCCAGGAGTGTCCCCCTCTCCCCTGCCCTTGCAC |
| | | CCAGCCAGGTGCCCACAGGAAGACCCTTCTTCCTTGCTCTTTGTCCCTCCTCGTGGGCTGCCTGGGTGTGCTCTTCCACTG |
| | | TCTCTGCCCAGCCCCATCGCCTCCCGCTTCAGCTCTGCCTTCTCCGCAGATCTGGGTATACACGTGGGCAATGTAATTGGG |
| | | GGTTCACTTCTTCATAGATGGCTCATATACACTTTATCACCCTTGTAGCTTTCCTGTATATTAGGGGAAGCTTAGAGGGGAAG |
| | | AGGGGAGAAAGACGGGGCAGTGGCGAGAGCCGATGGGTGGTAGGGAGAAATGGAAGTCCAGCTGTCCAAAGTGGGCACT |
| | | GTCCTAGGCCTAAGATGCCTCCAGGGAGCTGTTGCCCTTAAAGGGGAGCTGCCTTTGTTTATATGATCGTCCTGGG |
| >ENST00000561323.1:: | 249 | GCCCGCCCCGTCCGCTAAGTGCCTGGGCTCTCCCGCTCGCGTCCCAGTCTGCGGGCCTCCGGGGCAGCGGCGAGGCCG |
| chr15: | | GAGCGTCGCGGCGGAGAGGACGAGACCGGGACAAGACCAGGGCAGGAGGGAGCCGGCCAGCCGCGAGAACCCCGCACG |
| 99645240- | | CCCGGCAAGATGCTGTCCTGGCGGCTGCAGACGGGCCCCGAGAAGGCCGAGCTCCAGGAGCTCAACGCCCGGCTCTATG |
| 99675794(+) | | ACTACGTGTGTCGGGTGCGGGAGCTGGAGCGCGAAAACCTACTCCTGGAGGAGGAGCTGCGCGGCCGGCGCGGGCGAGA |
| | | GGGCCTGTGGGCCGAGGGGCAGGCCCGCTGCGCCGAGGAGGCGCGCAGCTTGCGGCAGCAGCTGGACGAGCTGAGCTG |
| | | GGCCACTGCGCTGGCGGAGGGCGAGCGGGACGCTCTGCGGCGCGAGCTGCGGGAGCTGCAGCGCCTGGATGCGGAGGA |
| | | GCGCGCCGCCCGCGGCCGCCTGGACGCCGAGCTGGGTGCGCAGCAGCGCGAGCTGCAGGAGGCGCTGGGCGCGCGCG |
| | | CCGCCCTCGAGGCGCTGCTGGGCCGGCTGCAGGCCGAGCGCCGAGGCCTCGACGCGGCCCACGAACGCGACGTGAGGG |
| | | AGCTGCGCGCGCGCGCCGCCAGCCTTACCATGCATTTCCGCGCCCGCGCCACCGGCCCCGCCGCGCCGCCGCCACGCCT |
| | | GCGGGAGGTGCACGACAGCTACGCACTGCTGGTGGCCGAGTCGTGGCGGGAGACGGTGCAGCTGTACGAGGACGAGGTG |
| | | CGCGAGCTGGAGGAGGCGCTGCGGCGCGGCCAGGAGAGCAGACTCCAGGCGGAGGAAGAGACGCGGCTGTGCGCGCAG |
| | | GAGGCAAGGCGCTGCGGCGCGAGGCGCTCGGGTTGGAGCAGCTGCGCGCGCGGCTGGAGGACGCGCTGCTGCGGATGC |
| | | GCGAGGAGTACGGGATACAGGCCGAGGAGCGGCAGAGAGTGATTGACTGCCTGGAGGATGAGAAGGCAACCCTCACCTTG |
| | | GCCATGGCTGACTGGCTGCGGGACTATCAGGACCTCCTGCAGGTGAAGACCGGCCTCAGTCTGGAGGTGGCGACCTACCG |
| | | GGCCTTATTGGAAGGAGAAAGTAATCCAGAGATAGTGATCTGGGCTGAGCACGTTGAAAACATGCCGTCAGAATTCAGAAAC |
| | | AAATCCTATCACTATACCGACTCACTACTACAGAGGGAAAATGAAAGGAATCTATTTTCAAGGCAGAAAGCACCTTTGGCAAG |
| | | TTTCAATCACAGCTCGGCACTGTATTCTAACCTGTCAGGGCACCGTGGATCTCAGACGGGCACATCTATTGGAGGTGATGCC |
| | | AGAAGAGGCTTCTTGGGCTCGGGATATTCTTCCTCGGCCACTACCCAGCAGGAAAACTCATACGGAAAAGCCGTCAGCAGT |
| | | CAAACCAACGTCAGAACTTTCTCTCCAACCTATGGCCTTTTAAGAAATACTGAGGCTCAAGTGAAAACATTCCCTGACAGACC |
| | | AAAAGCCGGAGATACAAGGGAGGTCCCCGTTTACATAGGTGAAGATTCCACAATTGCCCGCGAGTCGTACCGGGATCGCCG |
| | | AGACAAGGTGGCAGCAGGTGCTTCGGAAAGCACACGGTCAAATGAGAGGACCGTCATTCTGGGAAAGAAAACAGAAGTGAA |
| | | AGCCACGAGGGAGCAAGAAAGAAACAGACCAGAAACCATCCGAACAAAGCCAGAAGAGAAAATGTTCGATTCTAAAGAGAA |
| | | GGCTTCCGAGGAGAGAAACCTAAGATGGGAAGAATTGACAAAGTTAGATAAGGAAGCGAGACAGAGAGAAAGCCAGCAGAT |
| | | GAAGGAGAAGGCTAAGGAGAAGGACTCACCGAAGGAGAAGAGCGTGCGAGAGAGAGAGGTGCCGATTAGTCTAGAAGTAT |
| | | CCCAGGACAGAAGAGCAGAGGTGTCCCCGAAAGGTTTGCAGACGCCTGTGAAGGATGCTGGTGGTGGGACCGGTAGAGAG |
| | | GCAGAAGCAAGAGAGCTACGGTTCAGGTTGGGCACCAGTGATGCCACTGGTTCTCTGCAAGGCGATTCCATGACAGAAACC |
| | | GTAGCAGAAAACATCGTTACCAGTATCCTGAAGCAGTTCACTCAGTCTCCAGAGACAGAAGCATCTGCTGATTCTTTTCCAGA |
| | | CACAAAAGTCACTTACGTGGACAGGAAAGAGCTTCCTGGGGAAAGGAAAACAAAGACTGAAATAGTTGTGGAGTCTAAACTG |
| | | ACTGAGGATGTTGATGTTTCCGATGAAGCTGGCCTGGACTACCTTTTAAGCAAGGATATTAAGGAAGTGGGGCTGAAAGGCA |
| | | AGTCAGCCGAGCAGATGATAGGAGACATCATCAACCTCGGCCTGAAAGGGAGGGAGGGGAGAGCAAAGGTCGTCAACGTG |
| | | GAGATCGTGGAGGAGCCCGTGAGTTATGTCAGCGGGGAGAAGCCGGAGGAGTTTTCCGTCCCATTCAAAGTGGAGGAGGT |
| | | CGAAGATGTGTCGCCAGGCCCCTGGGGGTTGGTTAAGGAGGAGGAAGGTTATGGAGAAAGCGATGTCACATTCTCAGTTAA |
| | | TCAGCATCGAAGGACCAAGCAGCCTCAGGAGAACACGACTCACGTGGAAGAAGTGACAGAGGCAGGTGATTCAGAGGGCG |
| | | AGCAGAGTTATTTTGTGTCCACTCCAGATGAACACCCCGGGGGGCACGACAGAGATGACGGCTCGGTGTACGGGCAGATC |
| | | CACATCGAGGAGGAATCCACCATCAGGTACTCTTGGCAGGATGAAATCGTGCAGGGGACTCGAAGGAGGACACAGAAGGA |
| | | CGGTGCAGTGGGCGAGAAGGTTGTGAAGCCCTTGGATGTCCCAGCGCCCTCTCTGGAGGGGGACCTGGGTTCCACTCACT |
| | | GGAAAGAACAAGCTAGAAGCGGTGAATTTCATGCCGAACCCACAGTCATTGAAAAAGAAATTAAAATACCCCACGAATTCCA |
| | | CACCTCCATGAAGGGCATCTCCTCCAAGGAGCCCCGGCAGCAGCTGGTGGAGGTCATCGGGCAGCTGGAGGAAACCCTTC |
| | | CCGAGCGCATGAGGGAGGAGCTGTCCGCCCTCACCAGAGAGGGGCAGGGTGGGCCGGGGAGCGTTTCCGTGGATGTCAA |
| | | GAAGGTCCAGGGTGCTGGTGGCAGTTCCGTGACCCTGGTTGCTGAAGTCAACGTCTCACAAACTGTGGATGCCGATCGGTT |
| | | AGACCTGGAGGAGCTGAGCAAAGATGAGGCCAGTGAGATGGAGAAGGCTGTGGAGTCGGTGGTTCGGGAGAGCCTGAGC |
| | | AGGCAACGCAGCCCAGCGCCTGGCAGCCCAGATGAGGAAGGTGGAGCGGAGGCCCCGGCTGCTGGCATTCGCTTTAGGC |
| | | GTTGGGCCACCCGGGAGCTGTACATCCCTTCAGGCGAGAGCGAGGTTGCTGGTGGGGCCTCTCACAGCTCGGGACAGCG |
| | | CACTCCCCAGGGCCCAGTGTCGGCCACTGTGGAGGTCAGCAGCCCCACAGGCTTTGCCCAGTCACAGGTGCTGGAGGATG |
| | | TGAGCCAGGCTGCAAGGCACATAAAACTCGGCCCCTCTGAAGTCTGGAGGACTGAGCGAATGTCATATGAAGGACCCACTG |
| | | CAGAAGTGGTGGAGGTAAGTGCGGGAGGTGACCTAAGTCAGGCAGCGAGCCCGACCGGAGCCAGCCGGTCTGTGAGGCA |
| | | TGTCACGCTGGGTCCCGGTCAAAGTCCACTGTCCAGAGAAGTCATCTTCCTAGGCCCTGCCCCTGCCTGTCCAGAGGCATG |
| | | GGGCTCGCCAGAACCTGGCCCAGCAGAGTCTTCTGCAGATATGGACGGATCAGGGAGGCACAGCACATTTGGCTGCAGAC |
| | | AATTTCATGCTGAAAAGGAGATTATTTTTCAGGGCCCCATTTCTGCTGCAGGGAAGGTTGGTGATTATTTTGCAACAGAAGAG |
| | | TCAGTGGGTACCCAGACTTCTGTCAGGCAACTCCAGTTAGGCCCTAAAGAAGGGTTCAGTGGGCAAATCCAGTTCACAGCT |
| | | CCACTTTCAGACAAGGTGGAGTTGGGTGTCATAGGAGATTCTGTACACATGGAAGGGTTGCCAGGGAGCAGCACATCCATC |
| | | AGGCACATCAGCATTGGGCCTCAGAGGCATCAGACCACCCAGCAGATAGTTTACCATGGGCTGGTTCCCCAACTGGGGGAA |
| | | TCTGGTGACTCAGAGAGCACTGTGCACGGAGAGGGCTCAGCAGATGTGCACCAGGCCACTCACAGTCATACCTCGGGTAG |
| | | ACAAACCGTTATGACTGAAAAGAGCACCTTCCAAAGTGTCGTTTCTGAATCTCCCCAGGAGGATAGTGCAGAGGACACATCA |
| | | GGGGCAGAAATGACATCGGGTGTTAGCAGATCCTTTAGGCACATTCGACTAGGTCCTACAGAAACGGAAACCTCTGAACAC |
| | | ATTGCCATCCGTGGACCCGTGTCCAGAACATTTGTGCTTGCTGGTTCAGCGGACTCCCCTGAGCTAGGCAAGTTAGCAGAC |
| | | AGCAGCAGAACGCTAAGGCACATTGCACCAGGGCCCAAAGAAACTTCGTTTACCTTTCAGATGGATGTGAGTAACGTAGAG |
| | | GCGATCCGCAGCCGGACACAGGAAGCGGGAGCTCTCGGTGTGTCTGACCGTGGTTCCTGGAGAGACGCGGACAGTAGGA |
| | | ATGACCAGGCAGTTGGTGTGAGCTTTAAGGCCTCTGCTGGGGAAGGAGACCAGGCCCACAGAGAACAGGGCAAGGAGCAG |
| | | GCCATGTTTGATAAGAAGGTGCAGCTCCAGAGAATGGTAGACCAAAGGTCGGTGATTTCAGATGAAAAGAAAGTTGCCCTCC |
| | | TCTATCTAGACAATGAGGAGGAGGAGAATGATGGGCATTGGTTTTAATAAGCAGAAACATTTTGTTTTAATGGCAGCCTGTTG |
| | | GCGACGTGCCAACATCCAAAGGCCTTAACTTATTTTAAGAGGCCGAGGGAGTCTATGAAAATCTCCCCTTTTTTACTTTTTTA |
| | | AAGAGTACTCCCGGCATGGTCAATTTCCTTTATAGTTAATCCGTAAAGGTTTCCAGTTAATTCATGCCTTAAAAGGCACTGCA |
| | | ATTTTATTTTTGAGTTGGGACTTTTACAAAACACTTTTTTCCCTGGAGTCTTCTCTCCACTTCTGGAGATGAATTTCTATGTTTT |
| | | GCACCTGGTCACAGACATGGCTTGCATCTGTTTGAAACTACAATTAATTATAGATGTCAAAACATTAACCAGATTAAAGTAATA |
| | | TATTTAAGAGTAAATTTTGCTTGCATGTGCTAATATGAAATAACAGACTAACATTTTAGGGGAAAAATAAATACAATTTAGACTC |
| | | TAAAAAGTCTTTTCAAAAAGAAATGGGAAATAGGCAGACTGTTTATGTTAAAAAAATTCTTGCTAAATGATTTCATCTTTAGGAA |
| | | AAAATTACTTGCCATATAGAGCTAAATTCATCTTAAGACTTGAATGAATTGCTTTCTATGTACAGAACTTTAAACAATATAGTAT |
| | | TTATGGCGAGGACAGCTGTAGTCTGTTGTGATATTTCACATTCTATTTGCACAGGTTCCCTGGCACTGGTAGGGTAGATGATT |
| | | ATTGGGAATCGCTTACAGTACCATTTCATTTTTTGGCACTAGGTCATTAAGTAGCACACAGTCTGAATGCCCTTTTCTGGAGT |
| | | GGCCAGTTCCTATCAGACTGTGCAGACTTGCGCTTCTCTGCACCTTATCCCTTAGCACCCAAACATTTAATTTCACTGGTGGG |
| | | AGGTAGACCTTGAAGACAATGAAGAGAATGCCGATACTCAGACTGCAGCTGGACCGGCAAGCTGGCTGTGTACAGGAAAAT |
| | | TGGAAGCACACAGTGGACTGTGCCTCTTAAAGATGCCTTTCCCAACCCTCCATTCATGGGATGCAGGTCTTTCTGAGCTCAA |
| | | GGGTGAAAGATGAATACAATAACAACCATGAACCCACCTCACGGAAGCTTTTTTTGCACTTTGAACAGAAGTCATTGCAGTTG |
| | | GGGTGTTTTGTCCAGGGAAACAGTTTATTAAATAGAAGGATGTTTTGGGGAAGGAACTGGATATCTCTCCTGCAGCCCAGCA |
| | | CCGAGATACCCAGGACGGGCCTGGGGGGCGAGAAAGGCCCCCATGCTCATGGGCCGCGGAGTGTGGACCTGTAGATAGG |
| | | CACCACCGAGTTTAAGATACTGGGATGAGCATGCTTCATTGGATTCATTTTATTTTACACGTCAGTATTGTTTTAAAGTTTCTG |
| | | TCTGTAAAGTGTAGCATCATATATAAAAAGAGTTTCGCTAGCAGCGCATTTTTTTTAGTTCAGGCTAGCTTCTTTCACATAATG |
| | | CTGTCTCAGCTGTATTTCCAGTAACACAGCATCATCGCACTGACTGTGGCGCACTGGGGAATAACAGTCTGAGCTAGCACCA |
| | | CCCTCAGCCAGGCTACAACGACAGCACTGGAGGGTCTTCCCTCTCAGATTCACCTGGAGGCCCTCAGACCCCCAGGGTGC |
| | | ACGTCTCCCCAGGTCCTGGGAGTGGCTACCGCAGGTAGTTTCTGGAGAGCACGTTTTCTTCATTGATAAGTGGAGGAGAAA |
| | | TGCAGCACAGCTTTCAAGATACTATTTTAAAAACACCATGAATCAGATAGGGAAAGAAAGTTGATTGGAATAGCAAGTTTAAA |
| | | CCTTTGTTGTCCATCTGCCAAATGAACTAGTGATTGTCAGACTGGTATGGAGGTGACTGCTTTGTAAGGTTTTGTCGTTTCTA |
| | | ATACAGACAGAGATGTGCTGATTTTGTTTTAGCTGTAACAGGTAATGGTTTTTGGATAGATGATTGACTGGTGAGAATTTGGT |
| | | CAAGGTGACAGCCTCCTGTCTGATGACAGGACAGACTGGTGGTGAGGAGTCTAAGTGGGCTCAGTTTGATGTCAGTGTCTG |
| | | GGCTCATGACTTGTAAATGGAAGCTGATGTGAACAGGTAATTAATATTATGACCCACTTCTATTTACTTTGGGAAATATCTTGG |
| | | ATCTTAATTATCATCTGCAAGTTTCAAGAAGTATTCTGCCAAAAGTATTTACAAGTATGGACTCATGAGCTATTGTTGGTTGCT |
| | | AAATGTGAATCACGCGGGAGTGAGTGTGCCCTTCACACTGTGACATTGTGACATTGTGACAAGCTCCATGTCCTTTAAAATC |
| | | AGTCACTCTGCACACAAGAGAAATCAACTTCGTGGTTGGATGGGGCCGGAACACAACCAGTCTTTTTGTATTTATTGTTACTG |
| | | AGACAAAACAGTACTCACTGAGTGTTTTTCAGTTTCCTACTGGTGGTTTTGATATTGTTTGTTTAAGATGTATATTTAGAATGA |
| | | CATCATCTAAGAAGCTGATTTTGCTAAACTCCTGTTCCCTACAATGGGAAATGTCACAAGAATGTGCAAAAATAAAAATCTGA |
| | | GGAAAAAA |
| >ENST00000602784.1:: | 250 | GCGGCCTTTGGTGCCCACGACTTCCATCGTATCTACATCAGCTGAACCTGAAACTCAGGGGACTCTGGAACACCATGGACC |
| chr16: | | CTGGGGGCTGTGCCCCCATCTCCTCCCCACCCCAGGTCAGAGCTGCAGCCTAGGGGGCACTGCCCTACAGAAAAGGTCTG |
| 30785390- | | CCTGAGAGGCCTGAGGAGCCCAGAGCACTTGACTGAGCTTCCCGGAAACTGGCCCTAACCTGTCTGTCTCCGTGGATGCAT |
| 30786509(+) | | CCTAACCCTAAGGAAAATTCCCCAGGCTGTGATCTACCCTAGAGAAGGCTCGCTCCCTGCCTACTGGCTCACAAATGAGGA |
| | | CCAGTGAGCCATGTCCTTGTTCCTTGTTTGAGACTGGGCTGCAGGCCCCAGGAAGACTTTCCTTCACCCACCATCCCCCTAA |
| | | CCTCGGCAGGGCTTCTGTCCTGTGGAGTTCCCTGGACACCTTGGTCTGGCTCTTGTGCCAAGGGCTGAAGGAGGTACCCTC |
| | | TTGGCAGATGGGGGCATCACTTGCTTCCTTTGGGAAGCTCTAAGGTTGCTGCAGTCACCTTCCTCATCTTGCAGGTGCTGAA |
| | | CCAACATCATCAGTTTCTATTCTAATCAGGCCCCTTCCCAATCTCCATTTCTCTGCCAAGCCCATTTACCCCCACCTCATGCA |
| | | TCCCAAGGCTCTACTGGGTCCCTGGACCTAACCCTGCTTTCATCCTGGTGGCCTTAACTACAGTGGAGGTGGAACTTCCCA |
| | | GGAGGGGAAGGGACAGACCAGCCCCAGCCGCTGGGCCAACTTCCAATCATTCCAGCTAGAAGAGCTTCCCCCTGACACCC |
| | | TGTGACTGAGCCTGTGTCCTGTCTGCCTGCCCAGCCATGCTCCATCGGCTGTGAGGGCAGTGCCCGGAGAGGCCAGAGGG |
| | | TTGGAGCTGCAGGGACCCGTTTGGACCCACAGCCTCTGTTCTAGAGATGCTTGTATAGGCTGTTAATTGTGATGAATAAACG |
| | | TTCAACCCTCGGCCTGC |
| >ENST00000574306.1:: | 251 | AGCTGATCGGAGCCTGGAGCCGGTGTGTGCTGGGTGCCGAGAAGAGACAGCGCCGCCGGCCGTGGGGAGCGGACGCAG |
| chr17: | | TGATTTGCTCCCCCTCGTGCAGCAACCCCCACACCCAGCACCAGGAGCCTGTTCCTCTCACGCCCTCACCTGGCTGAGCCG |
| 1614804- | | CAGTAGTTCTTCAGTGGCAAGCTTTATGTCCTGACCCAGCTAAAGCTGCCAGTTGAAGAACTGTTGCCCTCTGCCCCTGGCT |
| 1619545(−) | | TCGAGGAGGAGGAGGAGCTGCTTTCCCCATCATCTGGAAGGTGACAGAAATGGGCTGGGAAGGTCCGAACAGCAGGGTGG |
| | | ATGATACGTTTTGGGCAAGTTGGAGAGCCTTTGCCCAGATTGGCCCAGCAAGGAGCGGTTTTAGATTAGAGACACTGGCTG |
| | | GATTGAGGAGTAGAAGGCTCAAACAACCCAAGGTTAGTTGGTCTTTGTGTGACAGTGGGAAGAAGTGGAGAGAACACTCTG |
| | | TCTCCCCGACTTCCTTCTTGACCCTCCTTCCGTATCTTTCATTCTTCCCCACTGGCCTTTCCAGATGAGGTCCAAAAGTAAGC |
| | | TCACTCTTCAGGGAAAAGTGATACTAAACCCAGGCGTGAGGAGTCATCCTTCTGCTCTTACTTTTCCCTGCATTTCCTTAGGC |
| | | TGGCCCAGACTCTAGATTCCTGGGCACTTACCTGTATCTGAGACCCTTAGACCTCCTGTCTCAGTTTCTCCACCTGTGAGAT |
| | | GGGCAGTCACACAGATGAGTGTGCATGAGGGAATCAGAAGCCCATCTGGGTTGACTCAGAACCCGGGCTCTCCCCCTGCT |
| | | GTGGGTGGAAACAAAACACCTTTTCACAGAAGCTGTTGTCCCTCCCCCATCCCTGATACCATCACCCAGAAGCTGGGTGGG |
| | | GAATGGGAAGCTGGAGGAGGGGGTTAGAGTTAGACCAATGCGAGGAGCTAGTAAGAGCTCGCCTCTCCGTGACTTCCCCC |
| | | CATGCAAGGGTGGCCCAAGGCTTCCCCTGATATCCAAAGCAGGACAGCAGCCCCTTGGTGGGATTCTAACTGCTGCTACTC |
| | | TGTTTCTTTCCTCACTTTGCTTTCCAAGGTGGTATGTGATCCCCAGCTCAGGCCTGTGCAGACAGGAAATTCTCCCCTGCAG |
| | | CAAGTAGGGGAGGTGGGTTGTGGGATGTGACCTCCTTCCAGATATCAGGCAGTGAGTGTAAACCTGCCACCTCCAGCCCTG |
| | | ATCCATTCTCACCTAGCGGCTACAGGAAGCTGTGTCTGTTCGATTTGGTGGGAGGAGATGTGCAGGGAGCTGTATCTTGTC |
| | | CTCCGCTTGTGAAAAACTCAAGGATGTGGAGAAGAGTAGACCGTGGAACCCTGCTCTTCTGCAGCCAAGCTGAGGGGCAGG |
| | | ATGCGTGTGGGACAGTGGTAGAGAAGCAGGGGATAGACTCATAGGCTGCAACAAAGGTGACTCTGTCCCTGGACACTGCCT |
| | | CCGTACTTTCTCCTTGCTTCACTGGCCACAGCATCTCCCTCCAGCCCTCGCTATGTGCCTCTGCCATCTTCACCCATCATGG |
| | | AGCAGAGGTGAGGAGAGGCAGCCTGGGAATATGGAGACCAGTGAAGGACCAGGCCTGGAGAGCACAGGGTCCTACCTGG |
| | | GCATCCAGCAGAGGAGCCCCTAAAGGCCAGGAGCACCCCAAGAGGAGGGAGGGCAGCCAGCCTCCATTGACGGCGAGCC |
| | | TCCAGCCCTCTCCTACTTTGATCACCATTTCTCTCCAGGCTTTCTGCCTCCGAGATGTGGCACCATAGTGCGGTGCCCTGTG |
| | | GCTTCACCGCCCTACTTCCACCTCCGCCCAGCCTGTAATGTTTATATAAGCAGCCTCAAGGACCAAGAACCATCTGCGAAAG |
| | | GACACACACAGGAAATTCATAAAAGAAATCTGAATGGATAAAACCATGAAAAAAAGTATGCTTCATTAGTAATTAAAGAAAGG |
| | | CAAATAGAGCTGGAAGCATTTTTCCCTTAGCAAACCATAACAGAAAAAAATAAGACCCAATATTGGCAAAGAGACTACTGAAA |
| | | AAACATTCCCATACATTGCGTGTGGGAGTATACATCGGTGCAGGCTTCCTGGATGACAGTTGGGTGATATGTGTCATGTGGC |
| | | CTAAAAGCCTCCATGTCATTTGACCTACGAATTCTATCTTTGGGAATTTATCCTAAGAAAATACTTAAGGATTTAGTTAGTGAT |
| | | AAGATGTTCATCCCAGCATTGCAATGGAGAAAAATGGGAAGCAATGGTTTGGTTGGGAATTTATTCCTTTTCTGCTGTAACGA |
| | | AAGTTTGCAATAGGGGATTGCTTAAGTAAATTATTGTATCTCCATCCAGATGGTGGAGTACCGCGCAGACATTAAAAGTCATG |
| | | TAAAAGAACATCTGACTGAAAGAAAAATGCTCCTTGAATATTAAAAGGTTGTAAAAATAGTGCATGTTATGTGATTTCAATTTT |
| | | GTTTTTTAAAATATGGGTGTATGCTTGTATACGTAGAGCAGATAAAAAAGACGGAAGGCATACTAAAAAATGTTGAGTGGTTA |
| | | TCTTTGTATGGTGGAACAAAGTCACTGTAATTTTCATCTTTGGTTTTTCTGTAATTTCCAAATTTTCCACATTTTGTATTTCATAT |
| | | AATATAATTTAA |
| >ENST00000413077.1:: | 252 | GGCTTCCGTTCGCGGACTTTGGCCCAGGCGCGTGGGCGCTGCCGAGAGGCTGGGAGCCCGGGAACCGGTTCCTGGACTT |
| chr17: | | GAGCGGAGCGAGAAAGAGCGCGCCGCCTGCACGACTGGACTGGAGCGGCCGGGTGCCCTGGAGGCCCCTGGGAATTGC |
| 5015226- | | AGTCCTTGCGCAGGTGGAGAGTGAGCCCTTATGAGAAAACTCAAGACTCAGAAACAATAAAGGTTACAGAGATGAAATTGAC |
| 5017672(+) | | AGAGCCTGAATTTCAGGTTATGATTCTGTATGGTTTCTTTGGTGCTGCATTAGACCTGAGCTCTGATTGAAAATTCTTCCACG |
| | | CTCGCTACATTCCAAAACTTTATCTCCAATATGAATTTCCAGATGCCTCACAAGGTCTGAGCTCCATCTGAAGGCTTTCCCAT |
| | | GCTTGTCACATTCATAGGGCTTTTCTCCACTATGAATTCTATGATGTCGAATAAAGTGTGAGCTCTGAT |
| >ENST00000568641.1:: | 253 | GGCTGGGGGCGGAAGGAGGAGCCAGGCGAAGCGGCGCCTCAGCTGAGAGGACCGGCGGACCCTGCAGAGGCCCCCTGC |
| chr17: | | CCCTCTGGCTCCGCCCCCACCCGGGTCGCTAGAAATACAGCCGTAGCCCCGCCCACCGCCCACTGCGCTCTGACCCAGAC |
| 5402758- | | CCGGCTGACCCACCTACCCGCGATCCTGCCCATGGCTGACGGGCTCTTTCGGCGCAGACCCTGGGGTCTCGAGCAGATTC |
| 5404465(−) | | GCCCGGACCCCGAGTCCGAAGGCCTGTTTGACAAGCCTCCCCCGGAAGACCCTCCCGCTGCCCGCGGGCCCAGGTCGGC |
| | | GTCGGCCGCGGGCAAGAAGGCTGGTCGGCGCGCGGGCGGGAGGGCGCAGGGGGGCCGCGCCGGGCAGCCCCCGAAGG |
| | | CCGCATCGCGCCCCCCGCCCAAGAAGGAGGCGCCTCCACTGGACGAGGGCTGCTATCTCGACCATTTTCCGCACCTCTCC |
| | | ATCTTCATCTACGCAGCCATCGCCTTCTCCATCACCTCCTGCATCTTTACCTATATCCATTTACAGCTTGCCTGAGTGGCCAG |
| | | CGCGGGACGGGGTGGGCGCAGGACCGAGCGGGGAGGGAAAGGGAAAACGGGGCTCGGCATTTTGTGTTTTAGAACAGCG |
| | | CTGCACCCCCTTCATGTAGCTTTCGATGCTTGTTTCTTTCCGTCTTTGTTGTCACTATCTTTGTCTATCAGTACGAAAGTACAA |
| | | AGTAGCTGCCGGCAATGAAATAGGGGTGCTGTTTGCACCTGCAGGTTAGGGGTGGAGGCGTTTAGAATTTTGGGGTGTGAT |
| | | TGAGCCCCGTTTATAATTAGAATGCCCCTGGACCCCTACCACTCTGTGACGTGGGGGCACGCGCAGGGATCCCATCATTTT |
| | | GTGTTTGGGGAGCTCAGAGTGCGCCCAATCTTGGAATCTTTAAGGGATGAGCCAGACCCAGACCCGCGGCCTTCTAGAGAG |
| | | GGTCCGGCAGGGAGGGTCGGCGCCCTGGCCCGGGGTGGGCCGGAGCCCTGTGATGCTGCATCGCCCCCAGGAGGAGCC |
| | | AGCTGTGCCCCAGAGTTGGCGCGGCCGAGAGAGGACAAGAGCGCGCAGCAGGCGAAGCTGGAGGGGGGACTCGACTTT |
| | | GTTGTCGCTGCCCGGAGGAGTCGAGACTGGTACCCGGAGGAGCTGTCTCACCAGGAGACCACGTCCTGGAAGTGTCCGGG |
| | | ACTCGCGGGACCTGTGGCTGCAGACCCCGCCGGCACGCAGGCCCAGAGCTGGCGCACTCCTGAGGATGAGACTCTGGGG |
| | | GCCCTAGCCGGGGTCCACGGGAGGGCTGTCCTTGGGGACTCTAGGATGGCTTCGTTCTGGCCCGGCTCACTTCTGGAGCT |
| | | GTGAGACCCAAGACAAAAGGGGCTGAGGGATTTCTCATTGACAAGAGTTCGTGCGGGAAAACCACCTGATCCCTAGGGATT |
| | | TGTCATCTTAAGACTCAAAAGGCTTAATACCAGGAACCACCTTGGCAAGATATTTACCCACCGGCCATCTCTGTTTACTCATG |
| | | AATGTTAAATGTTAAAACGCAGCGCTCTAACCCTGCATATTATTTACTTGCAAATGTCTGTAATCTGTAATTGTGATGCCTCTG |
| | | ATGGAATAAATTATCTTTTT |
| >ENST00000566166.1:: | 254 | GCCGTGGGGCGGCAGAGCGGGTGGGAAGGACGCCTGGGAGCTGGACCCAGTCTCAGCGTGGCACTTCCCACAGGGCCG |
| chr19: | | CCCAAGAGTGTCCCCGGCCGTGCAGTGCGCCCTGAGCCTCCCGCGCCGGCCCCCGCGGCCCTGGAACCCGCGCCGGTG |
| 7982512- | | GTGGCGCTGGTGTTGGCAGCCTTCGTGCTGGGCGCCGCGCTGGCCGCCGGGCTGGGTCTCGTCTGTGCGCACTCAGCGC |
| 7983975(+) | | CCCACGCCCCTGGCCCGCCCGCGAGAGCCTCGCCCAGCGGTCCCCAGCCCAGGAGGTCCCAGTGAGGAAGGGATGGTGC |
| | | GCCCCCAACATGGTCCGGAGATACACCCAGCTACCAATTCGGGACCAGGACCAACAGGACCGGACCCGCCTCCCTGGACC |
| | | TCGGACCTGATGAGGCCACGACCCCTGCGCTTCTCTCCTCCCCCTGTCCCTCCCACCTGTGCTCAAAATAAACCTCTGGACT |
| | | GAC |
| >ENST00000462995.1:: | 255 | GCTCCCGCTCCTGGCTCTGTAGCTGAGAGAAGCCCTGGCAGGTCAGTGGCAGGCACTGTCACGCTGAGTCCTATGCTGGC |
| chr19: | | AGCGGGGAACCTTGGGGAGAACACGGGACACCGCGGAAGCCGGGAAATGGATTCAGTAGCCTTTGAGGATGTGGCGGTG |
| 12462604- | | AACTTCACCCAGGAAGAGTGGGCTTTGCTGGGTCCATCACAGAAGAGTCTGTACAGAGATGTGATGTGGGAAACCATTAGG |
| 12476475(−) | | AACCTGGACTGTATAGGAATGAAATGGGAAGACACAAACATTGAAGATCAGCACAGAAATCCCAGGAGGAGCCTAAGGTAAT |
| | | TTGCACTCACAAGAGAAAGACATATATCTGTGGAGCAATTCTTAGGATGACAGGAAATTATACAACTAGGCAAAGAAAATGAA |
| | | CGAGCCCAGTACAAACTTATTTATTCCTATACAATTTTCTCCAGAAACATATACTTAAATGTGATATAACTATTCAGTGTTTGCA |
| | | AAAATAGTTCCCTTAGAAACAATATTAAGAATTCGGCTGGGCACGG |
| >ENST00000587837.1:: | 256 | ACTTGCCTGGACGCTGCGCCACATCCCACCGGCCCTTACACTGTGGTGTCCAGCAGCATCCGGCTTCATGGGGGGACTTG |
| chr19: | | AACCCTGCAGCAGGCTCCTGCTCCTGCCTCTCCTGCTGGCTGTCTCCGTCCTGTCCAGGCCCAGGCCCAGAGCGATTGCA |
| 36395302- | | GTTGCTCTACGGTGAGCCCGGGCGTGCTGGCAGGGATCGTGATGGGAGACCTGGTGCTGACAGTGCTCATTGCCCTGGCC |
| 36399197(−) | | GTGTACTTCCTGGGCCGGCTGGTCCCTCGGGGGCGAGGGGCTGCGGAGGCAGCGACCCGGAAACAGCGTATCACTGAGA |
| | | CCGAGTCGCCTTATCAGGAGCTCCAGGGTCAGAGGTCGGATGTCTACAGCGACCTCAACACACAGAGGCCGTATTACAAAT |
| | | GAGCCCGAATCATGACAGTCAGCAACATGATACCTGGATCCAGCCATTCCTGAAGCCCACCCTGCACCTCATTCCAACTCCT |
| | | ACCGCGATACAGACCCACAGAGTGCCATCCCTGAGAGACCAGACCGCTCCCCAATACTCTCCTAAAATAAACATGAAGCACA |
| | | AAAACA |
| >ENST00000602172.1:: | 257 | ACTTTGTCGTACTCCTTCCTTGCTGACTAAGAGGAACAGAACACAGAGCAGCCTGGCGGTGTCCTACCAACAAGCCTCCGTT |
| chr19: | | TCTCCTTCCTGTACACTAGGGCTCCTGAAACTCACCTGATGAAGTCTCCGTCTGTCACCCAGGCTGGAGTGTAATGGAGCAA |
| 48758931- | | TCTCGGCTCACTGCAACCTCTGCCTCCCAGGTTCAAGCGATTCTCTTGCCTCAGCCTCCCGAGTAGCTGGAATTATAGGTGC |
| 48761456(+) | | ATGCCACCACGCTCGGCTAATTTTTTGTATTTTTAGTAAAGACGGAGTTTCACCATGTTGGTCAGGATGGTCTCCGTCTCTTG |
| | | ACCTCGAGATCAGCCCGCCTCGGCCTCCCAAAGTGCTGGGATTACAGGCGTGAGCCACTGTGCCCGGCCTTGGTTTCTAAA |
| | | TACTATTTTGAAGTATCTGTGGAGAAATGGTTACTTCGAGGGCTGGGGCAGGAATATATAAGATAACATTGGAACAAATTCTG |
| | | ACGCCAGAACATGAAGAAATATTTGAAAAAGGATGGGGCATATTGACTGAACACAGAAGACAAACATGCATGACCTCAGCCT |
| | | AATCGTGAGAAAACATCAAAGAAATCCAAATTGATAAACATTCTGTAAAATAATTGGCCATGCTCAGCAAAAGTGTCACAATCA |
| | | CAAAAGACAAGGGAAGAATAAGAAACGGTCACAGCCTGGAGGAATCTAAGGACATAACAACTGAGTGCAATGTGAGATCCT |
| | | CGATTCGATCTTGGAGAAGAAAGAGGACGTTAAGAGAAATATTGACAAAATTCAAATAAGGTCTGCACATTAGGTAATGGTAA |
| | | GATACGTTGACATCAATTTCCTGGTTGGATACTTGTATTACATATACTATTATGTATATAATTATTATATTACAATTACATATACA |
| | | GTTGTATATTATATATAATACAATAAATGTATATATATGATTATAATACAATAAATGTATATATACAGTTTTATATATAACAACTAT |
| | | TATACATATAATACAATTATATATATAGCTAACATCAGGATAGGCTGGTTGAGGATTTATATGAATCTTTGCTAATTTTGCATGT |
| | | TTAAAATTATTTCAACATAAAAAAATTATATGGGTCATGTAGTTTTTTGTGCAGTTTAATTCTAGGCACCATAGTTTCTGTTTTAA |
| | | TGGGTGTATTTTCTTCTTTCAAGGAATTGGGATAGTCTGTTGTATTAAAAACCTATTCATTTTTGTACATTAAGATTCTTCTGGC |
| | | CAGGCGCAATGGCTCAAGCCTATAATCCCAGCACTTGGGAACCTGAGGCAGGCAGATCACCTGAGGTCAGGAGTTCAAGAC |
| | | CAGCATGTCCAACATGGTGAAACCCCATCTCTACTAAAAATACAAAAAAAATTAGCCAGGCATGGTGGCACATGCCTGTAATC |
| | | CCAGCTACTTGGGAGGCTGAGGAGGAGACTCACTAGAACTCAGGAAGCAGAGGTTGCAGTGAGCCAAGACTGCGCCACTG |
| | | CACTCTGGCCTAAGCAACAGAGGGAGACTCCATCTCAAAAAAAAAAAAAGATTATTTCTCTAACTTACTGGTTTATCTTTTATA |
| | | GCTTATTTCAGGTGATTTGTTGTGTTTGTCTATAGAGTAAAATAACATGTCTAATGAGTTATAATTTAATGTCCTTCCATTCAAT |
| | | CACTGTATTTCAAATTTATTCCAATATTTTGGAATAAGCTTCCAATATTTTAACTTAAATAGGGAAGGTCATACTTGGTATTTTC |
| | | CTGACCTCAGCTGGAATGCCTTCATAGTGTTCACATGAACACATTTGGCCTGAGAGAGACTTATTATGCCGAGAATGTGTGT |
| | | AACCCTTTTTACTTAGGAATTGTGGGTGGAGTTTTTCCCCCCGCCCAAGAATGATTGTTAATTTCCGTCCATCATTTTCCTTTG |
| | | ATTGTTCTTCATCTCGAAAACAAATAATAAAATAATAAAATAAATAAATAAAA |
| >ENST00000493504.1:: | 258 | CCCTTCGCGGCCGAGATGGACCCTGGGCTCGGCGTCCTCCGGAAAACTGCACTGTGAGGCGGGGCGATGGGGAAGGGCC |
| chr19: | | GAAGCCCCGCCAGCCCAGCTAAAGCAGACGGCTCGCACTGCGACCCGAAGCGCGAGTCATGGCTGGGGCCTTAGTGGCA |
| 59086826- | | ACAAGTGCTGAGATCAGCGGGCTCAGTAGCAGGAACACAAGCACCCCCTTGCGGTGTCTGGAGGAAGCGGCGCGGACAGT |
| 59095764(+) | | CTCCCGCTCCACCGTGTCGGGCCTGAAGAGGCCGCTGAACGCTCGCGATTACTGAGGCATCTGGTAGGATCGCAAGGCCA |
| | | TCAACCTACAGGTGAAGCTCGAGGCGCTGTGGCGCTTTGAAGCCGGAGAGAAGCTCAGTAGAATCGGGAAGGCGCTGGGG |
| | | CTGTCCAACTCCACCTTGGCCACCATTCGTGACAACAAGGAAAAGATCTGGGCGAGTTCCCAGGAGGCCACACTGCAGGCG |
| | | GCCACCAAGTTGACGCGTGGCCGCAGCCTGGTGATGGAGAACATGGAGCAGCTGCTGAGCATGCGGATGGAGGACCAGA |
| | | GCCAGCGCAACATGCCCCTCAGTGTGGTGCTCATTCGGGAGAAAGCCCACAGCCCGTTCGAGAACCGCAAGCAGGAGCAG |
| | | GGCGAGGGCGCCTAGCCCGAGAGCTTCGGGGCCGGTCGAGGGTGATTCGCTGGCTTTAAGGCGCGCCACAGCTTGAGCA |
| | | GCATCGGGGCCAGCGGCCCAAGAACGCAGCGTGCAAGAATCCAGCGTTAATGCGCAGGGTGATCCCGGAGGGCGGCTAC |
| | | ACACTCTGGCTGGTGTTCACCATGAATAAGCTCTGGCTGGTGTTCACCATGAATAAGCCCAGGTTCCTCTGTAAGCGCCTGC |
| | | CGAGAGGACGTTCATTTTCCTGTGGAAGATGAGTCGGCCCCTGGGTCAAAGCCGCCGGAGACCTCCTGACCCTATTGCTCG |
| | | GTGGCAACGCAGCCGGCGACTTCAAGCTGAAGGCCCTGCTGGGGTACCCCTCGGAGAACCCGTGTTTCCTCAAGGGCTCC |
| | | TTCAAGCCCAACCTGCCCTTGGTGTGGTGCTCGCACAAGAAAGCCTGGGTACAGCCTGGGTGACAGAGCGAGACTCCGTCT |
| | | CAAAAAAAAAAAAAAAAGCCTGGGTAACCATGAGCCTCTTCCAGGAGTGGTTCCTGCACTTCTGCTCTACAGTGGAGGGCGC |
| | | TGCACCCAGTACGACCTTCCCTACAAGGGGCTGCCCATCCTGGACAGCACTCCCGGCCACCCTGGGTGTTGAGACAGAGT |
| | | CTGGCTCTGTCGCGCAGGCTGGAGTGCGGTGGCGCGATCTCGGCTCACTGCAACCTCCGCCTCCCGGGTTCACGCCATTC |
| | | TCCTGCCTCAGCCTCCCAGGTAGCTGGGACTACAGGCGCCCGCCACCACGCCCGGCTAATTTTTTTGTATTTTTAGTAGAGA |
| | | CGGGGTTTCACCGTGTTAGCCAGAATGGTCTCGGTCTCCTGACCCCATGATCCACCCGCCTTGGCGTCCCAAAGTGCTGGG |
| | | ATTACAGGCGTGAGCCATGGTGCCTGGCCCTTTAATTCCCCCAATATTTTTTTATCTGCATTAATTTATTATTTCAAGTGAACT |
| | | TCCATCTCAAATACATGGTTCTCAGACGCTTCCATGAACTCTCAAGTGTGACTTATTACAGCCTGGGGCTCCTTGGAGCACA |
| | | GTGTGCCAGTGACCTAGTGGAAGGCCATGAGAAAGCCAGGTTCACTGTTCACTGTGCAGCATCTCCCTCCATGGTCTTCCT |
| | | GGAGCCTATTCCCACTGCACTGAGCATTCCGTGTGTCCAGAGGTAGGGTTAGCAGGGATAGATGGACTTCCTGCTTATTTTC |
| | | TGTTCACATTTCCACTGACCTTCAATCCTGTAGACTGCAAAGATTGAGCCAGCTGAGCCCCCAAAGAGACTCAGCAGTGCAG |
| | | AATGTGGGCCTGAGTGGGAGGACTGCCCAGCCGCACACCTTCCAGCTCTGTTCTTCCCGGGCCCTGGATGCTTCTTGCCAC |
| | | GCTGTCCTCTGAAATGACGCTCTGCCCCAGCAGACCTGTGGTAGGTGAGCTAGTGAGGAAGCACACCTGGGCTCTTCCCAC |
| | | CAGGCATCCCAGCCCTGGTGTGGGGTATCCGCATGGTTGCAGGCATCCCCTGGCCAGGCCTGGGTGCTCATTCTCCCTCT |
| | | CTCTTCTTGTCTCCTCCCCTCTCCCTGCTTTCTCTGCCCTGTCCTCCTCCTTTTTGCTGTTTTCCCCCGGACTCCTGCCATGC |
| | | CCCCAGACTTTCTTTTTCCCACGGATATGGGCTCATTCCAAGGAAAGGGTGGCATTTGGACACAAGGCCCTAGATCCCACGA |
| | | TGGAGGACTGGGGGGTACCTGATGCCCCTCAGAAAGAGGCCTGCAGGGACAGCACATGGGAAAGCCCCATGGCTTTGTGA |
| | | TCCATCTGGGGATGTGATCCATTTGGGTAAGGACTTGGGTTTCAGGGCATGAGTTGGCTTCCTTGCAGGATGCAGGTCCTTA |
| | | GGTGGGTGGCCTCTGTCTCCAGCTGTAGGGCCCTGCCAGGAAGCCTCTATATGAGCCTACCTCCCTCCTGCAGGACCAGA |
| | | GAGGGGTTGTTATGAACAGCCCAGGGGATTGGTTGCACTAAGCTTGTCTTGAAGCTTTGGCTGGGGAGTCCAGGTGCCCAT |
| | | GTCTCTCACCTGCTCCCCATACACATCTCTGCACACCTGGCTGAGGCATTCCCAGACCTAACCTCAGATAATGTGCATGTGA |
| | | TGAACACTCCCAAGTGGCTAGGCCTCTTGCACCTGAGCAGGTGGATTCTGCCCCAGCACTGGGGCTTTCTCTGGGCTGTCC |
| | | ATCATGGGTATATCTCTGGATTCCAGGATTGCTAGTTAGCACCTCACATTTGAGGGTCTGTGCTATTCAGTCTAGAATCAGAG |
| | | TTGGATGAGAAATCAACTTTGAACACCACCTTTGGGGTGGCTGCATTGGCTCACACCTGTAATCTCAGCACTTTAGGAGGCT |
| | | GAGGCAGGAGGATAGCTTGAGGCCAGGAGTTCGAGACTAACCGGAGCAACATAGTAAGGCCACATCTGTACAAAAAAAAGA |
| | | AAGAAAAAATTACCCAGGTTTGGTGGCATGCACTTGTAGTCCCAGTTGTGGGCTGAGGATTATGCAGGTGACAAGGCAAGA |
| | | GACTGAAGGCACAAACTGTTTCAGTATAATAAAGAAAATAGAATAATAATAGTCATAATTATATATAGAGATGATCATGAACAA |
| | | TTATCAATCATTATAATAAACATTATTAATCATTAGCTTTTA |
| >ENST00000495241.1:: | 259 | GGAGCCTCGGGCCGGGCTGCGTGAGGGAGGAGGGTTCATCATGCCTAGTGGCGTATAAGAAGACCCCGCCACCGGTCCC |
| chr20: | | TCCACGCACCACTTCAAAGCCGTTCATCTCAGTCACAGTCCAGAGCAGTACTGAGTCTGCCCAGGACACCTACCTGGACAG |
| 35089880- | | CCAGGACCACAAGAGCGAGGTGACTAGCCAGTCGGGCCTGAGCAACTCGTCGGACAGCCTGGACAGCAGTACCCGACCG |
| 35129000(+) | | CCCAGCGTGACACGGGGTGGAGTCGCCCCAGCCCCTGAGGCCCCAGAGCCACCCCCAAAACATGCAGCTCTGAAAAGTGA |
| | | ACAAGGGACGCTGACCAGCTCTGAGTCCCACCCCGAGGCCGCCCCCAAAAGGAAACTGTCATCGATAGGAATACAAAAGCA |
| | | GCGTCCCCTCTCACAGTATGTCCTCCCGACGGGACACAGACTCGGATACCCAGGATGCCAATGACTCAAGCTGTAAGTCAT |
| | | CTGAGAGGAGCCTCCCGGACTGTACCCCTCACCCCAACTCCATCAGCATCGATGCCGGTCCCCGGCAGGCCCCCAAGATT |
| | | GCCCAGATCAAGCGCAACCTCTCCTATGGAGACAACAGCGACCCTGCCCTAGAGGCGTCCTCGCTGCCCCCACCCGACCC |
| | | CTGGCTCGAGACCTCCTCCAGCTCCCCAGCAGAGCCGGCACAGCCAGGGGCCTGCCGCCGAGACGGCTACTGGTTCCTAA |
| | | AGCTACTGCAGGCAGAAACAGAGCGGCTGGAAGGCTGGTGCTGCCAGATGGACAAGGAGACCAAAGAGAACAA |
| >ENST00000435315.2:: | 260 | ACAAGGAGAGGGGCTTTGTGTCTTGTTTCACACTGTGTCCCCAGGCTTAGAATATTCTGGCATCAGAAGGCACTCAGTAAAC |
| chr21: | | ATAGAGAGGAAGAAAGGAAGAAGATGAAATCAGGAGTACAGATTAAAGTGTTATCTTTGGAAGACAGGCAAGACATCCTTTG |
| 16134030- | | GGACAGAAGGGAAGGAGAGAAAGCTGGGGAACATGCAGATAATACAAGAGGTGCGGTTTGTGACTGATGGCTGCTATTTTC |
| 16135411(−) | | TCTGTGAGACTGAATCCAGACAACTTTGGACATTCCTTCATTTGAAGAATTTTTACTGAACAATTACAGACCATTGATTGATAC |
| | | AGTGAAGATCAGAATGGCATAGTTTTCCTGTCTTCATGGAGATGAATGAATGAACTGTATTTTTTATTAGCCTAAGCATGAATT |
| | | CTTCCCAAATTTCCTCCATGAACCATAGGATCCTACCTCTTAACCATTTTATGAAAATTCTATGGTGAATTGAGGTCCTCAAGT |
| | | TGTCTGACAATATCTGATTTTGCCACGGTCATGTGGGAAAAGTAGGCATGTGAGCAAGTCATAGTTTTACTTGTTTGGAAGAT |
| | | TCTTCTAATTATGTCAAGATGAAAGGTTTTATACTGTGTAGAAGGCAGTGTGAGAGAAGTGGACAGTAGAACTCCTATTTGGT |
| | | GGGCCAGTGGCTATATTTTAAAATATGAAGGA |
| >ENST00000418510.1:: | 261 | AATGGGCTGCATTAAAAGCAAAGAAAACAAAAGTCCAGCCATTAAATACCTGAAAATACTCCAGAGCCTGTCAGTACAAGGG |
| chr22: | | TGAGCCATGGGAGCAGAACCCACTGCAGTGTCACCATGTCCATCATCTTCAGCAAAGGGAACAGCAGTTAATTTCAGCCGTC |
| 26043227- | | TTTCCATTCCATTATACCATTTGGAGGATCCTCAGGGGTAACACCTTTTGGAGGTGGGCATCTTCCTCATTCTCAGTGGTGCC |
| 26045199(+) | | AAGTTCATATCCTGCTGGCTTAACACGTGGTGTTACTATATTTGTGGCCTTATATGATTATGAAGCTAGAACTACAGAAGACC |
| | | TTTCATTTAAGAAGGGTGAAAAATTTCAAATAATTAACAATACAGAAGGAGACTGGTGGGAAGCAAGATCAATCACTACAGGA |
| | | AAGAATGGTTATATCCTGAGCAGTTATGTAGCGCCTGCAGATTCCATTCAGGCAGAAGAATGGTATTTTGGCAAAATGGGGA |
| | | GAAAAGATGCTGAAAGATTACTTCTGAATCCTGGAAAGAACAAAACAAAACAAAAACGAGAGAGCGAAACTACTAAAGGTGC |
| | | TTATTCCCTCTCTATTCGTGATTGGGATGAGGTAAGGGGTGACAATGTGAAACACCACAAAATTAGGAAACTTGACAATGGTA |
| | | GATACTATATCACAACCAGAGAACAACTTGATACTCTGCAGAAATTGGCAAAACACTACACAGAACATGCTGATGGTTTATGC |
| | | CACAAGTTAACAACTGTGTGTCCAACTGTGAAACCTCAGATTCAAGGTCTAGCAAAAGATGCTTGGGAAATCCCTTGATAATC |
| | | TTTGCGACTAGAGGTTAAACTAGGACAAGGATGTTTTGGCAAAGTGTGGATGGGAATATGGAATGGAACCACAAAAGTAGCA |
| | | ATCAAAACACTAAAACCAGGTACAATGATGCCAGAAGCTTTTCTTCAAGAAGCTCAGGTAATGAAAAAAATAAGACATGGTAA |
| | | ACTTGTTCCACTATATGCTGTTGTTTCTGAAGAGCCAATTTACATTGTCACTGAATTGATGTCAAAAGGAAGCTTATTCAATTT |
| | | CCTTAAGGAAGGAGATGGAAAGTATTTGAAGCTTCCACAAATGGTTGATATGCCTGCTCAGATTGCTGATGGTATGGCATATA |
| | | TTAAAAGAATGAACTATATTCACCGAGATCTCTGGGCTGCTAATATTCTTGTAGGAGAAAATCTTCTGTGCAAAATAGCAGATT |
| | | TTGGTTTAGCAAGGTTAATTGAAGACAATGAATACACATCAAGACAAGGTGCAGAATTTCCAATCAAATGGACAGCTCCTGAA |
| | | GTTGCACTGTATGGTGGGTTTACAATAAAGTCTGGTGTCTGCTCATTTGGAATTCTACAGACAGAACTGGTAACAAAGGGCA |
| | | GAGTGCCATATCCAGGTATGGTGAACCATGAAATACTGGAACAGGTGGAGCGAGGATACAGGATGCCTTGCCCTCAGGGCT |
| | | GTCCAGAATCCCTCCATGAATTGATGAATCTGTGTTGGAAGAAGGACCCTGATGAAAGACCAACATTTGAATATGTTCAGTCC |
| | | TTCTTGGGAGACTACTTCACTGCTACAGAGCCATAGTACCAGCCAGGAGAAAACTTC |
| >ENST000004 | 262 | TAACCTCAGTCCTCCTGCCCGGAGGGCATGTGTTTGCTGGAGATAGTGGAGTCGGTGGCAAGAAAGTGCCAGAGGGAGGT |
| 93023.1:: | | GCGCGGGCGGGAAAGCATCTGACGGTCTCCTGGGTTGCCACCCATGGCCAGCTGGGGCATCTTCTCACCTGCCCCAGCAA |
| chr22:37576210- | | AGCTTCCTGGGATCCCTGTCTGCTCTCAGCTGTGGCTGTTTTCAGAGGGGCTTCATCCTTCCAAGGCCTGTGTGGAGCAGA |
| 37581988(−) | | AGCCTCCATGGACTGGGTGCCCGGAGACTCAGCATGCAGGCCCTGCCCTGCCAGTCTCCTCCCGATCTTGGGGAACTTGC |
| | | TGCTCTGTGGAGGTCTCAGTCTTCTTGTCTGATAGATGGGGACAATAACCTTCCCTCTGGAGTTAGGTCAGGAGTGCTGTGG |
| | | CTGGAGGACGCAGCACCCGCCCACCCCAGGCATCGGCACATAGTAGATGTTGAGTGAAGTATAGTTCCTGTCCCTCCTTCC |
| | | TTGTTCCAGGTCACCATGGGGACAGCCGCCCTGGGTCCCGTCTGGGCAGCGCTCCTGCTCTTTCTCCTGATGTGTGAGATC |
| | | CCTATGGTGGAGCTCACCTTTGACAGAGCTGTGGCCAGCGGCTGCCAACGGTGCTGTGACTCTGAGGACCCCCTGGATCC |
| | | TGCCCATGTATCCTCAGCCTCTTCCTCCGGCCGCCCCCACGCCCTGCCTGAGATCAGACCCTACATTAATATCACCATCCTG |
| | | AAGGGTGACAAAGGGGACCCAGGCCCAATGGGCCTGCCAGGGTACATGGGCAGGGAGGGTCCCCAAGGGGAGCCTGGCC |
| | | CTCAGGGCAGCAAGGGTGACAAGGGGGAGATGGGCAGCCCCGGCGCCCCGTGCCAGAAGCGCTTCTTCGCCTTCTCAGT |
| | | GGGCCGCAAGACGGCCCTGCACAGCGGCGAGGACTTCCAGACGCTGCTCTTCGAAAGGGTCTTTGTGAACCTTGATGGGT |
| | | GCTTTGACATGGCGACCGGCCAGTTTGCTGCTCCCCTGCGTGGCATCTACTTCTTCAGCCTCAATGTGCACAGCTGGAATTA |
| | | CAAGGAGACGTACGTGCACATTATGCATAACCAGAAAGAGGCTGTCATCCTGTACGCGCAGCCCAGCGAGCGCAGCATCAT |
| | | GCAGAGCCAGAGTGTGATGCTGGACCTGGCCTACGGGGACCGCGTCTGGGTGCGGCTCTTCAAGCGCCAGCGCGAGAAC |
| | | GCCATCTACAGCAACGACTTCGACACCTACATCACCTTCAGCGGCCACCTCATCAAGGCCGAGGACGACTGAGGGCCTCTG |
| | | GGCCACCCTCCCGGCTGGAGAGCTCAGGTGCTGGTCCCGTCCCCTGCAGGGCTCAGTTTGCACTGCTGTGAAGCAGGAAG |
| | | GCCAGGGAGGTCCCCGGGGACCTGGCATTCTGGGGAGACCCTGCTTCTATCTTGGCTGCCATCATCCCTCCCAGCCTATTT |
| | | CTGCTCCTCTCTTCTCTCTTGGACCTATTTTAAGAAGCTTGCTAACCTAAATATTCTAGAACTTTCCCAGCCTCGTAGCCCAG |
| | | CACTTCTCAAACTTGGAAATGCATGCGAATCACCCGGGGTTCGTGTTAAATGCAGATTCTGACTCAGCAGGTCTGAGTGGGT |
| | | CCAGGATTCTGTGTTTCTCATATGTTCCTGGGTGATGCTGATGGGGTCAGTCTATGAACCACACTGGAGCAACCAGGTTCTA |
| | | GGACTTTCTCAATATTCTAGTACTTTCTGAACATTCTGGAATCCTCCCCACATTCTAGAATTCTCCCAACATTTTTTTTTCTTGA |
| | | GACAGAGTCTTGCTCTGTTGCCCAGGCTAGAGTGCAGTGGTGCAATCTCAGTTCACTGCAACCTCTGCCTCCCGGGTTCAA |
| | | GCGATTCTTCTGCCTCAGCCTCCCTAGTGGCTGGGATTACAGGCGCCTGCTACCATGCCTGGCTAATTTTTGTATTTTTAGTA |
| | | GAGATGGGGTTTCACCATATTGGCCAGGCTGGTCTTGAACTCCTGACTTCAGGTGACCCACCCGCCTCGGCCTCTCAAAAT |
| | | GCTGGGATTACAGGTGTGAGCCACCGTGCCTGGCCAATTCCAACATTCTTAAATTCTCTCATCCCTCCAGGGCTCCCCGTGC |
| | | TATGTTCTCTTTACCCCTTCCCCCTCTTCTCTTGCTCAGGCCTGCACCACTGCAGCCACCGTTCATTTATTCATTCATTAAACA |
| | | CTGAGCACTCACTCTGTGCTGGGTCCCGGGAAGGGTGAGGGGGTCAGACACAGGCCCTGCCCCTGCCCTCAGTGACTGGC |
| | | CAGTCCAGCCCAGGCGGGGAGAGATGTGTACATAGGTTTTAAAGCAGACCCAGAGCTCATGGGGGCCTGTGTTCTGGGTG |
| | | TTCAGGTGCTGCTGGTCCTCCATTACCCACTGCTCCCCAAGGCTGGTGGGACGGGGTCCCGGTGGCAGGGGCAGGTATCT |
| | | CCTTCCCGTTCCTCATCCACCTGCCCAGTGCTCATCGTTACAGCAAACCCCAGGGGGCCTTGGCCAGGTCAAGGGTTCTGT |
| | | GAGGAGAGGACCCAGGAGTGTGGGGGCATTTGGGGGGTGAAGTGGCCCCCGAAGAATGGAACCCACACCCATAGCTCTC |
| | | CCCACAGCTGATACGGCATCCTGCGAGAAGACCTGCCCTCCTCACTGGGATCCCCTTCCTGCCTCCTCCCAGGGCTCTGCC |
| | | AGGGCCTTGCTCAGTCCCTTCCACCAAAGTCATCTGAACTTCCGTTTCCCCAGGGCCTCCAGCTGCCCTCAGACACTGATGT |
| | | CTGTCCCCAGGTGCTCTCTGCCCCTCATGCCCCTCTCACCGGCCCAGTGCCCCGACTCTCCAGGCTTTATCAAGGTGCTAA |
| | | GGCCCGGGTGGGCAGCTCCTCGTCTCAGAGCCCTCCTCCGGCCTGGTGCTGCCTTTACAAACACCTGCAGGAGAAGGGCC |
| | | ACGGAAGCCCCAGGCTTTAGAGCCCTCAGCAGGTCTGGGGAGCTAGAGCAAAGGAGGGACCTCAGGCCTTCCGTTTCTTC |
| | | TTCCAGGGTGGGGTGGCCTGGTGTTCCCCTAGCCTTCCAAACCCAGGTGGCCTGCCCTTCTCCCCAGAGGGAGGCGGCCT |
| | | CCGCCCATTGGTGCTCATGCAGACTCTGGGGCTGAGGTGCCCCGGGGGGTGATCTCTGGTGCTCACAGCCGAGGAGCCGT |
| | | GGCTCCATGGCCAGATGACGGAAACAGGGTCTGACCAAGTGCCAGGAAGACCTGTGCTATAAACCACCCTGCCTGATCCTG |
| | | CCCCTGCCTGACCCCGCCACGCCCTGCCGTCCAGCATGATTAAAGAATGCTGTCTCCTC |
| >ENST00000446986.1:: | 263 | GCTCGCCCTCGGCACCTGACCCGGCGAGAGGAAGGGGCGGGTCCAGAGAACACCCGGGACTTGAACGCTGGACCCTGAG |
| chrX: | | AGAGGCCTCGGAGAGGGGAGGAAGGGTGAGGTCACGATGTGCCTGGACAAAAACCCTTGGAGCAGGACCAGGACTAGGA |
| 118599996- | | CCGTGTGCTGTCCTGGAGCCAGTGAAACCAGACGAAATTAAGGGAACAGCAGACGAAGGAACTGCCACCGTGCCCCAGAC |
| 118602225(−) | | CCTGGTTTGCAAATACAGTTCGACAAAGATCTTGCCAGGAATTGGCCTGGGAGAGTCCAAAGGACTGGGCTGGGGGGGG |
| | | GAAATGATTTTAGCCAGAAGACAAATATAGAGGTTGTACCCAGAAGGTTAAGAGGCTTAGGAAGGTTAAGAACTACGTTAAC |
| | | ATTTGGCTCCCAGGACCCCAGGTAAAGTGCCCTTGGATTGAATAGATTTCTGAACTCCAGAAACATTTAGAGGAATCAATAGA |
| | | ATTAGATTGGCAGCCATTGTGCCAATGGACAAGTGCTTACTGAGCCCCTTAATCCCTTCACTGGGTAAGCATTTGCCACACA |
| | | TGTCCTCAAAAACCTACTTGTTAGCTTAAGTGGACTCCCTCAAGTTCAGAACAGGAGCAAATAGTGTAATCACAAGTTCCGTG |
| | | TCTGAATAATCATAACAATGTTTATAAAAATAAGGACGACTGTTGAAAACTGCAACTGCAGCTGAACCTTATGTGCCCAGACT |
| | | TCAGAATTCCCATAGGCAAAATGCCAGCAGGCCTTTGCACACTGAACACATAGTGTTCATATCCTTCACTTTCCTTTCCTGCC |
| | | CTCTCCTATCTTCTCCAGATCTGCCTGTGAAAGGACCCAATTCTAGGACCCCCCAGGATGGTTAGGCCCTTCCTCTGAAAGT |
| | | ACACTTTGCAATTTGTTAGTCTCCTCAAGAACCCCAGCGATCGAAACTGATGCACACAAATTGCATGTGTTCAAAGGAGCAAT |
| | | TTTGAATCTAGCTTTGTTGATCCTGGGAGAAATCTTTGACCTGTGTTAGTGAAAGGCTATGGTGTTTCTCAGCTGATTATAATA |
| | | GCGAGGTAGCTGAATGCAAAAAAACAGTTTCAGAGAATGGTAGAGTATATCCTCCAAAGGTGATACTCGCTTCTCTATTCTG |
| | | GTGAGAGAAACAAGAGCTAAAGCAAAATTCTACAGAGGGCTTTATTTGGAGAGAGGTGGATTTTATTTATAATGCATTTAAAA |
| | | TTCAAAATCTTTCTAGTTTGAACTCTTGACATTCTGATTAATGCATTTTTATTCAACTGTTAATTATTCCTCGATACACCTCGCC |
| | | ATCACTTGTGTATCCCTTTTCTACATGTGACCTTTCCCTGGCCACATCTGCTACTCTCTTTGAGCATGATGCCTCTACTGTATT |
| | | CTATTCCAGAGCTCATTGTTTTGTCAGTTTTTTTTCTCTCCACAAAAAAAGATGATCAAATAATTACATAGAATTTTTCCCTTCT |
| | | CAGTCTTTGGGTAACTAAGTGTGGTTTTATTGACGATGAAATACAAGATGGGTCATTTAAAAGCAACTTCTTGTCATCACTTG |
| | | GTATTTTGCTAAATCCACGGTGAGCTGGAGCTCCCGTAGTCACCTATCACCGCGTGTATTTTAAGGAGTCAGATATTCTCGAA |
| | | ACTGTTAAGATCATGAAAGACAAAGAGACTAAGGAACTGTCACAGATTAAAGGAGACTAAAGATACTTGACAACTGAATGTAA |
| | | TATATGACACTCAACCGGCTCCTGTACCAGAAAAAGGACATTGGTTATATTGGTGAGGTTTGAATATCTGTAGATTACCAATA |
| | | CTATTCTTGGTTATGGTGGTTATGTAAGATTTAGAGAAGTTGAATAAAGGATATAAGGAC |

TABLE 10
DE SCZ nORF peptide sequences

| | | | | | SEQ | | |
|---|---|---|---|---|---|---|---|
| norf_ | n_ | n_ | n_ | n_ | ID | | de_ |
| id | chr | start | end | strand | NO: | V13 | dim |
| 0iakH2 | chr1 | 47645100 | 47645604 | + | 264 | MRISLQGMYYRANFTGKGME | up |
| | | | | | | DLKG | |
| 0q7uH6 | chr1 | 1.1E+08 | 1.1E+08 | + | 265 | MAGEALQGPGTSGPAGEGRQ | up |
| | | | | | | RVATMIKGFS | |
| 0zkdH6 | chr1 | 1.68E+08 | 1.68E+08 | + | 266 | MATAGSGGDSGDRELRPGRA | down |
| | | | | | | GASAAESAPEGHGAWRHPND | |
| | | | | | | PSTQMALL | |
| 12ogH1 | chr1 | 1.77E+08 | 1.77E+08 | + | 267 | MQDPYTLKGGEKQAEFFFLA | down |
| | | | | | | VLSLLPEMNGTHKTPRR | |
| 2aykH2 | chr1 | 95087473 | 95088658 | − | 268 | MLPFPRCLLFPLILRRAGLC | up |
| | | | | | | AAGSSTRPGSMTPSANALPR | |
| | | | | | | STGLTLPQACAFCDSLPQQF | |
| | | | | | | LWISLSSGMCWGHRQVRPGF | |
| | | | | | | LGSECSLPVADGTGRHVPRI | |
| | | | | | | PRLQVGWCQESSTPSFPGPR | |
| | | | | | | RTSTVVHKGTKRMLKNPGWI | |
| 2v5xH1 | chr1 | 2.1E+08 | 2.1E+08 | − | 269 | MTHSGWQGSGGAGPGPRAPA | down |
| | | | | | | RSRCRPGLPSLPCRPNPLEA | |
| | | | | | | RGCGSALRPWDLAARTPIPC | |
| | | | | | | FCDHSWALGKLLGPKRREGR | |
| | | | | | | KGSERRRRGPGGQQPQPAEE | |
| 2vnjH1 | chr1 | 2.13E+08 | 2.13E+08 | − | 270 | MMTGRSEGEKKTELLLREVG | down |
| | | | | | | RSRPRRLTSSMRNMRAWSKK | |
| | | | | | | TPCCGERSGS | |
| 5sz1H1 | chr3 | 64670949 | 64785258 | + | 271 | MCLGTLRPTNDSALMEQPSV | up |
| | | | | | | WGKVLEVHSFRKPLTSLYHA | |
| | | | | | | LSEKLRIRKEQDPQETLMSG | |
| 9izeH2 | chr5 | 43575997 | 43602916 | − | 272 | MSSLGSHQNKETGLKTLIPE | up |
| | | | | | | SILPHIQNEIHAQRCQEESR | |
| | | | | | | QTGLSRLYKKHDASICFW | |
| a87hH2 | chr6 | 31553973 | 31554980 | + | 273 | MGEKLKPEEGQECLRWQHFS | down |
| | | | | | | SARQHWPVWSLSILQATSSG | |
| a87hH3 | chr6 | 31554008 | 31555009 | + | 274 | MSEVATLLFSQTALASLESV | down |
| | | | | | | HPAGHKLWMRNLRQVTSP | |
| a87hH4 | chr6 | 31554088 | 31555065 | + | 275 | MDEELEASHQPLIISPKRAR | down |
| | | | | | | TRVPDLQASP | |
| aexwH1 | chr6 | 1.08E+08 | 1.08E+08 | + | 276 | MKCEYKSRDCRCWCLQPLIQ | up |
| | | | | | | QKSGSVHSLARRRSF | |
| axtlH1 | chr7 | 296640 | 297004 | + | 277 | MRGGEADTALRQQPPHPGRH | up |
| | | | | | | GHDDLRLPHGKHGPSPLRDF | |
| azswH1 | chr7 | 29186332 | 29186528 | + | 278 | MKSIGGVPFRTYLQAQDLHL | down |
| | | | | | | ESEWCLIPTV | |
| dnxdH2 | chr11 | 10879807 | 10899979 | + | 279 | MIAERLGRKQLFSNFTSPVG | down |
| | | | | | | ADACRECRFLGTARYELSIP | |
| | | | | | | QKSSHFVSSHPILLTNQRLG | |
| | | | | | | IICECRTTGFAVFI | |
| dol2H3 | chr11 | 18416109 | 18418402 | + | 280 | MLQPLPPIPDLIATRPRRPP | up |
| | | | | | | DVHSRFLLVPSPIWQL | |
| ewspH2 | chr12 | 9220574 | 9221353 | − | 281 | MITTRRMSLQLLSTMLLAAK | down |
| | | | | | | VSHSHSSKRQSELLQLAPQT | |
| | | | | | | FSFIKRWVNICQKHQITHTA | |
| | | | | | | HYYRKTHLLERAADSVKPQR | |
| | | | | | | VHRIAYG | |
| frtoH1 | chr14 | 52327436 | 52344364 | + | 282 | MARLHRSAPERGKPRSSDQA | down |
| | | | | | | SGAKPDLPVSLRL | |
| fs1rH2 | chr14 | 55034681 | 55168789 | + | 283 | MERVRADCCAAVAAQAREPD | up |
| | | | | | | PGPLPPALPGALAGRLRRAA | |
| | | | | | | RPRTRGQQPRNH | |
| geqkH1 | chr15 | 61057284 | 61057457 | + | 284 | MPTVEAIFEREGLWLKSTVW | up |
| | | | | | | FLTVYIYESK | |
| hglvH5 | chr17 | 5015331 | 5017507 | + | 285 | MHDWTGAAGCPGGPWELQSL | down |
| | | | | | | RRWRVSPYEKTQDSETIKVT | |
| | | | | | | EMKLTEPEFQVMILYGFFGA | |
| | | | | | | ALDLSSD | |
| hubwH1 | chr17 | 5403122 | 5403643 | − | 286 | MPLDPYHSVTWGHAQGSHHF | up |
| | | | | | | VFGELRVRPILESLRDEPDP | |
| | | | | | | DPRPSREGPAGRVGALARGG | |
| | | | | | | PEPCDAASPPGGASCAPELA | |
| | | | | | | RPREDKSAQQAKLEGGTRLC | |
| | | | | | | CRCPEESRLVPGGAVSPGDH | |
| | | | | | | VLEVSGTRGTCGCRPRRHAG | |
| | | | | | | PELAHS | |
| iu75H2 | chr19 | 48758950 | 48759608 | + | 287 | MLTKRNRTQSSLAVSYQQAS | down |
| | | | | | | VSPSCTLGLLKLT | |
| iu75H4 | chr19 | 48758982 | 48759643 | + | 288 | MGGVLPTSLRFSFLYTRAPE | down |
| | | | | | | THLMKSPSVTQAGV | |
| iwy5H2 | chr19 | 59086839 | 59092651 | + | 289 | MWTLGSASSGKLHCEAGRWG | down |
| | | | | | | RAEAPPAQLKQTARTATRSA | |
| | | | | | | SHGWGLSGNKC | |
| iwy5H7 | chr19 | 59086942 | 59092775 | + | 290 | MRPEARVMAGALVATSAEIS | down |
| | | | | | | GLSSRNTSTPLRCLEEAART | |
| | | | | | | VSRSTVSGLKRPLNARDY | |
| jsksH2 | chr21 | 16134421 | 16135215 | − | 291 | MQIIQEVRFVTDGCYFLCET | down |
| | | | | | | ESRQLWTFLHLKNFY | |
| tracer_ | chr7 | 1.52E+08 | 1.52E+08 | + | 292 | TPPRPPCRRPGSEEAHSLRG | up |
| 102180 | | | | | | RCARSSTCFSKHRSPRVPPR | |
| | | | | | | PARALEPAQRAVRGAGCEQG | |
| | | | | | | LATPTLTPPHPTLLFSGSSR | |
| | | | | | | FAQWIRE | |
| tracer_ | chr7 | 1.52E+08 | 1.52E+08 | + | 293 | VLLETPQPPGPAPPGARTRT | up |
| 102181 | | | | | | RPESGAWRWVRAGSSHPHPH | |
| | | | | | | PTPPHPAFFRFIKVCAVDPR | |
| | | | | | | MKPAWKIPSLETEPDRGRCT | |
| | | | | | | GRTLSGFSNQAAILAPQVWD | |
| | | | | | | FWVFSWLASC | |
| tracer_ | chr1 | 2.34E+08 | 2.34E+08 | + | 294 | LGYGHTVPLSDGGKAFCIIY | down |
| 10338 | | | | | | SVIGIPFTLLFLTAVVQRIT | |
| | | | | | | VHVTRRPVLYFHIRWGFSKQ | |
| | | | | | | VVAIVHAVLLGFVTVSCFFF | |
| | | | | | | IPAAVFSVLEDDWNFLESFY | |
| | | | | | | FCFISLSTIGLGDYVPGEGY | |
| | | | | | | NQKFRELYKIGITCYLLLGL | |
| | | | | | | IAMLVVLETFCELHELKKFR | |
| | | | | | | KMFYVKKDKDEDQVHIIEHD | |
| | | | | | | QLSFSSITDQAAGMKEDQKQ | |
| | | | | | | NEPFVATQSSACVDGPANH | |
| tracer_ | chr8 | 1.07E+08 | 1.07E+08 | + | 295 | TRAGPETEHVCKRSAAPAAA | up |
| 104995 | | | | | | GLELGCVRLSPFTSRPQELS | |
| | | | | | | AGAAPPSPAERGALAAAGPA | |
| | | | | | | AAHRSPLLATRKRTQPPAAT | |
| | | | | | | TVDSLYGRESWESRPSEGFP | |
| | | | | | | NPKGPSPRRHSRGPRFTLLP | |
| | | | | | | LAGDAVIVACGLGSTMAPKG | |
| | | | | | | QRGREAGTETSI | |
| tracer_ | chr8 | 1.31E+08 | 1.31E+08 | − | 296 | LCERRRLGFSKEKKLRPGI | down |
| 105388 | | | | | | | |
| tracer_ | chr8 | 1.45E+08 | 1.45E+08 | − | 297 | LPPLSGQEVRHGAEHQECAG | down |
| 105819 | | | | | | EADHRAPAAAAHQPADGSSD | |
| | | | | | | NGLWRSSLTFAIGQ | |
| tracer_ | chr9 | 95726523 | 95738515 | + | 298 | MHHHVVTHIAKIRKKEVVFR | down |
| 107920 | | | | | | YCTKFSPEEKLARLQKTVPP | |
| | | | | | | KWLYFEPAGQGRDFQGNHLP | |
| | | | | | | CASSCRPTPDPSTEPGACAR | |
| | | | | | | RPSASSSERLVASGSPWPAS | |
| | | | | | | T | |
| tracer_ | chrX | 1.19E+08 | 1.19E+08 | − | 299 | MCLDKNPWSRTRTRTVCCPG | down |
| 112653 | | | | | | ASETRRN | |
| tracer_ | chr11 | 69240563 | 69242419 | + | 300 | MVKREKDFCFQIRCFLRLGA | down |
| 18675 | | | | | | EFSCPWRSSSSGPLAGPHPL | |
| | | | | | | QSGQIPCSQGKWPMPAVLVC | |
| | | | | | | NIGPACWNLTLFLPDTQQNK | |
| | | | | | | PQGRDQCLQPESRRKRSDR | |
| tracer_ | chr14 | 67708244 | 67736504 | + | 301 | VENKKALSFVKNRRRETKRT | up |
| 31960 | | | | | | VEKAFPVCIM | |
| tracer_ | chr15 | 29363239 | 29394122 | + | 302 | MAVIPESAWKHPDYVDDGLS | down |
| 34251 | | | | | | GVCNGLEQPRKQQRSDLNGP | |
| | | | | | | VDNNNIPETKKVASFPSFVA | |
| | | | | | | VPGPCEPEDLIDGIIFAANY | |
| | | | | | | LGSTQLLSERNPSKNIRMMQ | |
| | | | | | | AQEAVSRVKNSEGDAQTLTE | |
| | | | | | | VDLFISTQRIKVLNADTQET | |
| | | | | | | MMDHALRTISYIADIGNIVV | |
| | | | | | | LMARRRMPRSASQDCIETTP | |
| | | | | | | GAQEGKKQYKMICHVFESED | |
| | | | | | | VSKPLPGHSPPKVHSPGRLQ | |
| | | | | | | DPGAVETTLRWKASMLLLMF | |
| | | | | | | PVDQ | |
| tracer_ | chr1 | 46016779 | 46032696 | + | 303 | LSPGHSTLFTLCACAKVKAA | down |
| 3540 | | | | | | VKYALSVGYRHIDCAAIYGN | |
| | | | | | | EPEIGEALKEDVGPGKAVPR | |
| | | | | | | EELFVTSKLWNTKHHPEDVE | |
| | | | | | | PALRKTLADLQLEYLDLYLM | |
| | | | | | | HWPYAFE | |
| tracer_ | chr15 | 75315958 | 75337062 | + | 304 | LARILNPDSFIEPRPGRLPE | down |
| 36936 | | | | | | LEATRPHMEPKASCPAAAPL | |
| | | | | | | MERKFHVLVGVTGSVAALKL | |
| | | | | | | PLLVSKLLDIPGLEVAVVTT | |
| | | | | | | ERAKHFYSPQDIPVTLYSDA | |
| | | | | | | DEWEIWKSRSDPVLHIDLRR | |
| | | | | | | WADLLLVAPLDANTLGKVAS | |
| | | | | | | GICDNLLVSDVLVPSSVPGP | |
| | | | | | | HTQFAELQTSLYKETCCCGA | |
| | | | | | | PVCPGRSCSAIPHIAPSALT | |
| | | | | | | FSVAHGHSPGPDGSV | |
| tracer_ | chr15 | 99645480 | 99673263 | + | 305 | VCRVRELERENLLLEEELRG | up |
| 38082 | | | | | | RRGREGLWAEGQARCAEEAR | |
| | | | | | | SLRQQLDELSWATALAEGER | |
| | | | | | | DALRRELRELQRLDAEERAA | |
| | | | | | | RGRLDAELGAQQRELQEALG | |
| | | | | | | ARAALEALLGRLQAERRGLD | |
| | | | | | | AAHERDVRELRARAASLTMH | |
| | | | | | | FRARATGPAAPPPRLREVHD | |
| | | | | | | SYALLVAESWRETVQLYEDE | |
| | | | | | | VRELEEALRRGQESRLQAEE | |
| | | | | | | ETRLCAQEAEALRREALGLE | |
| | | | | | | QLRARLEDALLRMREEYGIQ | |
| | | | | | | AEERQRVIDCLEDEKATLTL | |
| | | | | | | AMADWLRDYQDLLQVKTGLS | |
| | | | | | | LEVATYRALLEGESNPEIVI | |
| | | | | | | WAEHVENMPSEFRNKSYHYT | |
| | | | | | | DSLLQRENERNLFSRQKAPL | |
| | | | | | | ASFNHSSALYSNLSGHRGSQ | |
| | | | | | | TGTSIGGDARRGFLGSGYSS | |
| | | | | | | SATTQQENSYGKAVSSQTNV | |
| | | | | | | RTFSPTYGLLRNTEAQVKTF | |
| | | | | | | PDRPKAGDTREVPVYIGEDS | |
| | | | | | | TIARESYRDRRDKVAAGASE | |
| | | | | | | STRSNERTVILGKKTEVKAT | |
| | | | | | | REQERNRPETIRTKPEEKMF | |
| | | | | | | DSKEKASEERNLRWEELTKL | |
| | | | | | | DKEARQRESQQMKEKAKEKD | |
| | | | | | | SPKEKSVREREVPISLEVSQ | |
| | | | | | | DRRAEVSPKGLQTPVKDAGG | |
| | | | | | | GTGREAEARELRFRLGTSDA | |
| | | | | | | TGSLQGDSMTETVAENIVTS | |
| | | | | | | ILKQFTQSPETEASADSFPD | |
| | | | | | | TKVTYVDRKELPGERKTKTE | |
| | | | | | | IVVESKLTEDVDVSDEAGLD | |
| | | | | | | YLLSKDIKEVGLKGKSAEQM | |
| | | | | | | IGDIINLGLKGREGRAKVVN | |
| | | | | | | VEIVEEPVSYVSGEKPEEFS | |
| | | | | | | VPFKVEEVEDVSPGPWGLVK | |
| | | | | | | EEEGYGESDVTFSVNQHRRT | |
| | | | | | | KQPQENTTHVEEVTEAGDSE | |
| | | | | | | GEQSYFVSTPDEHPGGHDRD | |
| | | | | | | DGSVYGQIHIEEESTIRYSW | |
| | | | | | | QDEIVQGTRRRTQKDGAVGE | |
| | | | | | | KVVKPLDVPAPSLEGDLGST | |
| | | | | | | HWKEQARSGEFHAEPTVIEK | |
| | | | | | | EIKIPHEFHTSMKGISSKEP | |
| | | | | | | RQQLVEVIGQLEETLPERMR | |
| | | | | | | EELSALTREGQGGPGSVSVD | |
| | | | | | | VKKVQGAGGSSVTLVAEVNV | |
| | | | | | | SQTVDADRLDLEELSKDEAS | |
| | | | | | | EMEKAVESVVRESLSRQRSP | |
| | | | | | | APGSPDEEGGAEAPAAGIRF | |
| | | | | | | RRWATRELYIPSGESEVAGG | |
| | | | | | | ASHSSGQRTPQGPVSATVEV | |
| | | | | | | SSPTGFAQSQVLEDVSQAAR | |
| | | | | | | HIKLGPSEVWRTERMSYEGP | |
| | | | | | | TAEVVEVSAGGDLSQAASPT | |
| | | | | | | GASRSVRHVTLGPGQSPLSR | |
| | | | | | | EVIFLGPAPACPEAWGSPEP | |
| | | | | | | GPAESSADMDGSGRHSTFGC | |
| | | | | | | RQFHAEKEIIFQGPISAAGK | |
| | | | | | | VGDYFATEESVGTQTSVRQL | |
| | | | | | | QLGPKEGFSGQIQFTAPLSD | |
| | | | | | | KVELGVIGDSVHMEGLPGSS | |
| | | | | | | TSIRHISIGPQRHQTTQQIV | |
| | | | | | | YHGLVPQLGESGDSESTVHG | |
| | | | | | | EGSADVHQATHSHTSGRQTV | |
| | | | | | | MTEKSTFQSVVSESPQEDSA | |
| | | | | | | EDTSGAEMTSGVSRSFRHIR | |
| | | | | | | LGPTETETSEHIAIRGPVSR | |
| | | | | | | TFVLAGSADSPELGKLADSS | |
| | | | | | | RTLRHIAPGPKETSFTFQMD | |
| | | | | | | VSNVEAIRSRTQEAGALGVS | |
| | | | | | | DRGSWRDADSRNDQAVGVSF | |
| | | | | | | KASAGEGDQAHREQGKEQAM | |
| | | | | | | FDKKVQLQRMVDQRSVISDE | |
| | | | | | | KKVALLYLDNEEEENDGHWF | |
| tracer_ | chr16 | 30785454 | 30785684 | + | 306 | LEHHGPWGLCPHLLPTPGQS | down |
| 41430 | | | | | | CSLGGTALQKRSA | |
| tracer_ | chr17 | 1617237 | 1619451 | − | 307 | VQQPPHPAPGACSSHALTWL | down |
| 44600 | | | | | | SRSSSSVASFMS | |
| tracer_ | chr19 | 7982552 | 7983974 | + | 308 | LDPVSAWHFPQGRPRVSPAV | down |
| 55120 | | | | | | QCALSLPRRPPRPWNPRRWW | |
| | | | | | | RWCWQPSCWAPRWPPGWVSS | |
| | | | | | | VRTQRPTPLARPREPRPAVP | |
| | | | | | | SPGGPSEEGMVRPQHGPEIH | |
| | | | | | | PATNSGPGPTGPDPPPWTSD | |
| | | | | | | LMRPRPLRFSPPPVPPTCAQ | |
| | | | | | | NKPLD | |
| tracer_ | chr19 | 12462819 | 12476403 | − | 309 | MLAAGNLGENTGHRGSREMD | up |
| 55894 | | | | | | SVAFEDVAVNFTQEEWALLG | |
| | | | | | | PSQKSLYRDVMWETIRNLDC | |
| | | | | | | IGMKWEDTNIEDQHRNPRRS | |
| | | | | | | LR | |
| tracer_ | chr19 | 36395470 | 36399113 | − | 310 | LQQAPAPASPAGCLRPVQAQ | down |
| 57824 | | | | | | AQSDCSCSTVSPGVLAGIVM | |
| | | | | | | GDLVLTVLIALAVYFLGRLV | |
| | | | | | | PRGRGAAEAATRKQRITETE | |
| | | | | | | SPYQELQGQRSDVYSDLNTQ | |
| | | | | | | RPYYK | |
| tracer_ | chr2 | 1.45E+08 | 1.45E+08 | − | 311 | LAKTTNKTSQIEPACCRSRA | down |
| 65443 | | | | | | PSPCELPSDPLLSMKQPIMA | |
| | | | | | | DGPRCKRRKQANPRRKNVVN | |
| | | | | | | YDNVVDTGSETDEEDKLHIA | |
| | | | | | | EDDGIANPLDQETSPASVPN | |
| | | | | | | HESSPHVSQALLPREEEEDE | |
| | | | | | | IREGGVEHPWHNNEILQASV | |
| | | | | | | DGPGKCLKSLGLFLYHLHIF | |
| | | | | | | N | |
| tracer_ | chr2 | 1.6E+08 | 1.6E+08 | + | 312 | VENCVCTLRNLSYRLELEVP | up |
| 65704 | | | | | | QARLLGLNELDDLLGKESPS | |
| | | | | | | KDSEPSCWGKKKKKKKRTPQ | |
| | | | | | | EDQWDGVGPIPGLSKSPKGV | |
| | | | | | | EMLWHPSVVKPYLTLLAESS | |
| | | | | | | NPATLEGSAGSLQNLSAGNW | |
| | | | | | | KFAAYIRGRPKRKGLPILVE | |
| | | | | | | LLRMDNDRVVSSVATALRNM | |
| | | | | | | ALDVRNKELIGKYAMRDLVN | |
| | | | | | | RLPGGNGPSVLSDETMAAIC | |
| | | | | | | CALHEVTSKNMENAKALADS | |
| | | | | | | GGIEKLVNITKGRGDRSSLK | |
| | | | | | | VVKAAAQVLNTLWQYRDLRS | |
| | | | | | | IYKKDGWNQNHFITPVSTLE | |
| | | | | | | RDRFKSHPSLSTTNQQMSPI | |
| | | | | | | IQSVGSTSSSPALLGIRDPR | |
| | | | | | | SEYDRTQPPMQYYNSQGDAT | |
| | | | | | | HKGLYPGSSKPSPIYISSYS | |
| | | | | | | SPAREQNRRLQHQQLYYSQD | |
| | | | | | | DSNRKNFDAYRLYLQSPHSY | |
| | | | | | | EDPYFDDRVHFPASTDYSTQ | |
| | | | | | | YGLKSTTNYVDFYSTKRPSY | |
| | | | | | | RAEQYPGSPDSWVHQDAQQR | |
| | | | | | | NSFFLTLFRLR | |
| tracer_ | chr2 | 1.97E+08 | 1.97E+08 | + | 313 | VNTRFGAAGVWYRAERRCTR | up |
| 66780 | | | | | | HSSVRKNRNEERNGHDPGRG | |
| | | | | | | HQDLDPDNEGELRHTRKREA | |
| | | | | | | PHVKNNAIISLRKDLNEDDH | |
| | | | | | | HHECLNVTQLLKYYGHGANS | |
| | | | | | | PISTDLFTYLCPALLYQIDS | |
| | | | | | | RLCIEHFDKLLVEDINKDKN | |
| | | | | | | LVPEDEANIGASAWICGIIS | |
| | | | | | | ITVISLLSLLGVILVPIINQ | |
| | | | | | | GCFKFLLTFLVALAVGTMSG | |
| | | | | | | DALLHLLPHSQGGHDHSHQH | |
| | | | | | | AHGHGHSHGHESNKFLEEYD | |
| | | | | | | AVLKGLVALGGIYLLFIIEH | |
| | | | | | | CIRMFKHYKQQRGKQKWFMK | |
| | | | | | | QNTEESTIGRKLSDHKLNNT | |
| | | | | | | PDSDWLQLKPLAGTDDSVVS | |
| | | | | | | EDRLNETELTDLEGQQESPP | |
| | | | | | | KNYLCIEEEKIIDHSHSDGL | |
| | | | | | | HTIHEHDLHAAAHNHHGENK | |
| | | | | | | TVLRKHNHQWHHKHSHHSHG | |
| | | | | | | PCHSGSDLKETGIANIAWMV | |
| | | | | | | IMGDGIHNFSDGLAIGAAFS | |
| | | | | | | AGLTGGISTSIAVFCHELPH | |
| | | | | | | ELGDFAVLLKAGMTVKQAIV | |
| | | | | | | YNLLSAMMAYIGMLIGTAVG | |
| | | | | | | QYANNITLWIFAVTAGMFLY | |
| | | | | | | VALVDMLPEMLHGDGDNEEH | |
| | | | | | | GFCPVGQFILQNLGLLFGFA | |
| | | | | | | IMLVIALYEDKIVFDIQF | |
| tracer_ | chr2 | 2.34E+08 | 2.34E+08 | + | 314 | LALQTMFHRSPCPRCGTSRP | down |
| 68382 | | | | | | SCRREKAPWRAPAPAGSPWT | |
| | | | | | | PVPQPPPSRQDPRTAPRRSG | |
| | | | | | | WTRAAGRWGPALSRPS | |
| tracer_ | chr20 | 35089900 | 35128670 | + | 315 | VREEGSSCLVAYKKTPPPVP | down |
| 70164 | | | | | | PRTTSKPFISVTVQSSTESA | |
| | | | | | | QDTYLDSQDHKSEVTSQSGL | |
| | | | | | | SNSSDSLDSSTRPPSVTRGG | |
| | | | | | | VAPAPEAPEPPPKHAALKSE | |
| | | | | | | QGTLTSSESHPEAAPKRKLS | |
| | | | | | | SIGIQKQRPLSQYVLPTGHR | |
| | | | | | | LGYPGCQ | |
| tracer_ | chr22 | 26043469 | 26044392 | + | 316 | VPSSYPAGLTRGVTIFVALY | up |
| 73466 | | | | | | DYEARTTEDLSFKKGEKFQI | |
| | | | | | | INNTEGDWWEARSITTGKNG | |
| | | | | | | YILSSYVAPADSIQAEEWYF | |
| | | | | | | GKMGRKDAERLLLNPGKNKT | |
| | | | | | | KQKRESETTKGAYSLSIRDW | |
| | | | | | | DEVRGDNVKHHKIRKLDNGR | |
| | | | | | | YYITTREQLDTLQKLAKHYT | |
| | | | | | | EHADGLCHKLTTVCPTVKPQ | |
| | | | | | | IQGLAKDAWEIP | |
| tracer_ | chr22 | 37578227 | 37581489 | − | 317 | MGTAALGPVWAALLLFLLMC | up |
| 74268 | | | | | | EIPMVELTFDRAVASGCQRC | |
| | | | | | | CDSEDPLDPAHVSSASSSGR | |
| | | | | | | PHALPEIRPYINITILKGDK | |
| | | | | | | GDPGPMGLPGYMGREGPQGE | |
| | | | | | | PGPQGSKGDKGEMGSPGAPC | |
| | | | | | | QKRFFAFSVGRKTALHSGED | |
| | | | | | | FQTLLFERVFVNLDGCFDMA | |
| | | | | | | TGQFAAPLRGIYFFSLNVHS | |
| | | | | | | WNYKETYVHIMHNQKEAVIL | |
| | | | | | | YAQPSERSIMQSQSVMLDLA | |
| | | | | | | YGDRVWVRLFKRQRENAIYS | |
| | | | | | | NDFDTYITFSGHLIKAEDD | |
| tracer_ | chr3 | 50375003 | 50378045 | − | 318 | VRPLWRLHLGRRAQRPAVRA | down |
| 77921 | | | | | | LQVHLPLPLPRARLPGLLRA | |
| | | | | | | PGPGLGTRGGAGHERGERGA | |
| | | | | | | EGVWEGRGWAGHSAGILEGC | |
| | | | | | | LGAARKERSNCRFPGGTERL | |
| | | | | | | ILRGGWEGSLDCRLRKYSTR | |
| | | | | | | CKRTRAQADPGSAHAHFPHL | |
| | | | | | | PALASGQRPLLCACPDAGTR | |
| | | | | | | PRSLW | |
| tracer_ | chr1 | 1.62E+08 | 1.62E+08 | + | 319 | MGPREARGAALGGVVLRCDT | up |
| 7857 | | | | | | RLHPQKRDTPLQFAFYKYSR | |
| | | | | | | AVRRFDWGAEYTVPEPEVEE | |
| | | | | | | LESYWCEAATATRSVRKRSP | |
| | | | | | | WLQLPGPGSPLDPASTTAPA | |
| | | | | | | PWAAALAPGNRPLSFRKPPV | |
| | | | | | | SRSVPLVTSVRNTTSTGLQF | |
| | | | | | | PASGAPTAGPPACAPPTPLE | |
| | | | | | | QSAGALKPDVDLLLREMQLL | |
| | | | | | | KGLLSRVVLELKEPQALREL | |
| | | | | | | RGTPETPTSHFAVSPGTPET | |
| | | | | | | TPVES | |
| tracer_ | chr4 | 1.74E+08 | 1.74E+08 | − | 320 | LRKSSHQVDADLSTWVKETP | up |
| 86132 | | | | | | TSRGAKCPRTPSSCRPAGKS | |
| | | | | | | TRRNTRTLPSISRNSPRSVR | |
| | | | | | | RDGRPCLQRRSRSLKIWQKV | |
| | | | | | | TKLAMTGR | |
| tracer_ | chr5 | 54603939 | 54720600 | + | 321 | MEGASRVCRQGRVALPAEED | up |
| 87517 | | | | | | YLPLKPRVGKAAKEYPFILD | |
| | | | | | | AFQREAIQCVDNNQSVLVSA | |
| | | | | | | HTSAGKTVCAEYAIALALRE | |
| | | | | | | KQRVIFTSPIKALSNQKYRE | |
| | | | | | | MYEEFQDVGLMTGDVTINPT | |
| | | | | | | ASCLVMTTEILRSMLYRGSE | |
| | | | | | | VMREVAWVIFDEIHYMRDSE | |
| | | | | | | RGVVWEETIILLPDNVHYVF | |
| | | | | | | LSATIPNARQFAEWICHLHK | |
| | | | | | | QPCHVIYTDYRPTPLQHYIF | |
| | | | | | | PAGGDGLHLVVDENGDFRED | |
| | | | | | | NFNTAMQVLRDAGDLAKGDQ | |
| | | | | | | KGRKGGTKGPSNVFKIVKMI | |
| | | | | | | MERNFQPVIIFSFSKKDCEA | |
| | | | | | | YALQMTKLDFNTDEEKKMVE | |
| | | | | | | EVFSNAIDCLSDEDKKLPQV | |
| | | | | | | EHVLPLLKRGIGIHHGGLLP | |
| | | | | | | ILKETIEILFSEGLIKALFA | |
| | | | | | | TETFAMGINMPARTVLFTNA | |
| | | | | | | RKFDGKDFRWISSGEYIQMS | |
| | | | | | | GRAGRRGMDDRGIVILMVDE | |
| | | | | | | KMSPTIGKQLLKGSADPLNS | |
| | | | | | | AFHLTYNMVLNLLRVEEINP | |
| | | | | | | EYMLEKSFYQFQHYRAIPGV | |
| | | | | | | VEKVKNSEEQYNKIVIPNEE | |
| | | | | | | SVVIYYKIRQQLAKLGKEIE | |
| | | | | | | EYIHKPKYCLPFLQPGRLVK | |
| | | | | | | VKNEGDDFGWGVVVNFSKKS | |
| | | | | | | NVKPNSGELDPLYVVEVLLR | |
| | | | | | | CSKESLKNSATEAAKPAKPD | |
| | | | | | | EKGEMQVVPVLVHLLSAISS | |
| | | | | | | VRLYIPKDLRPVDNRQSVLK | |
| | | | | | | SIQEVQKRFPDGIPLLDPID | |
| | | | | | | DMGIQDQGLKKVIQKVEAFE | |
| | | | | | | HRMYSHPLHNDPNLETVYTL | |
| | | | | | | CEKKAQIAIDIKSAKRELKK | |
| | | | | | | ARTVLQMDELKCRKRVLRRL | |
| | | | | | | GFATSSDVIEMKGRVACEIS | |
| | | | | | | SADELLLTEMMFNGLFNDLS | |
| | | | | | | AEQATALLSCFVFQENSSEM | |
| | | | | | | PKLTEQLAGPLRQMQECAKR | |
| | | | | | | IAKVSAEAKLEIDEETYLSS | |
| | | | | | | FKPHLMDWYTWATGATFAHI | |
| | | | | | | CKMTDVFEGSIIRCMRRLEE | |
| | | | | | | LLRQMCQAAKAIGNTELENK | |
| | | | | | | FAEGITKIKRDIVFAASLYL | |
| tracer_ | chr7 | 40027519 | 40108945 | + | 322 | VKDVKKIKIEHAPSPSSGGT | up |
| 98198 | | | | | | LKNDKAKTKPPLQVTKVENN | |
| | | | | | | LIVDKATKKAVIVGKESKSA | |
| | | | | | | ATKEESVSLKEKTKPLTPSI | |
| | | | | | | GAKEKEQHVALVTSTLPPLP | |
| | | | | | | LPPMLPEDKEADSLRGNISV | |
| | | | | | | KAVKKEVEKKLRCLLADLPL | |
| | | | | | | PPELPGGDDLSKSPEEKKTA | |
| | | | | | | TQLHSKRRPKICGPRYGETK | |
| | | | | | | EKDIDWGKRCVDKFDIIGII | |
| | | | | | | GEGTYGQVYKARDKDTGEMV | |
| | | | | | | ALKKVRLDNEKEGFPITAIR | |
| | | | | | | EIKILRQLTHQSIINMKEIV | |
| | | | | | | TDKEDALDFKKDKGAFYLVF | |
| | | | | | | EYMDHDLMGLLESGLVHFNE | |
| | | | | | | NHIKSFMRQLMEGLDYCHKK | |
| | | | | | | NFLHRDIKCSNILLNNRGQI | |
| | | | | | | KLADFGLARLYSSEESRPYT | |
| | | | | | | NKVITLWYRPPELLLGEERY | |
| | | | | | | TPAIDVWSCGCILGELFTKK | |
| | | | | | | PIFQANQELAQLELIRHEEN | |
| | | | | | | EVSDKQI | |
| tracer_ | chr7 | 77787614 | 77807381 | − | 323 | MESGFGFRILGGDEPGQPIL | up |
| 99385 | | | | | | IGAVIAMGSADRDGRLHPGD | |
| | | | | | | ELVYVDGIPVAGKTHRYVID | |
| | | | | | | LMHHAARNGQVNLTVRRKVL | |
| | | | | | | CGGEPCPENGRSPGSVSTHH | |
| | | | | | | SSPRSDYATYTNSNHAAPSS | |
| | | | | | | NASPPEGFASHSLQTSDVVI | |
| | | | | | | HRKENEGFGFVIISSLNRPE | |
| | | | | | | SGSTIIFQSSFSSLQAVEQK | |
| | | | | | | RG | |
| tracer_ | chr7 | 87133558 | 87160896 | − | 324 | MKAIGSRLAVITQNIANLGT | down |
| 99541 | | | | | | GIIISFIYGWQLTLLLLAIV | |
| | | | | | | PIIAIAGVVEMKMLSGQALK | |
| | | | | | | DKKELEGSGKIATEAIENFR | |
| | | | | | | TVVSLTQEQKFEHMYAQSLQ | |
| | | | | | | VPYRNSLRKAHIFGITFSFT | |
| | | | | | | QAMMYFSYAGCFRFGAYLVA | |
| | | | | | | HKLMSFEDVLLVFSAVVFGA | |
| | | | | | | MAVGQVSSFAPDYAKAKISA | |
| | | | | | | AHIIMIIEKTPLIDSYSTEG | |
| | | | | | | LMPNTLEGNVTFGEVVFNYP | |
| | | | | | | TRPDIPVLQGLSLEVKKGQT | |
| | | | | | | LALVGSSGCGKSTVVQLLER | |
| | | | | | | FYDPLAGKVLLDGKEIKRLN | |
| | | | | | | VQWLRAHLGIVSQEPILFDC | |
| | | | | | | SIAENIAYGDNSRVVSQEEI | |
| | | | | | | VRAAKEANIHAFIESLPNKY | |
| | | | | | | STKVGDKGTQLSGGQKQRIA | |
| | | | | | | IARALVRQPHILLLDEATSA | |
| | | | | | | LDTESEKVVQEALDKAREGR | |
| | | | | | | TCIVIAHRLSTIQNADLIVV | |
| | | | | | | FQNGRVKEHGTHQQLLAQKG | |
| | | | | | | IYFSMVSVQAGTKRQ | |

Potential Structures of Identified nORFs

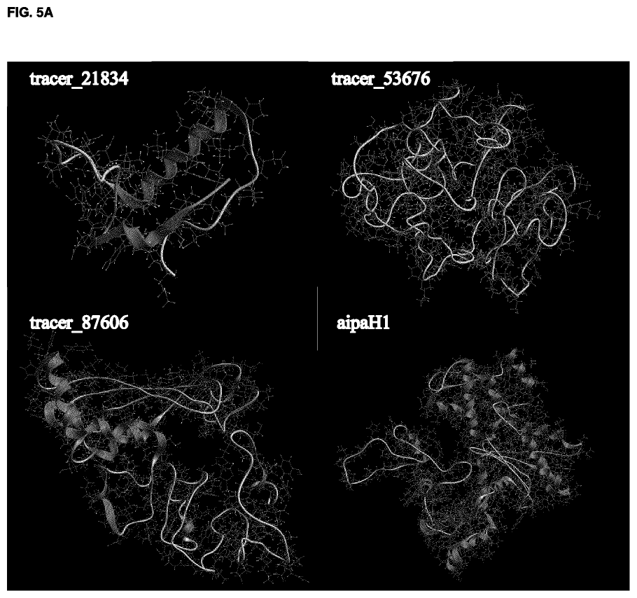

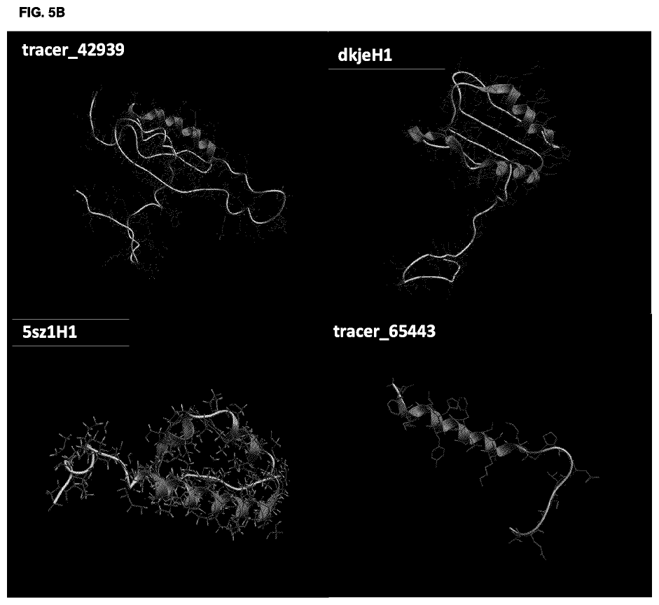

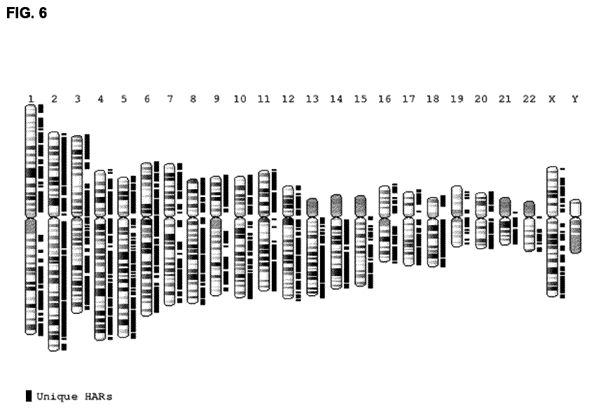

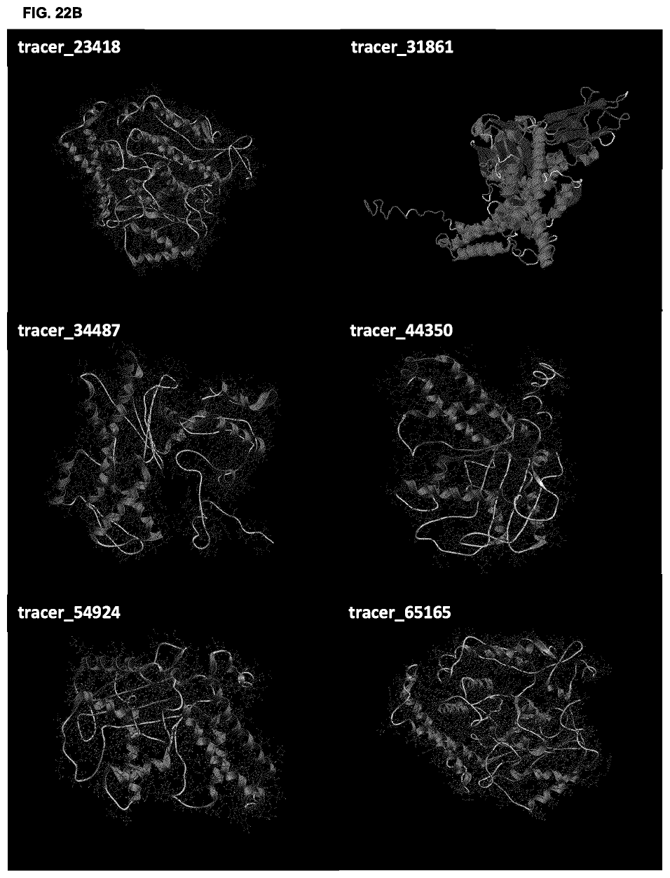

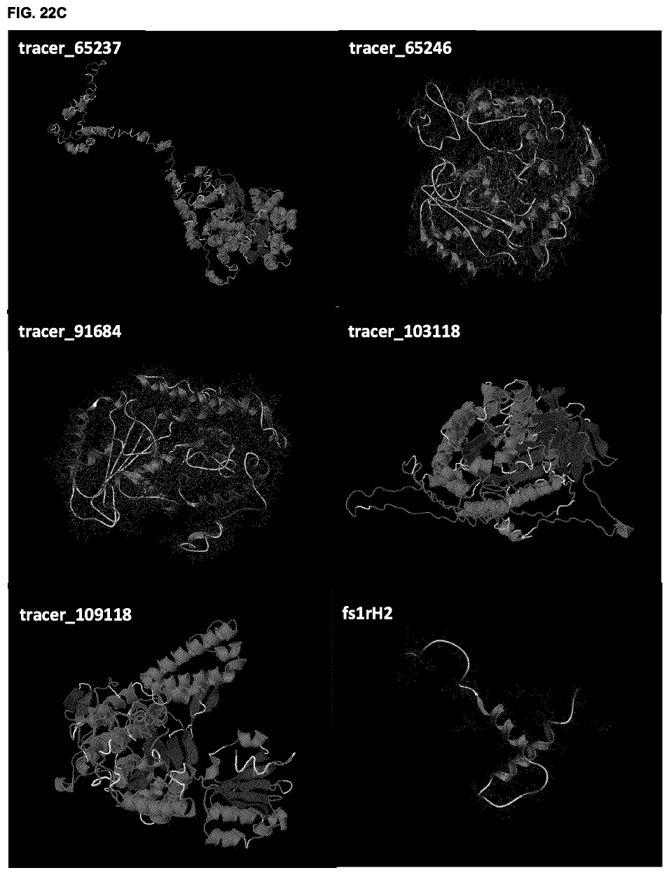

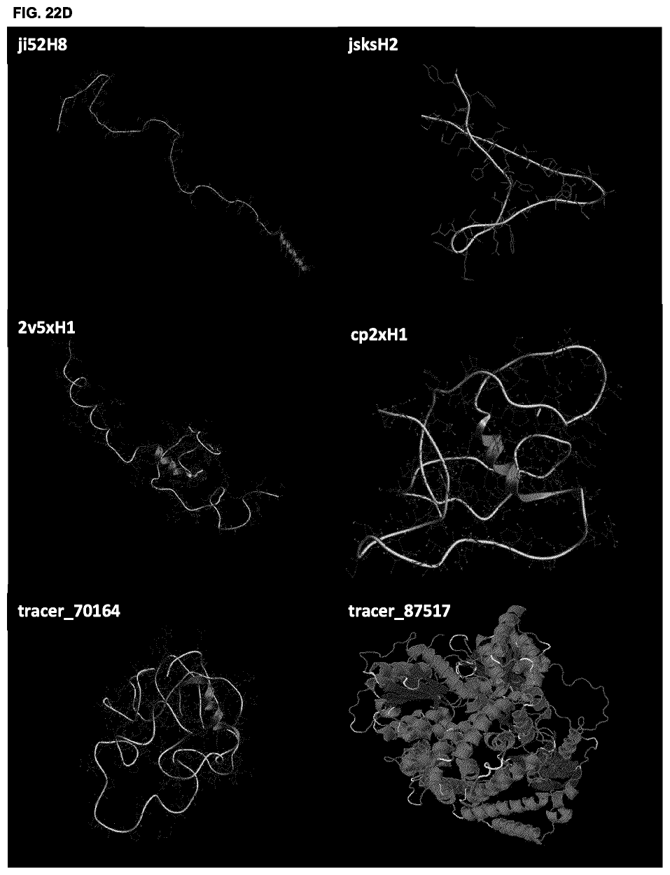

To infer whether these nORFs could form potential structures, we predicted the putative structures of the 21 nORFs identified as translated, as well as DE nORFs which included nORFs that were associated with pHARs and present in SCZ loci, using 1-TASSER and Raptor-X. For 1-TASSER, the model with the highest confidence score was chosen as the nORF structure. FIG. 5 shows representative structures of four nORFs out of the 21 nORFs for which we had evidence of translation, and representative nORF structures for those associated with pHAR and SCZ and BD loci. All other remaining structures are displayed in FIGS. 22A-22D. In addition to identifying potential functions from structure, this analysis examines the druggability as well as the interactome of these nORF end products.

### Discussion

The lack of adequate and targetable SCZ and BD-specific signatures in protein-coding and noncoding genes, led us to investigate nORFs within the human genome. We curated 248,135 nORFs and investigated 1,340 neuropsychiatric samples from the PsychENCODE consortium and identified 3,103 nORFs as transcribed, with 56 and 40 nORFs differentially expressed in SCZ and BD, respectively. Additionally, DHS1, TF and histone modification enrichments were found within the transcribed nORFs, and SCZ specific loci were found enriched with transcribed and DE nORFs.

A number of nORFs differentially expressed in SCZ and BD were identified as being associated with HARs and as having their expression correlated with that of associated TEs differentially expressed in SCZ and BD. The association of 13 DE nORFs with HARs, especially those that are also associated with SCZ and BD loci, suggests that HARs may play a role in the pathophysiology of SCZ and BD, and that these DE nORFs may have advantageous functions that they have been selected for either as a result of or in tandem with their associated HARs. This reinforces the idea that susceptible genes for the two disorders may have been positively selected for in human-specific evolution.

The type of HAR associated with each DE nORF gives a glimpse into the evolutionary background of their regulatory relationships and of, by extension, the disorders in question. The depletion of vHARs in DE nORF-HARs with respect to pHARs and mHARs in the SCZ datasets (FIG. 20) reinforces past conclusions that pHAR- and mHAR-associated genes (and therefore nORFs) are under greater selective constraint than vHAR-associated genes. The same cannot be said for DE nORF-HARs in the BD datasets.

The results of the enrichment analysis (FIG. 2C) reveal that for SCZ more HAR-associated nORFs show significant enrichment with the imputed regions until nominal P<10⁻⁷ except the results for vHAR-associated nORFs, and for BD, HAR-associated nORFs as a whole are less significantly enriched with disorder-linked loci, and none of the vHAR-associated nORFs show significant enrichment. These results show that nORFs-HARs not only play a role in SCZ but may also in BD. More importantly, SCZ loci are strongly enriched in nORFs near the pHARs. The nORFs fs1rH2 and tracer_70164, which are DE in SCZ, have functions to localize at post-synapses and the postsynaptic density. The DE nORF tracer_65443, which is differentially expressed in SCZ, is a retained intron within ZEB2. ZEB2 is a DNA-binding transcriptional corepressor that binds to E-boxes. It is involved in the transforming growth factor beta (TGFβ) signaling pathway and is largely found in tissues derived from the neural crest: many symptoms of ZEB2 deficiency can be explained by aberrant development of the neural crest-derived structures. It is highly conserved throughout evolution, and the ZEB2-associated DE nORF tracer_65443 is associated with many HARs, of all three conservation backgrounds. The DE nORF tracer_65443 and ZEB2 are also within a SCZ-associated locus, illustrating the importance of both the DE nORF and the gene in SCZ.

Similar patterns can be written for nORFs DE in BD. The DE nORF tracer_42939 is within the SLC7A60S gene, which is highly conserved in vertebrates. Despite the gene's conservation in vertebrates, it is associated with a pHAR, suggesting that some event may have occurred around the divergence of primates that resulted in human-lineage-specific rapid evolution of that locus, possibly resulting in altered CNS development and a susceptibility to BD. As mentioned previously, SLC7A60S is within a SCZ-associated locus; its detection as a DE nORF-HAR in BD and its relevance to SCZ suggests a genetic commonality and may contribute towards explaining phenotypic similarities between the disorders.

The correlation of expression of DE nORFs and DE TEs indicates the possibility of TE-based regulation of the DE nORFs, especially since the majority of DE-TEs found in this analysis are distinct of the DE nORF (the exception is tracer_18675 and its L1MC2 TE). A particularly interesting DE nORF-DE-TE combination is that of eveeH1 and its ERV1 LTR TE. The DE nORF eveeH1 is within the ZNF84 gene, which codes for a KRAB/FPB domain-containing protein. The KRAB/FPB domain may regulate gene expression through TE regulation; eveeH1 may thus be an initial regulation point from which a cascade of TE-based regulation occurs. Since it is differentially regulated in BD, it could be responsible, at least in part, for TE-based differential regulation across the genome that contributes to the BD phenotype. Further investigation into the specific function of eveeH1 and other DE nORFs may elucidate more fully their role in SCZ and BD. The exact relationships between HARs, TEs and nORFs remain to be elucidated; further work utilizing ChIP-seq and whole genome bisulfite sequencing data could shine a light on them. Furthermore, analysis of more RNAseq data—from a large number of disorder samples in particular—would help clarify how HARs and TEs regulate nORF expression in these two mental disorders.

We also demonstrated evidence of translation for 21 nORFs from the database, and for three new ones identified from the transcriptome of a smaller subset of neuropsychiatric samples. Of the 21 nORFs, some were found significantly different between disorders for metadata categories such as gender, incidence of psychosis and suicide. We predicted structures for the 21 nORFs and for those that are associated with pHARs and disorder-loci. This approach could offer a new strategy to expedite the identification of novel drug candidates and novel diagnostic signatures for preemptive interventions, for example to prevent suicide or mitigate psychosis.

To summarize, we introduce how novel regions of the genome, nORFs, merit systematic analysis within disease systems to uncover novel targets for the development of diagnostic and therapeutic strategies. From an evolutionary point-of-view, these results indicate that the genomic features responsible for SCZ and BD arose at least after the divergence of mammals from other vertebrates, or that nORFs associated with pHARs may have arisen in primates and then been subject to increased evolution in the human lineage, only to result in SCZ and BD susceptibility in modern humans when dysfunctional.

### Code Availability

Codes for this work can be obtained from: github.com/PrabakaranGroup/norfs_in_neuropsychiatric disorders.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of treating schizophrenia or bipolar disorder in a subject comprising:
(a) identifying a sequence of a novel open reading frame (nORF) associated with the schizophrenia or bipolar disorder, wherein the sequence of the nORF is distinct from a canonical open reading frame (cORF) of a gene, wherein the nORF is present in (i) an overlapping region of the cORF in an alternate reading frame, (ii) a 5′ untranslated region (UTR) of the cORF, (iii) a 3′ UTR of the cORF, (iv) an intronic region of the cORF, (v) an intergenic region of the cORF, or (vi) a region not associated with the cORF or the gene, and wherein the nORF has increased expression relative to the nORF in a subject without schizophrenia or bipolar disorder; and
(b) administering to the subject an inhibitor that reduces expression of the nORF to treat the schizophrenia or bipolar disorder.

2. A method of treating schizophrenia or bipolar disorder in a subject comprising administering to the subject an inhibitor that reduces expression of a nORF; wherein the subject has previously been identified with a sequence of the nORF associated with the schizophrenia or bipolar disorder, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the nORF has increased expression relative to the nORF in a subject without schizophrenia or bipolar disorder.

3. The method of any one of claim 1 or 2, wherein the inhibitor comprises a small molecule, a polynucleotide, or a polypeptide.

4. The method of claim 3, wherein the polynucleotide comprises a miRNA, an antisense RNA, an shRNA, or an siRNA.

5. The method of claim 3, wherein the polypeptide comprises an antibody or antigen-binding fragment thereof.

6. The method of claim 5, wherein the antigen-binding fragment thereof is an scFv.

7. The method of any one of claims 3 to 6, wherein the inhibitor is encoded by a vector.

8. The method of claim 7, wherein the vector is a viral vector.

9. The method of claim 8, wherein viral vector is selected from the group consisting of a Retroviridae family virus, an adenovirus, a parvovirus, a coronavirus, a rhabdovirus, a paramyxovirus, a picornavirus, an alphavirus, a herpes virus, and a poxvirus.

10. The method of claim 9, wherein the parvovirus viral vector is an adeno-associated virus (AAV) vector.

11. The method of claim 10, wherein the viral vector is a Retroviridae family viral vector.

12. The method of claim 11, wherein the Retroviridae family viral vector is a lentiviral vector.

13. The method of claim 11, wherein the Retroviridae family viral vector is an alpharetroviral vector or a gammaretroviral vector.

14. The method of any one of claims 10 to 13, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

15. The method of any one of claims 10 to 14, wherein the viral vector is a pseudotyped viral vector.

16. The method of claim 15, wherein the pseudotyped viral vector is selected from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

17. The method of claim 16, wherein the pseudotyped viral vector is a lentiviral vector.

18. The method of any one of claims 15 to 17, wherein the pseudotyped viral vector comprises one or more envelope proteins from a virus selected from vesicular stomatitis virus (VSV), RD114 virus, murine leukemia virus (MLV), feline leukemia virus (FeLV), Venezuelan equine encephalitis virus (VEE), human foamy virus (HFV), walleye dermal sarcoma virus (WDSV), Semliki Forest virus (SFV), Rabies virus, avian leukosis virus (ALV), bovine immunodeficiency virus (BIV), bovine leukemia virus (BLV), Epstein-Barr virus (EBV), Caprine arthritis encephalitis virus (CAEV), Sin Nombre virus (SNV), Cherry Twisted Leaf virus (ChTLV), Simian T-cell leukemia virus (STLV), Mason-Pfizer monkey virus (MPMV), squirrel monkey retrovirus (SMRV), Rous-associated virus (RAV), Fujinami sarcoma virus (FuSV), avian carcinoma virus (MH2), avian encephalomyelitis virus (AEV), Alfa mosaic virus (AMV), avian sarcoma virus CT10, and equine infectious anemia virus (EIAV).

19. The method of claim 18, wherein the pseudotyped viral vector comprises a VSV-G envelope protein.

20. A method of treating schizophrenia or bipolar disorder in a subject comprising:
(a) identifying a sequence of a nORF associated with the schizophrenia or bipolar disorder, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the nORF has decreased expression relative to the nORF in a subject without schizophrenia or bipolar disorder; and
(b) administering to the subject an activator that increases expression of nORF to treat the schizophrenia or bipolar disorder.

21. A method of treating schizophrenia or bipolar disorder in a subject comprising administering to the subject an activator that increases expression of a nORF; wherein the subject has previously been identified with a sequence of the nORF associated with the schizophrenia or bipolar disorder, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the nORF has decreased expression relative to the nORF in a subject without schizophrenia or bipolar disorder.

22. The method of claim 20 or 21, wherein the activator comprises a small molecule, a polynucleotide, or a polypeptide.

23. The method of claim 22, wherein the polynucleotide comprises an antisense RNA.

24. The method of claim 22, wherein the polypeptide comprises an antibody or antigen-binding fragment thereof.

25. The method of claim 24, wherein the antigen-binding fragment thereof is an scFv.

26. The method of any one of claims 20 to 25, wherein the activator is encoded by a vector.

27. The method of claim 26, wherein the vector is a viral vector.

28. A method of treating schizophrenia or bipolar disorder in a subject comprising:
(a) identifying a sequence of a nORF associated with the schizophrenia or bipolar disorder, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the nORF has decreased expression relative to the nORF in a subject without schizophrenia or bipolar disorder; and
(b) providing a protein encoded by the nORF to the subject treat the schizophrenia or bipolar disorder.

29. A method of treating schizophrenia or bipolar disorder in a subject comprising providing a protein encoded by a nORF to the subject; wherein the subject has previously been identified with a sequence of the nORF associated with schizophrenia or bipolar disorder, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, and wherein the nORF has decreased expression relative to the nORF in a subject without schizophrenia or bipolar disorder.

30. The method of claim 28 or 29, wherein the method comprises restoring the encoded protein product of the nORF.

31. The method of claim 30, wherein the therapy comprises providing the protein product or a polynucleotide encoding the protein product.

32. The method of claim 31, wherein the method comprises providing a vector comprising the polynucleotide encoding the protein product.

33. The method of claim 32, wherein the vector is a viral vector.

34. The method of claim 33, wherein viral vector is selected from the group consisting of a Retroviridae family virus, an adenovirus, a parvovirus, a coronavirus, a rhabdovirus, a paramyxovirus, a picornavirus, an alphavirus, a herpes virus, and a poxvirus.

35. The method of claim 34, wherein the parvovirus viral vector is an AAV vector.

36. The method of claim 35, wherein the viral vector is a Retroviridae family viral vector.

37. The method of claim 36, wherein the Retroviridae family viral vector is a lentiviral vector.

38. The method of claim 36, wherein the Retroviridae family viral vector is an alpharetroviral vector or a gammaretroviral vector.

39. The method of any one of claims 34 to 37, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

40. The method of any one of claims 33 to 39, wherein the viral vector is a pseudotyped viral vector.

41. The method of claim 40, wherein the pseudotyped viral vector is selected from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

42. The method of claim 41, wherein the pseudotyped viral vector is a lentiviral vector.

43. The method of any one of claims 39 to 42, wherein the pseudotyped viral vector comprises one or more envelope proteins from a virus selected from vesicular stomatitis virus VSV, RD114 virus, MLV, FeLV, VEE, HFV, WDSV, SFV, Rabies virus, ALV, BIV, BLV, EBV, CAEV, SNV, ChTLV, STLV, MPMV, SMRV, RAV, FuSV, MH2, AEV, AMV, avian sarcoma virus CT10, and EIAV.

44. The method of claim 43, wherein the pseudotyped viral vector comprises a VSV-G envelope protein.

45. The method of any one of claims 1 to 44, wherein the encoded protein product of the nORF is less than about 100 amino acids.

46. The method of any one of claims 1 to 45, further comprising performing a statistical analysis between the nORF and the schizophrenia or bipolar disorder.

47. The method of claim 46, wherein the statistical analysis measures a positive or negative association between the nORF and the schizophrenia or bipolar disorder.

48. The method of any one of claims 1 to 47, wherein the nORF has a positive or negative association with a transposable element.

49. The method of any one of claims 1 to 48, wherein the nORF has a positive or negative association with a human accelerated region.

50. The method of any one of claims 1 to 49, wherein the nORF is selected from Table 4.

51. The method of claim 50, wherein the nORF has at least 85%, 90%, 95%, 97%, 99%, or 100% sequence identify to any one of SEQ ID NOs: 1-21.

52. The method of claim 51, wherein the nORF has the sequence of any one of SEQ ID NOs: 1-21.

53. The method of any one of claims 1 to 49, wherein the disease is bipolar disorder and the nORF is selected from Table 7.

54. The method of claim 53, wherein the nORF has at least 85%, 90%, 95%, 97%, 99%, or 100% sequence identify to any one of SEQ ID NOs: 124-163.

55. The method of claim 54, wherein the nORF has the sequence of any one of SEQ ID NOs: 124-163.

56. The method of any one of claims 1 to 49, wherein the disease is bipolar disorder and the nORF is selected from Table 8.

57. The method of claim 56, wherein the nORF has at least 85%, 90%, 95%, 97%, 99%, or 100% sequence identify to any one of SEQ ID NOs: 164-207.

58. The method of claim 57, wherein the nORF has the sequence of any one of SEQ ID NOs: 164-207.

59. The method of any one of claims 1 to 49, wherein the disease is schizophrenia and the nORF is selected from Table 9.

60. The method of claim 59, wherein the nORF has at least 85%, 90%, 95%, 97%, 99%, or 100% sequence identify to any one of SEQ ID NOs: 208-263.

61. The method of claim 60, wherein the nORF has the sequence of any one of SEQ ID NOs: 208-263.

62. The method of any one of claims 1 to 49, wherein the disease is schizophrenia and the nORF is selected from Table 10.

63. The method of claim 62, wherein the nORF has at least 85%, 90%, 95%, 97%, 99%, or 100% sequence identify to any one of SEQ ID NOs: 264-324.

64. The method of claim 63, wherein the nORF has the sequence of any one of SEQ ID NOs: 264-324.
